(19) United States
(12) Patent Application Publication
(10) Pub. No.: US 2011/0098324 A1
(43) Pub. Date: Apr. 28, 2011

(54) PROLYL HYDROXYLASE INHIBITORS

(76) Inventors: James A. Brackley, III, Collegeville, PA (US); Antony Nicholas Shaw, Collegeville, PA (US); Rosanna Tedesco, Collegeville, PA (US); Yonghui Wang, Shanghai (CN); Kenneth J. Wiggall, Collegeville, PA (US); Hongyi Yu, Collegeville, PA (US)

(21) Appl. No.: 12/997,128

(22) PCT Filed: Jun. 23, 2009
(86) PCT No.: PCT/US09/48193

§ 371 Date: Dec. 9, 2010

Related U.S. Application Data
(60) Provisional application No. 61/075,458, filed on Jun. 25, 2008.

(51) Int. Cl. A61K 31/44; C07D 211/72; A61P 7/06

(52) U.S. Cl. 514/350; 546/299

(57) ABSTRACT

The invention described herein relates to certain pyrimidinedione N-substituted glycine derivatives of formula (I)

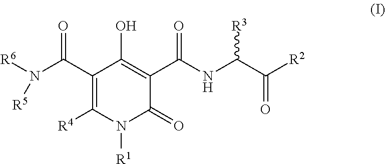

which are antagonists of HIF prolyl hydroxylases and are useful for treating diseases benefiting from the inhibition of this enzyme, anemia being one example.

### FIELD OF THE INVENTION

This invention relates to certain heteroaromatic N-substituted glycine derivatives that are inhibitors of HIF prolyl hydroxylases, and thus have use in treating diseases benefiting from the inhibition of this enzyme, anemia being one example.

### BACKGROUND OF THE INVENTION

Anemia occurs when there is a decrease or abnormality in red blood cells, which leads to reduced oxygen levels in the blood. Anemia occurs often in cancer patients, particularly those receiving chemotherapy. Anemia is often seen in the elderly population, patients with renal disease, and in a wide variety of conditions associated with chronic disease.

Frequently, the cause of anemia is reduced erythropoietin (Epo) production resulting in prevention of erythropoiesis (maturation of red blood cells). Epo production can be increased by inhibition of prolyl hydroxylases that regulate hypoxia inducible factor (HIF).

One strategy to increase erythropoietin (Epo) production is to stabilize and thus increase the transcriptional activity of the HIF. HIF-alpha subunits (HIF-1 alpha, HIF-2alpha, and HIF-3alpha) are rapidly degraded by proteosome under normoxic conditions upon hydroxylation of proline residues by prolyl hydroxylases (EGLN1, 2, 3). Proline hydroxylation allows interaction with the von Hippel Lindau (VHL) protein, a component of an E3 ubiquitin ligase. This leads to ubiquitination of HIF-alpha and subsequent degradation. Under hypoxic conditions, the inhibitory activity of the prolyl hydroxylases is suppressed, HIF-alpha subunits are therefore stabilized, and HIF-responsive genes, including Epo, are transcribed. Thus, inhibition of prolyl hydroxylases results in increased levels of HIF-alpha and thus increased Epo production.

The compounds of this invention provide a means for inhibiting these hydroxylases, increasing Epo production, and thereby treating anemia. Ischemia, stroke, and cytoprotection may also benefit by administering these compounds.

### SUMMARY OF THE INVENTION

In the first instance, this invention relates to a compound of formula (I):

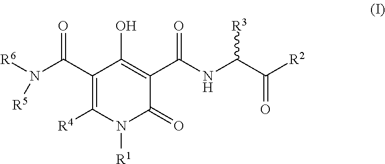

wherein:

R¹ is H, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R² is —NR⁷R⁸ or —OR⁹;

R³ is H or C₁₋C₄alkyl;

R⁴ is hydrogen or OH;

R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl; or R⁵ and R⁶ taken together with the nitrogen to which they are attached form a 5- or 6- or 7-membered saturated ring optionally containing one other heteroatom selected from oxygen, nitrogen and sulphur;

R⁷ and R⁸ are each independently selected from the group consisting of hydrogen, C₁₋C₁₀ alkyl, C₂₋C₁₀ alkenyl, C₂₋C₁₀ alkynyl, C₃-C₈ cycloalkyl, C₃-C₈ heterocycloalkyl, aryl and heteroaryl;

R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;

where any carbon or heteroatom of R¹, R³, R⁵, R⁶, R⁷, R⁸, R⁹ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —NR⁷R⁸, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R¹² is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;

or a pharmaceutically acceptable salt or solvate thereof.

In a second aspect of the present invention, there is provided a compound of formula (I) or a salt or solvate thereof for use in mammalian therapy, e.g. treating amenia. An example of this therapeutic approach is that of a method for treating anemia caused by increasing the production of erythropoietin (Epo) by inhibiting HIF prolyl hydroxylases comprising administering a compound of formula (I) to a patient in need thereof, neat or admixed with a pharmaceutically acceptable excipient, in an amount sufficient to increase production of Epo.

In a third aspect of the present invention, there is provided a pharmaceutical composition comprising a compound of formula (I) or a salt, solvate, or the like thereof, and one or more of pharmaceutically acceptable carriers, diluents and excipients.

In a fourth aspect, there is provided the use of a compound of formula (I) or a salt or solvate thereof in the preparation of a medicament for use in the treatment of a disorder mediated by inhibiting HIF prolyl hydroxylases, such as an anemia, that can be treated by inhibiting HIF prolyl hydroxylases.

### DETAILED DESCRIPTION OF THE INVENTION

For the avoidance of doubt, unless otherwise indicated, the term “substituted” means substituted by one or more defined groups. In the case where groups may be selected from a number of alternative groups the selected groups may be the same or different.

The term “independently” means that where more than one substituent is selected from a number of possible substituents, those substituents may be the same or different.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms, so for example, as used herein, the terms “C₁₋C₄ alkyl” and “C₁₋C₁₀ alkyl” refers to an alkyl group having at least 1 and up to 4 or 10 carbon atoms respectively. Examples of such branched or straight-chained alkyl groups useful in the present invention include, but are not limited to, methyl, ethyl, n-propyl, isopropyl, isobutyl, n-butyl, t-butyl, n-pentyl, isopentyl, n-hexyl, n-heptyl, n-octyl, n-nonyl, and n-decyl, and branched analogs of the latter 5 normal alkanes.

When the term “alkenyl” (or “alkenylene”) is used it refers to straight or branched hydrocarbon chains containing the specified number of carbon atoms and at least 1 and up to 5 carbon-carbon double bonds. Examples include ethenyl (or ethenylene) and propenyl (or propenylene).

When the term “alkynyl” (or “alkynylene”) is used it refers to straight or branched hydrocarbon chains containing the specified number of carbon atoms and at least 1 and up to 5 carbon-carbon triple bonds. Examples include ethynyl (or ethynylene) and propynyl (or propynylene).

When “cycloalkyl” is used it refers to a non-aromatic, saturated, cyclic hydrocarbon ring containing the specified number of carbon atoms. So, for example, the term “C₃₋C₈ cycloalkyl” refers to a non-aromatic cyclic hydrocarbon ring having from three to eight carbon atoms. Exemplary “C₃-C₈ cycloalkyl” groups useful in the present invention include, but are not limited to, cyclopropyl, cyclobutyl, cyclopentyl, cyclohexyl, cycloheptyl and cyclooctyl.

The term “C₅-C₈cycloalkenyl” refers to a non-aromatic monocyclic carboxycyclic ring having the specified number of carbon atoms and up to 3 carbon-carbon double bonds. “Cycloalkenyl” includes by way of example cyclopentenyl and cyclohexenyl.

Where “C₃-C₈ heterocycloalkyl” is used, it means a non-aromatic heterocyclic ring containing the specified number of ring atoms being, saturated or having one or more degrees of unsaturation and containing one or more heteroatom substitutions selected from O, S and/or N. Such a ring may be optionally fused to one or more other “heterocyclic” ring(s) or cycloalkyl ring(s). Examples of “heterocyclic” moieties include, but are not limited to, aziridine, thiirane, oxirane, azetidine, oxetane, thietane, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

“Aryl” refers to optionally substituted monocyclic and polycarbocyclic unfused or fused groups having 6 to 14 carbon atoms and having at least one aromatic ring that complies with Hiickel's Rule. Examples of aryl groups are phenyl, biphenyl, naphthyl, anthracenyl, phenanthrenyl and the like.

“Heteroaryl” means an optionally substituted aromatic monocyclic ring or polycarbocyclic fused ring system wherein at least one ring complies with Hiickel's Rule, has the specified number of ring atoms, and that ring contains at least one heteratom selected from N, O, and/or S. Examples of “heteroaryl” groups include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl.

The term “optionally” means that the subsequently described event(s) may or may not occur, and includes both event(s), which occur, and events that do not occur.

The term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group, one acidic enough to form salts. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compounds of particular interest include those wherein:

R¹ is hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R² is −OR⁹;

R³ is H or C₁₋C₄alkyl;

R⁴ is hydrogen or OH;

R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl;

R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R³, R⁵, R⁶, R⁷, R⁸, R⁹ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —NR⁷R⁸, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁷, and R⁸ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl.

Of further interest are those compounds where:

R¹ is selected from the group consisting of hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;

R⁴ is H or OH;

R² is −OR⁹;

R³ is H;

R⁹ is H or a cation;

R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl;
where any carbon or heteroatom of R¹, R⁵ and R⁶ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —NR⁷R⁸, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁷, and R⁸ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, hetero aryl, and C₁₋C₁₀alkyl-heteroaryl;

or a pharmaceutically acceptable salt thereof.

Processes for preparing the compound of formula (I) are also within the ambit of this invention. To illustrate, a process for preparing a compound of formula (I)

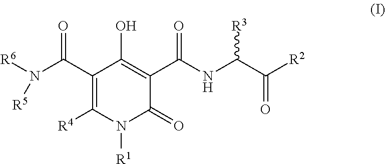

wherein R¹, R², R³, R⁴, R⁵ and R⁶ are the same as defined above for formula (I), the process comprising:

treating a compound of formula A:

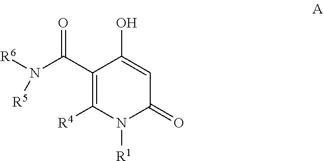

wherein R¹, R⁴, R⁵ and R⁶ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (I) where R² is —OEt;

or a process for preparing a compound of formula (I) wherein R¹, R², R³, R⁴, R⁵ and R⁶ are the same as defined above for formula (I), the process comprising treating a compound of formula B:

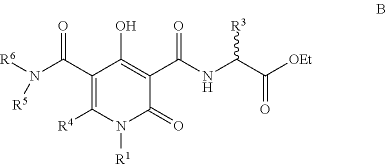

wherein R¹, R³, R⁴, R⁵ and R⁶ are the same as for those groups in formula (I) with an alkali such as sodium hydroxide, in an appropriate solvent, such as aqueous ethanol, at a suitable temperature such as room temperature, to form a compound of formula (I) where R² is —OH;

The compounds of formula (I) may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Certain of the compounds described herein may contain one or more chiral atoms, or may otherwise be capable of existing as two enantiomers. The compounds claimed below include mixtures of enantiomers as well as purified enantiomers or enantiomerically enriched mixtures. Also included within the scope of the invention are the individual isomers of the compounds represented by formula (I), or claimed below, as well as any wholly or partially equilibrated mixtures thereof. The present invention also covers the individual isomers of the claimed compounds as mixtures with isomers thereof in which one or more chiral centers are inverted. Also, it is understood that any tautomers and mixtures of tautomers of the claimed compounds are included within the scope of the compounds of formula (I) as disclosed herein above or claimed herein below.

Where there are different isomeric forms they may be separated or resolved one from the other by conventional methods, or any given isomer may be obtained by conventional synthetic methods or by stereospecific or asymmetric syntheses.

While it is possible that, for use in therapy, a compound of formula (I), as well as salts, solvates and the like, may be administered as a neat preparation, i.e. no additional carrier, the more usual practice is to present the active ingredient confected with a carrier or diluent. Accordingly, the invention further provides pharmaceutical compositions, which includes a compound of formula (I) and salts, solvates and the like, and one or more pharmaceutically acceptable carriers, diluents, or excipients. The compounds of formula (I) and salts, solvates, etc, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula (I), or salts, solvates etc, with one or more pharmaceutically acceptable carriers, diluents or excipients.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Pharmaceutical compositions may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula (I), depending on the condition being treated, the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of a compound of the present invention will depend upon a number of factors including, for example, the age and weight of the intended recipient, the precise condition requiring treatment and its severity, the nature of the formulation, and the route of administration, and will ultimately be at the discretion of the attendant prescribing the medication. However, an effective amount of a compound of formula (I) for the treatment of anemia will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, etc., may be determined as a proportion of the effective amount of the compound of formula (I) per se. It is envisaged that similar dosages would be appropriate for treatment of the other conditions referred to above.

### DEFINITIONS

rt—room temperature
DMF—dimethylformamide
THF—tetrahydrofuran
DBU—1,8-diazabicyclo[5.4.0]undec-7-ene
TFA—Trifluoroacetic acid
PyBOP—Benzotriazole-1-yl-oxy-tris-pyrrolidino-phosphonium hexafluorophosphate

### Chemical Background:

The compounds of this invention may be made by a variety of methods, including standard chemistry. Any previously defined variable will continue to have the previously defined meaning unless otherwise indicated. Illustrative general synthetic methods are set out below and then specific compounds of the invention as prepared are given in the examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I). Those skilled in the art will recognize if a stereocenter exists in compounds of formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

### Illustrated Methods of Preparation

Treatment of 7-chloro-2,2-dimethyl-4H,5H-pyrano[4,3-d][1,3]dioxin-4,5-dione, obtained by thermal condensation of acetone and malonyl chloride, with an appropriately substituted primary amine followed by ring opening/closing and methanolysis afforded the N(1)-substituted methyl 2,4-dihydroxy-6-oxo-1,6-dihydro-3-pyridinecarboxylate which was treated with ethyl 2-isocyanatocarboxylate, under either conventional thermal conditions or by microwave irradiation. The methyl ester was converted to an amide by treatment with an appropriately substituted amine and the ethyl ester was hydrolyzed under basic conditions to provide, after acidification, the compounds of the invention.

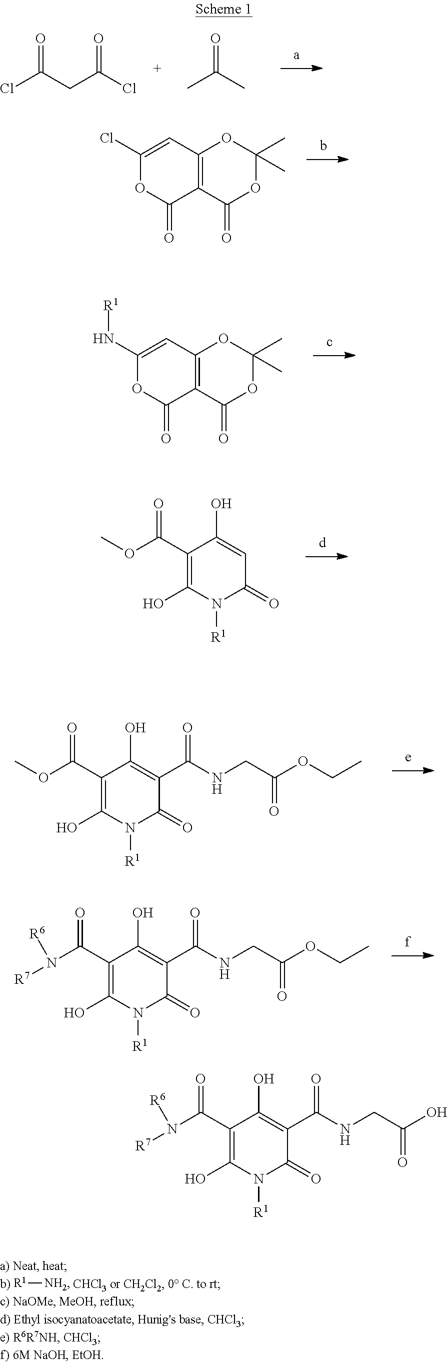

Dimethyl-1,3-acetonedicarboxylate, trimethylorthoformate and acetic anhydride were treated under reflux and then reacted with an appropriately substituted amine in the presence of sodium hydride to give the N(1)-substituted methyl 4-hydroxy-6-oxo-1,6-dihydro-3-pyridinecarboxylate. Conversion of the methyl ester to an mide by reaction with an appropriately substituted amine, followed by treatment with ethyl 2-isocyanatocarboxylate, under either conventional thermal conditions or by microwave irradiation and hydrolysis, provided, after acidification, the compounds of the invention.

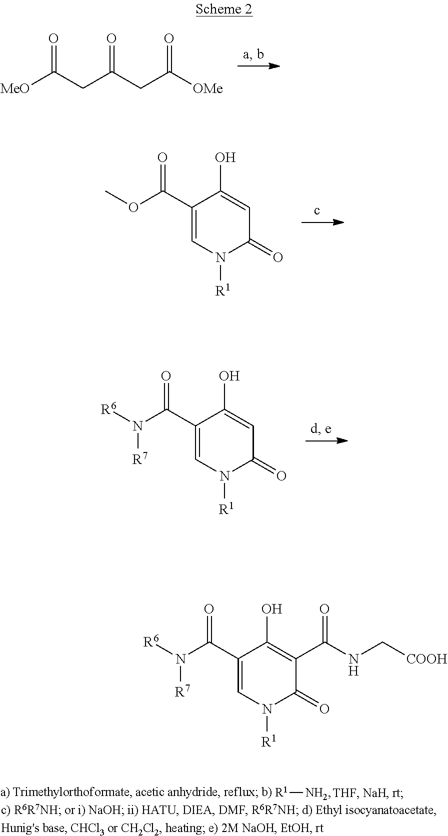

### EXPERIMENTALS

### Example 1

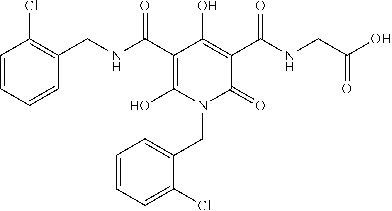

### N-{[1-[(2-chlorophenyl)methyl]-5-({[(2-chlorophenyl)methyl]amino}carbonyl)-2,4-dihydroxy-6-oxo-1,6-dihydro-3-pyridinyl]carbonyl}glycine

1a) 7-Chloro-2,2-dimethyl-4H,5H-pyrano[4,3-d][1,3]dioxin-4,5-dione. Malonyl dichloride (60.0 g, 426 mmol) in acetone (120 mL) was heated to gentle reflux for 15 minutes. The mixture was cooled over an ice bath and diluted with hexane (150 mL). The solid was collected, washed with hexane and dried in vacuo to give the title dione (21.0 g, 42%). ¹H NMR (400 MHz, CHLOROFORM-d) δ ppm 6.18 (s, 1H), 1.81 (s, 6H).

1b) 7-{[(2-Chlorophenyl)methyl]-amino}-2,2-dimethyl-4H,5H-pyrano[4,3-d][1,3]dioxin-4,5-dione. 2-Chlorobenzylamine (2.96 mL, 24.5 mmoles) in dichloromethane (20 mL) was added dropwise to a cooled solution of 7-chloro-2,2-dimethyl-4H,5H-pyrano[4,3-d][1,3]dioxin-4,5-dione (2.83 g, 12.3 mmoles) in dichloromethane (80 mL). Cooling was removed and the mixture was stirred for 30 minutes. The solid was collected, washed with dichloromethane and dried to give the title dione (2.85 g, 69%). ¹H NMR (400 MHz, DMSO-d₆) δ ppm 9.34-9.99 (m, 1H), 7.59-7.76 (m, 1H), 7.52-7.56 (m, 1H), 7.40-7.45 (m, 2H), 5.35 (s, 1H), 4.52 (d, J=29.56 Hz, 1H), 1.64 (s, 6H).

1c) Methyl 1-[(2-chlorophenyl)methyl]-4,6-dihydroxy-2-oxo-1,2-dihydro-3-pyridinecarboxylate. A mixture of 7-{[(2-chlorophenyl)methyl]amino}-2,2-dimethyl-4H,5H-pyrano[4,3-d][1,3]dioxin-4,5-dione (2.85 g, 8.49 mmoles) and 25% sodium methoxide in methanol (2.5 g, 46.3 mmoles) in methanol (50 mL) was heated under reflux for 2 hours. The mixture was acidified with 1N hydrochloric acid and extracted into ethyl acetate (×3). The combined extracts were washed with 1N hydrochloric acid, dried and evaporated to give a solid comprising the titled methyl ester (2.0 g, 76%). ¹H NMR (400 MHz, DMSO-d6) δ ppm 7.36-7.57 (m, 1H), 7.15-7.33 (m, 2H), 6.57-6.87 (m, 1H), 5.43 (dd, J=17.18, 2.27 Hz, 1H), 5.09 (d, J=23.75 Hz, 2H), 3.76 (d, J=7.58 Hz, 3H).

1d) Methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(2-chlorophenyl)methyl]-4,6-dihydroxy-2-oxo-1,2-dihydro-3-pyridinecarboxylate (2.0 g, 6.46 mmoles), N,N-diisopropylethylamine (2.26 mL, 12.92 mmoles) and ethyl isocyanatoacetate (1.34 mL, 7.75 mmoles) was sealed in a pressure flask and heated in a microwave reactor at 120° C. for 45 minutes. The cooled mixture was washed with 1 molar hydrochloric acid, the solvent evaporated and the residue purified by flash chromatography (1% acetic acid in dichloromethane) to give an oil that crystallized from ether. The solid was collected, washed with hexane and dried to give the titled methyl ester (800 mg, 28%). ¹H NMR (400 MHz, DMSO-d6) δ ppm 10.12 (t, J=5.05 Hz, 1H), 7.36-7.49 (m, 1H), 7.24 (dd, J=5.94, 3.41 Hz, 2H), 6.80 (dd, J=5.56, 3.79 Hz, 1H), 5.09 (s, 2H), 4.11 (q, J=7.07 Hz, 2H), 4.03 (d, J=5.31 Hz, 2H), 3.66 (s, 3H), 1.19 (t, J=7.07 Hz, 3H).

1e) N-{[1-[(2-chlorophenyl)methyl]-5-({[(2-chlorophenyl)methyl]amino}carbonyl)-2,4-dihydroxy-6-oxo-1,6-dihydro-3-pyridinyl]carbonyl}glycine. A mixture of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (350 mg, 0.798 mmoles) and 2-chlorobenzylamine (226 mg, 1.6 mmoles) in 1,4-dioxane (10 mL) was sealed in a pressure tube and heated in a microwave reactor at 150° C. for 30 minutes. The mixture was diluted with ethyl acetate and washed with 1 molar hydrochloric acid. The aqueous was extracted with ethyl acetate and the combined extracts washed with 1 molar hydrochloric acid. The organic solution was dried, evaporated and purified by flash chromatography (dichloromethane). The required fractions were evaporated and suspended in ethanol (10 mL), treated with 1 molar sodium hydroxide solution (10 ml) and 6 molar sodium hydroxide solution (5 ml) and stirred overnight. The mixture was acidified and extracted into ethyl acetate. The organic solution was washed with 1 molar hydrochloric acid, dried and the solvent evaporated. The residue formed a solid that was triturated with acetic acid and diluted with hexane, collected, washed with hexane and dried to give the title glycine (270 mg, 65%). ¹H NMR (400 MHz, DMSO-d6) δ ppm 12.79 (br. s., 1H), 9.93-10.20 (m, 1H), 9.55-9.93 (m, 1H), 7.44-7.53 (m, 2H), 7.32-7.43 (m, 3H), 7.19-7.32 (m, 2H), 6.93 (dd, J=7.45, 1.89 Hz, 1H), 5.16 (s, 2 H), 4.65 (d, J=5.81 Hz, 2H), 4.10 (d, J=5.31 Hz, 2H).

### Example 2

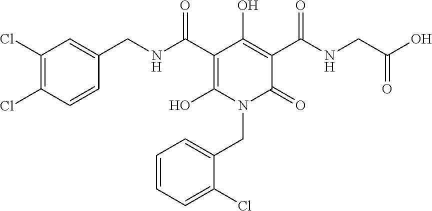

### N-{[1-[(2-chlorophenyl)methyl]-5-({[(3,4-dichlorophenyl)methyl]amino}carbonyl)-4,6-dihydroxy-2-oxo-1,2-dihydro-3-pyridinyl]carbonyl}glycine

A mixture of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (350 mg, 0.798 mmoles) and 3,4-dichlorobenzylamine (281 mg, 1.6 mmoles) in 1,4-dioxane (10 mL) was sealed in a pressure tube and heated in a microwave reactor at 150° C. for 30 minutes. The mixture was diluted with ethyl acetate and washed with 1 molar hydrochloric acid. The aqueous layer was extracted with ethyl acetate and the combined extracts washed with 1 molar hydrochloric acid. The organic solution was dried, evaporated and purified by flash chromatography (dichloromethane). The required fractions were evaporated and suspended in ethanol (10 mL), treated with 1 molar sodium hydroxide solution (10 ml) and 6 molar sodium hydroxide solution (5 ml) and stirred overnight. The mixture was acidified and extracted into ethyl acetate. The organic solution was washed with 1 molar hydrochloric acid, dried and evaporated. The residue formed a solid that was triturated with acetic acid and diluted with hexane, collected, washed with hexane and dried to give the title compound (370 mg, 83%). ¹H NMR (400 MHz, DMSO-d6) δ ppm 13.01 (br. s., 1H), 10.04 (s, 1H), 9.81 (s, 1H), 7.61 (d, J=8.08 Hz, 2H), 7.48 (d, J=7.33 Hz, 1H), 7.34 (dd, J=8.34, 2.02 Hz, 1H), 7.19-7.31 (m, J=14.68, 7.37, 7.37, 1.64 Hz, 2H), 6.92 (dd, J=7.45, 1.64 Hz, 1H), 5.15 (s, 2H), 4.55 (d, J=6.06 Hz, 2H), 4.09 (d, J=5.31 Hz, 2H).

### Example 3

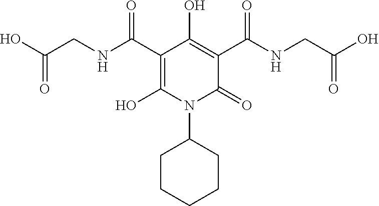

### 2,2′-{(1-cyclohexyl-4,6-dihydroxy-2-oxo-1,2-dihydropyridine-3,5-diyl)bis[(oxomethanediyl)imino]}diacetic acid

3a) 7-(Cyclohexylamino)-2,2-dimethyl-4H,5H-pyrano[4,3-d][1,3]dioxin-4,5-dione. A solution of 7-chloro-2,2-dimethyl-4H,5H-pyrano[4,3-d][1,3]dioxin-4,5-dione (2.97 g, 12.88 mmoles) in chloroform (45 mL) was treated dropwise with cyclohexylamine (2.95 mL, 25.88 mmoles) in chloroform (5 ml). The mixture was stirred for 1 hour, washed with water, 1 molar hydrochloric acid, dried and evaporated to give a pale yellow solid of the title dione (3.5 g, 95%). ¹H NMR (400 MHz, DMSO-d6) δ ppm 9.06 (d, J=19.71 Hz, 1H), 5.00-5.56 (m, J=121.77 Hz, 1 H), 3.61 (s, 1H), 1.77-1.98 (m, 2H), 1.50-1.77 (m, 8H), 1.03-1.44 (m, 5H).

3b) Methyl 1-cyclohexyl-2,4-dihydroxy-6-oxo-1,6-dihydro-3-pyridinecarboxylate. Sodium methoxide (25 mL of a 25% solution in methanol) was added to a solution of 7-(cyclohexylamino)-2,2-dimethyl-4H,5H-pyrano[4,3-d][1,3]dioxin-4,5-dione (7.5 g, 25.6 mmoles) in methanol (100 mL). The mixture was heated under reflux for 1 hour, evaporated, diluted with ethyl acetate and washed with 1 molar hydrochloric acid (×2). Flash chromatography (hexane to 80% ethyl acetate in hexane) gave an oil that yielded the title compound as an off-white solid on trituration with diethyl ether (2.4 g, 35%) ¹H NMR (400 MHz, DMSO-d6) δ ppm 7.98 (d, J=7.83 Hz, 1H), 3.71 (s, 3H), 3.51-3.57 (m, 2H), 3.40-3.53 (m, J=14.46, 7.29, 7.29, 3.54 Hz, 1H), 1.66-1.79 (m, 3H), 1.54 (d, J=12.38 Hz, 1H), 1.20-1.33 (m, 2H), 0.96-1.18 (m, 3H).

3c) 2,2′-{(1-Cyclohexyl-4,6-dihydroxy-2-oxo-1,2-dihydropyridine-3,5 diyl)bis[(oxomethanediyl)imino]}diacetic acid. A mixture of methyl 1-cyclohexyl-2,4-dihydroxy-6-oxo-1,6-dihydro-3-pyridinecarboxylate (350 mg, 1.31 mmoles), diisopropylethylamine (0.453 mL, 2.62 mmoles) and ethyl isocyanatoacetate (0.294 mL, 2.62 mmoles) in chloroform (8 mL) was sealed in a pressure flask and heated at 150° C. for 45 minutes in a microwave reactor. The mixture was washed with 1N hydrochloric acid and purified by flash chromatography (dichloromethane 10 mins then to 5% MeOH in dichloromethane over 5 minutes. The fractions were combined and the solvent evaporated, the residue taken up in ethanol (5 mL) after which 6 molar sodium hydroxide solution (2 mL) was added. The mixture was stored 2 hours, acidified and extracted into ethyl acetate. The aqueous layer was extracted with ethyl acetate. The combined extracts were washed with 1N hydrochloric acid, dried and the solvent evaporated. The residual solid was slurried in hexane, collected and dried to give the title diacetic acid (80 mg, 15%). ¹H NMR (400 MHz, DMSO-d6) δ ppm 12.99 (s, 2H), 9.81 (br. s., 2H), 4.85 (s, 1H), 4.09 (d, J=5.56 Hz, 4H), 2.26-2.44 (m, 2H), 1.81 (d, J=12.63 Hz, 2H), 1.53-1.70 (m, 3H), 1.31 (q, J=13.22 Hz, 2H), 1.10-1.22 (m, 1H).

### Example 4

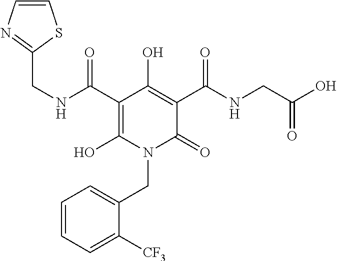

### N-[(4,6-dihydroxy-2-oxo-5-{[(1,3-thiazol-2-ylmethyl)amino]carbonyl}-1-{[2-(trifluoromethyl)phenyl]methyl}-1,2-dihydro-3-pyridinyl)carbonyl]glycine

4a) 2,2-Dimethyl-7-({[2-(trifluoromethyl)phenyl]methyl}amino)-4H,5H-pyrano[4,3-d][1,3]dioxin-4,5-dione. Diisopropylethylamine (3.46 mL, 20 mmoles) in chloroform (5 mL) was added dropwise to a cooled solution of 7-chloro-2,2-dimethyl-4H,5H-pyrano[4,3-d][1,3]dioxin-4,5-dione (2.5 g, 10.8 mmoles) followed by a solution of 2-trifluorobenzylamine (3.5 g, 20 mmoles) in chloroform (5 mL). Cooling was removed and the mixture was stirred at room temperature for 2 hours. The mixture was washed with water (×2), which gave a solid precipitate which was collected, washed with diethyl ether and hexane then dried to afford the title dione (3.4 g, 46%). ¹H NMR (400 MHz, DMSO-d6) δ ppm 9.62 (d, J=17.43 Hz, 1H), 7.75 (d, J=8.08 Hz, 2 H), 7.58 (d, J=8.08 Hz, 2H), 4.33-4.73 (m, 2H), 1.64 (s, 6H).

4b) Methyl 2,4-dihydroxy-6-oxo-1-{[2-(trifluoromethyl)phenyl]methyl}-1,6-dihydro-3-pyridinecarboxylate. Sodium (800 mg, 34.8 mmoles) was dissolved in methanol (80 mL) under argon. 2,2-Dimethyl-7-({[2-(trifluoromethyl)phenyl]methyl}amino)-4H,5H-pyrano[4,3-d][1,3]dioxin-4,5-dione (3.4 g, 9.21 mmoles) was added and the mixture heated at 65° C. for 2 hours then cooled and stirred overnight. The mixture was diluted with 1 molar hydrochloric acid and extracted into ethyl acetate (×2). The combined extracts were washed with 1 molar hydrochloric acid, dried and evaporated. Purification by flash chromatography (dichloromethane-5% methanol-dichloromethane-0.1% acetic acid) gave the title pyridinecarboxylate (650 mg, 21%). ¹H NMR (400 MHz, DMSO-d6) δ ppm 12.89 (s, 1H), 7.68 (d, J=8.08 Hz, 2H), 7.41 (d, J=8.08 Hz, 2 H), 5.29 (s, 1H), 5.13 (s, 2H), 3.74 (s, 3H).

4c) Methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate. Methyl 2,4-dihydroxy-6-oxo-1-{[2-(trifluoromethyl)phenyl]methyl}-1,6-dihydro-3-pyridinecarboxylate (650 mg, 1.89 mmoles), diisopropylethylamine (0.655 mL, 3.79 mmoles) and ethyl isocyanatoacetate (0.266 mL, 0.237 mmoles) in chloroform (5 mL) were sealed in a pressure tube and heated in a microwave reactor (120° C., 45 mins) The mixture was cooled, washed with 1 molar hydrochloric acid (×2), evaporated and purified by flash chromatography (dichloromethane-5% methanol in dichloromethane) to give the title pyridinecarboxylate as a solid from ether (500 mg, 56%). ¹H NMR (400 MHz, DMSO-d6) δ ppm 10.18 (t, J=5.43 Hz, 1H), 7.65 (d, J=8.08 Hz, 2H), 7.42 (d, J=8.08 Hz, 2H), 5.10 (s, 2H), 4.11 (q, J=7.24 Hz, 2H), 4.03 (d, J=5.56 Hz, 2H), 3.63 (s, 3H), 1.19 (t, J=7.07 Hz, 3H).

4d) N-[(4,6-dihydroxy-2-oxo-5-{[(1,3-thiazol-2-ylmethyl)amino]carbonyl}-1-{[2-(trifluoromethyl)phenyl]methyl}-1,2-dihydro-3-pyridinyl)carbonyl]glycine. A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (100 mg, 0.212 mmoles), 2-aminothiazole hydrochloride (60 mg, 0.424 mmoles) and diisopropylethylamine (73 μL, 0.424 mmoles) was sealed in a pressure tube and heated in a microwave reactor at 150° C. for 30 minutes. The mixture was diluted with 1 molar hydrochloric acid and extracted with ethyl acetate (×2). The combined extracts were washed with 1 molar hydrochloric acid and evaporated. The residue was slurried in ethanol and treated with 6 molar sodium hydroxide solution and stirred for 2 hours. The mixture was diluted with 1 molar hydrochloric acid and extracted with ethyl acetate (×2). Solvent was evaporated off and the residue was purified by preparative HPLC (20-80% acetonitrile-water-0.1% TFA) to give the title glycine (17 mg). ¹H NMR (400 MHz, DMSO-d6) δ ppm 12.94 (br. s., 1H), 10.22 (s, 1H), 9.78 (s, 1H), 7.76 (d, J=3.28 Hz, 1H), 7.62-7.74 (m, 3H), 7.49 (d, J=8.08 Hz, 2H), 5.20 (s, 2H), 4.89 (d, J=5.81 Hz, 2H), 4.10 (d, J=5.05 Hz, 2H).

### Example 5

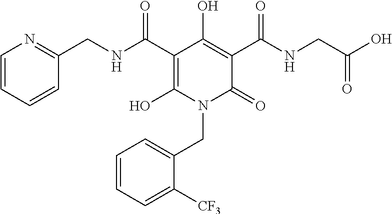

### N-[(4,6-dihydroxy-2-oxo-5-{[(2-pyridinylmethyl)amino]carbonyl}-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (100 mg, 0.212 mmoles) and 2-aminomethylpyridine (46 mg, 0.424 mmoles) were sealed in a pressure tube and heated in a microwave reactor at 150° C. for 30 minutes. The mixture was diluted with 1 molar hydrochloric acid and extracted with ethyl acetate (×2). The combined extracts were washed with 1 molar hydrochloric acid, sodium hydrogen carbonate solution, dried and evaporated. The residue was slurried in ethanol and treated with 6 molar sodium hydroxide solution and stirred for 2 hours. The mixture was diluted with 1 molar hydrochloric acid and extracted with ethyl acetate (×2). The combined extracts were washed with 1 molar hydrochloric acid, sodium hydrogen carbonate solution, dried and evaporated. The title compound was obtained as an off white solid from diethylether-hexane (18 mg, 16%). ¹H NMR (400 MHz, DMSO-d6) δ ppm 13.04 (br. s., 1 H), 10.25 (s, 1H), 9.81 (s, 1H), 8.66 (d, J=4.55 Hz, 1H), 8.04 (t, J=7.20 Hz, 1H), 7.69 (d, J=8.34 Hz, 2H), 7.58 (d, J=7.83 Hz, 1H), 7.42-7.55 (m, 3H), 5.21 (s, 2H), 4.79 (d, J=5.56 Hz, 2H), 4.10 (d, J=5.31 Hz, 2H).

### Example 6

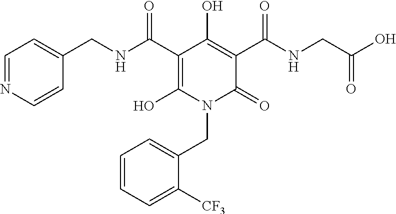

### N-[(4,6-dihydroxy-2-oxo-5-{[(4-pyridinylmethyl)amino]carbonyl}-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (200 mg, 0.424 mmoles) and 4-aminomethylpyridine (55 mg, 0.501 mmoles) were sealed in a pressure tube and heated in a microwave reactor at 150° C. for 30 minutes. The mixture was diluted with 1 molar hydrochloric acid and extracted with ethyl acetate (×2). The combined extracts were washed with 1 molar hydrochloric acid and evaporated. The residue was slurried in ethanol and treated with 6 molar sodium hydroxide solution and stirred for 2 hours. The mixture was acidified with 1 molar hydrochloric acid and extracted with ethyl acetate (×2). The residue was purified by preparative HPLC (20-80% acetonitrile-water-0.1% TFA) to give the title compound (34 mg, 15%). ¹H NMR (400 MHz, DMSO-d6) δ ppm 13.00 (br. s., 1H), 10.11 (s, 1H), 9.83 (s, 1H), 8.70 (d, J=5.56 Hz, 2H), 7.60-7.82 (m, 4H), 7.50 (s, 2H), 5.20 (s, 2H), 4.72 (d, J=5.81 Hz, 2H), 4.09 (d, J=5.31 Hz, 2H).

### Example 7

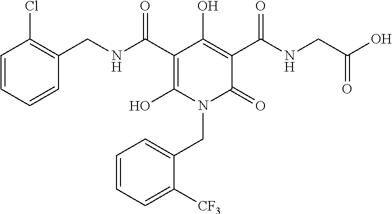

### N-[(5-({[(2-chlorophenyl)methyl]amino}carbonyl)-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (200 mg, 0.424 mmoles) and 2-chlorobenzylamine (72 mg, 0.51 mmoles) were sealed in a pressure tube and heated in a microwave reactor at 150° C. for 30 minutes. The mixture was diluted with 1 molar hydrochloric acid and extracted with ethyl acetate (×2). The combined extracts were washed with 1 molar hydrochloric acid and evaporated. The residue was slurried in ethanol and treated with 6 molar sodium hydroxide solution and stirred for 2 hours. The mixture was acidified with 1 molar hydrochloric acid and extracted with ethyl acetate (×2). The residue was purified by recrystallization from ethanol-water to give the title compound (180 mg, 77%). ¹H NMR (400 MHz, DMSO-d6) δ ppm 13.00 (s, 1H), 10.04 (s, 1H), 9.81 (s, 1H), 7.68 (d, J=8.08 Hz, 2H), 7.42-7.57 (m, 3H), 7.14-7.44 (m, 3H), 5.20 (s, 2H), 4.64 (d, J=6.06 Hz, 2H), 4.09 (d, J=5.31 Hz, 2 H).

### Example 8

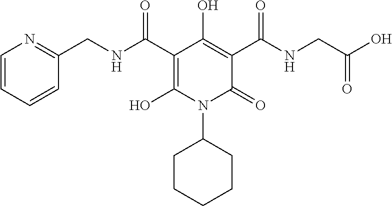

### N-[(1-cyclohexyl-4,6-dihydroxy-2-oxo-5-{[(2-pyridinylmethyl)amino]carbonyl}-1,2-dihydro-3-pyridinyl)carbonyl]glycine

8a) Methyl 1-cyclohexyl-5-({[2-(ethyloxy)-2-oxoethyl]amino}carbonyl)-2,4-dihydroxy-6-oxo-1,6-dihydro-3-pyridinecarboxylate. A mixture of methyl 1-cyclohexyl-2,4-dihydroxy-6-oxo-1,6-dihydro-3-pyridinecarboxylate (2.3 g, 8.61 mmoles), diisopropylethylamine (1.79 mL, 10.33 mmoles) and ethyl isocyanatoacetate (1.16 mL, 10.33 mmoles) in chloroform (8 mL) was sealed in a pressure flask and heated at 120° C. for 45 minutes in a microwave reactor. The mixture was washed with 1N hydrochloric acid and purified by flash chromatography (CH2Cl2 10 mins then to 5% MeOH in CH₂Cl₂ over 5 minutes) to give the title 3-pyridinecarboxylate (1.3 g, 38%). ¹H NMR (400 MHz, DMSO-d6) δ ppm 10.18 (s, 1H), 4.86 (br. s., 1H), 4.13 (q, J=7.16 Hz, 2H), 4.07 (d, J=5.56 Hz, 2H), 3.76 (s, 3H), 2.26-2.46 (m, 2H), 1.79 (d, J=12.88 Hz, 2H), 1.63 (d, J=12.63 Hz, 1H), 1.54 (d, J=11.62 Hz, 2H), 1.23-1.35 (m, 2H), 1.21 (t, J=7.07 Hz, 3H), 1.07-1.16 (m, 1H).

8b) N-[(1-cyclohexyl-4,6-dihydroxy-2-oxo-5-{[(2-pyridinylmethyl)amino]carbonyl}-1,2-dihydro-3-pyridinyl)carbonyl]glycine. A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (202 mg, 0.51 mmoles) and 2-aminomethylpyridine (83 mg, 0.764 mmoles) in 1,4-dioxane (3 mL) was sealed in a pressure tube and heated in a microwave reactor at 150° C. for 30 minutes. The reaction mixture was treated with 6 molar sodium hydroxide (3 ml) and ethanol (2 ml) and stirred for 2 hours. The mixture was acidified with 1 molar hydrochloric acid and extracted with ethyl acetate (×2) and evaporated. The resulting residue was purified by preparative HPLC (20-80% acetonitrile-water-0.1% TFA) to give the title compound (65 mg, 29%). ¹H NMR (400 MHz, DMSO-d6) δ ppm 12.99 (s, 1H), 10.27 (br. s., 1H), 9.87 (br. s., 1H), 8.43-8.78 (m, 1H), 7.81 (td, J=7.71, 1.77 Hz, 1H), 7.40 (d, J=7.83 Hz, 1H), 7.33 (dd, J=7.45, 5.94 Hz, 1H), 4.78-5.00 (m, 1H), 4.71 (d, J=5.31 Hz, 2H), 4.09 (d, J=5.56 Hz, 2H), 2.20-2.46 (m, 2H), 1.80 (d, J=12.63 Hz, 2H), 1.47-1.71 (m, 3H), 1.31 (q, J=12.63 Hz, 2H), 1.15 (q, J=12.88 Hz, 1H).

### Example 9

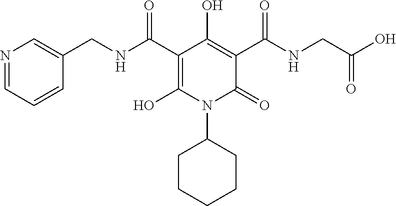

### N-[(1-cyclohexyl-4,6-dihydroxy-2-oxo-5-{[(3-pyridinylmethyl)amino]carbonyl}-1,2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (202 mg, 0.51 mmoles) and 3-aminomethylpyridine (83 mg, 0.764 mmoles) in 1,4-dioxane (3 mL) was sealed in a pressure tube and heated in a microwave reactor at 150° C. for 30 minutes. The mixture was diluted with water and extracted with ethyl acetate (×2). The combined extracts were washed with sodium hydrogen carbonate solution, brine and evaporated. The residue was slurried in ethanol and treated with 6 molar sodium hydroxide solution and stirred for 2 hours. The mixture was acidified with 1 molar hydrochloric acid, evaporated. The residue was purified by preparative HPLC (20-70% acetonitrile-water-0.1% TFA) gave the title compound (80 mg, 35%). ¹H NMR (400 MHz, DMSO-d6) δ ppm 12.99 (s, 1H), 10.07 (br. s., 1H), 9.81 (br. s., 1H), 8.60 (s, 1H), 8.52 (d, J=4.04 Hz, 1H), 7.83 (d, J=7.83 Hz, 1H), 7.44 (dd, J=7.71, 4.93 Hz, 1H), 4.84 (t, J=12.00 Hz, 1 H), 4.60 (d, J=5.81 Hz, 2H), 4.08 (d, J=5.56 Hz, 2H), 2.14-2.41 (m, 2H), 1.79 (d, J=13.39 Hz, 2 H), 1.46-1.69 (m, 3H), 1.29 (q, J=13.39 Hz, 2H), 1.13 (q, 1H).

### Example 10

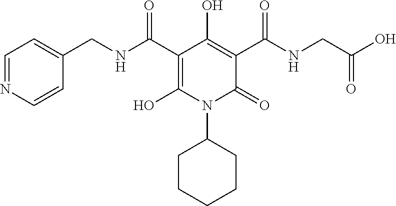

### N-[(1-cyclohexyl-4,6-dihydroxy-2-oxo-5-{[(4-pyridinylmethyl)amino]carbonyl}-1,2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (202 mg, 0.51 mmoles) and 4-aminomethylpyridine (83 mg, 0.764 mmoles) in 1,4-dioxane (3 mL) was sealed in a pressure tube and heated in a microwave reactor at 150° C. for 30 minutes. The mixture was diluted with water and extracted with ethyl acetate (×2). The combined extracts were washed with sodium hydrogen carbonate solution, brine and evaporated. The residue was slurried in ethanol and treated with 6 molar sodium hydroxide solution and stirred for 2 hours. The mixture was acidified with 1 molar hydrochloric acid and evaporated. The residue was dissolved in DMSO and purified by preparative HPLC (20-70% acetonitrile-water-0.1% TFA) gave the title compound (82 mg, 36%). ¹H NMR (400 MHz, DMSO-d6) δ ppm 13.03 (br. s., 1H), 10.14 (br. s., 1H), 9.81 (br. s., 1H), 8.71 (d, J=6.06 Hz, 2H), 7.67 (d, J=6.06 Hz, 2H), 4.79-5.01 (m, 1H), 4.73 (d, J=6.06 Hz, 2H), 4.09 (d, J=5.56 Hz, 2H), 2.21-2.44 (m, 2H), 1.81 (d, J=13.14 Hz, 2H), 1.63 (t, J=13.64 Hz, 3H), 1.30 (q, 2H), 1.14 (q, 1H).

### Example 11

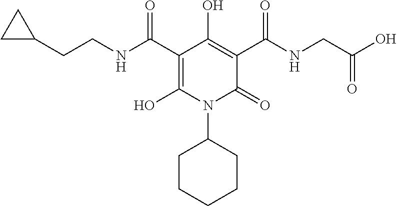

### N-[(1-cyclohexyl-5-{[(2-cyclopropylethyl)amino]carbonyl}-4,6-dihydroxy-2-oxo-1,2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (160 mg, 0.404 mmoles), 2-cyclopropylethylamine hydrochloride (74 mg, 0.609 mmoles) and diisopropylethylamine (120 μL, 0.609 mmoles) in 1,4-dioxane (3 mL) was sealed in a pressure tube and heated in a microwave reactor at 150° C. for 30 minutes. The reaction mixture was treated with 6 molar sodium hydroxide (3 ml) and ethanol (2 ml) and stirred for 2 hours. The mixture was acidified with 1 molar hydrochloric acid and extracted with ethyl acetate (×2). The residue was slurried in ethanol and collected to give the title compound (57 mg). ¹H NMR (400 MHz, DMSO-d6) δ ppm 12.98 (s, 1 H), 9.75 (br. s., 2H), 4.84 (s, 1H), 4.08 (d, J=5.56 Hz, 2H), 3.43 (q, J=6.82 Hz, 2H), 2.19-2.45 (m, 2H), 1.80 (d, J=12.63 Hz, 2H), 1.52-1.70 (m, 3H), 1.46 (q, J=6.91 Hz, 2H), 1.30 (q, J=12.80 Hz, 2H), 1.14 (q, 1H), 0.61-0.77 (m, 1H), 0.43 (ddd, J=8.02, 5.62, 4.04 Hz, 2H), 0.08 (td, J=5.12, 4.42 Hz, 2H).

### Example 12

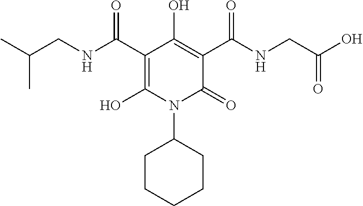

### N-[(1-cyclohexyl-4,6-dihydroxy-5-{[(2-methylpropyl)amino]carbonyl}-2-oxo-1,2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (160 mg, 0.404 mmoles) and isobutylamine (60 μL, 0.604 mmoles) in 1,4-dioxane (3 mL) was sealed in a pressure tube and heated in a microwave reactor at 150° C. for 30 minutes. The reaction mixture was treated with 6 molar sodium hydroxide (3 ml) and ethanol (2 ml) and stirred for 2 hours. The mixture was acidified with 1 molar hydrochloric acid and extracted with ethyl acetate (×2). The mixture was dried and evaporated to give a residue that was slurried in ether and collected to give the title compound (20 mg, 12%). ¹H NMR (400 MHz, DMSO-d6) δ ppm 12.98 (s, 1H), 9.68 (br. s., 2H), 4.85 (s, 1H), 4.08 (d, J=5.56 Hz, 2H), 3.21 (t, J=6.32 Hz, 2H), 2.16-2.46 (m, 2H), 1.88 (dt, J=13.52, 7.07 Hz, 1H), 1.80 (d, J=13.14 Hz, 2H), 1.50-1.70 (m, 3H), 1.30 (q, J=13.14 Hz, 2H), 1.15 (q, 1H), 0.91 (d, J=6.82 Hz, 6H).

### Example 13

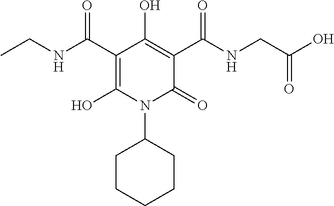

### N-({1-cyclohexyl-5-[(ethylamino)carbonyl]-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (160 mg, 0.404 mmoles) and ethylamine (400 μL of a 2.0 molar solution in methanol) in 1,4-dioxane (3 mL) was sealed in a pressure tube and heated in a microwave reactor at 150° C. for 30 minutes. The reaction mixture was treated with 6 molar sodium hydroxide (3 ml) and ethanol (2 ml) and stirred for 2 hours. The mixture was acidified with 1 molar hydrochloric acid and extracted with ethyl acetate (×2). The solution was dried and evaporated to give a residue that was purified by flash chromatography (10-100% ethyl acetate-hexane-1% acetic acid) to give a solid on evaporation of the required fractions. The solid was slurried in ether and collected to obtain the title compound (45 mg, 29%). ¹H NMR (400 MHz, DMSO-d6) δ ppm 12.97 (s, 1H), 9.91 (br. s., 2H), 4.84 (s, 1H), 4.08 (d, J=5.56 Hz, 2H), 3.39 (q, 2H), 2.24-2.43 (m, 2H), 1.80 (d, J=13.14 Hz, 2H), 1.49-1.71 (m, 3H), 1.30 (q, J=12.80 Hz, 2H), 0.96-1.22 (m, 4H).

### Example 14

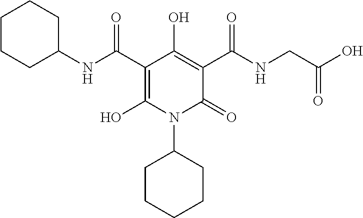

### N-({1-cyclohexyl-5-[(cyclohexylamino)carbonyl]-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (200 mg, 0.505 mmoles) and cyclohexylamine (87 μL, 0.757 mmoles) in 1,4-dioxane (3 mL) was sealed in a pressure tube and heated in a microwave reactor at 150° C. for 30 minutes. The reaction mixture was treated with 6 molar sodium hydroxide (3 ml) and ethanol (2 ml) and stirred for 2 hours. The mixture was acidified with 1 molar hydrochloric acid and extracted with ethyl acetate (×2). The mixture was dried and evaporated to give a residue that was slurried in ether and collected to give the title compound (145 mg, 66%). ¹H NMR (400 MHz, DMSO-d6) δ ppm 12.99 (s, 1H), 9.78 (br. s., 2H), 4.84 (s, 1H), 4.09 (d, J=5.56 Hz, 2H), 3.81 (s, 1H), 3.36 (s, 1H), 2.16-2.42 (m, 2H), 1.74-1.91 (m, 4H), 1.50-1.73 (m, 6H), 1.33-1.44 (m, 4H), 1.21-1.33 (m, 3H), 1.11-1.21 (m, 1H).

### Example 15

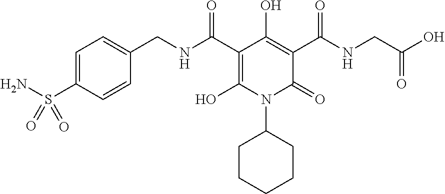

### N-({5-[({[4-(aminosulfonyl)phenyl]methyl}amino)carbonyl]-1-cyclohexyl-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (250 mg, 0.631 mmoles), 4-aminomethylbenzenesulfonamide hydrochloride (156 mg, 0.701 mmoles) and diisopropylethylamine (122 μL, 0.704 mmoles) in 1,4-dioxane (3 mL) was sealed in a pressure tube and heated in a microwave reactor at 150° C. for 30 minutes. The mixture was treated with 6 molar sodium hydroxide solution (2 mL) and stirred overnight. The mixture was acidified and extracted into ethyl acetate (×2). Combined extracts were washed with 1 molar hydrochloric acid, dried and evaporated and purified by preparative HPLC (40-80% acetonitrile-water-0.1% TFA). On evaporation of the required fractions the title compound was slurried in ethanol, collected, washed with ethanol and hexane, and dried to give the title compound (48 mg, 15%). ¹H NMR (400 MHz, DMSO-d6) δ ppm 12.98 (s, 1H), 9.43-10.40 (m, J=109.64 Hz, 2H), 7.80 (d, J=8.34 Hz, 2H), 7.51 (d, J=8.59 Hz, 2H), 7.34 (s, 2H), 4.73-5.00 (m, 1H), 4.63 (d, J=6.06 Hz, 2H), 4.08 (d, J=5.56 Hz, 2H), 2.24-2.43 (m, 2H), 1.80 (d, J=12.88 Hz, 2H), 1.48-1.70 (m, 3H), 1.29 (q, J=13.22 Hz, 2H), 1.13 (q, J=12.29 Hz, 1H).

### Example 16

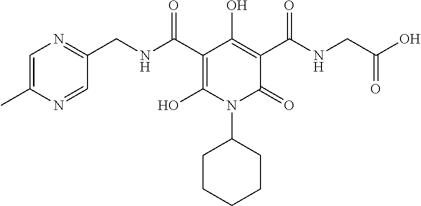

### N-{[1-cyclohexyl-4,6-dihydroxy-5-({[(5-methyl-2-pyrazinyl)methyl]-amino}carbonyl)-2-oxo-1,2-dihydro-3-pyridinyl]carbonyl}glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (200 mg, 0.505 mmoles), and [(5-methyl-2-pyrazinyl)methyl]amine (68 mg, 0.555 mmoles) in 1,4-dioxane (3 mL) was sealed in a pressure tube and heated in a microwave reactor at 150° C. for 30 minutes. The mixture was cooled and treated with ethanol and 6 molar sodium hydroxide solution (2 mL) and stirred overnight. The mixture was acidified an extracted into ethyl acetate then washed with 1 molar hydrochloric acid, evaporated and recrystallized from DMSO to give the title compound (135 mg, 58%). ¹H NMR (400 MHz, DMSO-d6) δ ppm 12.99 (s, 1H), 9.11-10.55 (m, J=137.68 Hz, 1H), 8.54 (s, 1H), 8.52 (s, 1H), 4.77-4.96 (m, 1H), 4.71 (d, J=5.56 Hz, 2H), 4.09 (d, J=5.56 Hz, 2H), 2.49 (s, 3 H), 2.34 (q, J=11.28 Hz, 2H), 1.80 (d, J=12.63 Hz, 2H), 1.50-1.71 (m, 3H), 1.30 (q, J=13.05 Hz, 2H), 1.14 (q, J=12.55 Hz, 1H).

### Example 17

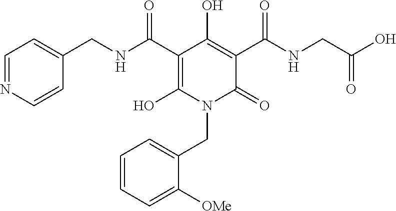

### N-[(4,6-dihydroxy-1-{[2-(methyloxy)phenyl]methyl}-2-oxo-5-{[(4-pyridinylmethyl)amino]carbonyl}-1,2-dihydro-3-pyridinyl)carbonyl]glycine

17a) 2,2-Dimethyl-7-({[2-(methyloxy)phenyl]methyl}amino)-4H,5H-pyrano[4,3-d][1,3]dioxin-4,5-dione. 7-Chloro-2,2-dimethyl-4H,5H-pyrano[4,3-d][1,3]dioxin-4,5-dione (9.9 g, 42.9 mmoles) was taken up in chloroform (50 mL) and cooled over an ice bath. N,N-Diisopropylethylamine (8.17 mL, 47.2 mmoles) in chloroform (20 mL) was added dropwise, followed by a solution of 2-methoxybenzylamine (6.17 mL) in chloroform (30 mL). Cooling was removed and the mixture was stirred at room temperature overnight. The mixture was washed with water, dried and evaporated to give the title 4,5-dione (8.0 g, 56%). ¹H NMR (400 MHz, DMSO-d6) δ ppm 9.45 (d, J=41.94 Hz, 1H), 7.17-7.47 (m, 2H), 7.04 (d, J=7.58 Hz, 1H), 6.95 (t, J=6.82 Hz, 1H), 5.27 (d, J=11.37 Hz, 1H), 4.44 (s, 1H), 4.34 (s, 1H), 3.82 (s, 3H), 1.64 (s, 6H).

17b) Methyl 2,4-dihydroxy-1-{[2-(methyloxy)phenyl]methyl}-6-oxo-1,6-dihydro-3-pyridinecarboxylate. Sodium (2.0 g, 87 mmoles) was dissolved in methanol (100 mL) under argon. 2,2-Dimethyl-7-({[2-(methyloxy)phenyl]methyl}amino)-4H,5H-pyrano[4,3-d][1,3]dioxin-4,5-dione (8.0 g, 24.15 mmoles) was added and the mixture heated at 65° C. for 2 hours. The mixture was evaporated to low volume and partitioned between ethyl acetate and 1 molar hydrochloric acid. The organic layer was washed with 1 molar hydrochloric acid, dried and evaporated. Trituration with acetonitrile gave a solid that was collected, washed with acetonitrile, hexane, dried in vacuo to give the title methyl ester (4.3 g, 58%). ¹H NMR (400 MHz, DMSO-d6) δ ppm 12.91 (s, 1H), 10.78 (br. s., 1H), 7.22 (t, J=7.83 Hz, 1H), 7.00 (d, J=8.08 Hz, 1H), 6.84 (t, J=7.07 Hz, 1H), 6.56 (d, J=7.07 Hz, 1H), 5.41 (s, 1H), 5.02 (s, 2H), 3.84 (s, 3H), 3.75 (s, 3H).

17c) Methyl 5-({[2-(ethyloxy)-2-oxoethyl]amino}carbonyl)-2,4-dihydroxy-1-{[2-(methyloxy)phenyl]methyl}-6-oxo-1,6-dihydro-3-pyridinecarboxylate. A mixture of methyl 2,4-dihydroxy-1-{[2-(methyloxy)phenyl]methyl}-6-oxo-1,6-dihydro-3-pyridinecarboxylate (4.3 g, 14.09 mmoles), N,N-diisopropylethylamine (2.68 mL, 15.5 mmoles) and ethyl isocyanatoacetate 1.74 mL, 15.5 mmoles) in chloroform (20 mL) was sealed in a pressure flask and heated at 120° C. for 45 minutes in a microwave reactor. The mixture was washed with 1N hydrochloric acid. A significant amount of the bis-glycinamide by-product (˜30%) was present. The product was purified by recrystallization from acetone to give the title compound, which still contained around 10% of the bis-glycinamide by-product (1.73 g, 28%). ¹H NMR (400 MHz, DMSO-d6) δ ppm 10.11 (s, 1H), 8.41 (br. s., 1H), 7.12-7.26 (m, 1H), 6.99 (d, J=7.58 Hz, 1H), 6.83 (td, J=7.52, 0.88 Hz, 1H), 6.65 (dd, J=7.45, 1.39 Hz, 1H), 5.04 (s, 2H), 4.11 (q, J=6.99 Hz, 2H), 4.05 (d, J=5.31 Hz, 2H), 3.84 (s, 3H), 3.72 (s, 3H), 1.19 (t, J=6.82 Hz, 3H).

17d) N-[(4,6-dihydroxy-1-{[2-(methyloxy)phenyl]methyl}-2-oxo-5-{[(4-pyridinylmethyl)amino]carbonyl}-1,2-dihydro-3-pyridinyl)carbonyl]glycine. A mixture of methyl 5-({[2-(ethyloxy)-2-oxoethyl]amino}carbonyl)-2,4-dihydroxy-1-{[2-(methyloxy)phenyl]methyl}-6-oxo-1,6-dihydro-3-pyridinecarboxylate (250 mg, 0.576 mmoles) and (4-pyridinylmethyl)amine (64 μL, 0.63 mmoles) in 1,4-dioxane (3 mL) was sealed in a pressure flask and heated at 150° C. for 30 minutes in a microwave reactor. LCMS indicated the reaction was complete, however there was a bis glycinamide impurity present. The mixture was purified by flash chromatography (0-10% methanol in dichloromethane). The appropriate fractions were combined and evaporated. The residue was treated with 1 molar sodium hydroxide solution in ethanol and stirred overnight. It was then acidified with 1 molar hydrochloric acid to give a solid that was collected, washed with water, ethanol, then ether and hexanes and dried to give the title compound as the monohydrate (160 mg, 58%). ¹H NMR (400 MHz, DMSO-d6) δ ppm 12.86 (br. s., J=2.27 Hz, 1H), 10.06 (s, 1 H), 9.84 (s, 1H), 8.57 (d, J=5.81 Hz, 2H), 7.41 (d, J=5.56 Hz, 2H), 7.22 (dd, J=7.45 Hz, 1H), 7.01 (d, J=7.83 Hz, 1H), 6.84 (dd, J=7.33 Hz, 1H), 6.70 (d, J=6.82 Hz, 1H), 5.08 (s, 2H), 4.61 (d, J=6.06 Hz, 2H), 4.07 (d, J=5.05 Hz, 2H), 3.85 (s, 3H). Elam. An.: C₂₃H₂₂N₄O₈H₂O Found: C=55.06, H=4.60, N=11.06. Requires: C=55.18, H=4.84, N=11.20.

### Example 18

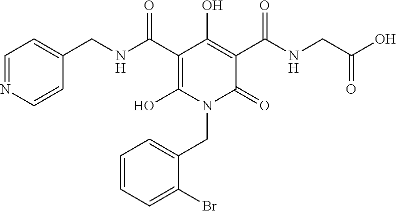

### N-[(1-[(2-bromophenyl)methyl]-4,6-dihydroxy-2-oxo-5-{[(4-pyridinylmethyl)amino]carbonyl}-1,2-dihydro-3-pyridinyl)carbonyl]glycine

18a) 7-{[(2-Bromophenyl)methyl]amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (6.46 g, 28 mmoles) was taken up in chloroform (50 mL) and cooled over an ice bath. N,N-Diisopropylethylamine (5.33 mL, 30.8 mmoles) in chloroform (25 mL) was added dropwise, followed by a solution of 2-bromobenzylamine (6.85 g, 30.8 mmoles) in chloroform (25 mL). Cooling was removed and the mixture was stirred at room temperature overnight. The mixture was washed with water, dried and evaporated to give the title dione (8.4 g, 79%). ¹H NMR (400 MHz, DMSO-d6) δ ppm 9.35-9.82 (m, J=56.84 Hz, 1H), 7.67 (d, J=8.08 Hz, 1H), 7.44 (s, 2H), 7.30 (s, 1H), 5.31 (s, 1H), 4.50 (d, J=35.37 Hz, 2H), 1.64 (s, 6H).

18b) Methyl 1-[(2-bromophenyl)methyl]-2,4-dihydroxy-6-oxo-1,6-dihydro-3-pyridinecarboxylate. Sodium (1.6 g, 69.6 mmoles) was dissolved in dry methanol (80 mL) under nitrogen. 7-{[(2-bromophenyl)methyl]amino}-2,2-dimethyl-4H,5H-pyrano[4,3-d][1,3]dioxin-4,5-dione (8.4 g, 22.09 mmoles) was added and the mixture was heated at 65° C. for 2 hours. The mixture was evaporated to low volume and partitioned between ethyl acetate and 1 molar hydrochloric acid. The organic layer was washed with 1 molar hydrochloric acid, dried and evaporated. Trituration with acetonitrile gave a solid that was collected, washed with acetonitrile, hexane and dried in vacuo to give the title methyl ester (2.8 g, 36%). ¹H NMR (400 MHz, DMSO-d₆) δ ppm 12.95 (s, 1H), 9.03 (br. s., 1H), 7.64 (d, J=7.83 Hz, 1H), 7.31 (dd, J=7.45 Hz, 1H), 7.20 (dd, J=7.71 Hz, 1H), 6.69 (d, J=29.30 Hz, 1H), 5.27-5.57 (m, 1H), 5.06 (d, J=5.81 Hz, 2 H), 3.74 (s, 3H).

18c) Methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(2-bromophenyl)methyl]-2,4-dihydroxy-6-oxo-1,6-dihydro-3-pyridinecarboxylate, (2.9 g, 8.19 mmoles), N,N-diisopropylethylamine (1.56 mL, 9.01 mmoles) and ethyl isocyanatoacetate (1.01 mL, 9.01 mmoles) in chloroform (100 mL) was sealed in a pressure flask and heated at 100° C. for 3 hours. LCMS showed reaction was complete. The mixture was washed with 1 molar hydrochloric acid (×2), dried and evaporated to a brown foam. The foam was triturated with diethyl ether for 2 hours to afford a tan powder which was collected and washed with ether and hexanes to give the title methyl ester (1.95 g, 49%). ¹H NMR (400 MHz, DMSO-d6) δ ppm 10.13 (t, J=5.18 Hz, 1H), 7.61 (dd, J=7.96, 1.14 Hz, 1H), 7.29 (td, J=7.45, 1.26 Hz, 1H), 7.17 (td, J=7.58, 1.77 Hz, 1H), 6.76 (dd, J=7.71, 1.39 Hz, 1H), 5.03 (s, 2H), 4.11 (q, J=7.07 Hz, 2H), 4.03 (d, J=5.31 Hz, 2H), 3.66 (s, 3H), 1.19 (t, J=7.20 Hz, 3H).

18d) N-[(1-[(2-bromophenyl)methyl]-4,6-dihydroxy-2-oxo-5-{[(4-pyridinylmethyl)amino]carbonyl}-1,2-dihydro-3-pyridinyl)carbonyl]glycine. Methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (250 mg, 0.517 mmoles) and (4-pyridinemethyl)amine (58 μL, 0.569 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution and ethanol and stirred overnight. It was then acidified with 1 molar hydrochloric acid to give a solid that was collected, washed with water, ethanol and then ether and hexanes and dried to an off white solid, it being the title compound as the monohydrochloride dihydrate (210 mg, 76%). ¹H NMR (400 MHz, DMSO-d6) δ ppm 13.05 (br. s., 1H), 10.15 (s, 1H), 9.77 (s, 1H), 8.81 (d, J=6.57 Hz, 2H), 7.88 (d, J=6.57 Hz, 2H), 7.66 (d, J=7.83 Hz, 1H), 7.31 (dd, J=7.58 Hz, 1H), 7.22 (dd, J=7.58 Hz, 1H), 6.89 (d, J=7.83 Hz, 1H), 5.13 (s, 2H), 4.81 (d, J=5.81 Hz, 2H), 4.10 (d, J=4.80 Hz, 2H). Anal. C₂₂H₁₉BrN₄O₇.HCl.2H₂O. Found: C=44.02, H=3.87, N=9.20, Cl=5.75. Requires C=43.74, H=4.01, Br=13.24, Cl=5.87, N=9.28.

### Example 19

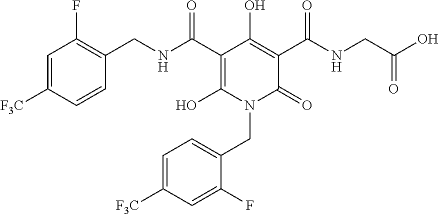

### N-({1-{[2-fluoro-4-(trifluoromethyl)phenyl]methyl}-5-[({[2-fluoro-4-(trifluoromethyl)phenyl]methyl}amino)carbonyl]-4,6-dihydroxy-2-oxo-1,2-dihydro-3-pyridinyl}carbonyl)glycine

19a) 7-({[2-Fluoro-4-(trifluoromethyl)phenyl]methyl}amino)-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (5.97 g, 25.9 mmoles) was taken up in chloroform (50 mL) and cooled over an ice bath. N,N-Diisopropylethylamine (4.48 mL, 25.9 mmoles) in chloroform (25 mL) was added dropwise, followed by a solution of 2-fluoro-4-trifluoromethylbenzylamine (5.0 g, 25.9 mmoles) in chloroform (25 mL). Cooling was removed and the mixture was stirred at room temperature for two hours. Addition of water gave a solid, and the mixture was stirred for 1 hour to ensure most product precipitated. The solid was collected, washed with water and azeotroped with methanol to give a free flowing powder, which was slurried in methanol, collected, washed with methanol and hexane, dried in vacuo to afford the title compound (9.3 g, 93%). ¹H NMR (400 MHz, DMSO-d6) δ ppm 9.59 (d, J=56.08 Hz, 1H), 7.54-7.87 (m, 3H), 5.34 (d, J=64.17 Hz, 1H), 4.59 (s, 2H), 1.64 (s, 6H).

19b) Methyl 1-{[2-fluoro-4-(trifluoromethyl)phenyl]methyl}-2,4-dihydroxy-6-oxo-1,6-dihydro-3-pyridinecarboxylate. Sodium (2.0 g, 87 mmoles) was dissolved in dry methanol under nitrogen. 7-({[2-fluoro-4-(trifluoromethyl)phenyl]methyl}amino)-2,2-dimethyl-4H,5H-pyrano[4,3-d][1,3]dioxin-4,5-dione (9.3 g, 24 mmoles) was added and the mixture was heated at 65° C. for 3 hours. The mixture was evaporated to low volume and partitioned between ethyl acetate and 1 molar hydrochloric acid. The organic layer was washed with 1 molar hydrochloric acid, dried and evaporated. Trituration with diethyl ether gave a solid that was collected and washed with diethyl ether and hexane to afford the title compound (4.75 g 55%). ¹H NMR (400 MHz, DMSO-d6) δ ppm 12.93 (s, 1H), 7.68 (d, J=9.09 Hz, 1H), 7.53 (d, J=8.08 Hz, 1H), 7.20 (s, 1H), 5.32 (s, 1H), 5.15 (s, 2H), 3.74 (s, 3H).

19c) Methyl 5-({[2-(ethyloxy)-2-oxoethyl]amino}carbonyl)-1-{[2-fluoro-4-(trifluoromethyl)phenyl]methyl}-2,4-dihydroxy-6-oxo-1,6-dihydro-3-pyridinecarboxylate. A mixture of methyl 1-{[2-fluoro-4-(trifluoromethyl)phenyl]methyl}-2,4-dihydroxy-6-oxo-1,6-dihydro-3-pyridinecarboxylate (4.7 g, 13 mmoles), N,N-diisopropylethylamine (2.70 mL, 15.6 mmoles) and ethyl isocyanatoacetate (1.75 mL, 15.6 mmoles) in chloroform (100 mL) was sealed in a pressure flask under nitrogen and heated at 110° C. for 3 hours. LCMS showed the reaction was complete. The mixture was washed with 1 molar hydrochloric acid (×2), dried and evaporated to give a light brown solid. The solid was triturated with diethyl ether, collected, washed with ether and hexanes to give the title compound (5.72 g, 90%). ¹H NMR (400 MHz, DMSO-d6) δ ppm 10.12 (s, 1H), 7.63 (d, J=10.11 Hz, 1H), 7.51 (d, J=8.08 Hz, 1H), 7.04-7.27 (m, J=7.71, 7.71 Hz, 1H), 5.12 (s, 2H), 4.11 (q, J=7.07 Hz, 2H), 4.02 (d, J=5.56 Hz, 2H), 3.63 (s, 3H), 1.19 (t, J=7.20 Hz, 3H).

19d) N-({1-{[2-fluoro-4-(trifluoromethyl)phenyl]methyl}-5-[({[2-fluoro-4-(trifluoromethyl)phenyl]methyl}amino)carbonyl]-4,6-dihydroxy-2-oxo-1,2-dihydro-3-pyridinyl}carbonyl)glycine. Methyl 5-({[2-(ethyloxy)-2-oxoethyl]amino}carbonyl)-1-{[2-fluoro-4-(trifluoromethyl)phenyl]methyl}-2,4-dihydroxy-6-oxo-1,6-dihydro-3-pyridinecarboxylate (300 mg, 0.612 mmoles) and 2-fluoro-4-(trifluoromethyl)benzylamine (142 mg, 0.734 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution (3 mL) and stirred overnight. The mixture was diluted with ethyl acetate and washed with 1 molar hydrochloric acid (×2) and evaporated to a solid that was triturated in diethyl ether, collected, washed with diethyl ether and hexanes and dried to afford the title compound as a pale yellow solid (237 mg, 62%). ¹H NMR (400 MHz, DMSO-d6) δ ppm 10.05 (s, 1H), 9.77 (s, 1H), 7.69 (d, J=10.11 Hz, 2H), 7.56-7.64 (m, 2H), 7.51 (d, J=8.08 Hz, 1 H), 7.33 (dd, J=7.71 Hz, 1H), 5.21 (s, 2H), 4.69 (d, J=5.81 Hz, 2H), 4.10 (d, J=5.31 Hz, 2H).

### Example 20

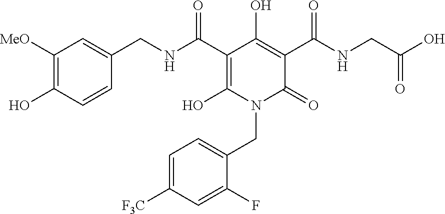

### N-({1-{[2-fluoro-4-(trifluoromethyl)phenyl]methyl}-4,6-dihydroxy-5-[({[4-hydroxy-3-(methyloxy)phenyl]methyl}amino)carbonyl]-2-oxo-1,2-dihydro-3-pyridinyl}carbonyl)glycine

Methyl 5-({[2-(ethyloxy)-2-oxoethyl]amino}carbonyl)-1-{[2-fluoro-4-(trifluoromethyl)phenyl]methyl}-2,4-dihydroxy-6-oxo-1,6-dihydro-3-pyridinecarboxylate (300 mg, 0.612 mmoles) and 4-(aminomethyl)-2-(methyloxy)phenol (94 mg, 0.612 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution (3 mL) and stirred overnight. The mixture was diluted with ethyl acetate and washed with 1 molar hydrochloric acid (×2) and evaporated to a solid that was triturated in diethyl ether, collected, washed with diethyl ether and hexanes and dried to afford the title compound as a tan solid (175 mg, 49%). ¹H NMR (400 MHz, DMSO-d6) δ ppm 13.00 (br. s., 1H), 9.86 (d, J=19.45 Hz, 2H), 8.96 (br. s., 1H), 7.68 (d, J=10.36 Hz, 1H), 7.50 (d, J=7.58 Hz, 1H), 7.31 (dd, J=7.71 Hz, 1H), 6.95 (d, J=1.77 Hz, 1H), 6.65-6.80 (m, 2H), 5.20 (s, 2H), 4.45 (d, J=5.81 Hz, 2 H), 4.09 (d, J=5.56 Hz, 2H), 3.75 (s, 3H).

### Example 21

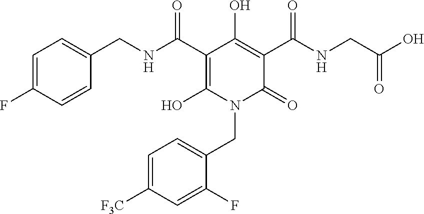

### N-[(5-({[(4-fluorophenyl)methyl]amino}carbonyl)-1-{[2-fluoro-4-(trifluoromethyl)phenyl]methyl}-4,6-dihydroxy-2-oxo-1,2-dihydro-3-pyridinyl)carbonyl]glycine

Methyl 5-({[2-(ethyloxy)-2-oxoethyl]amino}carbonyl)-1-{[2-fluoro-4-(trifluoromethyl)phenyl]methyl}-2,4-dihydroxy-6-oxo-1,6-dihydro-3-pyridinecarboxylate (300 mg, 0.612 mmoles) and 4-fluorobenzylamine (83 μL, 0.734 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution (3 mL) and stirred overnight. The mixture was diluted with ethyl acetate and washed with 1 molar hydrochloric acid (×2) and evaporated to a solid that was triturated in diethyl ether, collected, washed with diethyl ether and hexanes and dried to afford the title compound as a pale yellow solid (155 mg, 46%). ¹H NMR (400 MHz, DMSO-d6) δ ppm 12.99 (br. s., 1H), 10.01 (s, 1H), 9.82 (s, 1H), 7.69 (d, J=9.09 Hz, 1H), 7.50 (d, J=8.08 Hz, 1H), 7.40 (dd, J=8.59, 5.56

Hz, 2H), 7.32 (dd, J=7.71 Hz, 1H), 7.18 (t, J=8.97 Hz, 2H), 5.20 (s, 2H), 4.55 (d, J=5.81 Hz, 2 H), 4.09 (d, J=5.56 Hz, 2H).

### Example 22

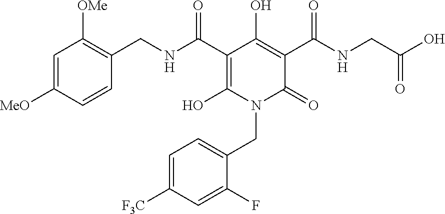

### N-[(5-[({[2,4-bis(methyloxy)phenyl]methyl}amino)carbonyl]-1-{[2-fluoro-4-(trifluoromethyl)phenyl]methyl}-4,6-dihydroxy-2-oxo-1,2-dihydro-3-pyridinyl)carbonyl]glycine

Methyl 5-({[2-(ethyloxy)-2-oxoethyl]amino}carbonyl)-1-{[2-fluoro-4-(trifluoromethyl)phenyl]methyl}-2,4-dihydroxy-6-oxo-1,6-dihydro-3-pyridinecarboxylate (300 mg, 0.612 mmoles) and 2,4-dimethoxybenzylamine (123 mg, 0.734 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution (3 mL) and stirred overnight. The mixture was diluted with ethyl acetate and washed with 1 molar hydrochloric acid (×2) and evaporated to a solid that was triturated in diethyl ether, collected, washed with diethyl ether and hexanes and dried to afford the title compound as a pale yellow solid (245 mg, 67%). ¹H NMR (400 MHz, DMSO-d6) δ ppm 13.01 (br. s., 1H), 9.87 (br. s., 2H), 7.69 (d, J=9.09 Hz, 1H), 7.50 (d, J=7.83 Hz, 1H), 7.31 (dd, J=7.71 Hz, 1H), 7.16 (d, J=8.34 Hz, 1H), 6.60 (d, J=2.53 Hz, 1H), 6.50 (dd, J=8.34, 2.27 Hz, 1H), 5.19 (s, 2H), 4.46 (d, J=5.56 Hz, 2H), 4.09 (d, J=5.31 Hz, 2H), 3.82 (s, 3H), 3.75 (s, 3H).

### Example 23

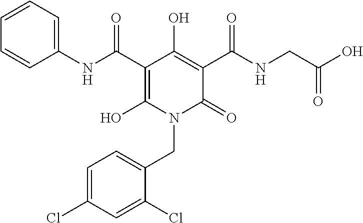

### N-({1-[(2,4-dichlorophenyl)methyl]-4,6-dihydroxy-2-oxo-5-[(phenylamino)carbonyl]-1,2-dihydro-3-pyridinyl}carbonyl)glycine

23a) 7-{[(2,4-Dichlorophenyl]methyl]-amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (4.0 g, 17.35 mmoles) was taken up in chloroform (30 mL) and cooled over an ice bath. N,N-Diisopropylethylamine (3.30 mL, 19.1 mmoles) in chloroform (25 mL) was added dropwise, followed by a solution of 2,4-dichlorobenzylamine (2.335 mL, 17.35 mmoles) in chloroform (25 mL). Cooling was removed and the mixture was stirred at room temperature for two hours. Addition of water gave a solid, and the mixture was stirred for 1 hour to ensure most product precipitated. The solid was collected, washed with water and azeotroped with methanol to give a free flowing powder, which was slurried in methanol, collected, washed with methanol and hexane, and dried in vacuo to afford the title compound (6.2 g, 97%). ¹H NMR (400 MHz, DMSO-d6) δ ppm 9.50 (s, 1H), 7.69 (s, 1H), 7.48 (s, 2H), 5.30 (d, J=32.08 Hz, 1H), 4.51 (d, J=30.06 Hz, 2 H), 1.64 (s, 6H).

23b) Methyl 1-[(2,4-dichlorophenyl)methyl]-2,4-dihydroxy-6-oxo-1,6-dihydro-3-pyridinecarboxylate. Sodium (1.8 g, 78 mmoles) was dissolved in dry methanol under nitrogen. 7-{[(2,4-dichlorophenyl)methyl]amino}-2,2-dimethyl-4H,5H-pyrano[4,3-d][1,3]dioxin-4,5-dione (6.2 g, 16.75 mmoles) was added and the mixture was heated at 65° C. for 3 hours. The mixture was evaporated to low volume and partitioned between ethyl acetate and 1 molar hydrochloric acid. A solid precipitated immediately and was collected and well washed with water, ethanol and hexane to afford the title compound (4.5 g 78%). ¹H NMR (400 MHz, DMSO-d6) δ ppm 12.93 (s, 1H), 7.64 (s, 1H), 7.36 (d, J=6.32 Hz, 1H), 6.80 (d, J=8.08 Hz, 1H), 5.30 (s, 1H), 5.06 (s, 2H), 3.73 (s, 3H).

23c) Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate. A mixture of 7-{[(2,4-dichlorophenyl)methyl]amino}-2,2-dimethyl-4H,5H-pyrano[4,3-d][1,3]dioxin-4,5-dione (4.45 g, 12.93 mmoles), N,N-diisopropylethylamine (2.68 mL, 15.5 mmoles) and ethyl isocyanatoacetate (1.74 mL, 15.5 mmoles) in chloroform (100 mL) was sealed in a pressure flask under nitrogen and heated at 110° C. for 3 hours. LCMS showed that the reaction was complete. The mixture was washed with 1 molar hydrochloric acid (×2), dried and evaporated to a light brown solid. The solid was triturated with diethyl ether, collected, and washed with ether and hexanes to give the title compound (4.87 g, 80%). ¹H NMR (400 MHz, DMSO-d6) δ ppm 10.12 (s, 1H), 7.60 (s, 1H), 7.34 (dd, J=8.46, 2.15 Hz, 1H), 6.81 (d, J=8.34 Hz, 1H), 5.04 (s, 2H), 4.11 (q, J=7.16 Hz, 2H), 4.02 (d, J=5.56 Hz, 2H), 3.63 (s, 3H), 1.19 (t, J=7.07 Hz, 3H).

23d) N-({1-[(2,4-dichlorophenyl)methyl]-4,6-dihydroxy-2-oxo-5-[(phenylamino)carbonyl]-1,2-dihydro-3-pyridinyl}carbonyl)glycine. Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (310 mg, 0.655 mmoles) and aniline (72 μL, 0.786 mmoles) in 1,4-dioxane (3 mL) were sealed in a pressure tube and heated in a microwave reactor at 150° C. for 30 minutes. LCMS indicated the reaction was complete. The reaction mixture was treated with 1 molar sodium hydroxide solution (3 mL) and stirred overnight. The mixture was diluted with ethyl acetate and washed with 1 molar hydrochloric acid (×2) and evaporated to give a solid that was triturated in ethanol collected, washed with diethyl ether and hexanes, and dried to afford the title compound as a pale yellow solid (200 mg, 60%). ¹H NMR (400 MHz, DMSO-d6) δ ppm 13.10 (br. s., 1H), 11.72 (s, 1H), 9.70 (s, 1H), 7.67 (d, J=2.27 Hz, 1H), 7.57 (d, J=7.58 Hz, 2H), 7.28-7.46 (m, 3H), 7.19 (dd, J=7.33 Hz, 1H), 7.02 (d, J=8.34 Hz, 1H), 5.16 (s, 2H), 4.12 (d, J=2.27 Hz, 2H).

### Example 24

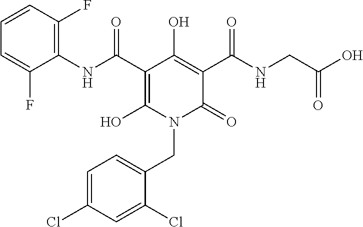

### N-[(1-[(2,4-dichlorophenyl)methyl]-5-{[(2,6-difluorophenyl)amino]carbonyl}-4,6-dihydroxy-2-oxo-1,2-dihydro-3-pyridinyl)carbonyl]glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (310 mg, 0.655 mmoles) and 2,6-difluoroaniline (79 μL, 0.786 mmoles) in 1,4-dioxane (3 mL) were sealed in a pressure tube and heated in a microwave reactor at 150° C. for 30 minutes. LCMS indicated the reaction was complete. The mixture was treated with 1 molar sodium hydroxide solution (3 mL) and stirred overnight. The mixture was diluted with ethyl acetate and washed with 1 molar hydrochloric acid (×2) and evaporated to a solid that was triturated in ethanol collected, washed with diethyl ether and hexanes, and dried to afford the title compound as a pale yellow solid (76 mg, 21%). ¹H NMR (400 MHz, DMSO-d6) δ ppm 12.93 (br. s., 1H), 11.06 (s, 1H), 9.72 (s, 1H), 7.67 (s, 1H), 7.42 (ddd, J=14.91, 8.34, 6.32 Hz, 1H), 7.35 (dd, J=8.46, 2.15 Hz, 1H), 7.22 (dd, J=8.08 Hz, 2H), 7.04 (d, J=8.34 Hz, 1H), 5.16 (s, 2H), 4.11 (s, 2H).

### Example 25

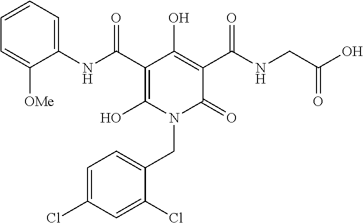

### N-{[1-[(2,4-dichlorophenyl)methyl]-4,6-dihydroxy-5-({[2-(methyloxy)phenyl]amino}carbonyl)-2-oxo-1,2-dihydro-3-pyridinyl]carbonyl}glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmoles) and 2-anisidine (57 μL, 0.507 mmoles) in chloroform (4 mL) were sealed in a pressure tube and heated in a microwave reactor at 150° C. for 30 minutes. LCMS indicated the reaction was complete. The mixture was washed with 1 molar hydrochloric acid, evaporated, taken up in ethanol and treated with 6 molar sodium hydroxide solution and stirred for 3 hours. The mixture was acidified, giving a solid that was collected, washed with water then ethanol and hexane. The solid was dried in vacuo to give the title compound (172 mg, 76%). ¹H NMR (400 MHz, DMSO-d6) δ ppm 13.07 (br. s., 1H), 11.94 (s, 1H), 9.69 (s, 1H), 8.13 (d, J=7.83 Hz, 1H), 7.68 (d, J=2.27 Hz, 1H), 7.33 (dd, J=8.46, 2.15 Hz, 1H), 7.06-7.24 (m, 2H), 6.85-7.07 (m, 2H), 5.15 (s, 2H), 4.11 (d, J=3.54 Hz, 2H), 3.86 (s, 3H).

### Example 26

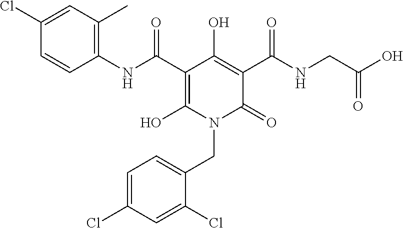

### N-({5-{[(4-chloro-2-methylphenyl)amino]carbonyl}-1-[(2,4-dichlorophenyl)methyl]-4,6-dihydroxy-2-oxo-1,2-dihydro-3-pyridinyl}carbonyl)glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmoles) and 2-methyl-4-chloroaniline (60 μL, 0.507 mmoles) in chloroform (4 mL) were sealed in a pressure tube and heated in a microwave reactor at 150° C. for 30 minutes. LCMS indicated the reaction was complete. The mixture was washed with hydrochloric acid, evaporated, taken up in ethanol and treated with 6 molar sodium hydroxide solution. The mixture was stirred overnight, then acidified to give a solid that was collected, washed with water, ethanol and hexanes. The solid was dried in vacuo to give the title compound (152 mg, 65%). ¹H NMR (400 MHz, DMSO-d6) δ ppm 12.98 (br. s., 1H), 11.64 (s, 1H), 9.72 (s, 1H), 7.91 (d, J=8.59 Hz, 1H), 7.67 (d, J=2.02 Hz, 1H), 7.39 (d, J=2.27 Hz, 1H), 7.34 (dd, J=8.46, 2.15 Hz, 1H), 7.30 (dd, J=8.72, 2.40 Hz, 1H), 7.01 (d, J=8.34 Hz, 1H), 5.15 (s, 2H), 4.11 (s, 2H), 2.27 (s, 3H).

### Example 27

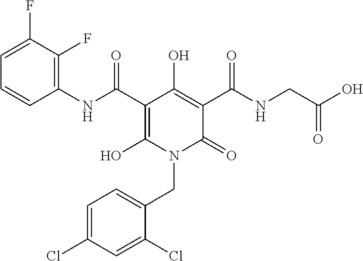

### N-[(1-[(2,4-dichlorophenyl)methyl]-5-{[(2,3-difluorophenyl)amino]carbonyl}-4,6-dihydroxy-2-oxo-1,2-dihydro-3-pyridinyl)carbonyl]glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (218 mg, 0.461 mmoles) and 2,3-difluoroaniline (56 μL, 0.553 mmoles) in chloroform (4 mL) were sealed in a pressure tube and heated in a microwave reactor at 150° C. for 30 minutes. LCMS indicated the reaction was complete. The ester crystallized on standing overnight. The crystals were collected and washed with dichloromethane. The solid was taken up in ethanol and treated with 6 molar sodium hydroxide solution, and stirred for 4 hours. The mixture gave a solid on acidification. It was collected, washed with water, and ethanol and hexanes and dried in vacuo to give the title compound (160 mg, 64%). ¹H NMR (400 MHz, DMSO-d6) δ ppm 12.20 (s, 1H), 9.79 (s, 1H), 7.84-8.18 (m, 1H), 7.67 (d, J=2.27 Hz, 1H), 7.34 (dd, J=8.46, 2.15 Hz, 1H), 7.09-7.28 (m, 2 H), 6.97 (d, J=8.59 Hz, 1H), 5.16 (s, 2H), 4.09 (s, 2H).

### Example 28

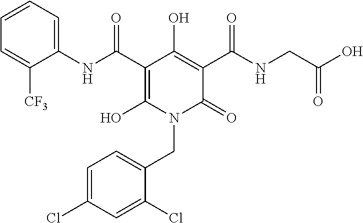

### N-{[1-[(2,4-dichlorophenyl)methyl]-4,6-dihydroxy-2-oxo-5-({[2-(trifluoromethyl)phenyl]amino}carbonyl)-1,2-dihydro-3-pyridinyl]carbonyl}glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (315 mg, 0.666 mmoles) and 2-(trifluoromethyl)aniline (99 μL, 0.799 mmoles) in chloroform (4 mL) were sealed in a pressure tube and heated in a microwave reactor at 150° C. for 30 minutes. LCMS indicated the reaction was complete. The mixture was evaporated, slurried in ethanol and treated with 6 molar sodium hydroxide solution (5 mL) and stirred overnight. The mixture was diluted with ethyl acetate and washed with 1 molar hydrochloric acid (×2) and evaporated to a solid that was recrystallised from ethanol, collected, washed with ethanol, hexanes and dried to afford a lilac solid (150 mg, 39%). ¹H NMR (400 MHz, DMSO-d6) δ ppm 12.94 (br. s., 1H), 11.93 (s, 1H), 9.71 (s, 1H), 8.00 (d, J=8.08 Hz, 1H), 7.78 (d, J=7.83 Hz, 1H), 7.73 (dd, J=7.71 Hz, 1H), 7.67 (d, J=2.27 Hz, 1H), 7.44 (dd, J=7.71 Hz, 1H), 7.35 (dd, J=8.34, 2.02 Hz, 1H), 7.01 (d, J=8.34 Hz, 1H), 5.16 (s, 2H), 4.11 (s, 2H).

### Example 29

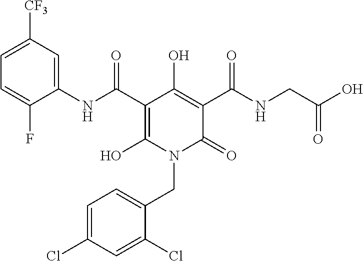

### N-{[1-[(2,4-dichlorophenyl)methyl]-5-({[2-fluoro-5-(trifluoromethyl)phenyl]amino}carbonyl)-4,6-dihydroxy-2-oxo-1,2-dihydro-3-pyridinyl]carbonyl}glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (305 mg, 0.644 mmoles) and 2-fluoro-5-(trifluoromethyl)aniline (101 μL, 0.773 mmoles) in chloroform (4 mL) were sealed in a pressure tube and heated in a microwave reactor at 150° C. for 30 minutes. LCMS indicated the reaction was complete. The mixture was evaporated, taken up in ethanol and treated with 1 molar sodium hydroxide solution (3 mL) and stirred overnight. The mixture was diluted with ethyl acetate and washed with 1 molar hydrochloric acid (×2) and evaporated to a solid that was recrystallised from ethanol. It was collected, washed with hexanes and dried to afford a pale pink solid (200 mg, 52%). ¹H NMR (400 MHz, DMSO-d6) δ ppm 12.71 (br. s., 1H), 12.41 (s, 1H), 9.82 (s, 1H), 8.68 (d, J=5.81 Hz, 1H), 7.66 (d, J=2.02 Hz, 1H), 7.45-7.58 (m, 2H), 7.34 (dd, J=8.46, 2.15 Hz, 1H), 6.94 (d, J=8.59 Hz, 1H), 5.15 (s, 2H), 4.08 (s, 2H).

### Example 30

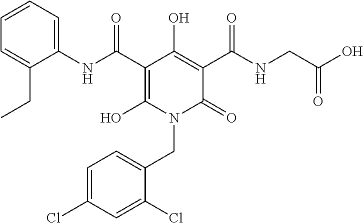

### N-[(1-[(2,4-dichlorophenyl)methyl]-5-{[(2-ethylphenyl)amino]carbonyl}-4,6-dihydroxy-2-oxo-1,2-dihydro-3-pyridinyl)carbonyl]glycine

Methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (210 mg, 0.444 mmoles) and 2-ethylaniline (66 μL, 0.532 mmoles) in chloroform (4 mL) were sealed in a pressure tube and heated in a microwave reactor at 150° C. for 30 minutes. LCMS indicated the reaction was complete. The mixture was evaporated, taken up in ethanol and treated with 6 molar sodium hydroxide solution. The mixture was stirred for several hours until hydrolysis was complete. The solution was acidified, extracted into ethyl acetate, washed with 1 molar hydrochloric acid, dried and evaporated to a solid. The solid was slurried in ethanol, collected, washed with ethanol and hexane to give the title compound (148 mg, 62%). ¹H NMR (400 MHz, DMSO-d6) δ ppm 13.04 (br. s., 1H), 11.58 (s, 1H), 9.76 (s, 1H), 7.78 (d, J=7.33 Hz, 1H), 7.68 (d, J=2.27 Hz, 1H), 7.35 (dd, J=8.46, 2.15 Hz, 1H), 7.31 (dd, J=7.45, 1.39 Hz, 1H), 7.26 (ddd, J=7.64, 1.64 Hz, 1H), 7.20 (ddd, J=7.45, 1.26 Hz, 1H), 7.03 (d, J=8.34 Hz, 1H), 5.17 (s, 2H), 4.12 (d, J=4.04 Hz, 2H), 2.63 (q, J=7.58 Hz, 2H), 1.16 (t, J=7.58 Hz, 3H).

### Example 31

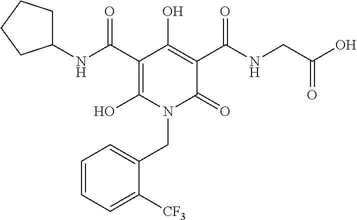

### N-[(5-[(cyclopentylamino)carbonyl]-4,6-dihydroxy-2-oxo-1-{[2-(trifluoromethyl)phenyl]methyl}-1,2-dihydro-3-pyridinyl)carbonyl]glycine

{[2-(Trifluoromethyl)phenyl]methyl}-1,6-dihydro-3-pyridinecarboxylate (236 mg, 0.5 mmoles) and cyclopentylamine (74 μL, 0.75 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give the title compound as a solid which recrystallized from ethanol to give the title compound (55 mg, 22%). ¹H NMR (400 MHz, DMSO-d6) δ ppm 13.01 (s, 1H), 9.85 (s, 1H), 9.59 (s, 1H), 7.77 (d, J=7.58 Hz, 1H), 7.58 (dd, J=7.71 Hz, 1H), 7.47 (dd, J=7.71 Hz, 1H), 5.28 (s, 2H), 4.22 (td, J=13.33, 6.95 Hz, 1H), 4.10 (d, J=5.56 Hz, 2H), 1.83-2.05 (m, 2H), 1.43-1.77 (m, 6H).

### Example 32

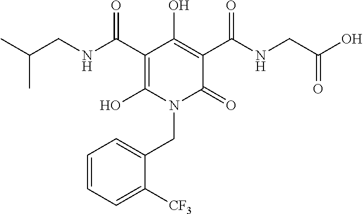

### N-[(4,6-dihydroxy-5-{[(2-methylpropyl)amino]carbonyl}-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (236 mg, 0.5 mmoles) and isobutylamine (75 μL, 0.75 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give the title compound as a solid which recrystallized from ethanol-hexane to give the title compound (50 mg, 21%). ¹H NMR (400 MHz, DMSO-d6) δ ppm 12.99 (s, 1H), 9.87 (s, 1H), 9.67 (s, 1H), 7.77 (d, J=7.58 Hz, 1H), 7.58 (dd, J=7.71 Hz, 1H), 7.47 (dd, J=7.58 Hz, 1H), 7.02 (d, J=7.83 Hz, 1H), 5.29 (s, 2H), 4.09 (d, J=5.31 Hz, 2H), 3.22 (dd, J=6.32 Hz, 2H), 1.71-2.02 (m, J=19.77, 12.95, 6.44 Hz, 1H), 0.90 (d, J=6.57 Hz, 6H).

### Example 33

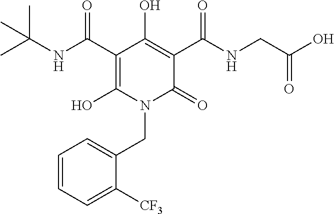

### N-[(5-{[(1,1-dimethylethyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (236 mg, 0.5 mmoles) and t-butylamine (79 μL, 0.75 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give the title compound as a solid which recrystallized from ethanol to give the title compound (115 mg, 47%). ¹H NMR (400 MHz, DMSO-d6) δ ppm 13.05 (br. s., 1H), 9.84 (s, 1H), 9.72 (s, 1H), 7.77 (d, J=7.58 Hz, 1H), 7.59 (dd, J=7.58 Hz, 1H), 7.47 (dd, J=7.58 Hz, 1H), 7.01 (d, J=7.83 Hz, 1H), 5.28 (s, 2H), 4.11 (d, J=5.56 Hz, 2H), 1.41 (s, 9H).

### Example 34

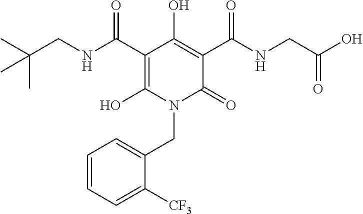

### N-[(5-{[(2,2-dimethylpropyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (236 mg, 0.5 mmoles) and neopentylamine (88 μL, 0.75 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give the title compound as a solid which recrystallized from ethanol-water to give the title compound (220 mg, 88%). ¹H NMR (400 MHz, DMSO-d6) δ ppm 13.01 (br. s., 1H), 9.86 (s, 1H), 9.74 (s, 1H), 7.77 (d, J=7.58 Hz, 1H), 7.59 (dd, J=7.45 Hz, 1H), 7.47 (dd, J=7.71 Hz, 1H), 7.03 (d, J=7.83 Hz, 1H), 5.29 (s, 2H), 4.10 (d, J=5.31 Hz, 2H), 3.22 (d, J=6.32 Hz, 2H), 0.93 (s, 9H).

### Example 35

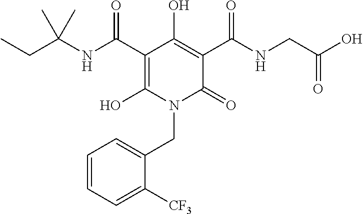

### N-[(5-{[(1,1-dimethylpropyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (236 mg, 0.5 mmoles) and t-amylamine (88 μL, 0.75 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give the title compound as a solid which recrystallized from acetic acid-water to give the title compound (210 mg, 84%). ¹H NMR (400 MHz, DMSO-d6) δ ppm 13.01 (br. s., 1H), 9.85 (s, 1H), 9.68 (s, 1H), 7.77 (d, J=7.58 Hz, 1H), 7.59 (dd, J=7.45 Hz, 1H), 7.47 (dd, J=7.58 Hz, 1H), 7.02 (d, J=7.83 Hz, 1H), 5.28 (s, 2 H), 4.11 (d, J=5.56 Hz, 2H), 1.74 (q, J=7.33 Hz, 2H), 1.37 (s, 6H), 0.87 (t, J=7.45 Hz, 3H).

### Example 36

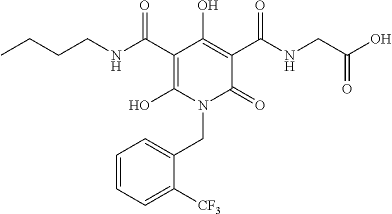

### N-[(5-[(butylamino)carbonyl]-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmoles) and n-butylamine (95 μL, 0.94 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, the aqueous extracted with ethyl acetate and the combined extracts washed with 1 molar hydrochloric acid (×2). The solution was dried and evaporated to give the title compound as an oil which crystallized from ethanol to give the title compound (160 mg, 52%). ¹H NMR (400 MHz, DMSO-d6) δ ppm 12.99 (s, 1H), 9.88 (s, 1H), 9.62 (s, 1H), 7.77 (d, J=7.58 Hz, 1H), 7.58 (dd, J=7.45 Hz, 1H), 7.47 (dd, J=7.58 Hz, 1H), 7.01 (d, J=7.83 Hz, 1H), 5.29 (s, 2H), 4.09 (d, J=5.56 Hz, 2H), 3.37 (dt, J=6.82 Hz, 2H), 1.54 (dt, J=14.53, 7.45, 7.33 Hz, 2H), 1.32 (tq, J=7.42, 7.20 Hz, 2H), 0.90 (t, J=7.33 Hz, 3H).

### Example 37

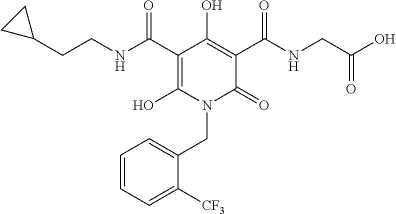

### N-[(5-{[(2-cyclopropylethyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmoles), diisopropylethylamine (165 μL, 0.953 mmoles) and 2-cyclopropylethylamine hydrochloride (116 mg, 0.953 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, the aqueous extracted with ethyl acetate and the combined extracts washed with 1 molar hydrochloric acid (×2). The solution was dried and evaporated to give the title compound as an oil that crystallized from ethanol to give the title compound (110 mg, 35%). ¹H NMR (400 MHz, DMSO-d6) δ ppm 12.96 (br. s., 1H), 9.88 (s, 1 H), 9.66 (s, 1H), 7.77 (d, J=7.58 Hz, 1H), 7.58 (dd, J=7.58 Hz, 1H), 7.47 (dd, J=7.58 Hz, 1H), 7.00 (d, J=7.83 Hz, 1H), 5.29 (s, 2H), 4.09 (d, J=5.56 Hz, 2H), 3.45 (dt, 2H), 1.47 (dt, J=7.07 Hz, 2H), 0.62-0.75 (m, 1H), 0.08 (dt, J=6.00, 4.48, 4.17 Hz, 2H).

### Example 38

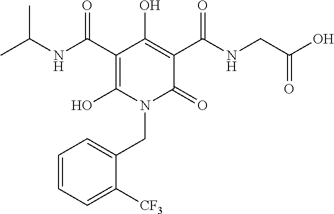

### N-[(4,6-dihydroxy-5-{[(1-methylethyl)amino]carbonyl}-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmoles) and isopropylamine (81 μL, 0.94 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, the aqueous extracted with ethyl acetate and the combined extracts washed with 1 molar hydrochloric acid (×2). The solution was dried and evaporated to give a solid that recrystallized from acetic acid to give the title compound (175 mg, 59%). ¹H NMR (400 MHz, DMSO-d6) δ ppm 13.00 (br. s., 1H), 9.85 (s, 1H), 9.47 (s, 1 H), 7.77 (d, J=7.58 Hz, 1H), 7.58 (dd, J=7.58 Hz, 1H), 7.47 (dd, J=7.58 Hz, 1H), 7.01 (d, J=7.83 Hz, 1H), 5.28 (s, 2H), 3.91-4.27 (m, 3H), 1.23 (d, J=6.57 Hz, 6H).

### Example 39

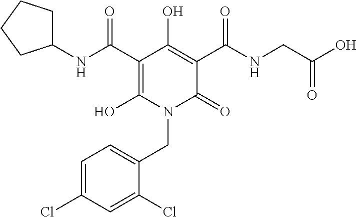

### N-({5-[cyclopentylamino)carbonyl]-1-[(2,4-dichlorophenyl)methyl]-4,6-dihydroxy-2-oxo-1,2-dihydro-3-pyridinyl}carbonyl)glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and cyclopentylamine (0.063 mL, 0.634 mmol) in chloroform (3.0 mL) was sealed in a pressure tube and heated in a microwave reactor at 150° C. for 30 minutes. The reaction mixture was washed with 1 molar hydrochloric acid (×2), dried and evaporated. The residue was dissolved in ethanol (2 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid, dried and evaporated to a solid that recrystallized from ethanol to give the title compound (180 mg, 85%). ¹H NMR (400 MHz, DMSO-d₆) δ ppm 13.03 (br. s., 1H), 9.84 (s, 1H), 9.58 (s, 1H), 7.67 (d, J=2.02 Hz, 1H), 7.33 (dd, J=8.34, 2.02 Hz, 1H), 6.95 (d, J=8.59 Hz, 1H), 5.10 (s, 2H), 4.22 (dt, J=13.01, 6.57 Hz, 1H), 4.10 (d, J=5.31 Hz, 2H), 1.84-2.13 (m, 2H), 1.34-1.81 (m, 6H).

### Example 40

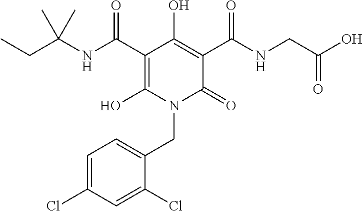

### N-[(1-[(2,4-dichlorophenyl)methyl]-5-{[(1,1-dimethylpropyl)amino]carbonyl}-4,6-dihydroxy-2-oxo-1,2-dihydro-3-pyridinyl)carbonyl]glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and t-amylamine (75 μL, 0.634 mmoles) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, and the organic layer then washed with 1 molar hydrochloric acid (×2). The solution was dried and evaporated to give a solid which recrystallized from ethanol to give the title compound (140 mg, 66%). ¹H NMR (400 MHz, DMSO-d₆) δ ppm 13.05 (br. s., 1H), 9.84 (s, 1H), 9.68 (s, 1H), 7.66 (d, J=2.02 Hz, 1H), 7.33 (dd, J=8.34, 2.27 Hz, 1H), 6.95 (d, J=8.34 Hz, 1H), 5.10 (s, 2H), 4.11 (d, J=5.31 Hz, 2H), 1.73 (q, J=7.33 Hz, 2H), 1.36 (s, 6H), 0.87 (t, J=7.45 Hz, 3H).

### Example 41

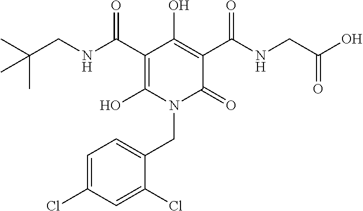

### N-[(1-[(2,4-dichlorophenyl)methyl]-5-{[(2,2-dimethylpropyl)amino]carbonyl}-4,6-dihydroxy-2-oxo-1,2-dihydro-3-pyridinyl)carbonyl]glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and neopentylamine (0.075 ml, 0.634 mmol) in chloroform (3.0 mL) was sealed in a pressure tube and heated in a microwave reactor at 150° C. for 30 minutes. The reaction mixture was washed with 1 molar hydrochloric acid, dried and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate, the combined organic layers then washed with 1 molar hydrochloric acid (×2). The solution was dried and evaporated to give a solid which recrystallized from ethanol to give the title compound (187 mg, 88%). ¹H NMR (400 MHz, DMSO-d6) δ ppm 13.01 (br. s., 1H), 9.85 (s, 1 H), 9.73 (s, 1H), 7.66 (s, 1H), 7.34 (d, J=8.34 Hz, 1H), 6.97 (d, J=8.34 Hz, 1H), 5.12 (s, 2H), 4.10 (d, J=4.80 Hz, 2H), 3.22 (d, J=5.81 Hz, 2H), 0.92 (s, 9H).

### Example 42

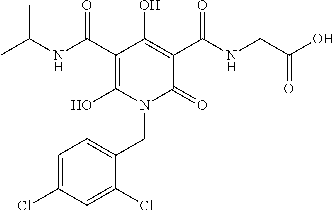

### N-[(1-[(2,4-dichlorophenyl)methyl]-4,6-dihydroxy-5-{[(1-methylethyl)amino]carbonyl}-2-oxo-1,2-dihydro-3-pyridinyl)carbonyl]glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and isopropylamine (0.054 mL, 0.634 mmol) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid (×2). The solution was dried and evaporated to give a solid that recrystallized from ethanol to give the title compound (150 mg, 75%). ¹H NMR (400 MHz, DMSO-d₆) δ ppm 13.03 (br. s., 1H), 9.84 (s, 1H), 9.46 (s, 1 H), 7.66 (d, J=2.02 Hz, 1H), 7.33 (dd, J=8.34, 2.27 Hz, 1H), 6.95 (d, J=8.34 Hz, 1H), 5.10 (s, 2 H), 3.98-4.31 (m, J=5.56 Hz, 3H), 1.22 (d, J=6.57 Hz, 6H).

### Example 43

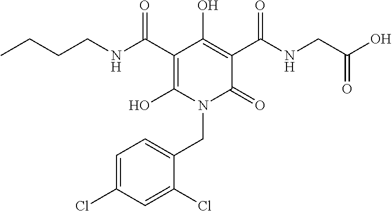

### N-({5-[(butylamino)carbonyl]-1-[(2,4-dichlorophenyl)methyl]-4,6-dihydroxy-2-oxo-1,2-dihydro-3-pyridinyl}carbonyl)glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and n-butylamine (0.063 ml, 0.634 mmol) in chloroform (3.0 mL) was sealed in a pressure tube and heated in a microwave reactor at 150° C. for 30 minutes. The reaction mixture was washed with 1 molar hydrochloric acid, dried and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid (×2). The solution was dried and evaporated to give a solid which crystallized from ethanol to give the title compound (155 mg, 75%). ¹H NMR (400 MHz, DMSO-d6) δ ppm 13.02 (br. s., 1H), 9.87 (s, 1 H), 9.61 (s, 1H), 7.66 (d, J=2.02 Hz, 1H), 7.33 (dd, J=8.46, 2.15 Hz, 1H), 6.95 (d, J=8.34 Hz, 1 H), 5.11 (s, 2H), 4.09 (d, J=5.56 Hz, 2H), 3.37 (dt, J=6.74 Hz, 2H), 1.54 (tt, 2H), 1.32 (tq, J=7.43 Hz, 2H), 0.90 (t, J=7.33 Hz, 3H).

### Example 44

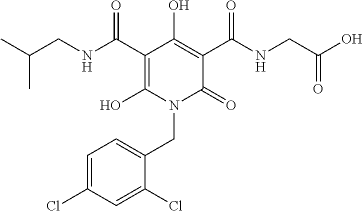

### N-[(1-[(2,4-dichlorophenyl)methyl]-4,6-dihydroxy-5-{[(2-methylpropyl)amino]carbonyl}-2-oxo-1,2-dihydro-3-pyridinyl)carbonyl]glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (200 mg, 0.423 mmol) and isobutylamine (0.063 ml, 0.634 mmol) in chloroform (3.0 mL) was sealed in a pressure tube and heated in a microwave reactor at 150° C. for 30 minutes. The reaction mixture was washed with 1 molar hydrochloric acid, dried and evaporated. The residue was dissolved in ethanol (5 mL) treated with 6 molar sodium hydroxide (3 ml) and stirred overnight. The mixture was partitioned between ethyl acetate and 1 molar hydrochloric acid. The aqueous layer was extracted with ethyl acetate and the combined organic solutions washed with 1 molar hydrochloric acid (×2), dried and evaporated to give a solid that recrystallized from ethanol to give the title compound (181 mg, 88%). ¹H NMR (400 MHz, DMSO-d6) δ ppm 13.01 (br. s., 1H), 9.87 (s, 1 H), 9.66 (s, 1H), 7.66 (d, J=2.02 Hz, 1H), 7.33 (dd, J=8.34, 2.02 Hz, 1H), 6.96 (d, J=8.34 Hz, 1 H), 5.12 (s, 2H), 4.10 (d, J=5.31 Hz, 2H), 3.22 (dd, J=6.44 Hz, 2H), 1.71-1.95 (m, J=20.08, 13.39, 6.69 Hz, 1H), 0.90 (d, J=6.57 Hz, 6H).

### Example 45

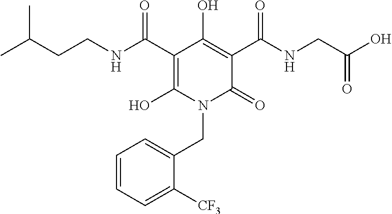

### N-[(4,6-dihydroxy-5-{[(3-methylbutyl)amino]carbonyl}-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (280 mg, 0.593 mmoles) and isoamylamine (0.103 mL, 0.889 mmol) in chloroform (3.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred for 2 hours. The mixture was partitioned between ethyl acetate and 1 molar hydrochloric acid, the organic solution washed with 1 molar hydrochloric acid dried and evaporated. Recrystallization from ethanol-water gave the title compound (205 mg, 69%). ¹H NMR (400 MHz, DMSO-d6) δ ppm 12.99 (br. s., 1H), 9.87 (s, 1H), 9.60 (s, 1H), 7.77 (d, J=7.58 Hz, 1H), 7.58 (dd, J=7.58 Hz, 1H), 7.47 (dd, J=7.58 Hz, 1H), 7.01 (d, J=7.83 Hz, 1H), 5.28 (s, 2H), 4.09 (d, J=5.31 Hz, 2H), 3.39 (dt, J=6.57 Hz, 2H), 1.51-1.69 (m, 1H), 1.45 (dt, J=7.07 Hz, 2H), 0.90 (d, J=6.57 Hz, 6H).

### Example 46

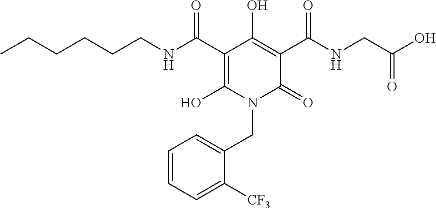

### N-[(5-[(hexylamino)carbonyl]-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (280 mg, 0.593 mmoles) and hexylamine (0.117 mL, 0.889 mmol) in chloroform (3.0 mL) was sealed in a pressure tube and heated in a microwave reactor at 150° C. for 30 minutes. The reaction mixture was washed with 1 molar hydrochloric acid, dried and evaporated. The residue was dissolved in ethanol (5 mL) treated with 6 molar sodium hydroxide (2 ml) and stirred overnight. The mixture was partitioned between ethyl acetate and 1 molar hydrochloric acid, the organic solution washed with 1 molar hydrochloric acid, dried and evaporated. Recrystallization from ethanol-water gave the title compound (210 mg, 69%). ¹H NMR (400 MHz, DMSO-d6) δ ppm 12.99 (br. s., 1H), 9.88 (s, 1 H), 9.62 (s, 1H), 7.77 (d, J=7.58 Hz, 1H), 7.58 (dd, J=7.45 Hz, 1H), 7.47 (dd, J=7.71 Hz, 1H), 7.01 (d, J=7.83 Hz, 1H), 5.28 (s, 2H), 4.09 (d, J=5.56 Hz, 2H), 3.36 (dt, J=6.74 Hz, 2H), 1.44-1.68 (m, 2H), 1.12-1.41 (m, 6H), 0.71-0.96 (m, 3H).

### Example 47

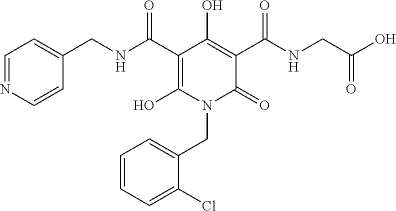

### N-[(1-[(2-chlorophenyl)methyl]-4,6-dihydroxy-2-oxo-5-{[(4-pyridinylmethyl)amino]carbonyl}-1,2-dihydro-3-pyridinyl)carbonyl]glycine

(4-Pyridinylmethyl)amine (80 mg, 738 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 216 mg, 492 mmol) in chloroform (3 ml). The solution was heated in a microwave (150° C., 30 min) After dilution with chloroform (15 ml), the mixture was washed with 1N HCl (3 ml, 2×). The chloroform was removed in-vacuo and the residue was dissolved in ethanol (7 ml) and treated with 6N NaOH (4 ml) and stirred at rt. Upon completion of the saponification the solution was cooled and made acidic to Litmus by addition of 6N HCl. The solution was cooled and stirred until precipitation was complete. The solid was washed with water, ethanol and diethyl ether then dried in-vacuo to yield the title compound as a white solid. Yield: 192 mg, 79.0%, MS (ES+): [M+H]⁺=487.1; ¹H NMR (400 MHz, DMSO-d₆) δ ppm 4.08 (d, J=5.05 Hz, 2H), 4.73 (d, J=5.05 Hz, 2H), 5.17 (s, 2H), 6.93 (dd, J=7.07, 2.02 Hz, 1H), 7.28 (td, J=4.93, 2.27 Hz, 2H), 7.48 (dd, J=7.33, 1.77 Hz, 1H), 7.73 (br. s., 2H), 8.73 (d, J=5.05 Hz, 2H), 9.81 (br. s., 1H), 10.11 (br. s., 1H), 13.00 (br. s., 1H).

### Example 48

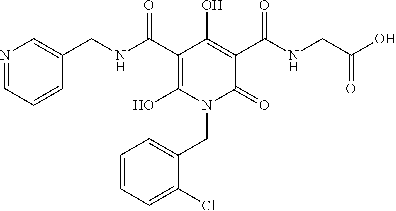

### N-[(1-[(2-chlorophenyl)methyl]-4,6-dihydroxy-2-oxo-5-{[(3-pyridinylmethyl)amino]carbonyl}-1,2-dihydro-3-pyridinyl)carbonyl]glycine

(3-Pyridinylmethyl)amine (80 mg, 0.738 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 216 mg, 0.492 mmol) in chloroform (3 ml). The solution was heated in a microwave (150° C., 30 min) After dilution with chloroform (15 ml) the mixture was washed with 1N HCl (3 ml, 2×). The chloroform was removed in-vacuo and the residue was dissolved in ethanol (7 ml) and treated with 6N NaOH (4 ml) and stirred at rt. Upon completion of the saponification the solution was diluted with ethyl acetate (15 ml) and washed with 1N HCl (6 ml, 3×). The organic was dried and concentrated to yield a pink-white solid. The solid was added to 15 ml EtOH and refluxed 10 min. After cooling, the solid was collected and dried in-vacuo to yield the title compound as an off white solid. Yield: 98 mg, 40.1%, MS (ES+): [M+H]⁺=487.1, ¹H NMR (400 MHz, DMSO-d₆) δ ppm 4.07 (d, J=5.56 Hz, 2H), 4.59 (d, J=5.81 Hz, 2H), 5.14 (s, 2H), 6.89 (dd, J=7.33, 1.52 Hz, 1H), 7.21-7.32 (m, J=7.33, 7.11, 6.99, 6.99, 1.64 Hz, 2H), 7.41-7.51 (m, 2H), 7.86 (d, J=7.83 Hz, 1H), 8.52 (d, J=3.79 Hz, 1H), 8.61 (s, 1H), 9.83 (br. s., 1H), 10.05 (br. s., 1H), 12.96 (br. s., 1H)

### Example 49

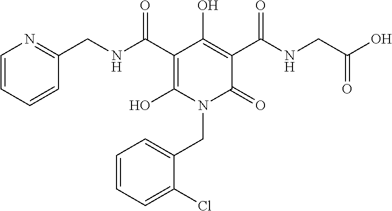

### N-[(1-[(2-chlorophenyl)methyl]-4,6-dihydroxy-2-oxo-5-{[(2-pyridinylmethyl)amino]carbonyl}-1,2-dihydro-3-pyridinyl)carbonyl]glycine

(2-Pyridinylmethyl)amine (80 mg, 0.738 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 216 mg, 0.492 mmol) in chloroform (3 ml). The solution was heated in a microwave (150° C., 30 min) After dilution with chloroform (15 ml) the mixture was washed with 1N HCl (3 ml, 2×). The chloroform was removed in-vacuo and the residue was dissolved in ethanol (7 ml) and treated with 6N NaOH (4 ml) and stirred at rt. Upon completion of the saponification the solution was cooled and made acidic to Litmus by addition of 6N HCl. The solution was cooled and stirred until precipitation was complete. The solid was washed with water, ethanol and diethyl ether then dried in-vacuo to yield the title compound as a white solid. Yield: 97 mg, 39.7%, MS (ES+): [M+H]⁺=487.1, ¹H NMR (400 MHz, DMSO-d₆) δ ppm 4.09 (d, J=5.56 Hz, 2H), 4.72 (d, J=5.56 Hz, 2H), 5.16 (s, 2H), 6.87-6.93 (m, 1H), 7.27 (qd, J=6.48, 6.32 Hz, 2 H), 7.34 (dd, J=7.33, 5.31 Hz, 1H), 7.42 (d, J=7.58 Hz, 1H), 7.49 (dd, J=7.33, 1.77 Hz, 1H), 7.83 (td, J=7.83, 1.52 Hz, 1H), 8.57 (d, J=4.55 Hz, 1H), 9.84 (br. s., 1H), 10.28 (br. s., 1H), 12.99 (br. s., 1H)

### Example 50

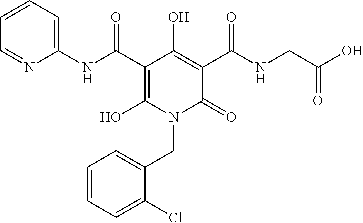

### N-({1-[(2-chlorophenyl)methyl]-4,6-dihydroxy-2-oxo-5-[(2-pyridinylamino)carbonyl]-1,2-dihydro-3-pyridinyl}carbonyl)glycine

2-Pyridinamine (64.3 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was cooled and made acidic to Litmus by addition of 6N HCl. The solution was cooled and stirred until precipitation was complete. The solid was recrystallized from hot ethanol to give an impure solid. Recrystallization from ethanol water did not improve purity. The remaining solid was dissolved in dimethyl sulfoxide (1.5 ml) purified by HPLC (0.1% TFA, acetonitrile, 0.1% TFA water) to yield the title compound as a white solid. Yield: 55 mg, 33.0%, MS (ES+): [M+H]⁺=473.1, ¹H NMR (400 MHz, DMSO-d₆) δ ppm 4.00 (d, J=5.31 Hz, 2H), 5.18 (s, 2H), 6.78-6.85 (m, 1H), 7.20-7.30 (m, 3H), 7.44-7.51 (m, 1H), 7.90 (br. s., 1H), 8.05 (br. s., 1H), 8.26 (d, J=5.05 Hz, 1H), 10.12 (br. s., 1H), 13.37 (br. s., 1H)

### Example 51

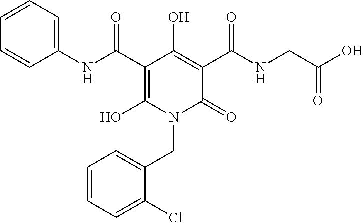

### N-({1-[(2-chlorophenyl)methyl]-4,6-dihydroxy-2-oxo-5-[(phenylamino)carbonyl]-1,2-dihydro-3-pyridinyl}carbonyl)glycine

Aniline (63.7 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 18 mg, 8.29%, MS (ES+): [M+H]⁺=472.1, ¹H NMR (400 MHz, DMSO-d₆) δ ppm 4.08 (d, J=4.04 Hz, 2H), 5.19 (s, 2H), 6.91 (d, J=7.07 Hz, 1H), 7.13 (t, J=7.33 Hz, 1H), 7.23-7.32 (m, J=7.45, 7.20, 7.07, 7.07 Hz, 2H), 7.36 (t, J=7.96 Hz, 2H), 7.49 (dd, J=7.33, 1.77 Hz, 1H), 7.58 (d, J=7.58 Hz, 2H), 9.83 (br. s., 1H), 11.79 (br. s., 1H), 12.99 (br. s., 1H)

### Example 52

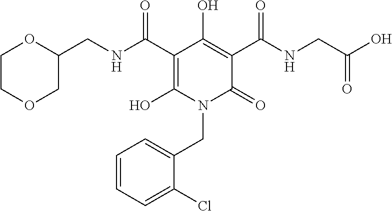

### N-[(1-[(2-chlorophenyl)methyl]-5-{[(1,4-dioxan-2-ylmethyl)amino]carbonyl}-4,6-dihydroxy-2-oxo-1,2-dihydro-3-pyridinyl)carbonyl]glycine

(1,4-dioxan-2-ylmethyl)amine (80 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 22 mg, 9.54%, MS (ES+): [M+H]⁺=496.1, ¹H NMR (400 MHz, DMSO-d₆) δ ppm 3.19-3.51 (m, 3H), 3.53-3.81 (m, 6H), 4.09 (d, J=5.31 Hz, 2H), 5.15 (s, 2H), 6.90 (d, J=7.33 Hz, 1H), 7.22-7.33 (m, J=7.29, 7.29, 7.29, 7.29, 1.64 Hz, 2H), 7.49 (dd, J=7.71, 1.64 Hz, 1H), 9.68 (br. s., 1H), 9.81 (br. s., 1H), 13.01 (br. s., 1 H)

### Example 53

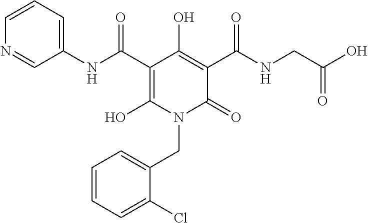

### N-({1-[(2-chlorophenyl)methyl]-4,6-dihydroxy-2-oxo-5-[(3-pyridinylamino)carbonyl]-1,2-dihydro-3-pyridinyl}carbonyl)glycine

3-pyridinamine (64.3 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 42 mg, 19.1%, MS (ES+): [M+H]⁺=473.1, ¹H NMR (400 MHz, DMSO-d₆) δ ppm 4.01 (br. s., 2H), 5.18 (s, 2H), 6.77-6.86 (m, 1H), 7.25 (dd, J=5.31, 2.53 Hz, 2H), 7.43-7.49 (m, 1H), 7.90 (dd, J=8.21, 5.94 Hz, 1H), 8.45-8.54 (m, 2H), 9.36 (s, 1H), 10.09 (br. s., 1H), 12.64 (s, 1H)

### Example 54

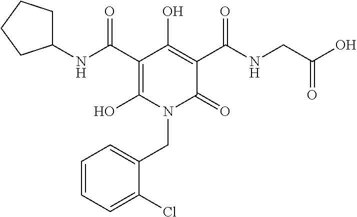

### N-({1-[(2-chlorophenyl)methyl]-5-[(cyclopentylamino)carbonyl]-4,6-dihydroxy-2-oxo-1,2-dihydro-3-pyridinyl}carbonyl)glycine

Cyclopentylamine (58 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 98 mg, 45.0%, MS (ES+): [M+H]⁺=464.1, ¹H NMR (400 MHz, DMSO-d₆) δ ppm 1.25-1.45 (m, J=12.25, 6.32, 6.32, 6.19 Hz, 2H), 1.44-1.72 (m, 4H), 1.72-2.00 (m, 2H), 3.87 (d, J=4.80 Hz, 2H), 4.00-4.20 (m, J=6.69, 6.69, 6.69, 6.69, 6.57 Hz, 1H), 5.09 (s, 2H), 6.70 (dd, J=5.43, 3.92 Hz, 1H), 7.23 (dd, J=5.81, 3.28 Hz, 2H), 7.40-7.47 (m, 1H), 9.99 (br. s., 2H), 12.54 (br. s., 1H)

### Example 55

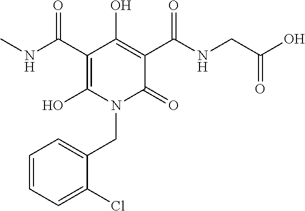

### N-({1-[(2-chlorophenyl)methyl]-4,6-dihydroxy-5-[(methylamino)carbonyl]-2-oxo-1,2-dihydro-3-pyridinyl}carbonyl)glycine

Methylamine hydrochloride (46.2 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) and diisopropylethylamine (88 mg, 0.684 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 120 mg, 63.6%, MS (ES+): [M+H]⁺=410.1, ¹H NMR (400 MHz, DMSO-d₆) δ ppm 2.91 (d, J=4.55 Hz, 3 H), 4.08 (d, J=5.56 Hz, 2H), 5.15 (s, 2H), 6.88 (dd, J=7.33, 1.77 Hz, 1H), 7.21-7.34 (m, J=7.39, 7.18, 7.18, 7.18, 1.64 Hz, 2H), 7.48 (dd, J=7.71, 1.64 Hz, 1H), 9.49 (br. s., 1H), 9.89 (br. s., 1H), 12.99 (br. s., 1H)

### Example 56

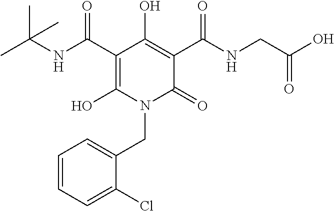

### N-[(1-[(2-chlorophenyl)methyl]-5-{[(1,1-dimethylethyl)amino]carbonyl}-4,6-dihydroxy-2-oxo-1,2-dihydro-3-pyridinyl)carbonyl]glycine

(1,1-Dimethylethyl)amine (50 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 98 mg, 47.0%, MS (ES+): [M+H]⁺=452.1, ¹H NMR (400 MHz, DMSO-d₆) δ ppm 1.41 (s, 9H), 2.08 (s, 1H), 4.11 (d, J=5.31 Hz, 2H), 5.13 (s, 2H), 6.89 (dd, J=7.33, 1.52 Hz, 1H), 7.16-7.38 (m, J=7.45, 7.17, 7.03, 7.03, 1.64 Hz, 2 H), 7.49 (dd, J=7.58, 1.52 Hz, 1H), 9.73 (br. s., 1H), 9.83 (br. s., 1H), 13.06 (br. s., 1H)

### Example 57

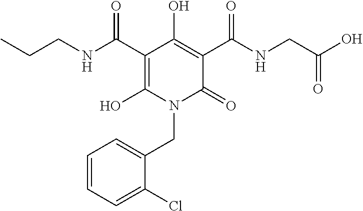

### N-({1-[(2-chlorophenyl)methyl]-4,6-dihydroxy-2-oxo-5-[(propylamino)carbonyl]-1,2-dihydro-3-pyridinyl}carbonyl)glycine

n-Propylamine (40 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 150 mg, 74.4%, MS (ES+): [M+H]⁺=438.1, ¹H NMR (400 MHz, DMSO-d₆) δ ppm 0.89 (t, J=7.45 Hz, 3H), 1.48-1.64 (m, J=7.45, 7.23, 7.23, 7.23, 7.23 Hz, 2H), 3.33 (q, J=6.57 Hz, 2H), 4.09 (d, J=5.31 Hz, 2H), 5.15 (s, 2H), 6.89 (dd, J=7.33, 1.77 Hz, 1H), 7.19-7.36 (m, J=7.52, 7.20, 7.04, 7.04, 1.77 Hz, 2H), 7.48 (dd, J=7.71, 1.64 Hz, 1H), 9.63 (br. s., 1H), 9.88 (br. s., 1H), 13.01 (br. s., 1H)

### Example 58

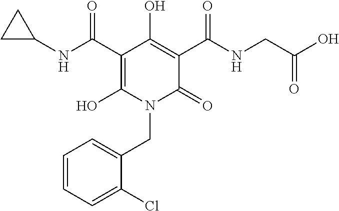

### N-({1-[(2-chlorophenyl)methyl]-5-[(cyclopropylamino)carbonyl]-4,6-dihydroxy-2-oxo-1,2-dihydro-3-pyridinyl}carbonyl)glycine

Cyclopropylamine (29 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 36 mg, 23.9%, MS (ES+): [M+H]⁺=436.1, ¹H NMR (400 MHz, DMSO-d₆) δ ppm 0.63-0.72 (m, 2H), 0.74-0.83 (m, 2H), 2.89 (td, J=7.26, 3.92 Hz, 1H), 4.08 (d, J=5.31 Hz, 2H), 5.13 (s, 2H), 6.87 (dd, J=7.33, 1.52 Hz, 1H), 7.22-7.33 (m, J=7.29, 7.29, 7.29, 7.29, 1.64 Hz, 2H), 7.48 (dd, J=7.45, 1.64 Hz, 1H), 9.50 (br. s., 1H), 9.84 (br. s., 1H), 13.00 (br. s., 1H)

### Example 59

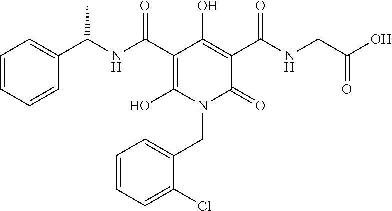

### N-{[1-[(2-chlorophenyl)methyl]-4,6-dihydroxy-2-oxo-5-({[(1S)-1-phenylethyl]amino}carbonyl)-1,2-dihydro-3-pyridinyl]carbonyl}glycine

(1S)-1-phenylethanamine (62 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 63 mg, 36.5%, MS (ES+): [M+H]⁺=500.1, ¹H NMR (400 MHz, DMSO-d₆) δ ppm 1.53 (d, J=6.82 Hz, 3H), 4.10 (d, J=5.56 Hz, 2H), 5.06-5.21 (m, 3H), 6.90 (dd, J=7.45, 1.64 Hz, 1H), 7.20-7.33 (m, 3H), 7.33-7.43 (m, 4H), 7.48 (dd, J=7.71, 1.64 Hz, 1H), 9.77 (br. s., 1H), 9.97 (br. s., 1H), 13.08 (br. s., 1H)

### Example 60

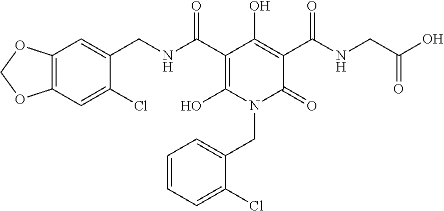

### N-({5-({[(6-chloro-1,3-benzodioxol-5-yl)methyl]amino}carbonyl)-1-[(2-chlorophenyl)methyl]-4,6-dihydroxy-2-oxo-1,2-dihydro-3-pyridinyl}carbonyl)glycine

[(6-Chloro-1,3-benzodioxol-5-yl)methyl]amine (95 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 95 mg, 48.8%, MS (ES+): [M+H]⁺=564.1, ¹H NMR (400 MHz, DMSO-d₆) δ ppm 4.08 (d, J=5.31 Hz, 2H), 4.52 (d, J=6.06 Hz, 2H), 5.14 (s, 2H), 6.06 (s, 2H), 6.90 (d, J=7.07 Hz, 1H), 6.99 (s, 1H), 7.11 (s, 1H), 7.21-7.32 (m, J=7.45, 7.23, 7.12, 7.12, 1.89 Hz, 2H), 7.48 (dd, J=7.45, 1.64 Hz, 1H), 9.81 (br. s., 1H), 9.94 (br. s., 1H), 13.00 (br. s., 1H)

### Example 61

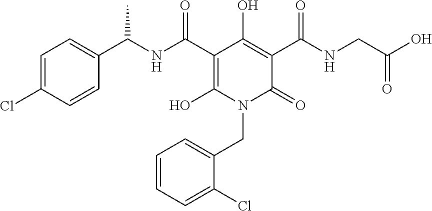

### N-({5-({[(1S)-1-(4-chlorophenyl)ethyl]amino}carbonyl)-1-[(2-chlorophenyl)methyl]-4,6-dihydroxy-2-oxo-1,2-dihydro-3-pyridinyl}carbonyl)glycine

[(1S)-1-(4-chlorophenyl)ethyl]amine (80 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 46 mg, 24.6%, MS (ES+): [M+H]⁺=534.1, ¹H NMR (400 MHz, DMSO-d₆) δ ppm 1.51 (d, J=6.82 Hz, 3H), 4.10 (d, J=5.31 Hz, 2H), 5.04-5.22 (m, 3H), 6.90 (d, J=7.33 Hz, 1H), 7.20-7.35 (m, J=7.56, 7.56, 7.56, 7.39, 1.52 Hz, 2 H), 7.42 (s, 4H), 7.48 (dd, J=7.71, 1.39 Hz, 1H), 9.75 (br. s., 1H), 9.95 (br. s., 1H), 13.02 (br. s., 1H)

### Example 62

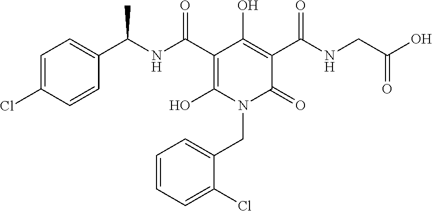

### N-({5-({[(1R)-1-(4-chlorophenyl)ethyl]amino}carbonyl)-1-[(2-chlorophenyl)methyl]-4,6-dihydroxy-2-oxo-1,2-dihydro-3-pyridinyl}carbonyl)glycine

[(1R)-1-(4-chlorophenyl)ethyl]amine (80 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 56 mg, 30.0%, MS (ES+): [M+H]⁺=534.1, ¹H NMR (400 MHz, DMSO-d₆) δ ppm 1.51 (d, J=7.07 Hz, 3H), 4.09 (d, J=5.31 Hz, 2H), 5.14 (s, 3H), 6.89 (d, J=7.33 Hz, 1H), 7.18-7.37 (m, J=7.42, 7.42, 7.42, 7.42, 1.64 Hz, 2H), 7.42 (s, 4H), 7.48 (dd, J=7.58, 1.52 Hz, 1H), 9.76 (br. s., 1H), 9.95 (br. s., 1H), 13.02 (br. s., 1H)

### Example 63

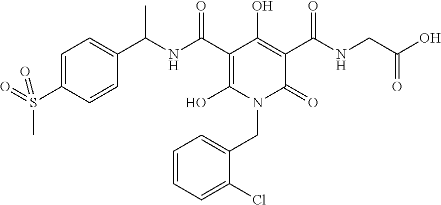

### N-({1-[(2-chlorophenyl)methyl]-4,6-dihydroxy-5-[({1-[4-(methylsulfonyl)phenyl]ethyl}amino) carbonyl]-2-oxo-1,2-dihydro-3-pyridinyl}carbonyl)glycine

1-[4-(methylsulfonyl)phenyl]ethanamine (102 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 136 mg, 68.1%, MS (ES+): [M+H]⁺=578.1, ¹H NMR (400 MHz, DMSO-d₆) δ ppm 1.56 (d, J=7.07 Hz, 3H), 3.21 (s, 3H), 4.10 (d, J=5.05 Hz, 2H), 5.15 (s, 2H), 5.23 (qd, 1H), 6.91 (dd, J=7.45, 1.64 Hz, 1H), 7.24-7.33 (m, J=7.58, 7.58, 7.45, 5.94 Hz, 2H), 7.49 (dd, J=7.71, 1.64 Hz, 1H), 7.67 (d, J=8.34 Hz, 2H), 7.92 (d, J=8.34 Hz, 2H), 9.73 (br. s., 1H), 10.02 (br. s., 1H), 13.05 (br. s., 1H)

### Example 64

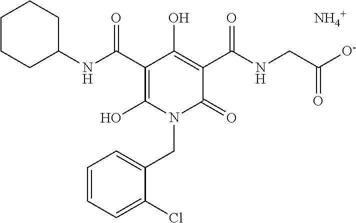

### Ammonium [({1-[(2-chlorophenyl)methyl]-5-[(cyclohexylamino)carbonyl]-4,6-dihydroxy-2-oxo-1,2-dihydro-3-pyridinyl}carbonyl)amino]acetate

Cyclohexylamine (68 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 122 mg, 55.5%, MS (ES+): [M+H]⁺=478.1, ¹H NMR (400 MHz, DMSO-d₆) δ ppm 0.96-1.40 (m, 5H), 1.52 (dd, J=8.46, 3.66 Hz, 1H), 1.62 (dd, J=8.97, 3.92 Hz, 2H), 1.79 (d, J=9.60 Hz, 2H), 3.69 (d, J=5.56 Hz, 1H), 3.85 (br. s., 2H), 5.09 (s, 2H), 6.70 (dd, J=5.68, 3.66 Hz, 1H), 7.29 (br. s., 4H), 7.19-7.25 (m, 2H), 7.41-7.46 (m, 1H), 9.82 (br. s., 1H), 10.20 (br. s., 1H)

### Example 65

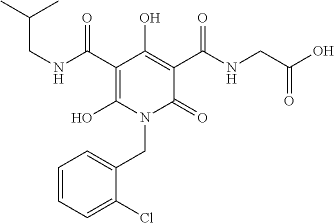

### N-[(1-[(2-chlorophenyl)methyl]-4,6-dihydroxy-5-{[(2-methylpropyl)amino]carbonyl}-2-oxo-1,2-dihydro-3-pyridinyl)carbonyl]glycine

(2-methylpropyl)amine (50 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 119 mg, 56.6%, MS (ES+): [M+H]⁺=452.1, ¹H NMR (400 MHz, DMSO-d₆) δ ppm 0.86 (d, J=6.57 Hz, 6H), 1.62-1.80 (m, J=13.33, 6.82, 6.67, 6.67, 6.67 Hz, 1H), 3.03 (t, J=5.81 Hz, 2H), 3.87 (d, J=4.80 Hz, 2 H), 5.10 (s, 2H), 6.72 (dd, J=5.56, 3.79 Hz, 1H), 7.15 (br. s., 4H), 7.20-7.27 (m, 2H), 7.40-7.46 (m, 1H), 9.94 (br. s., 1H), 12.53 (br. s., 1H)

### Example 66

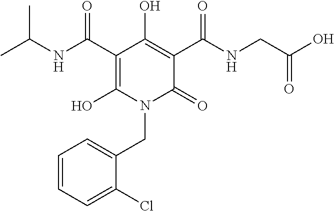

### N-[(1-[(2-chlorophenyl)methyl]-4,6-dihydroxy-5-{[(1-methylethyl)amino]carbonyl}-2-oxo-1,2-dihydro-3-pyridinyl)carbonyl]glycine

(1-methylethyl)amine (40 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 35 mg, 19.6%, MS (ES+): [M+H]⁺=438.1, ¹H NMR (400 MHz, DMSO-d₆) δ ppm 1.10 (d, J=6.57 Hz, 6H), 3.88 (d, J=5.05 Hz, 2H), 3.91-4.01 (m, 1H), 5.09 (s, 2H), 6.72 (dd, J=5.56, 3.79 Hz, 1H), 7.13 (br. s., 4 H), 7.23 (dd, J=5.68, 3.41 Hz, 2H), 7.39-7.47 (m, 1H), 9.95 (br. s., 1H), 12.56 (br. s., 1H)

### Example 67

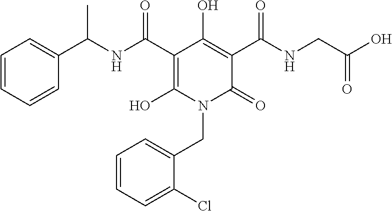

### N-[(1-[(2-chlorophenyl)methyl]-4,6-dihydroxy-2-oxo-5-{[(1-phenylethyl)amino]carbonyl}-1,2-dihydro-3-pyridinyl)carbonyl]glycine

(1-Phenylethyl)amine (62 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 65 mg, 37.7%, MS (ES+): [M+H]⁺=500.1, ¹H NMR (400 MHz, DMSO-d₆) δ ppm 1.39 (d, J=6.82 Hz, 3H), 3.90 (d, J=5.05 Hz, 2H), 5.03 (qd, J=7.12, 6.95 Hz, 1H), 5.11 (s, 2H), 6.74 (dd, J=5.31, 3.79 Hz, 1H), 6.95-7.18 (m, 4H), 7.23 (dd, J=6.06, 3.28 Hz, 4H), 7.32 (d, J=4.29 Hz, 3H), 7.41-7.48 (m, 1 H), 10.02 (br. s., 1H), 12.59 (br. s., 1H)

### Example 68

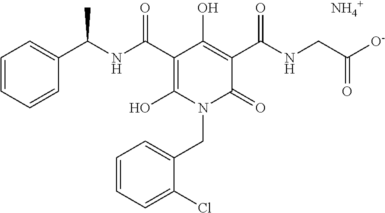

### Ammonium ({[1-[(2-chlorophenyl)methyl]-4,6-dihydroxy-2-oxo-5-({[(1R)-1-phenylethyl]amino}carbonyl)-1,2-dihydro-3-pyridinyl]carbonyl}amino)acetate

[(1R)-1-phenylethyl]amine (62 mg, 0.684 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 200 mg, 0.456 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 85 mg, 49.7%, MS (ES+): [M+H]⁺=500.1, ¹H NMR (400 MHz, DMSO-d₆) δ ppm 1.38 (d, J=6.06 Hz, 3H), 3.87 (br. s., 2H), 5.02 (quin, J=7.01 Hz, 1H), 5.11 (s, 2H), 6.66-6.77 (m, 1H), 7.02 (br. s., 4H), 7.23 (dd, J=5.81, 3.54 Hz, 3H), 7.31 (d, J=4.29 Hz, 4H), 7.41-7.48 (m, 1H), 9.85 (d, J=9.85 Hz, 1H), 10.63 (br. s., 1H)

### Example 69

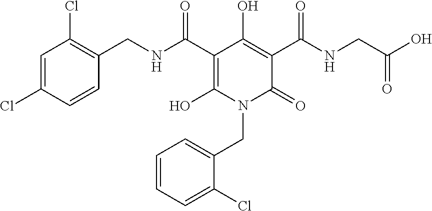

### N-{[1-[(2-chlorophenyl)methyl]-5-({[(2,4-dichlorophenyl)methyl]amino}carbonyl)-4,6-dihydroxy-2-oxo-1,2-dihydro-3-pyridinyl]carbonyl}glycine

1-(2,4-dichlorophenyl)methylamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 20 mg, 9.62%, MS (ES+): [M+H]⁺=554.0, ¹H NMR (400 MHz, DMSO-d₆) δ ppm 3.71 (d, J=4.55 Hz, 2H), 4.42 (br. s., 2H), 5.12 (s, 2H), 6.73 (dd, J=5.68, 3.66 Hz, 1H), 7.17-7.28 (m, 2H), 7.34-7.49 (m, 3H), 7.58 (s, 1H), 9.96 (br. s., 1 H), 10.25 (br. s., 1H)

### Example 70

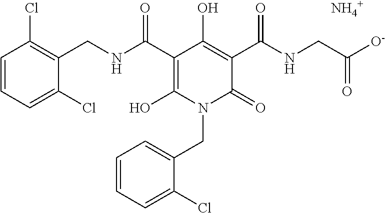

### Ammonium ({[1-[(2-chlorophenyl)methyl]-5-({[(2,6-dichlorophenyl)methyl]amino}carbonyl)-4,6-dihydroxy-2-oxo-1,2-dihydro-3-pyridinyl]carbonyl}amino)acetate

1-(2,6-dichlorophenyl)methylamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 18 mg, 9.12%, MS (ES+): [M+H]⁺=554.0, ¹H NMR (400 MHz, DMSO-d₆) δ ppm 3.84 (br. s., 2H), 4.64 (br. s., 2H), 5.04 (s, 2H), 6.58-6.72 (m, 1H), 7.16-7.25 (m, 2H), 7.34 (t, J=8.08 Hz, 1H), 7.38-7.44 (m, 1 H), 7.47 (d, J=8.08 Hz, 2H), 9.75 (br. s., 1H), 10.31 (br. s., 1H), 16.07 (s, 1H)

### Example 71

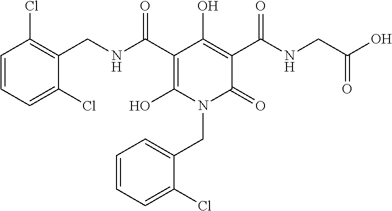

### N-{[1-[(2-chlorophenyl)methyl]-5-({[(2,6-dichlorophenyl)methyl]amino}carbonyl)-4,6-dihydroxy-2-oxo-1,2-dihydro-3-pyridinyl]carbonyl}glycine

1-(2,6-dichlorophenyl)methylamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 43 mg, 22.4%, MS (ES+): [M+H]⁺=554.0, ¹H NMR (400 MHz, DMSO-d₆) δ ppm 3.89 (br. s., 2H), 4.66 (br. s., 2H), 5.05 (s, 2H), 6.64-6.72 (m, 1H), 6.96 (s, 1H), 7.09 (s, 1H), 7.17-7.26 (m, 2H), 7.30-7.39 (m, 1H), 7.39-7.45 (m, 1 H), 7.48 (d, J=8.08 Hz, 2H), 9.22-10.64 (m, 2H), 12.60 (br. s., 1H)

### Example 72

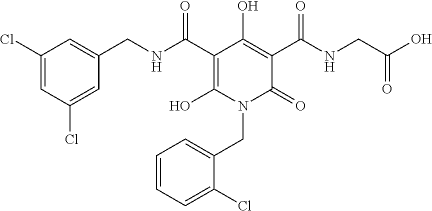

### N-{[1-[(2-chlorophenyl)methyl]-5-({[(3,5-dichlorophenyl)methyl]amino}carbonyl)-4,6-dihydroxy-2-oxo-1,2-dihydro-3-pyridinyl]carbonyl}glycine

1-(3,5-dichlorophenyl)methylamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 66 mg, 30.8%, MS (ES+): [M+H]⁺=554.0, ¹H NMR (400 MHz, DMSO-d₆) δ ppm 3.81 (d, J=4.80 Hz, 2H), 4.36 (br. s., 2H), 5.11 (s, 2H), 6.65-6.82 (m, 1H), 7.22 (dd, J=5.81, 3.54 Hz, 2H), 7.33 (s, 2H), 7.42 (dd, J=5.68, 3.66 Hz, 2H), 8.08 (br. s., 4H), 9.69 (br. s., 1H), 10.26 (br. s., 1H), 16.15 (br. s., 1H)

### Example 73

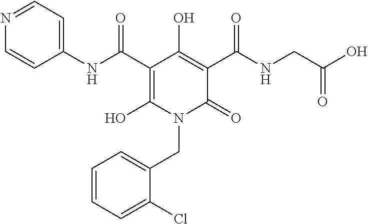

### N-({1-[(2-chlorophenyl)methyl]-4,6-dihydroxy-2-oxo-5-[(4-pyridinylamino)carbonyl]-1,2-dihydro-3-pyridinyl}carbonyl)glycine

4-pyridinamine (48 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA acetonitrile, 0.1% TFA water) to yield the title compound as a white solid. Yield: 32 mg, 19.6%, MS (ES+): [M+H]⁺=473.1, ¹H NMR (400 MHz, DMSO-d₆) δ ppm 3.98 (d, J=5.31 Hz, 2H), 5.17 (s, 2H), 6.78 (dd, J=5.31, 4.04 Hz, 1H), 7.18-7.32 (m, 2H), 7.42-7.55 (m, 1H), 8.08 (d, J=6.82 Hz, 2H), 8.54 (d, J=7.33 Hz, 2H), 10.21 (t, J=5.56 Hz, 1H), 13.38 (s, 1H)

### Example 74

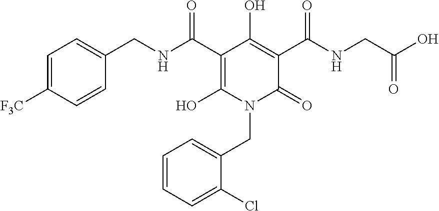

### N-({1-[(2-chlorophenyl)methyl]-4,6-dihydroxy-2-oxo-5-[({[4-(trifluoromethyl)phenyl]methyl}amino)carbonyl]-1,2-dihydro-3-pyridinyl}carbonyl)glycine

1-[4-(trifluoromethyl)phenyl]methanamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 64 mg, 33.5%, MS (ES+): [M+H]⁺=454.1, ¹H NMR (400 MHz, DMSO-d₆) δ ppm 3.92 (d, J=4.80 Hz, 2H), 4.49 (d, J=5.31 Hz, 2H), 5.12 (s, 2 H), 6.76 (dd, J=5.56, 3.79 Hz, 1H), 7.00 (br. s., 1H), 7.12 (br. s., 1H), 7.18-7.33 (m, 2H), 7.43 (dd, J=5.68, 3.66 Hz, 1H), 7.51 (d, J=8.08 Hz, 2H), 7.67 (d, J=8.08 Hz, 2H), 10.07 (br. s., 2H), 12.61 (br. s., 1H)

### Example 75

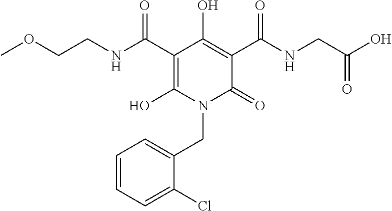

### N-{[1-[(2-chlorophenyl)methyl]-4,6-dihydroxy-5-({[2-(methyloxy)ethyl]amino}carbonyl)-2-oxo-1,2-dihydro-3-pyridinyl]carbonyl}glycine

2-(methyloxy)ethanamine (39 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 75 mg, 46.1%, MS (ES+): [M+H]⁺=454.9, ¹H NMR (400 MHz, DMSO-d₆) δ ppm 3.23 (s, 3H), 3.28-3.54 (m, 4H), 3.76 (d, J=5.05 Hz, 2H), 5.09 (s, 2H), 6.70 (dd, J=5.43, 3.92 Hz, 1H), 7.10-7.27 (m, 2H), 7.36-7.47 (m, 1H), 7.64 (br. s., 4H), 9.08-9.87 (m, 1H), 10.23 (br. s., 1H), 15.94-16.54 (m, 1H)

### Example 76

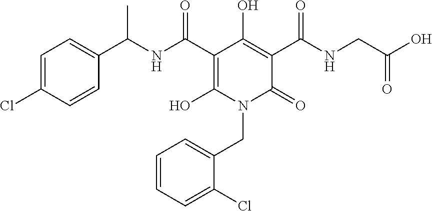

### N-({5-({[1-(4-chlorophenyl)ethyl]amino}carbonyl)-1-[(2-chlorophenyl)methyl]-4,6-dihydroxy-2-oxo-1,2-dihydro-3-pyridinyl}carbonyl)glycine

1-(4-chlorophenyl)ethanamine (80 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 78 mg, 41.0%, MS (ES+): [M+H]⁺=534.1, ¹H NMR (400 MHz, DMSO-d₆) δ ppm 1.36 (d, J=5.81 Hz, 3H), 3.83 (d, J=4.55 Hz, 2H), 5.00 (t, J=6.95 Hz, 1H), 5.12 (s, 2H), 6.72 (dd, J=5.68, 3.66 Hz, 1H), 7.17-7.29 (m, 2H), 7.29-7.40 (m, 4H), 7.41-7.49 (m, 1H), 9.86 (br. s., 1H), 10.20-10.89 (m, 1H), 15.91-16.19 (m, 1H)

### Example 77

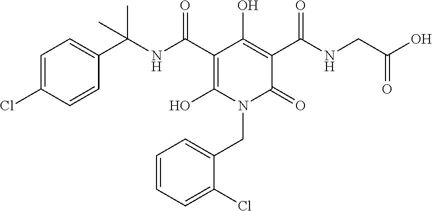

### N-{[1-[(2-chlorophenyl)methyl]-5-({[1-(4-chlorophenyl)-1-methylethyl]amino}carbonyl)-4,6-dihydroxy-2-oxo-1,2-dihydro-3-pyridinyl]carbonyl}glycine

2-(4-chlorophenyl)-2-propanamine (87 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 32 mg, 16.9%, MS (ES+): [M+H]⁺=548.1, ¹H NMR (400 MHz, DMSO-d₆) δ ppm 1.59 (s, 6H), 3.88 (d, J=4.04 Hz, 2H), 5.13 (s, 2H), 6.69-6.81 (m, 1H), 7.21-7.29 (m, 2H), 7.30-7.39 (m, 4H), 7.42-7.49 (m, 1H), 9.50-11.10 (m, 2 H), 12.55 (br. s., 1H), 16.23 (br. s., 1H)

### Example 78

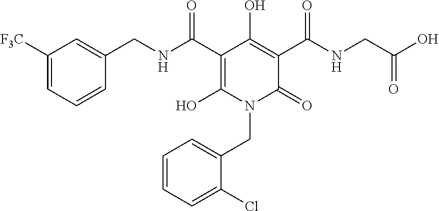

### N-({1-[(2-chlorophenyl)methyl]-4,6-dihydroxy-2-oxo-5-[({[3-(trifluoromethyl)phenyl]methyl}amino)carbonyl]-1,2-dihydro-3-pyridinyl}carbonyl)glycine

1-[3-(trifluoromethyl)phenyl]methanamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 76 mg, 39.7%, MS (ES+): [M+H]⁺=454.1, ¹H NMR (400 MHz, DMSO-d₆) δ ppm 3.92 (br. s., 2H), 4.49 (d, J=4.55 Hz, 2H), 5.11 (s, 2H), 6.70-6.83 (m, 1H), 6.97 (br. s., 1H), 7.09 (br. s., 1H), 7.22 (dd, J=5.81, 3.54 Hz, 3H), 7.39-7.47 (m, 1H), 7.52-7.64 (m, 3H), 7.66 (s, 1H), 10.04 (br. s., 1H), 12.62 (br. s., 1H)

### Example 79

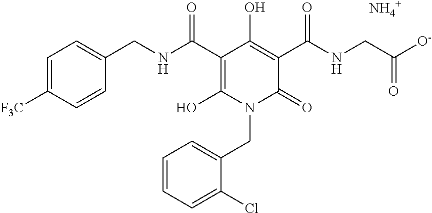

### Ammonium [({1-[(2-chlorophenyl)methyl]-4,6-dihydroxy-2-oxo-5-[({[4-(trifluoromethyl)phenyl]methyl}amino)carbonyl]-1,2-dihydro-3-pyridinyl}carbonyl)amino]acetate

1-[4-(Trifluoromethyl)phenyl]methanamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 26 mg, 13.1%, MS (ES+): [M+H]⁺=454.1, ¹H NMR (400 MHz, DMSO-d₆) δ ppm 3.80 (d, J=5.31 Hz, 2H), 4.47 (br. s., 2H), 5.11 (s, 2H), 6.73 (dd, J=5.56, 3.79 Hz, 1H), 7.15-7.29 (m, 2H), 7.37-7.47 (m, 1H), 7.50 (d, J=8.08 Hz, 2H), 7.66 (d, J=7.83 Hz, 2H), 9.82 (br. s., 1H), 10.29 (br. s., 1H)

### Example 80

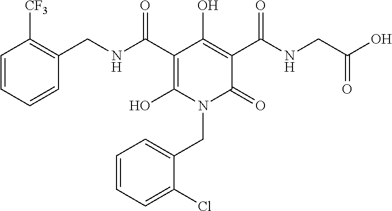

### N-({1-[(2-chlorophenyl)methyl]-4,6-dihydroxy-2-oxo-5-[({[2-(trifluoromethyl)phenyl]methyl}amino)carbonyl]-1,2-dihydro-3-pyridinyl}carbonyl)glycine

1-[2-(Trifluoromethyl)phenyl]methanamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 50 mg, 26.1%, MS (ES+): [M+H]⁺=454.1, ¹H NMR (400 MHz, DMSO-d₆) δ ppm 3.91 (d, J=4.04 Hz, 2H), 4.59 (d, J=4.55 Hz, 2H), 5.11 (s, 2 H), 6.75 (dd, J=5.31, 3.79 Hz, 1H), 6.99 (br. s., 1H), 7.11 (br. s., 1H), 7.23 (dd, J=5.94, 3.41 Hz, 2H), 7.36-7.49 (m, 2H), 7.53-7.61 (m, 1H), 7.64 (t, J=7.45 Hz, 1H), 7.70 (d, J=7.83 Hz, 1H), 10.12 (br. s., 2H), 12.60 (br. s., 1H)

### Example 81

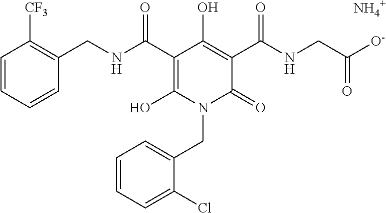

### Ammonium [({1-[(2-chlorophenyl)methyl]-4,6-dihydroxy-2-oxo-5-[({[2-(trifluoromethyl)phenyl]methyl}amino)carbonyl]-1,2-dihydro-3-pyridinyl}carbonyl)amino]acetate

1-[2-(Trifluoromethyl)phenyl]methanamine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 70 mg, 35.5%, MS (ES+): [M+H]⁺=454.1, ¹H NMR (400 MHz, DMSO-d₆) δ ppm 3.67 (d, J=3.03 Hz, 2H), 4.57 (br. s., 2H), 5.11 (s, 2H), 6.73 (dd, J=5.56, 3.79 Hz, 1H), 7.15-7.28 (m, 2H), 7.37-7.50 (m, 2H), 7.52-7.75 (m, 3H), 9.92 (br. s., 1H), 10.22 (br. s., 1H)

### Example 82

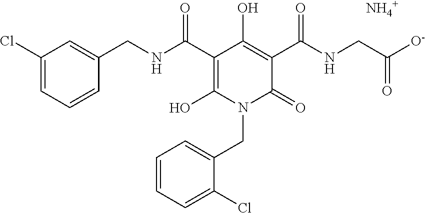

### Ammonium ({[1-[(2-chlorophenyl)methyl]-5-({[(3-chlorophenyl)methyl]amino}carbonyl)-4,6-dihydroxy-2-oxo-1,2-dihydro-3-pyridinyl]carbonyl}amino)acetate

[(3-Chlorophenyl)methyl]amine (73 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 134 mg, 70.8%, MS (ES+): [M+H]⁺=520.1, 1H NMR (400 MHz, DMSO-d₆) δ ppm 3.84 (d, J=2.78 Hz, 2H), 4.37 (d, J=1.77 Hz, 2H), 5.10 (s, 2H), 6.73 (dd, J=5.56, 3.79 Hz, 1H), 7.21-7.30 (m, 6 H), 7.30-7.37 (m, 3H), 7.39-7.45 (m, 2H), 9.65 (br. s., 1H), 10.30 (br. s., 1H), 15.97 (br. s., 1 H)

### Example 83

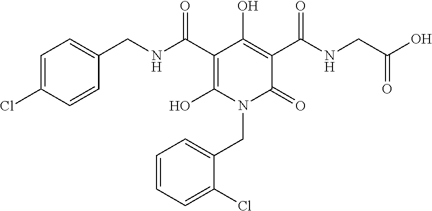

### N-{[1-[(2-chlorophenyl)methyl]-5-({[(4-chlorophenyl)methyl]amino}carbonyl)-4,6-dihydroxy-2-oxo-1,2-dihydro-3-pyridinyl]carbonyl}glycine

[(4-chlorophenyl)methyl]amine (73 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the ammonium salt of the title compound as a white solid. Yield: 89 mg, 47%, MS (ES+): [M+H]⁺=520.1, ¹H NMR (400 MHz, DMSO-d₆) δ ppm 4.07 (d, J=5.31 Hz, 2H), 4.54 (d, J=6.06 Hz, 2H), 5.14 (s, 2H), 6.85-6.92 (m, 1H), 7.01 (s, 1H), 7.14 (s, 1H), 7.21-7.32 (m, 3H), 7.38 (q, J=8.59 Hz, 4H), 7.47 (dd, J=7.58, 1.77 Hz, 1H), 9.84 (br. s., 1H), 10.03 (br. s., 1H), 13.00 (br. s., 1H)

### Example 84

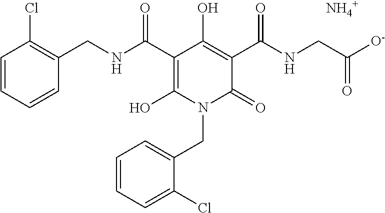

### Ammonium ({[1-[(2-chlorophenyl)methyl]-5-({[(2-chlorophenyl)methyl]amino}carbonyl)-4,6-dihydroxy-2-oxo-1,2-dihydro-3-pyridinyl]carbonyl}amino)acetate

[(2-Chlorophenyl)methyl]amine (73 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 36 mg, 19%, MS (ES+): [M+H]⁺=520.1, ¹H NMR (400 MHz, DMSO-d₆) δ ppm 3.77 (d, J=5.05 Hz, 2H), 4.43 (br. s., 2H), 5.10 (s, 2H), 6.73 (dd, J=5.43, 3.92 Hz, 1H), 7.19-7.25 (m, 2H), 7.25-7.34 (m, 2H), 7.42 (dd, J=5.31, 3.79 Hz, 3H), 7.71 (br. s., 5H), 9.67 (br. s., 1H), 10.27 (br. s., 1 H), 16.04 (br. s., 1H)

### Example 85

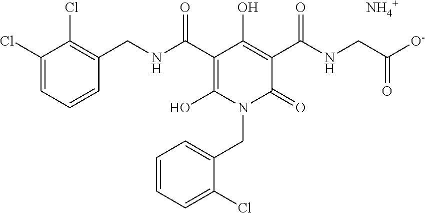

### Ammonium ({[1-[(2-chlorophenyl)methyl]-5-({[(2,3-dichlorophenyl)methyl]amino}carbonyl)-4,6-dihydroxy-2-oxo-1,2-dihydro-3-pyridinyl]carbonyl}amino)acetate

[(2,3-Dichlorophenyl)methyl]amine (90 mg, 0.513 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 130 mg, 64.5%, MS (ES+): [M+H]⁺=554.0, ¹H NMR (400 MHz, DMSO-d₆) δ ppm 3.63 (d, J=4.55 Hz, 2H), 4.43 (br. s., 2H), 5.10 (s, 2H), 6.62 (br. s., 4H), 6.71-6.80 (m, 1H), 7.19-7.25 (m, 2 H), 7.34 (q, J=7.49 Hz, 2H), 7.39-7.45 (m, 1H), 7.51 (d, J=7.33 Hz, 1H), 9.77 (br. s., 1H), 10.20 (br. s., 1H)

### Example 86

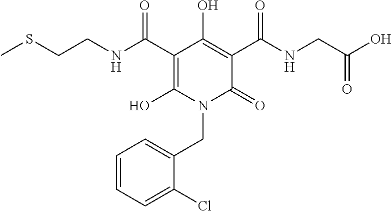

### N-{[1-[(2-chlorophenyl)methyl]-4,6-dihydroxy-5-({[2-(methylthio)ethyl]amino}carbonyl)-2-oxo-1,2-dihydro-3-pyridinyl]carbonyl}glycine

2-(Methylthio)ethanamine (39 mg, 0.430 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 160 mg, 0.364 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA acetonitrile, 0.1% TFA water) to yield the title compound as a white solid. Yield: 61 mg, 35.3%, MS (ES+): [M+H]⁺=469.1, ¹H NMR (400 MHz, DMSO-d₆) δ ppm 2.09 (s, 3H), 2.69 (t, J=6.69 Hz, 2H), 3.57 (q, J=6.40 Hz, 2H), 4.09 (d, J=5.56 Hz, 2H), 5.15 (s, 2H), 6.90 (dd, J=7.58, 1.77 Hz, 1H), 7.22-7.33 (m, J=7.39, 7.11, 6.96, 6.96, 1.64 Hz, 2H), 7.49 (dd, J=7.45, 1.64 Hz, 1H), 9.73 (br. s., 1H), 9.84 (br. s., 1H), 13.03 (br. s., 1H)

### Example 87

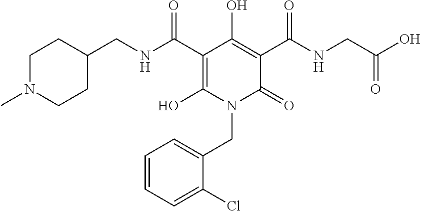

### N-{[1-[(2-chlorophenyl)methyl]-4,6-dihydroxy-5-({[(1-methyl-4-piperidinyl)methyl]amino}carbonyl)-2-oxo-1,2-dihydro-3-pyridinyl]carbonyl}glycine

1-(1-Methyl-4-piperidinyl)methanamine (41 mg, 0.316 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 160 mg, 0.360 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 29 mg, 15.5%, MS (ES+): [M+H]⁺=507.1, ¹H NMR (400 MHz, DMSO-d₆) δ ppm 1.38 (q, J=12.63 Hz, 2H), 1.84 (d, J=11.87 Hz, 3H), 2.73 (s, 3H), 2.89 (d, J=7.07 Hz, 2H), 3.30 (t, J=5.81 Hz, 2H), 3.42 (d, J=11.87 Hz, 2H), 4.08 (d, J=5.31 Hz, 2H), 5.15 (s, 2H), 6.89 (d, J=7.07 Hz, 1H), 7.19-7.34 (m, J=7.52, 7.23, 7.09, 7.09, 1.64 Hz, 2H), 7.49 (dd, J=7.45, 1.64 Hz, 1H), 9.35 (br. s., 1H), 9.69 (br. s., 1H), 9.85 (br. s., 1H), 13.02 (br. s., 1H)

### Example 88

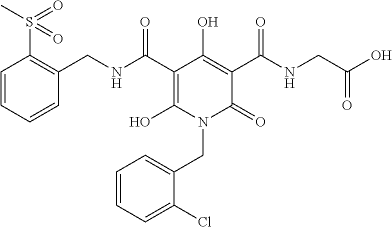

### N-({1-[(2-chlorophenyl)methyl]-4,6-dihydroxy-5-[({[2-(methylsulfonyl)phenyl]methyl}amino) carbonyl]-2-oxo-1,2-dihydro-3-pyridinyl}carbonyl)glycine

[2-(Methylsulfonyl)phenyl]methanaminium chloride (121 mg, 0.547 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 160 mg, 0.360 mmol) and diisopropylethylamine (71 mg, 0.547 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 66 mg, 31.8%, MS (ES+): [M+H]⁺=564.1, ¹H NMR (400 MHz, DMSO-d₆) δ ppm 3.32 (s, 3H), 4.09 (d, J=5.31 Hz, 2H), 4.93 (d, J=6.32 Hz, 2H), 5.15 (s, 2H), 6.92 (dd, J=7.45, 1.39 Hz, 1H), 7.21-7.33 (m, J=7.42, 7.42, 7.42, 7.42, 1.64 Hz, 2H), 7.48 (dd, J=7.71, 1.64 Hz, 1H), 7.55-7.64 (m, 2H), 7.69-7.77 (m, 1H), 7.95 (d, J=7.83 Hz, 1H), 9.80 (br. s., 1H), 10.10 (br. s., 1H), 13.05 (br. s., 1H)

### Example 89

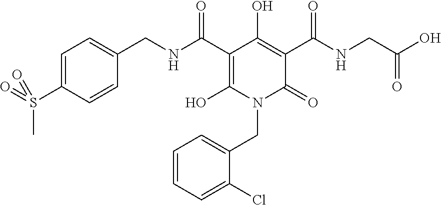

### N-({1-[(2-Chlorophenyl)methyl]-4,6-dihydroxy-5-[({[4-(methylsulfonyl)phenyl]methyl}amino) carbonyl]-2-oxo-1,2-dihydro-3-pyridinyl}carbonyl)glycine

[4-(Methylsulfonyl)phenyl]methanaminium chloride (121 mg, 0.547 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 160 mg, 0.360 mmol) and diisopropylethylamine (71 mg, 0.547 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 53 mg, 25.5%, MS (ES+): [M+H]⁺=564.1, ¹H NMR (400 MHz, DMSO-d₆) δ ppm 3.19 (s, 3H), 4.10 (d, J=5.31 Hz, 2H), 4.67 (d, J=5.81 Hz, 2H), 5.15 (s, 2H), 6.92 (dd, J=7.45, 1.64 Hz, 1H), 7.21-7.34 (m, J=7.45, 7.17, 7.03, 7.03, 1.64 Hz, 2H), 7.48 (dd, J=7.45, 1.64 Hz, 1H), 7.60 (d, J=8.34 Hz, 2H), 7.90 (d, J=8.34 Hz, 2H), 9.81 (br. s., 1H), 10.11 (br. s., 1H), 13.04 (br. s., 1H)

### Example 90

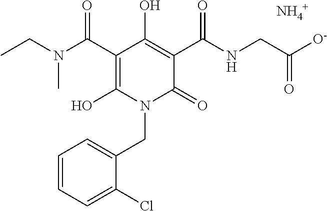

### Ammonium {[(1-[(2-chlorophenyl)methyl]-5-{[ethyl(methyl)amino]carbonyl}-4,6-dihydroxy-2-oxo-1,2-dihydro-3-pyridinyl)carbonyl]amino}acetate

Ethyl(methyl)amine (29 mg, 0.466 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 50 mg, 28.7%, MS (ES+): [M+H]⁺=438.1, ¹H NMR (400 MHz, DMSO-d₆) δ ppm 1.07 (t, J=7.07 Hz, 3 H), 2.93 (s, 3H), 3.37 (q, J=6.99 Hz, 2H), 3.93 (d, J=5.05 Hz, 2H), 5.05 (s, 2H), 5.33 (br. s., 4 H), 6.77 (dd, J=5.43, 3.92 Hz, 1H), 7.17-7.29 (m, 2H), 7.40-7.46 (m, 1H), 10.15 (t, J=5.43 Hz, 1H)

### Example 91

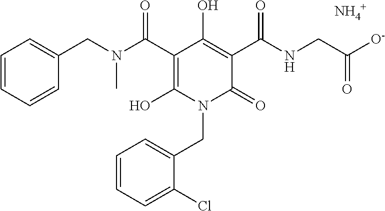

### Ammonium {[(1-[(2-chlorophenyl)methyl]-4,6-dihydroxy-5-{[methyl(phenylmethyl)amino]carbonyl}-2-oxo-1,2-dihydro-3-pyridinyl)carbonyl]amino}acetate

Methyl(phenylmethyl)amine (42 mg, 0.349 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 150 mg, 0.342 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (acetonitrile, 0.1% NH₄OH water) to yield the title compound as a white solid. Yield: 50 mg, 28.7%, MS (ES+): [M+H]⁺=438.1, ¹H NMR (400 MHz, DMSO-d₆) δ ppm 2.83 (s, 3H), 3.93 (d, J=4.29 Hz, 2H), 4.59 (br. s., 2H), 5.07 (s, 2H), 6.75 (d, J=8.34 Hz, 1H), 7.13-7.28 (m, 3H), 7.28-7.40 (m, 4H), 7.43 (dd, J=7.58, 1.52 Hz, 1H), 10.16 (br. s., 1H)

### Example 92

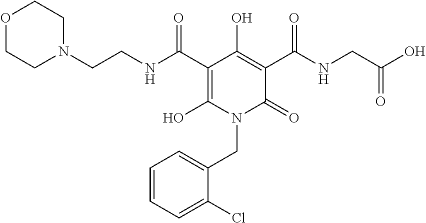

### N-{[1-[(2-chlorophenyl)methyl]-4,6-dihydroxy-5-({[2-(4-morpholinyl)ethyl]amino}carbonyl)-2-oxo-1,2-dihydro-3-pyridinyl]carbonyl}glycine

2-(4-Morpholinyl)ethanamine (45 mg, 0.343 mmol) was added to a solution of methyl 1-[(2-chlorophenyl)methyl]-5-({[2-(ethyloxy)-2-oxoethyl]amino}carbonyl)-2,4-dihydroxy-6-oxo-1,6-dihydro-3-pyridinecarboxylate (prepared by following the procedure of example 1d, 160 mg, 0.360 mmol) and diisopropylethylamine (71 mg, 0.547 mmol) in chloroform (3 ml). The solution solidified upon standing. The mixture was heated in a microwave (150° C., 30 min). The solution was washed with 1N HCl (2 ml). The organic layer was concentrated in-vacuo and the residue dissolved in ethanol (6 ml) and treated with 6N NaOH. The mixture was stirred at rt over night. Upon completion of the saponification the solution was diluted with ethyl acetate (20 ml) and washed with 1N HCl (7 ml). The organic layer was concentrated in-vacuo and the residue was dissolved in dimethyl sulfoxide (1.5 ml) and purified by HPLC (0.1% TFA, acetonitrile, 0.1% TFA water) to yield the title compound as a white solid. Yield: 40 mg, 21.3%, MS (ES+): [M+H]⁺=509.1, ¹H NMR (400 MHz, DMSO-d₆) δ ppm 2.98-4.57 (m, 14H), 5.17 (s, 2H), 6.88 (dd, J=7.33, 1.77 Hz, 1H), 7.28 (dq, J=6.57, 6.40 Hz, 2H), 7.49 (dd, J=7.45, 1.64 Hz, 1H), 9.69 (br. s., 1H), 9.82 (br. s., 1H), 13.11 (br. s., 1H)

### Example 93

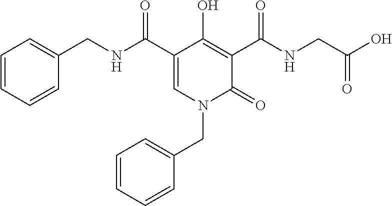

### N-[(4-hydroxy-2-oxo-1-(phenylmethyl)-5-{[(phenylmethyl)amino]carbonyl}-1,2-dihydro-3-pyridinyl)carbonyl]glycine

93a) Ethyl 4-hydroxy-6-oxo-1-(phenylmethyl)-1,6-dihydro-3-pyridinecarboxylate. Into a 500 mL round-bottomed flask were added ethyl 1,3 acetondicarboxylate (25 g, 124 mmol) and trimethyl orthformate (13.12 g, 124 mmol). Acetic anhydride (23.33 ml, 247 mmol) was then added. The mixture was stirred and heated at 120° C. for two hours, then cooled down to room temperature. After removing all volatile material under reduced pressure, the residue was dissolved with THF (150 mL). Benzylamine (13.25 g, 124 mmol) was added slowly. The reaction mixture was stirred at room temperature for overnight. LCMS showed ring-opened intermediate. Sodium hydride (2.98 g, 124 mmol) was added portionwise and the mixture was stirred at rt for 30 min. After removal of THF, the residue was cooled down to 0° C., and added ethyl acetate (200 mL) and 50 ml of 10% HCl solution (to adjust pH of aqueous layer around 2). Two layers were separated, and the aqueous layer was extracted again with ethyl acetate (125 mL, 2×). Combined organic layers were dried and concentrated under vacuum. Crude product was obtained (32 g, 85%) and used without further purification.

93b) 4-Hydroxy-6-oxo-1-(phenylmethyl)-1,6-dihydro-3-pyridinecarboxylic acid. Into a 100 mL round-bottomed flask was added ethyl 4-hydroxy-6-oxo-1-(phenylmethyl)-1,6-dihydro-3-pyridinecarboxylate (8.20 g, 30 mmol) in N,N-Dimethylformamide (DMF) (15 ml). 2N NaOH (15 mL) was added. The reaction mixture was stirred at room temperature for 2 hours and diluted with water (25 mL) and ethyl acetate (50 mL). Two layers were separated. The aqueous layer was adjusted to pH=2 using 6N HCl, and extracted with ethyl acetate (50 mL 3×). Combined organic layers were dried and concentrated under vacuum. Crude product (4.1 g, 55%) was obtained and used for next step without further purification.

93c) 4-Hydroxy-6-oxo-N,1-bis(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (540 mg, 2.2 mmol), EDC (464 mg, 2.42 mmol), and HOBt (371 mg, 2.42 mmol) in N,N-Dimethylformamide (2 ml) and DCM (8 mL). Reaction mixture was stirred at rt for 30 minutes before benzylamine (236 mg, 2.2 mmol) was added. Then reaction mixture was stirred at rt for overnight. LCMS showed that the reaction was complete. Reaction mixture was concentrated, and the residue was dissolved in 5 mL DMSO and purified with Gilson preparative HPLC to give desired product (170 mg, 23%).

93d) N-[(4-Hydroxy-2-oxo-1-(phenylmethyl)-5-{[(phenylmethyl)amino]carbonyl}-1,2-dihydro-3-pyridinyl)carbonyl]glycine. Into a 5 mL microwave tube were added 4-hydroxy-6-oxo-N,1-bis(phenylmethyl)-1,6-dihydro-3-pyridinecarboxamide (170 mg, 0.508 mmol), ethyl N-(oxomethylidene)glycinate (65.6 mg, 0.508 mmol), and DIEA (0.089 mL, 0.508 mmol) in chloroform (2 mL) to give a brown suspension. The mixture was heated at 120° C. for 30 minutes. LC MS showed that the reaction was complete. Chloroform was removed. Ethanol (5 mL) and 300 uL of 2N NaOH solution were added to the residue. The resulted reaction was stirred at room temperature for 30 minutes. LC MS showed that the reaction was complete. Reaction mixture was concentrated and then added 2 mL of water and adjusted pH to 1˜2 with 6N HCl solution. Reaction mixture was concentrated and the residue was dissolved in DMSO (2 mL). DMSO solution was filtered and purified with Gilson preparative HPLC. Solid product (12 mg, 5.4%) was obtained. MS (ES+): m/z [M+H]⁺=436.0; ¹H NMR (400 MHz, DMSO-d₆) δ ppm 4.10 (d, J=5.31 Hz, 2H) 4.50 (d, J=6.57 Hz, 2H) 5.26 (s, 2H) 7.24 (s, 1H) 7.27-7.39 (m, 9H) 8.73 (s, 1H) 10.36 (s, 1H)

### Example 94

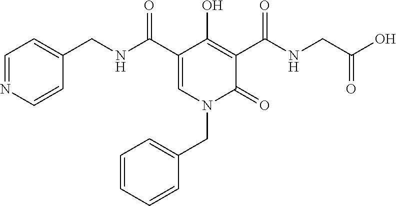

### N-[(4-hydroxy-2-oxo-1-(phenylmethyl)-5-{[(4-pyridinylmethyl)amino]carbonyl}-1,2-dihydro-3-pyridinyl)carbonyl]glycine

94a) 4-Hydroxy-6-oxo-1-(phenylmethyl)-N-(4-pyridinylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (540 mg, 2.2 mmol), EDC (464 mg, 2.42 mmol), and HOBt (371 mg, 2.42 mmol) in N,N-Dimethylformamide (DMF) (2 ml) and 8 ml of DCM. Reaction mixture was stirred at room temperature for 30 minutes before (4-pyridinylmethyl)amine (238 mg, 2.2 mmol). Then reaction mixture was stirred at room temperature overnight. LCMS showed the reaction was complete. Reaction mixture was purified with Gilson preparative HPLC to give desired product (171 mg, 23%).

94b) N-[(4-hydroxy-2-oxo-1-(phenylmethyl)-5-{[(4-pyridinylmethyl)amino]carbonyl}-1,2-dihydro-3-pyridinyl)carbonyl]glycine. Into a 5 mL microwave tube were added 4-hydroxy-6-oxo-1-(phenylmethyl)-N-(4-pyridinylmethyl)-1,6-dihydro-3-pyridinecarboxamide (120 mg, 0.36 mmol), DIEA (0.062 mL, 0.36 mmol), and ethyl N-(oxomethylidene)glycinate (46.2 mg, 0.36 mmol) in chloroform (2 mL). The resulted mixture was heated and stirred at 120° C. for 30 minutes. LCMS showed that the reaction was completed. The reaction mixture was concentrated and then added 2 mL of ethanol and added 10 N NaOH solution (30 ul). The reaction mixture was stirred at room temperature for 15 minutes. LCMS showed that the reaction was complete. Reaction mixture was concentrated and then diluted with water (2 mL). The pH of resulted solution was adjusted with 6N HCl to pH around 1-2. The reaction mixture was concentrated and the residue was dissolved in DMSO (2 mL). The DMSO solution was filtered and purified with Gilson preparative HPLC. Desired product was obtained (35 mg, 22.4%). MS (ES+): m/z [M+H]⁺=437.1; ¹H NMR (400 MHz, DMSO-d₆) ppm 4.11 (d, J=5.56 Hz, 2H) 4.67 (d, J=6.06 Hz, 2H) 5.27 (s, 2H) 7.27-7.38 (m, 5H) 7.69 (br. s., 2H) 8.69 (s, 1H) 8.72 (d, J=8.34 Hz, 1H) 8.90 (s, 1H) 10.36 (s, 1H)

### Example 95

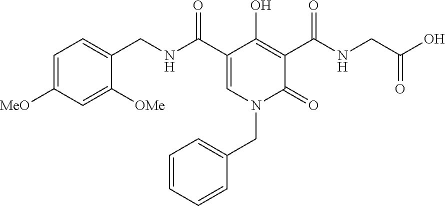

### N-{[5-[({[2,4-bis(methyloxy)phenyl]methyl}amino)carbonyl]-4-hydroxy-2-oxo-1-(phenylmethyl)-1,2-dihydro-3-pyridinyl]carbonyl}glycine

95a) N-{[2,4-bis(methyloxy)phenyl]methyl}-4-hydroxy-6-oxo-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (490 mg, 2 mmol), EDC (383 mg, 2 mmol), and HOBt (306 mg, 2 mmol) in N,N-Dimethylformamide (DMF) (2 ml) and 8 ml of DCM. Reaction mixture was stirred for 30 minutes before {[2,4-bis(methyloxy)phenyl]methyl}amine (334 mg, 2 mmol) was added. Then reaction mixture was stirred at room temperature overnight. LCMS showed that the reaction was complete. Reaction was purified with Gilson preparative HPLC to give desired product (150 mg, 18%).

95b) N-{[5-[({[2,4-bis(methyloxy)phenyl]methyl}amino)carbonyl]-4-hydroxy-2-oxo-1-(phenylmethyl)-1,2-dihydro-3-pyridinyl]carbonyl}glycine. Into a 2 mL microwave tube were added N-{[2,4-bis(methyloxy)phenyl]methyl}-4-hydroxy-6-oxo-1-(phenylmethyl)-1,6-dihydro-3-pyridinecarboxamide (150 mg, 0.38 mmol), ethyl N-(oxomethylidene)glycinate (49.1 mg, 0.38 mmol), and DIEA (0.066 mL, 0.38 mmol) in chloroform (2 mL). Reaction mixture was heated at 120° C. for 30 minutes. LCMS showed that the reaction was complete. Chloroform was removed. Ethanol (5 mL) and 300 uL of 2N NaOH solution were added to the residue. The resulted solution was stirred at room temperature for 30 minutes. LCMS showed that the reaction was complete. Reaction mixture was concentrated and then diluted with water (2 mL). The pH of resulted solution was adjusted with 6N HCl to pH around 1-2. Reaction mixture was concentrated and the residue was dissolved in DMSO (2 mL). DMSO solution was filtered and purified with Gilson preparative HPLC. Desired product (18 mg, 9%) was obtained. MS (ES+): m/z [M+H]⁺=495.9; ¹H NMR (400 MHz, DMSO-d₆) δ ppm 3.74 (s, 3H) 3.82 (s, 3H) 4.11 (s, 2H) 4.39 (s, 2H) 5.26 (s, 2H) 6.48 (d, J=2.27 Hz, 1H) 6.57 (d, J=2.27 Hz, 1H) 7.12 (d, J=8.59 Hz, 1H) 7.32 (d, J=13.39 Hz, 4H) 8.47 (s, 1H) 8.72 (s, 1H)

### Example 96

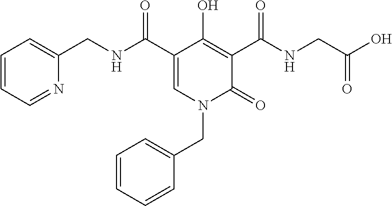

### N-[(4-hydroxy-2-oxo-1-(phenylmethyl)-5-{[(2-pyridinylmethyl)amino]carbonyl}-1,2-dihydro-3-pyridinyl)carbonyl]glycine

96a) 4-Hydroxy-6-oxo-1-(phenylmethyl)-N-(2-pyridinylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (540 mg, 2.2 mmol), EDC (422 mg, 2.2 mmol), and HOBt (337 mg, 2.2 mmol) in N,N-Dimethylformamide (DMF) (2 ml) and 8 ml of DCM. Reaction mixture was stirred at room temperature for 30 minutes before adding (2-pyridinylmethyl)amine (238 mg, 2.2 mmol). Then reaction mixture was stirred at room temperature overnight. LCMS showed the reaction was complete. Reaction mixture was concentrated and purified with Gilson preparative HPLC to give desired product (150 mg, 20%).

96b) N-[(4-hydroxy-2-oxo-1-(phenylmethyl)-5-{[(2-pyridinylmethyl)amino]carbonyl}-1,2-dihydro-3-pyridinyl)carbonyl]glycine. Into a 2 mL microwave tube were added 4-hydroxy-6-oxo-1-(phenylmethyl)-N-(2-pyridinylmethyl)-1,6-dihydro-3-pyridinecarboxamide (137 mg, 0.41 mmol), ethyl N-(oxomethylidene)glycinate (52.7 mg, 0.41 mmol), and DIEA (0.07 mL, 0.41 mmol) in chloroform (2 mL). Reaction mixture was heat at 120° C. for 30 minutes. LCMS showed that the reaction was complete. Chloroform was removed. Ethanol (5 mL) and 300 uL of 2N NaOH solution were added to residue. The resulted reaction mixture was stirred for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO (2 mL). DMSO solution was filtered and purified with Gilson preparative HPLC. Desired product was obtained (27 mg, 15%). MS (ES+): m/z [M+H]⁺=437.1; ¹H NMR (400 MHz, DMSO-d₆) δ ppm 4.12 (d, J=5.81 Hz, 2H) 4.67 (d, J=5.56 Hz, 2H) 5.27 (s, 2H) 7.27-7.39 (m, 4H) 7.52 (s, 1H) 7.96 (br. s., 1H) 8.60 (s, 1H) 8.75 (s, 1H) 8.95 (s, 1H) 10.37 (s, 1H)

### Example 97

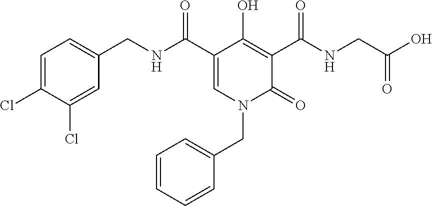

### N-{[5-({[(3,4-dichlorophenyl)methyl]amino}carbonyl)-4-hydroxy-2-oxo-1-(phenylmethyl)-1,2-dihydro-3-pyridinyl]carbonyl}glycine

97a) N-[(3,4-dichlorophenyl)methyl]-4-hydroxy-6-oxo-1-(phenylmethyl)-1,6-dihydro-3-pyridinecarboxamide. Into a 2 mL microwave tube were added methyl 4-hydroxy-6-oxo-1-(phenylmethyl)-1,6-dihydro-3-pyridinecarboxylate (391 mg, 1.51 mmol) and [(3,4-dichlorophenyl)methyl]amine (266 mg, 1.510 mmol. The reaction mixture was stirred and heated at 100° C. for two hours. Ethyl acetate (3 mL) was added into reaction mixture. The resulted reaction suspension was stirred and then filtered. Solid was collected and dried. The crude product was obtained and then used for next step without further purification (420 mg, 69%).

97b) N-{[5-({[(3,4-dichlorophenyl)methyl]amino}carbonyl)-4-hydroxy-2-oxo-1-(phenylmethyl)-1,2-dihydro-3-pyridinyl]carbonyl}glycine. Into a 2 mL microwave tube were added N-[(3,4-dichlorophenyl)methyl]-4-hydroxy-6-oxo-1-(phenylmethyl)-1,6-dihydro-3-pyridinecarboxamide (403 mg, 1.0 mmol) and ethyl N-(oxomethylidene)glycinate (129 mg, 1 mmol) and DIEA (129 mg, 1 mmol) in chloroform (1 mL). Reaction mixture was heated at 120° C. for 30 minutes. LCMS showed that the reaction was complete. Solvent was removed. Ethanol (1 mL) and 200 uL of 2N NaOH solution were added to residue. The resulted solution was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product was obtained (59 mg, 12%). MS (ES+): m/z [M+H]⁺=503.9; ¹H NMR (400 MHz, DMSO-d₆) δ ppm 4.11 (s, 2H) 4.48 (s, 2H) 5.26 (s, 2H) 7.27-7.39 (m, 5H) 7.59 (s, 1H) 7.58 (d, J=3.03 Hz, 2H) 8.72 (s, 2H) 10.35 (s, 1H) 12.98 (br. s., 1H)

### Example 98

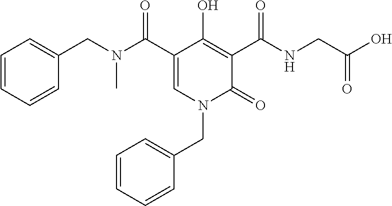

### N-{[4-hydroxy-5-{[methyl(phenylmethyl)amino]carbonyl}-2-oxo-1-(phenylmethyl)-1,2-dihydro-3-pyridinyl]carbonyl}glycine

98a) 4-Hydroxy-N-methyl-6-oxo-N,1-bis(phenylmethyl)-1,6-dihydro-3-pyridinecarboxamide. Into a 2 mL microwave tube were added methyl 4-hydroxy-6-oxo-1-(phenylmethyl)-1,6-dihydro-3-pyridinecarboxylate (391 mg, 1.51 mmol), and N-methyl-1-phenylmethanamine (183 mg, 1.51 mmol). The reaction mixture was heated at 105° C. and stirred for 30 minutes in microwave reactor. LCMS showed that the reaction was complete. Ethyl acetate (1 mL) was added into reaction mixture and reaction mixture was stirred and filtered. Solid was collected and dried. The crude product (350 mg, 67%) was obtained and used for next step without further purification.

98b) N-{[4-hydroxy-5-{[methyl(phenylmethyl)amino]carbonyl}-2-oxo-1-(phenylmethyl)-1,2-dihydro-3-pyridinyl]carbonyl}glycine. Into a 2 ml microwave reaction vessel were added 4-hydroxy-N-methyl-6-oxo-N,1-bis(phenylmethyl)-1,6-dihydro-3-pyridinecarboxamide (200 mg, 0.57 mmol), ethyl N-(oxomethylidene)glycinate (74.1 mg, 0.57 mmol), and DIEA (0.1 mL, 0.57 mmol) in chloroform (1.0 mL). Reaction mixture was heated at 120° C. for 30 minutes. LCMS showed that the reaction was complete. Solvent was removed. Ethanol (5 mL) and 300 uL of 2N NaOH solution were added to residue. The resulted solution was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (23 mg, 9%) was obtained. MS (ES+): m/z [M+H]⁺=449.8; ¹H NMR (400 MHz, DMSO-d₆) δ ppm 2.85 (s, 3H) 4.08 (d, J=5.81 Hz, 2H) 4.46 (s, 1H) 4.66 (s, 2H) 5.17 (s, 2H) 7.28 (d, J=7.07 Hz, 5H) 7.31-7.40 (m, 5H) 8.38 (s, 1H) 10.31 (s, 1H) 13.0 (br.s. 1H)

### Example 99

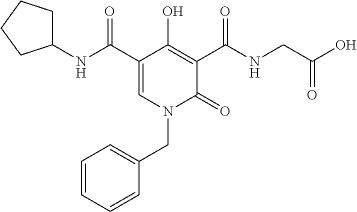

### N-{[5-[(cyclopentylamino)carbonyl]-4-hydroxy-2-oxo-1-(phenylmethyl)-1,2-dihydro-3-pyridinyl]carbonyl}glycine

99a) N-cyclopentyl-4-hydroxy-6-oxo-1-(phenylmethyl)-1,6-dihydro-3-pyridinecarboxamide. Into a 2 mL microwave tube were added methyl 4-hydroxy-6-oxo-1-(phenylmethyl)-1,6-dihydro-3-pyridinecarboxylate (519 mg, 2 mmol), and cyclopentaneamine (170 mg, 2 mmol). The reaction mixture was heated and stirred at 100° C. for 90 minutes. Crude product was then added into 2 mL of ethyl acetate. The resulted suspension was stirred and filtered. Solid was collected and dried. Crude product (270 mg, 43%) was obtained and used for next step without further purification.

99b) N-{[5-[(cyclopentylamino)carbonyl]-4-hydroxy-2-oxo-1-(phenylmethyl)-1,2-dihydro-3-pyridinyl]carbony}glycine. Into a 2 ml microwave tube were added N-cyclopentyl-4-hydroxy-6-oxo-1-(phenylmethyl)-1,6-dihydro-3-pyridinecarboxamide (270 mg, 0.86 mmol), DIEA (0.15 ml, 0.86 mmol), and ethyl N-(oxomethylidene)glycinate (112 mg, 0.86 mmol) in chloroform (1 ml). The resulted reaction mixture was heated at 120° C. for 30 minutes. Solvent was removed. Ethanol (2 mL) and 200 uL of 2N NaOH solution were added to the residue. Reaction was stirred at for 15 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (12.9 mg, 4%) was obtained. MS (ES+): m/z [M+H]⁺=413.8; ¹H NMR (400 MHz, DMSO-d₆) δ ppm 1.57 (br. s., 1H) 1.55 (d, J=4.55 Hz, 1H) 1.68 (br. s., 1H) 1.65 (d, J=7.07 Hz, 2H) 1.75 (s, 1H) 1.84-1.95 (m, 2H) 4.10 (d, J=5.81 Hz, 2H) 4.15-4.24 (m, 1H) 5.25 (s, 2H) 7.27-7.39 (m, 5H) 8.03 (d, J=7.58 Hz, 1H) 8.67 (s, 1H) 10.37 (br. s., 1H) 12.97 (br. s., 1H)

### Example 100

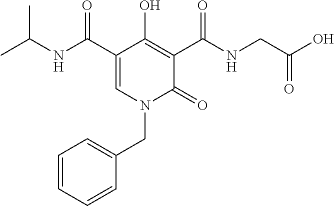

### N-{[4-hydroxy-5-{[(1-methylethyl)amino]carbonyl}-2-oxo-1-(phenylmethyl)-1,2-dihydro-3-pyridinyl]carbonyl}glycine

100a) 4-Hydroxy-N-(1-methylethyl)-6-oxo-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (540 mg, 2.2 mmol), EDC (422 mg, 2.2 mmol), and HOBt (337 mg, 2.2 mmol) in N,N-Dimethylformamide (DMF) (2 ml) and 8 ml of DCM. The resulted mixture was stirred for 30 minutes before isopropyl amine (130 mg, 2.2 mmol) was added. Then reaction mixture was stirred at room temperature overnight. LCMS showed that the reaction was complete. Reaction mixture was concentrated and purified with Gilson preparative HPLC to give desired product (201 mg, 32%).

100b) N-{[4-hydroxy-5-{[(1-methylethyl)amino]carbonyl}-2-oxo-1-(phenylmethyl)-1,2-dihydro-3-pyridinyl]carbonyl}glycine. Into a 2 mL microwave tube were added 4-hydroxy-N-(1-methylethyl)-6-oxo-1-(phenylmethyl)-1,6-dihydro-3-pyridinecarboxamide (198 mg, 0.69 mmol), ethyl N-(oxomethylidene)glycinate (89 mg, 0.69 mmol), and DIEA (0.12 mL, 0.69 mmol) in chloroform (1 mL). Reaction mixture was then heated at 120° C. for one hour in microwave reactor. LCMS showed that the reaction was complete. Solvent was removed. Ethanol (2 mL) and 1 mL of 2N NaOH solution were added to the residue. The mixture was stirred at rt for 15 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. The title product (18 mg, 7%) was obtained. MS (ES+): m/z [M+H]⁺=388.2; NMR N3875-81-A1: ¹H NMR (400 MHz, DMSO-d₆) δ ppm 1.16 (d, J=6.57 Hz, 6H) 4.04 (s, 1H) 4.10 (d, J=5.56 Hz, 2H) 5.26 (s, 2H) 7.28-7.40 (m, 5H) 7.93 (br. s., 1H) 8.68 (s, 1H) 10.38 (br. s., 1H) 12.98 (br. s., 1H)

### Example 101

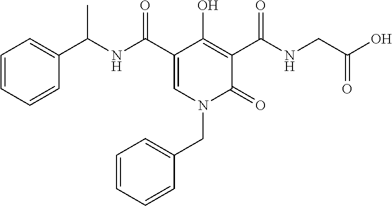

### N-{[4-hydroxy-2-oxo-5-{[(1-phenylethyl)amino]carbonyl}-1-(phenylmethyl)-1,2-dihydro-3-pyridinyl]carbonyl}glycine

101a) 4-Hydroxy-6-oxo-N-(1-phenylethyl)-1-(phenylmethyl)-1,6-dihydro-3-pyridinecarboxamide. Into a 2 mL microwave tube were added methyl 4-hydroxy-6-oxo-1-(phenylmethyl)-1,6-dihydro-3-pyridinecarboxylate (259 mg, 1.0 mmol) and (1-phenylethyl)amine (121 mg, 1.0 mmol). The reaction was stirred and heated at 100° C. for two hours. Ethyl acetate (3 mL) was added into reaction mixture. Reaction mixture was stirred and filtered. Solid was collected and dried. The desired product (122 mg, 35%) was obtained and used for next step without further purification.

101b) N-{[4-Hydroxy-2-oxo-5-{[(1-phenylethyl)amino]carbonyl}-1-(phenylmethyl)-1,2-dihydro-3-pyridinyl]carbonyl}glycine. Into a 2 mL microwave tube were added 4-hydroxy-6-oxo-N-(1-phenylethyl)-1-(phenylmethyl)-1,6-dihydro-3-pyridinecarboxamide (122 mg, 0.35 mmol), DIEA (45 mg, 0.35 mmol) and ethyl N-(oxomethylidene)glycinate (45.2 mg, 0.35 mmol) in DCM (1 mL). Reaction mixture was stirred and heated at 120° C. for two hours. Solvent was removed and the residue (dissolved in DMSO) was purified with HPLC system. Fractions were collected and concentrated. Ethyl alcohol (2 mL) and 100 ul of 10 N NaOH solutions were added into residue. Reaction mixture was stirred at for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (23.5 mg, 15%) was obtained. MS (ES+): m/z [M+H]⁺=449.8; ¹H NMR (400 MHz, DMSO-d₆) δ ppm 1.46 (d, J=7.07 Hz, 3H) 4.11 (d, J=5.56 Hz, 2H) 5.13 (d, J=7.33 Hz, 1H) 5.25 (s, 2H) 7.26 (s, 1H) 7.25 (d, J=7.07 Hz, 1H) 7.28-7.40 (m, 8H) 8.44 (s, 1H) 8.67 (s, 1H) 9.42-9.44 (m, 0H) 10.37 (br. s., 1H) 12.98 (br. s., 1H)

### Example 102

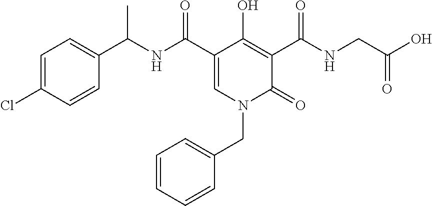

### N-{[5-({1-(4-chlorophenyl)ethyl]amino}carbonyl)-4-hydroxy-2-oxo-1-(phenylmethyl)-1,2-dihydro-3-pyridinyl)carbonyl}glycine

102a) N-[1-(4-Chlorophenyl)ethyl]-4-hydroxy-6-oxo-1-(phenylmethyl)-1,6-dihydro-3-pyridinecarboxamide. Into a 2 mL microwave tube were added methyl 4-hydroxy-6-oxo-1-(phenylmethyl)-1,6-dihydro-3-pyridinecarboxylate (311 mg, 1.2 mmol) and [1-(4-chlorophenyl)ethyl]amine (187 mg, 1.2 mmol). The reaction was stirred and heated at 100° C. for two hours. Ethyl acetate (3 mL) was added into reaction mixture. The resulted suspension was stirred and filtered. Solid was collected and dried. Crude product was further purified with Gilson preparative HPLC to give desired product (107 mg, 23%).

102b) N-{[5-({[1-(4-Chlorophenyl)ethyl]amino}carbonyl)-4-hydroxy-2-oxo-1-(phenylmethyl)-1,2-dihydro-3-pyridinyl]carbonyl}glycine. Into a 2 mL microwave tube were added N-[1-(4-chlorophenyl)ethyl]-4-hydroxy-6-oxo-1-(phenylmethyl)-1,6-dihydro-3-pyridinecarboxamide (107 mg, 0.28 mmol), and DIEA (36 mg, 0.28 mmol) and ethyl N-(oxomethylidene)glycinate (35.7 mg, 0.28 mmol) in chloroform (1 mL). Reaction mixture was stirred and heated at 120° C. for two hours. Reaction mixture was concentrated and residue (dissolved in DMSO) was purified with HPLC system. Fractions were collected and concentrated. Ethyl alcohol (5 mL) and then 100 ul of 10 N NaOH solution were added into residue. The resulted solution was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (24 mg, 18%) was obtained. MS (ES+): m/z [M+H]⁺=484.0; ¹H NMR (400 MHz, DMSO-d₆) δ ppm 1.45 (d, J=7.07 Hz, 3H) 4.11 (d, J=5.56 Hz, 2H) 5.11 (t, J=7.33 Hz, 1H) 5.24 (s, 2H) 7.31 (d, J=1.77 Hz, 2H) 7.27-7.32 (m, 1H) 7.33-7.42 (m, 6H) 8.47 (br. s., 1H) 8.65 (s, 1H) 10.37 (br. s., 1H) 13.00 (br. s., 1H)

### Example 103

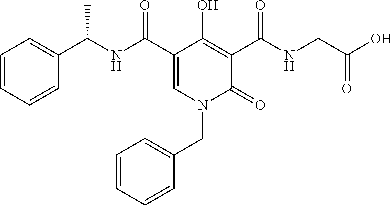

### N-{[4-hydroxy-2-oxo-5-({[(1S)-1-phenylethyl]amino}carbonyl)-1-(phenylmethyl)-1,2-dihydro-3-pyridinyl]carbonyl}glycine

103a) 4-Hydroxy-6-oxo-N-[(1S)-1-phenylethyl]-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (162 mg, 0.66 mmol), TEA (101 mg, 1.0 mmol), and HATU (276 mg, 0.726 mmol) in N,N-Dimethylformamide (DMF) (2 ml) and 8 ml of DCM. Reaction mixture was stirred for 30 minutes before [(1S)-1-phenylethyl]amine (80 mg, 0.66 mmol) was added. Then reaction mixture was stirred at room temperature overnight. LCMS showed that the reaction was complete. Reaction mixture was diluted with ethyl acetate, washed with 5% HCl solution and 5% NaHCO₃ solution. Organic layer was dried and concentrated under reduced pressure. The desired product (137 mg, 60%) was obtained and used for next step without further purification.

103b) N-{[4-Hydroxy-2-oxo-5-({[(1S)-1-phenylethyl]amino}carbonyl)-1-(phenylmethyl)-1,2-dihydro-3-pyridinyl]carbonyl}glycine. Into a 2 mL microwave tube were added 4-hydroxy-6-oxo-N-[(1S)-1-phenylethyl]-1-(phenylmethyl)-1,6-dihydro-3-pyridinecarboxamide (137 mg, 0.39 mmol), DIEA (50.8 mg, 0.39 mmol) and ethyl N-(oxomethylidene)glycinate (50.8 mg, 0.39 mmol) in chloroform (1 mL). Reaction mixture was stirred and heated at 120° C. for two hours. Reaction mixture was concentrated and residue (dissolved in DMSO) was purified with Gilson HPLC system. Fractions were collected and concentrated. Ethyl alcohol (5 mL) and 100 ul of 10 N NaOH solution were added to the residue. Reaction was stirred at rt for 30 minutes. LCMS showed that the reaction was complete. Reaction mixture was concentrated and diluted with 2 ml of water. The solution was cooled and then acidified to pH around 2 by addition of 6N HCl. The solution was cooled and stirred until precipitation was complete. The solid was washed, collected and dried under vacuum oven to give the titled compound (32 mg, 18%). MS (ES+): m/z [M+H]⁺=450.1; ¹H NMR (400 MHz, DMSO-d₆) δ ppm 1.46 (d, J=7.07 Hz, 3H) 4.11 (s, 2H) 5.12 (s, 1H) 5.25 (s, 2 H) 7.26 (s, 1H) 7.28-7.39 (m, 9H) 8.46 (s, 1H) 8.67 (s, 1H) 10.37 (s, 1H) 12.98 (s, 1H)

### Example 104

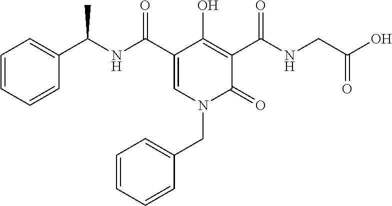

### N-{[4-hydroxy-2-oxo-5-({[(1R)-1-phenylethyl]amino}carbonyl)-1-(phenylmethyl)-1,2-dihydro-3-pyridinyl]carbonyl}glycine

104a) 4-Hydroxy-6-oxo-N-[(1R)-1-phenylethyl]-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (147 mg, 0.60 mmol), TEA (0.125 ml, 0.90 mmol), and HATU (251 mg, 0.66 mmol) in N,N-Dimethylformamide (DMF) (2 ml) and 8 ml of DCM. Reaction mixture was stirred for 30 minutes before amine [(1R)-1-phenylethyl]amine (72.7 mg, 0.6 mmol) was added. Then reaction mixture was stirred at room temperature overnight. LCMS showed that the reaction was complete. Reaction mixture was diluted with ethyl acetate, washed with 5% HCl solution and 5% NaHCO₃ solution. Organic layer was dried and concentrated under reduced pressure. The desired product (125 mg, 51%) was obtained and used for next step without further purification

104b) N-{[4-Hydroxy-2-oxo-5-({[(1R)-1-phenylethyl]amino}carbonyl)-1-(phenylmethyl)-1,2-dihydro-3-pyridinyl]carbonyl}glycine. Into a 2 mL microwave tube were added 4-hydroxy-6-oxo-N-[(1R)-1-phenylethyl]-1-(phenylmethyl)-1,6-dihydro-3-pyridinecarboxamide (125 mg, 0.36 mmol), DIEA (46.3 mg, 0.36 mmol) and ethyl N-(oxomethylidene)glycinate (46.3 mg, 0.36 mmol) in DCM (1 mL). Reaction mixture was stirred and heated at 120° C. for two hours. Reaction mixture was concentrated and residue (dissolved in DMSO) was purified with Gilson HPLC system. Fractions were collected and concentrated. Ethyl alcohol (5 mL) and 100 ul of 10 N NaOH solution were added to the residue. Reaction mixture was stirred for 30 minutes. LCMS showed that the reaction was complete. Reaction mixture was concentrated and diluted with 2 ml of water. The solution was cooled and then acidified to pH around 2 by addition of 6N HCl. The solution was cooled and stirred until precipitation was complete. The solid was washed, collected and dried under vacuum oven to yield the desired product (16.5 mg, 10%) was obtained. MS (ES+): m/z [M+H]⁺=450.1; ¹H NMR (400 MHz, DMSO-d₆) δ ppm 1.46 (d, J=6.82 Hz, 3H) 4.11 (s, 2H) 5.12 (s, 1H) 5.25 (s, 2H) 7.28-7.40 (m, 10H) 8.46 (s, 1H) 8.67 (s, 1H) 10.37 (s, 1H) 12.98 (s, 1 H)

### Example 105

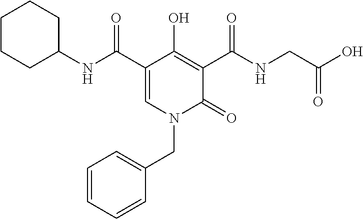

### N-{[5-[(Cyclohexylamino)carbonyl]-4-hydroxy-2-oxo-1-(phenylmethyl)-1,2-dihydro-3-pyridinyl]carbonyl}glycine

105a) N-Cyclohexyl-4-hydroxy-6-oxo-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (147 mg, 0.6 mmol), TEA (0.084 ml, 0.6 mmol), and HATU (228 mg, 0.6 mmol) in N,N-Dimethylformamide (DMF) (2 ml) and 8 ml of DCM. The reaction mixture was stirred for 30 minutes before cyclohexylamine (59.4 mg, 0.6 mmol) was added. Then reaction mixture was stirred at room temperature overnight. LCMS showed the reaction was complete. Reaction mixture was concentrated and purified with Gilson preparative HPLC to give desired product (57 mg, 29%).

105b) N-{[5-[(cyclohexylamino)carbonyl]-4-hydroxy-2-oxo-1-(phenylmethyl)-1,2-dihydro-3-pyridinyl]carbonyl}glycine. Into a 2 mL microwave reaction vessel were added 4-hydroxy-N-cyclohexyl-6-oxo-1-(phenylmethyl)-1,6-dihydro-3-pyridinecarboxamide (57 mg, 0.18 mmol), ethyl N-(oxomethylidene)glycinate (22.5 mg, 0.18 mmol), and DIEA (30.5 μl, 0.18 mmol) in chloroform (1 mL). Reaction mixture was heated at 120° C. with microwave reactor for 30 minutes. LCMS showed that the reaction was complete. The reaction mixture was concentrated and then added ethanol 2 mL and 200 uL of 2N NaOH solution. The resulted reaction mixture was stirred for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (20 mg, 26%) was obtained. MS (ES+): m/z [M+H]⁺=428.1; ¹H NMR (400 MHz, MeOD) δ ppm 1.38 (s, 5H) 1.73 (br. s., 5H) 3.87 (s, 1H) 4.18 (s, 2H) 5.23 (s, 2H) 7.28-7.39 (m, 5H) 8.54 (s, 1H)

### Example 106

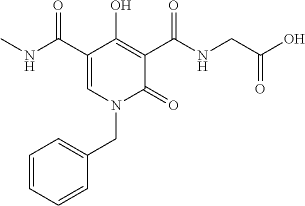

### N-{[4-Hydroxy-5-[methylamino)carbonyl]-2-oxo-1-(phenylmethyl)-1,2-dihydro-3-pyridinyl]carbonyl}glycine

106a) 4-Hydroxy-N-methyl-6-oxo-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (147 mg, 0.60 mmol), TEA (0.084 ml, 0.60 mmol), and HATU (228 mg, 0.60 mmol) in N,N-Dimethylformamide (DMF) (2 ml) and 8 ml of DCM. Reaction mixture was stirred at room temperature for 30 minutes before methanamine hydrochloride (39.3 mg, 0.60 mmol) and TEA (0.084 ml, 0.60 mmol) were added. Then reaction mixture was stirred at room temperature overnight. LCMS showed the reaction was complete. Reaction mixture was concentrated and purified with Gilson preparative HPLC to give pure product (54 mg, 35%).

106b) N-{[4-Hydroxy-5-[(methylamino)carbonyl]-2-oxo-1-(phenylmethyl)-1,2-dihydro-3-pyridinyl]carbonyl}glycine. Into a 2 mL microwave reaction vessel were added 4-hydroxy-N-methyl-6-oxo-1-(phenylmethyl)-1,6-dihydro-3-pyridinecarboxamide (54 mg, 0.21 mmol), ethyl N-(oxomethylidene)glycinate (27 mg, 0.21 mmol), and DIEA (27 mg, 0.21 mmol) in chloroform (1 mL). Reaction mixture was heated at 120° C. in microwave reactor for 30 minutes. LCMS showed the reaction was complete. Chloroform was removed. Ethanol (2 mL) and 200 uL of 2N NaOH solution were added into residue. The resulted reaction mixture was stirred for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (9 mg, 13%) was obtained. MS (ES+): m/z [M+H]⁺=360.1; ¹H NMR (400 MHz, MeOD) δ ppm 2.91 (s, 3H) 4.18 (s, 2H) 5.23 (s, 2H) 7.33-7.38 (m, 5H) 8.57 (s, 1H)

### Example 107

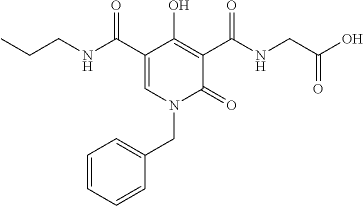

### N-({4-Hydroxy-2-oxo-1-(phenylmethyl)-5-[(propylamino)carbonyl]-1,2-dihydro-3-pyridinyl}carbonyl)glycine

107a) 4-Hydroxy-6-oxo-1-(phenylmethyl)-N-propyl-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (147 mg, 0.60 mmol), TEA (0.084 ml, 0.60 mmol), and HATU (228 mg, 0.60 mmol) in N,N-Dimethylformamide (DMF) (2 ml) and 8 ml of DCM. Reaction mixture was stirred at room temperature for 30 minutes before propylamine (35.4 mg, 0.60 mmol) was added. Then reaction mixture was stirred at room temperature overnight. LCMS showed the reaction was complete. Reaction mixture was concentrated and purified with Gilson preparative HPLC to give desired product (41 mg, 24%).

107b) N-({4-Hydroxy-2-oxo-1-(phenylmethyl)-5-[(propylamino)carbonyl]-1,2-dihydro-3-pyridinyl}carbonyl)glycine. Into a 2 mL microwave reaction vessel were added 4-hydroxy-6-oxo-1-(phenylmethyl)-N-propyl-1,6-dihydro-3-pyridinecarboxamide (41 mg, 0.14 mmol), ethyl N-(oxomethylidene)glycinate (18 mg, 0.14 mmol), and DIEA (18 mg, 0.14 mmol) in chloroform (1 mL). Reaction mixture was heated at 120° C. in microwave reactor for 30 minutes. LCMS showed that the reaction was complete. Chloroform was removed. Ethanol (2 mL) and 200 uL of 2N NaOH solution were added to the residue. The resulted reaction mixture was stirred for 30 minutes. LCMS showed that reaction was completed. Solvent was removed and diluted with 2 mL of water. The pH of the resulted solution was adjusted to 1˜2 with 6N HCl. The reaction mixture was concentrated and the residue was dissolved in DMSO (2 mL). The DMSO solution was filtered and purified with Gilson preparative HPLC. Desired product (11.5 mg, 21%) was obtained. MS (ES+): m/z [M+H]⁺=388.1; ¹H NMR (400 MHz, DMSO-d₆) δ ppm 0.88 (t, J=7.33 Hz, 3H) 1.46-1.57 (m, 2H) 3.19-3.28 (m, 2H) 4.05 (s, 2H) 5.26 (s, 2H) 7.26-7.38 (m, 5H) 8.14 (br. s., 1H) 8.68 (s, 1H) 10.37 (br. s., 1H)

### Example 108

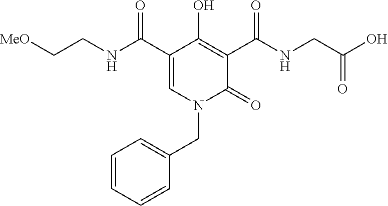

### N-{[4-Hydroxy-5-({[2-(methyloxy)ethyl]amino}carbonyl)-2-oxo-1-(phenylmethyl)-1,2-dihydro-3-pyridinyl]carbonyl}glycine

108a) 4-Hydroxy-N-[2-(methyloxy)ethyl]-6-oxo-1-(phenylmethyl)-1,6-dihydro-3-pyridinecarboxamide. Into a 50 mL round-bottomed flask were added 4-hydroxy-6-oxo-1-(phenylmethyl)-1,6-dihydro-3-pyridinecarboxylic acid (147 mg, 0.60 mmol), TEA (0.084 ml, 0.60 mmol), and HATU (228 mg, 0.60 mmol) in N,N-Dimethylformamide (DMF) (2 ml) and 8 ml of DCM. Reaction mixture was stirred at room temperature for 30 minutes before 2-methoxyethylamine (45.0 mg, 0.60 mmol) was added. Then reaction mixture was stirred at room temperature overnight. LCMS showed that the reaction was complete. Reaction mixture was concentrated and purified with Gilson preparative HPLC to give pure product (52 mg, 28%).

108b) N-{[4-Hydroxy-5-({[2-(methyloxy)ethyl]amino}carbonyl)-2-oxo-1-(phenylmethyl)-1,2-dihydro-3-pyridinyl]carbonyl}glycine. Into a 2 mL microwave reaction vessel were added 4-hydroxy-N-cyclohexyl-6-oxo-1-(phenylmethyl)-1,6-dihydro-3-pyridinecarboxamide (52 mg, 0.17 mmol), ethyl N-(oxomethylidene)glycinate (22.2 mg, 0.17 mmol), and DIEA (22.2 mg, 0.17 mmol) in chloroform (1 mL). Reaction mixture was heated at 120° C. for 30 minutes. LCMS showed that the reaction was complete. Chloroform was removed. Ethanol (2 mL) and 200 uL of 2N NaOH solution were added to the residue. The resulted reaction mixture was stirred for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (17 mg, 24%) was obtained. MS (ES+): m/z [M+H]⁺=404.2; ¹H NMR (400 MHz, DMSO-d₆) δ ppm 3.26 (s, 1H) 3.27 (s, 3H) 3.32 (br. s., 1H) 3.34 (s, 1H) 3.41-3.48 (m, 2H) 4.05 (s, 2H) 5.26 (s, 2H) 7.26-7.38 (m, 5H) 8.72 (s, 1H) 10.37 (s, 1H)

### Example 109

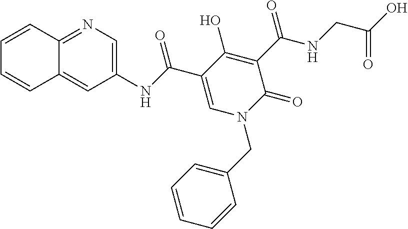

### N-({4-Hydroxy-2-oxo-1-(phenylmethyl)-5-[(3-quinolinylamino)carbonyl]-1,2-dihydro-3-pyridinyl}carbonyl)glycine

109a) 4-Hydroxy-6-oxo-1-(phenylmethyl)-N-3-quinolinyl-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.6 mmol), and 3H-[1,2,3]triazolo[4,5-b]pyridin-3-ol hydrate (92 mg, 0.6 mmol) in N,N-Dimethylformamide (DMF) (3 mL). The reaction mixture was stirred at room temperature for 30 minutes. 3-quinolinamine (87 mg, 0.6 mmol) was added. The reaction mixture was then stirred at room temperature overnight. LCMS showed that the reaction was complete. The reaction mixture was filtered and purified with Gilson preparative HPLC. Product (26 mg, 12%) was obtained.

109b) N-({4-Hydroxy-2-oxo-1-(phenylmethyl)-5-[(3-quinolinylamino)carbonyl]-1,2-dihydro-3-pyridinyl}carbonyl)glycine. Into a 2 mL microwave reaction vessel were added 4-hydroxy-6-oxo-1-(phenylmethyl)-N-3-quinolinyl-1,6-dihydro-3-pyridinecarboxamide (26 mg, 0.07 mmol), ethyl N-(oxomethylidene)glycinate (9.0 mg, 0.07 mmol), and DIEA (9.0 mg, 0.07 mmol) in chloroform (1 mL). Reaction mixture was heated at 120° C. for 30 minutes. LCMS showed that the reaction was complete. Chloroform was removed. Ethanol (2 mL) and 200 uL of 2N NaOH solution were added to the residue. The resulted reaction mixture was stirred for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (21 mg, 63%) was obtained. MS (ES+): m/z [M+H]⁺=473.3; ¹H NMR (400 MHz, DMSO-d₆) δ ppm 3.17 (s, 1H) 4.14 (d, J=5.56 Hz, 2H) 5.30 (s, 2H) 7.30-7.42 (m, 5H) 7.61 (t, J=7.45 Hz, 1H) 7.69 (dd, J=8.34, 1.52 Hz, 4H) 7.98 (t, J=7.58 Hz, 2H) 8.80 (d, J=2.27 Hz, 1H) 8.85 (s, 1H) 9.08 (d, J=2.53 Hz, 1H) 10.38 (br. s., 1H) 10.50 (br. s., 1H)

### Example 110

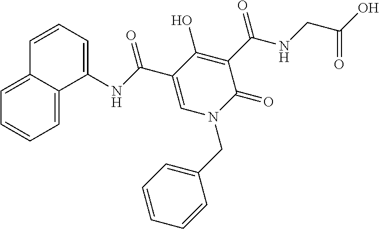

### N-{[4-Hydroxy-5-[(1-naphthalenylamino)carbonyl]-2-oxo-1-(phenylmethyl)-1,2-dihydro-3-pyridinyl]carbonyl}glycine

110a) 4-Hydroxy-N-1-naphthalenyl-6-oxo-1-(phenylmethyl)-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.6 mmol), and 3H-[1,2,3]triazolo[4,5-b]pyridin-3-ol hydrate (92 mg, 0.6 mmol) in N,N-Dimethylformamide (3 mL). The reaction mixture was stirred at room temperature for 30 minutes before 1-naphthalenylamine (86 mg, 0.6 mmol) was added. The reaction mixture was then stirred at room temperature overnight. LCMS showed the reaction was complete. The reaction mixture was filtered and purified with Gilson preparative HPLC. Desired product (32 mg, 14%) was obtained.

110b) N-{[4-Hydroxy-5-[(1-naphthalenylamino)carbonyl]-2-oxo-1-(phenylmethyl)-1,2-dihydro-3-pyridinyl]carbonyl}glycine. Into a 2 mL microwave reaction vessel were added 4-hydroxy-N-1-naphthalenyl-6-oxo-1-(phenylmethyl)-1,6-dihydro-3-pyridinecarboxamide (32 mg, 0.09 mmol), ethyl N-(oxomethylidene)glycinate (12 mg, 0.09 mmol), and DIEA (12 mg, 0.09 mmol) in chloroform (1 mL). Reaction mixture was heated at 120° C. for 30 minutes. LCMS showed that the reaction was complete. Chloroform was removed. Ethanol (2 mL) and 200 uL of 2N NaOH solution were added to the residue. The resulted reaction mixture was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (31 mg, 76%) was obtained. MS (ES+): m/z [M+H]⁺=472.2; ¹H NMR (400 MHz, DMSO-d₆) δ ppm 4.10 (br. s., 2H) 5.29 (br. s., 2H) 7.36 (d, J=2.27 Hz, 6H) 7.53 (s, 2H) 7.87 (m, 1H) 7.97 (s, 1H) 8.14 (br. s., 1H) 8.25 (br. s., 1H) 8.81 (s, 1H)

### Example 111

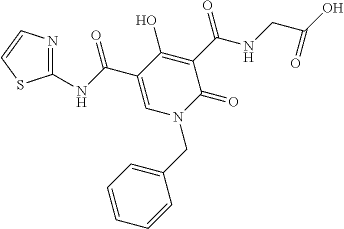

### N-({4-Hydroxy-2-oxo-1-(phenylmethyl)-5-[(1,3-thiazol-2-ylamino)carbonyl]-1,2-dihydro-3-pyridinyl}carbonyl)glycine

111a) 4-Hydroxy-6-oxo-1-(phenylmethyl)-N-1,3-thiazol-2-yl-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.6 mmol), and 3H-[1,2,3]triazolo[4,5-b]pyridin-3-ol hydrate (92 mg, 0.6 mmol) in N,N-Dimethylformamide (3 mL). The reaction mixture was stirred at room temperature for 30 minutes before 1,3-thiazol-2-amine (60 mg, 0.6 mmol) was added. The reaction mixture was then stirred at room temperature overnight. LCMS showed that the reaction was complete. The reaction mixture was filtered and purified with Gilson preparative HPLC. Desired product (18 mg, 9%) was obtained.

111b) N-({4-Hydroxy-2-oxo-1-(phenylmethyl)-5-[(1,3-thiazol-2-ylamino)carbonyl]-1,2-dihydro-3-pyridinyl}carbonyl)glycine. Into a 2 mL microwave reaction vessel were added 4-hydroxy-6-oxo-1-(phenylmethyl)-N-1,3-thiazol-2-yl-1,6-dihydro-3-pyridinecarboxamide (18 mg, 0.06 mmol), ethyl N-(oxomethylidene)glycinate (8 mg, 0.06 mmol), and DIEA (8 mg, 0.06 mmol) in chloroform (1 mL). Reaction mixture was heated at 120° C. for 30 minutes. LCMS showed that the reaction was complete. Chloroform was removed. Ethanol (2 mL) and 200 uL of 2N NaOH solution were added to the residue. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (10.5 mg, 44%) was obtained. MS (ES+): m/z [M+H]⁺=428.8; ¹H NMR (400 MHz, DMSO-d₆) δ ppm 3.78 (br. s., 2H) 5.13 (s, 2H) 7.25-7.36 (m, 1H) 7.30 (ddd, J=14.27, 7.33, 7.20 Hz, 6H) 8.46 (s, 1H) 9.11 (s, 1H) 9.87 (br. s., 1H)

### Example 112

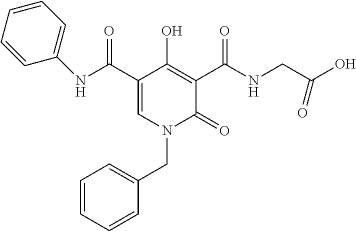

### N-{[4-Hydroxy-2-oxo-5-[(phenylamino)carbonyl]-1-(phenylmethyl)-1,2-dihydro-3-pyridinyl]carbonyl}glycine

112a) 4-Hydroxy-6-oxo-N-phenyl-1-(phenylmethyl)-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.6 mmol), and 3H-[1,2,3]triazolo[4,5-b]pyridin-3-ol hydrate (92 mg, 0.6 mmol) in N,N-Dimethylformamide (3 mL). The reaction mixture was stirred at room temperature for 30 minutes before aniline (56 mg, 0.6 mmol) was added. The reaction mixture was then stirred at room temperature overnight. LCMS showed that the reaction was complete. The reaction mixture was filtered and purified with Gilson preparative HPLC. Desired product (41 mg, 21%) was obtained.

112b) N-{[4-Hydroxy-2-oxo-5-[(phenylamino)carbonyl]-1-(phenylmethyl)-1,2-dihydro-3-pyridinyl]carbonyl}glycine. Into a 2 mL microwave reaction vessel were added 4-hydroxy-6-oxo-N-phenyl-1-(phenylmethyl)-1,6-dihydro-3-pyridinecarboxamide (41 mg, 0.13 mmol), ethyl N-(oxomethylidene)glycinate (17 mg, 0.13 mmol), and DIEA (17 mg, 0.13 mmol) in chloroform (1 mL). Reaction mixture was at 120° C. for 30 minutes. LCMS showed that the reaction was complete. Chloroform was removed. Ethanol (2 mL) and 200 uL of 2N NaOH solution were added to the residue. The resulted reaction mixture was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (14 mg, 26%) was obtained. MS (ES+): m/z [M+H]⁺=422.1; ¹H NMR (400 MHz, DMSO-d₆) δ ppm 3.90 (br. s., 2H) 5.16 (br. s., 2H) 7.04 (br. s., 1H) 7.25-7.37 (m, 8H) 7.67 (d, J=7.83 Hz, 2H) 8.45 (br s, 1H) 10.19 (br s, 1H)

### Example 113

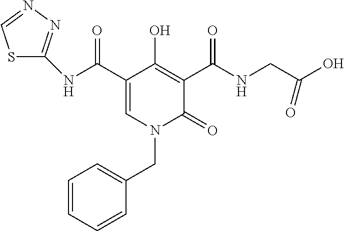

### N-({4-hydroxy-2-oxo-1-(phenylmethyl)-5-[(1,3,4-thiadiazol-2-ylamino)carbonyl]-1,2-dihydro-3-pyridinyl}carbonyl)glycine

113a) 4-hydroxy-6-oxo-1-(phenylmethyl)-N-1,3,4-thiadiazol-2-yl-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.60 mmol), and 3H-[1,2,3]triazolo[4,5-b]pyridin-3-ol hydrate (92 mg, 0.60 mmol) in N,N-Dimethylformamide (3 mL). The reaction mixture was stirred at room temperature for 30 minutes before 1,3,4-thiadiazol-2-amine (61 mg, 0.6 mmol) was added. The reaction mixture was then stirred at room temperature overnight. LCMS showed that the reaction was complete. The reaction mixture was filtered and purified with Gilson preparative HPLC. Desired product (11 mg, 6%) was obtained.

113b) N-({4-hydroxy-2-oxo-1-(phenylmethyl)-5-[(1,3,4-thiadiazol-2-ylamino)carbonyl]-1,2-dihydro-3-pyridinyl}carbonyl)glycine. Into a 2 mL microwave reaction vessel were added 4-hydroxy-6-oxo-1-(phenylmethyl)-N-1,3,4-thiadiazol-2-yl-1,6-dihydro-3-pyridinecarboxamide (11 mg, 0.03 mmol), ethyl N-(oxomethylidene)glycinate (6 mg, 0.04 mmol), and DIEA (6 mg, 0.04 mmol) in chloroform (1 mL). Reaction mixture was heated at 120° C. in microwave reactor for 30 minutes. LCMS showed that the reaction was complete. Chloroform was removed. Ethanol (2 mL) and 200 uL of 2N NaOH solution were added to residue. The resulted reaction mixture was stirred at rt for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (6 mg, 41%) was obtained. MS (ES+): m/z [M+H]⁺=430; ¹H NMR (400 MHz, DMSO-d₆) δ ppm 3.78 (br. s., 2 H) 5.13 (s, 2H) 7.25-7.36 (m, 1H) 7.30 (ddd, J=14.27, 7.33, 7.20 Hz, 6H) 8.46 (s, 1H) 9.11 (s, 1H) 9.87 (br. s., 1H)

### Example 114

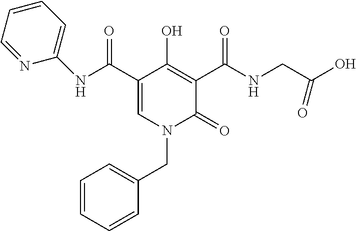

### N-({4-Hydroxy-2-oxo-1-(phenylmethyl)-5-[(2-pyridinylamino)carbonyl]-1,2-dihydro-3-pyridinyl}carbonyl)glycine

114a) 4-Hydroxy-6-oxo-1-(phenylmethyl)-N-2-pyridinyl-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.60 mmol), and 3H-[1,2,3]triazolo[4,5-b]pyridin-3-ol hydrate (92 mg, 0.60 mmol) in N,N-Dimethylformamide (3 mL). The reaction mixture was stirred at room temperature for 30 minutes before 2-aminepyridine (56 mg, 0.6 mmol) was added. The reaction mixture was then stirred at room temperature overnight. LCMS showed that the reaction was complete. The reaction mixture was filtered and purified with Gilson preparative HPLC. Product (48 mg, 25%) was obtained.

114b) N-({4-hydroxy-2-oxo-1-(phenylmethyl)-5-[(2-pyridinylamino)carbonyl]-1,2-dihydro-3-pyridinyl}carbonyl)glycine. Into a 2 mL microwave reaction vessel were added 4-hydroxy-6-oxo-1-(phenylmethyl)-N-2-pyridinyl-1,6-dihydro-3-pyridinecarboxamide (48 mg, 0.15 mmol), ethyl N-(oxomethylidene)glycinate (20 mg, 0.15 mmol), and DIEA (20 mg, 0.15 mmol) in chloroform (1 mL). Reaction mixture was heated at 120° C. in microwave reactor for 30 minutes. LCMS showed that the reaction was complete. Chloroform was removed. Ethanol (2 mL) and 200 uL of 2N NaOH solution were added to the residue. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (12.5 mg, 20%) was obtained. MS (ES+): m/z [M+H]⁺=422.1; ¹H NMR (400 MHz, DMSO-d₆) δ ppm 3.88 (br. s., 2H) 5.15 (br. s., 2H) 7.07 (br. s., 2H) 7.25-7.37 (m, 5H) 7.77 (s, 1H) 8.22 (d, J=8.34 Hz, 1H) 8.30 (br. s., 1H) 8.45 (br. s., 1H) 10.5 (br. s., 1H)

### Example 115

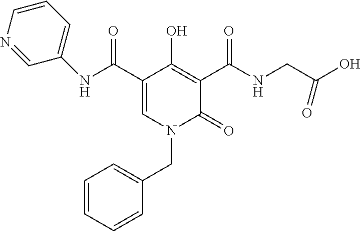

### N-({4-Hydroxy-2-oxo-1-(phenylmethyl)-5-[(3-pyridinylamino)carbonyl]-1,2-dihydro-3-pyridinyl}carbonyl)glycine

115a) 4-Hydroxy-6-oxo-1-(phenylmethyl)-N-3-pyridinyl-1,6-dihydro-3-pyridinecarboxamide. Into a 25 mL round-bottomed flask were added 4-hydroxy-6-oxo-1-(phenylmethyl)-1,6-dihydro-3-pyridinecarboxylic acid (147 mg, 0.6 mmol), EDC (115 mg, 0.6 mmol), and 3H-[1,2,3]triazolo[4,5-b]pyridin-3-ol hydrate (92 mg, 0.6 mmol) in N,N-Dimethylformamide (3 mL). The reaction mixture was stirred at room temperature for 30 minutes before 3-aminepyridine (56 mg, 0.6 mmol) was added. The reaction mixture was then stirred at room temperature overnight. LCMS showed that the reaction was complete. The reaction mixture was filtered and purified with Gilson preparative HPLC. Desired product (98 mg, 51%) was obtained.

115b) N-({4-Hydroxy-2-oxo-1-(phenylmethyl)-5-[3-pyridinylamino)carbonyl]-1,2-dihydro-3-pyridinyl}carbonyl)glycine. Into a 2 mL microwave reaction vessel were added 4-hydroxy-6-oxo-1-(phenylmethyl)-N-3-pyridinyl-1,6-dihydro-3-pyridinecarboxamide (98 mg, 0.30 mmol), ethyl N-(oxomethylidene)glycinate (39 mg, 0.30 mmol), and DIEA (39 mg, 0.30 mmol) in chloroform (1 mL). Reaction mixture was heated at 120° C. in microwave reactor for 30 minutes. LCMS showed that the reaction was complete. Chloroform was removed. Ethanol (2 mL) and 200 uL of 2N NaOH solution were added to the residue. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (15 mg, 12%) was obtained. MS (ES+): m/z [M+H]⁺=422.1; ¹H NMR (400 MHz, DMSO-d₆) δ ppm 4.13 (d, J=5.56 Hz, 2H) 5.29 (s, 2H) 7.28-7.40 (m, 5H) 7.50 (d, J=8.34 Hz, 1H) 8.24 (s, 1H) 8.37 (dd, J=4.80, 1.52 Hz, 1H) 8.83 (s, 1H) 8.93 (d, J=2.02 Hz, 1H) 10.36 (br. s., 1H)

### Example 116

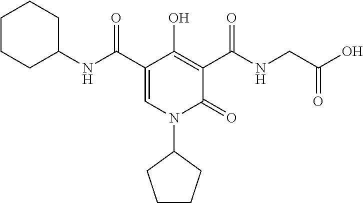

### N-({5-[1Cyclohexylamino)carbonyl]-1-cyclopentyl-4-hydroxy-2-oxo-1,2-dihydro-3-pyridinyl}carbonyl)glycine

116a) Methyl 1-cyclopentyl-4-hydroxy-6-oxo-1,6-dihydro-3-pyridinecarboxylate. Into a 500 mL round-bottomed flask were added acetondimethylacetate (6.97 g, 40 mmol), trimethyl orthformate (4.24 g, 40.0 mmol), and acetic anhydride (7.55 ml, 80 mmol). The resulted reaction mixture was stirred and heated at 120° C. for two hours. The mixture was then cooled down to room temperature. After removing all volatile material under reduced pressure, the residue was dissolved with THF (100 mL). Cyclopentylamine (3.41 g, 40.0 mmol) was added slowly. Reaction mixture was stirred at room temperature overnight. Sodium hydride (1.92 g, 80 mmol) was added portionwise and the resulted mixture was stirred at room temperature for 30 minutes. LCMS showed that the reaction was complete. The reaction mixture was concentrated and cooled down to 0° C., and added ethyl acetate (100 mL) and 30 ml of 10% HCl (to adjust pH of aqueous layer to pH 2). Two layers were separated and aqueous layer was extracted with ethyl acetate (75 mL×2). Combined organic layers were dried and solvent was removed. Crude product (3.1 g, 33%) was obtained and used without further purification.

116b) N-Cyclohexyl-1-cyclopentyl-4-hydroxy-6-oxo-1,6-dihydro-3-pyridinecarboxamide. Into a 2 mL microwave reaction vessel were added methyl 1-cyclopentyl-4-hydroxy-6-oxo-1,6-dihydro-3-pyridinecarboxylate (237 mg, 1.0 mmol), cyclohexylamine (99 mg, 1.0 mmol). This resulted reaction mixture was stirred and heated at 100° C. in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and then with brine (10 mL). Organic layer was dried over anhydrous Na₂SO₄. Removal of solvent yielded crude product (271 mg, 89%), which was used for next step without further purification.

116c) N-({5-[cyclohexylamino)carbonyl]-1-cyclopentyl-4-hydroxy-2-oxo-1,2-dihydro-3-pyridinyl}carbonyl)glycine. Into a 2 ml microwave reaction vessel were added N-cyclohexyl-1-cyclopentyl-4-hydroxy-6-oxo-1,6-dihydro-3-pyridinecarboxamide (152 mg, 0.5 mmol), TEA (0.07 mL, 0.5 mmol), and Ethyl N-(oxomethylidene)glycinate (65 mg, 0.5 mmol) in chloroform (1 mL). Reaction mixture was then heated at 120° C. in microwave reactor for 1 hour. LCMS showed that the reaction was complete. Chloroform was removed. Residue was dissolved in ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (24 mg, 12%) was obtained. MS (ES+): m/z [M+H]⁺=406.3; ¹H NMR (400 MHz, DMSO-d₆) δ ppm 1.30 (br. s., 5H) 1.65 (d, J=4.29 Hz, 6H) 1.81 (br. s., 6H) 2.07 (br. s., 2H) 3.81 (d, J=5.31 Hz, 1H) 4.12 (d, J=5.56 Hz, 2H) 5.04 (s, 1H) 7.99 (s, 1H) 8.38 (s, 1H) 10.49 (br. s., 1H) 12.99 (br. s., 1H)

### Example 117

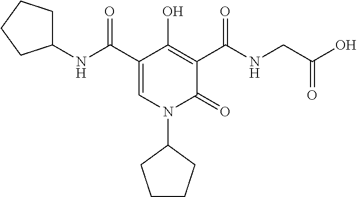

### N-({1-cyclopentyl-5-[cyclopentylamino)carbonyl]-4-hydroxy-2-oxo-1,2-dihydro-3-pyridinyl}carbonyl)glycine

117a) 1-dicyclopentyl-4-hydroxy-6-oxo-1,6-dihydro-3-pyridinecarboxamide. Into a 2 mL microwave reaction vessel were added methyl 1-cyclopentyl-4-hydroxy-6-oxo-1,6-dihydro-3-pyridinecarboxylate (237 mg, 1.0 mmol) and cyclopentylamine (85 mg, 1.0 mmol). This resulted reaction mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl (10 mL) agian, and then with brine (10 mL). Organic layer was dried over anhydrous Na₂SO₄. Removal of solvent yielded crude product (220 mg, 76%), which was used for next step without further purification.

117b) N-({1-cyclopentyl-5-[(cyclopentylamino)carbonyl]-4-hydroxy-2-oxo-1,2-dihydro-3-pyridinyl}carbonyl)glycine. Into a 2 ml microwave reaction vessel were added N-1-dicyclopentyl-4-hydroxy-6-oxo-1,6-dihydro-3-pyridinecarboxamide (145 mg, 0.5 mmol), TEA (0.07 mL, 0.5 mmol), and Ethyl N-(oxomethylidene)glycinate (64.6 mg, 0.5 mmol) in chloroform (1 mL). Reaction mixture was heated at 120° C. in microwave reactor for 1 hour. LCMS showed the reaction was complete. Chloroform was removed. Residue was dissolved in ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (36 mg, 18%) was obtained. MS (ES+): m/z [M+H]⁺=392.2; ¹H NMR (400 MHz, DMSO-d₆) δ ppm 1.50-1.67 (m, 7H) 1.70-1.92 (m, 6H) 2.06 (m., 2H) 3.35 (m, 2H) 4.12 (d, J=5.81 Hz, 2H) 4.19 (s, 1H) 5.04 (s, 1H) 8.03 (s, 1H) 8.37 (s, 1H) 10.48 (br. s., 1H) 12.99 (s, 1H)

### Example 118

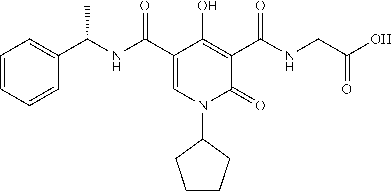

### N-{[1-Cyclopentyl-4-hydroxy-2-oxo-5-({[(1S)-1-phenylethyl]amino}carbonyl)-1,2-dihydro-3-pyridinyl]carbonyl}glycine

118a) 1-Cyclopentyl-4-hydroxy-6-oxo-N-[(1S)-1-phenylethyl]-1,6-dihydro-3-pyridinecarboxamide. Into a 2 mL microwave reaction vessel were added methyl 1-cyclopentyl-4-hydroxy-6-oxo-1,6-dihydro-3-pyridinecarboxylate (237 mg, 1.0 mmol) and [(1S)-1-phenylethyl]amine (121 mg, 1.0 mmol). This resulted reaction mixture was stirred and heated at 100° C. in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and then with brine (10 mL). Organic layer was dried over anhydrous Na₂SO₄. Removal of solvent yielded crude product (216 mg, 66%), which was used for next step without further purification.

118b) N-{[1-cyclopentyl-4-hydroxy-2-oxo-5-({[(1S)-1-phenylethyl]amino}carbonyl)-1,2-dihydro-3-pyridinyl]carbonyl}glycine. Into a 2 ml microwave reaction vessel were added 1-cyclopentyl-4-hydroxy-6-oxo-N-[(1S)-1-phenylethyl]-1,6-dihydro-3-pyridinecarboxamide (163 mg, 0.5 mmol), TEA (0.07 mL, 0.5 mmol), and Ethyl N-(oxomethylidene)glycinate (64.6 mg, 0.5 mmol) in chloroform (1 mL). Reaction mixture was heated at 120° C. in microwave reactor for 1 hour. LCMS showed that the reaction was complete. Chloroform was removed. Residue was dissolved in ethyl alcohol (2 mL) and then added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (42 mg, 20%) was obtained. MS (ES+): m/z [M+H]⁺=428.0; ¹H NMR (400 MHz, DMSO-d₆) δ ppm 1.47 (d, J=7.07 Hz, 3H) 1.64 (m, 3H) 1.77 (m, 4H) 2.05 (m, 2H) 4.12 (br. s., 2H) 5.05 (d, J=7.07 Hz, 1H), 5.11 (d, J=7.07 Hz, 1H) 7.26 (s, 1H) 7.24 (t, J=6.82 Hz, 1 H) 7.31-7.43 (m, 3H) 8.36 (s, 1H) 10.48 (br. s., 1H) 13.02 (br. s., 1H)

### Example 119

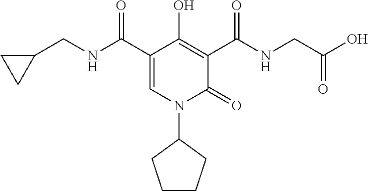

### N-[(1-Cyclopentyl-5-{[(cyclopropylmethyl)amino]carbonyl}-4-hydroxy-2-oxo-1,2-dihydro-3-pyridinyl)carbonyl]glycine

119a) 1-Cyclopentyl-N-(cyclopropylmethyl)-4-hydroxy-6-oxo-1,6-dihydro-3-pyridinecarboxamide. Into a 2 mL microwave reaction vessel were added methyl 1-cyclopentyl-4-hydroxy-6-oxo-1,6-dihydro-3-pyridinecarboxylate (237 mg, 1.0 mmol) and (cyclopropylmethyl)amine (71.1 mg, 1.0 mmol). This resulted reaction mixture was stirred and heated at 100° C. in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and then with brine (10 mL). Organic layer was dried and concentrated to give crude product (244 mg, 88%), which was used for next step without further purification.

119b) N-[(1-Cyclopentyl-5-{[(cyclopropylmethyl)amino]carbonyl}-4-hydroxy-2-oxo-1,2-dihydro-3-pyridinyl)carbonyl]glycine. Into a 2 ml microwave reaction vessel were added 1-cyclopentyl-N-(cyclopropylmethyl)-4-hydroxy-6-oxo-1,6-dihydro-3-pyridinecarboxamide (138 mg, 0.5 mmol), TEA (0.07 mL, 0.5 mmol), and Ethyl N-(oxomethylidene)glycinate (64.6 mg, 0.5 mmol) in chloroform (1 mL). Reaction mixture was heated at 120° C. in microwave reactor for 1 hour. LCMS showed that the reaction was complete. Chloroform was removed. Residue was dissolved in ethyl alcohol (2 mL) and then added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (104 mg, 55%) was obtained. MS (ES+): m/z [M+H]⁺=378.0; ¹H NMR (400 MHz, DMSO-d₆) δ ppm 0.23 (dd, J=4.67, 1.64 Hz, 2H) 0.42 (dd, J=7.96, 1.64 Hz, 2H) 1.03 (m, 1H) 1.66 (m, 3H) 1.81 (m, 3H) 2.09 (m, 2H) 3.18 (s, 1H) 3.16 (d, J=5.81 Hz, 1H) 4.13 (d, J=5.05 Hz, 2H) 5.05 (s, 1H) 8.22 (br.s., 1H) 8.41 (s, 1H) 10.49 (s, 1H) 12.99 (s, 1H)

### Example 120

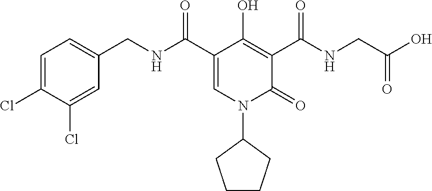

### N-{[1-cyclopentyl-5-({[(3,4-dichlorophenyl)methyl]amino}carbonyl)-4-hydroxy-2-oxo-1,2-dihydro-3-pyridinyl]carbonyl}glycine

120a) 1-Cyclopentyl-N-[(3,4-dichlorophenyl)methyl]-4-hydroxy-6-oxo-1,6-dihydro-3-pyridinecarboxamide. Into a 2 mL microwave reaction vessel were added methyl 1-cyclopentyl-4-hydroxy-6-oxo-1,6-dihydro-3-pyridinecarboxylate (237 mg, 1.0 mmol) and [(3,4-dichlorophenyl)methyl]amine (176 mg, 1.0 mmol). The resulted reaction mixture was heated at 100° C. and stirred in microwave reactor for one hour. LCMS showed that the reaction was complete. Reaction mixture was dissolved in ethyl acetate (35 mL) and 5% HCl solution (10 mL). Two layers were separated. Organic layer was then washed with 5% HCl solution (10 mL) again, and then with brine (10 mL). Organic layer was dried and concentrated to produce desired product (329 mg, 86%), which was used for next step without further purification.

120b) N-{[1-cyclopentyl-5-({[(3,4-dichlorophenyl)methyl]amino}carbonyl)-4-hydroxy-2-oxo-1,2-dihydro-3-pyridinyl]carbonyl}glycine. Into a 2 ml microwave reaction vessel were added 1-cyclopentyl-N-[(3,4-dichlorophenyl)methyl]-4-hydroxy-6-oxo-1,6-dihydro-3-pyridinecarboxamide (191 mg, 0.5 mmol), TEA (0.07 mL, 0.5 mmol), and Ethyl N-(oxomethylidene)glycinate (64.6 mg, 0.5 mmol) in chloroform (1 mL). Reaction mixture was then heated at 120° C. in microwave reactor for 1 hour. LCMS showed that the reaction was complete. Chloroform was removed. Residue was dissolved in ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (52 mg, 22%) was obtained. MS (ES+): m/z [M+H]⁺=481.9; ¹H NMR (400 MHz, DMSO-d₆) δ ppm 1.65 (m, 2H) 1.79 (m, 5H) 2.05 (m, 2 H) 4.12 (d, J=5.56 Hz, 2H) 4.48 (d, J=5.81 Hz, 2H) 5.04 (s, 1H) 7.32 (dd, J=8.34, 2.02 Hz, 1H) 7.56-7.60 (m, 2H) 8.40 (s, 1H) 8.76 (br. s., 1H) 10.46 (br. s., 1H) 12.98 (br. s., 1H)

### Example 121

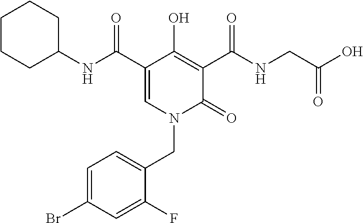

### N-({1-[(4-Bromo-2-fluorophenyl)methyl]-5-[cyclohexylamino)carbonyl]-4-hydroxy-2-oxo-1,2-dihydro-3-pyridinyl}carbonyl)glycine

121a) Methyl 1-[(4-bromo-2-fluorophenyl)methyl]-4-hydroxy-6-oxo-1,6-dihydro-3-pyridinecarboxylate. Into a 500 mL round-bottomed flask were added acetondimethylacetate (6.97 g, 40 mmol), acetic anhydride (7.55 ml, 80 mmol), and trimethylorthformate (4.24 g, 40 mmol). The resulted reaction mixture was stirred and heated at 120° C. for two hours, and then was cooled down to room temperature. After removing all volatile material in-vacuo, the residue was dissolved with THF (100 mL), [(4-bromo-2-fluorophenyl)methyl]amine (8.16 g, 40 mmol) was added slowly. The reaction mixture was stirred at room temperature overnight. Sodium hydride (1.92 g, 80 mmol) was added portionwise. The reaction was stirred at room temperature for 30 minutes. LCMS showed that the reaction was complete. The reaction mixture was concentrated, cooled down to 0° C., and added ethyl acetate (100 mL) and 30 ml of 10% HCl solution (to adjuste pH of aqueous layer around 2). Two layers were separated, and aqueous layer was extracted with ethyl acetate (75 mL×2). Combined organic layers were dried and concentrated to yield desired product (1.2 g, 8%), which was used for next step without further purification.

121b) 1-[(4-Bromo-2-fluorophenyl)methyl]-N-cyclohexyl-4-hydroxy-6-oxo-1,6-dihydro-3-pyridinecarboxamide. Into a 2 mL microwave reaction vessel were added methyl 1-[(4-bromo-2-fluorophenyl)methyl]-4-hydroxy-6-oxo-1,6-dihydro-3-pyridinecarboxylate (427 mg, 1.2 mmol) and cyclohexylamine (119 mg, 1.2 mmol). The resulted reaction mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and then with brine (10 mL). Organic layer was dried and concentrated to give the titled product (333 mg, 66%), which was used for next step without further purification.

121c) N-({1-[(4-bromo-2-fluorophenyl)methyl]-5-[(cyclohexylamino)carbonyl]-4-hydroxy-2-oxo-1,2-dihydro-3-pyridinyl}carbonyl)glycine. Into a 2 ml microwave reaction vessel were added 1-[(4-bromo-2-fluorophenyl)methyl]-N-cyclohexyl-4-hydroxy-6-oxo-1,6-dihydro-3-pyridinecarboxamide (106 mg, 0.25 mmol), DIEA (0.044 mL, 0.25 mmol), and ethyl N-(oxomethylidene)glycinate (32 mg, 0.25 mmol) in chloroform (2 mL). Reaction mixture was heated at 120° C. in microwave reactor for 1 hour. LCMS showed that the reaction was complete. Chloroform was removed. Residue was dissolved in ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (14 mg, 11%) was obtained. MS (ES+): m/z [M+H]⁺=524.1; ¹H NMR (400 MHz, DMSO-d₆) δ 6 ppm 1.31 (m., 5H) 1.54 (m., 1H) 1.66 (m, 2H) 1.84 (m, 2H) 3.80 (m, 1H) 4.08 (d, J=5.31 Hz, 2H) 5.26 (s, 2H) 7.20 (s, 1H) 7.41 (dd, J=8.46, 2.15 Hz, 1H) 7.59 (dd, J=9.98, 1.89 Hz, 1H) 8.01 (br. s., 1H) 8.69 (s, 1H) 10.25 (br. s., 1 H) 12.97 (br. s., 1H)

### Example 122

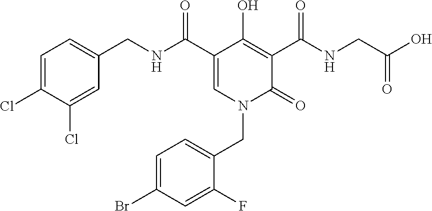

### N-{[1-[(4-Bromo-2-fluorophenyl)methyl]-5-({[(3,4-dichlorophenyl)methyl]amino}carbonyl)-4-hydroxy-2-oxo-1,2-dihydro-3-pyridinyl]carbonyl}glycine

122a) 1-[(4-Bromo-2-fluorophenyl)methyl]-N-[(3,4-dichlorophenyl)methyl]-4-hydroxy-6-oxo-1,6-dihydro-3-pyridinecarboxamide. Into a 2 mL microwave reaction vessel were added methyl 1-[(4-bromo-2-fluorophenyl)methyl]-4-hydroxy-6-oxo-1,6-dihydro-3-pyridinecarboxylate (0.427 g, 1.2 mmol) and [(3,4-dichlorophenyl)methyl]amine (211 mg, 1.2 mmol). The resulted reaction mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and brine (10 mL). Organic layer was dried and concentrated to give crude product (406 mg, 68%), which was used for next step without further purification.

122b) N-{[1-[(4-Bromo-2-fluorophenyl)methyl]-5-({[(3,4-dichlorophenyl)methyl]amino}carbonyl)-4-hydroxy-2-oxo-1,2-dihydro-3-pyridinyl]carbonyl}glycine. Into a 2 ml microwave reaction vessel were added 1-[(4-bromo-2-fluorophenyl)methyl]-N-[(3,4-dichlorophenyl)methyl]-4-hydroxy-6-oxo-1,6-dihydro-3-pyridinecarboxamide (125 mg, 0.250 mmol), DIEA (0.044 mL, 0.25 mmol), and ethyl N-(oxomethylidene)glycinate (32.3 mg, 0.250 mmol) in chloroform (2 mL). Reaction mixture was heated at 120° C. in microwave reactor for 1 hour. LCMS showed that the reaction was complete. Chloroform was removed. The residue was dissolved in ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (29 mg, 19%) was obtained. MS (ES+): m/z [M+H]⁺=601.4; ¹H NMR (400 MHz, DMSO-d₆) δ ppm 4.08 (d, J=5.31 Hz, 2H) 4.49 (d, J=6.32 Hz, 2H) 5.25 (s, 2H) 7.21 (t, J=8.21 Hz, 1H) 7.33 (dd, J=8.34, 2.02 Hz, 1H) 7.40 (dd, J=8.34, 1.77 Hz, 1H) 7.57-7.62 (m, 3H) 8.73 (s, 2H) 10.23 (s, 1H) 13.00 (br s, 1H)

### Example 123

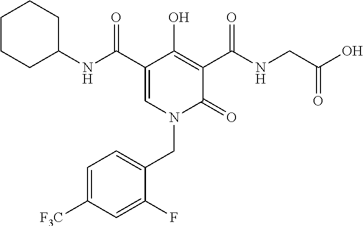

### N-[(5-[(Cyclohexylamino)carbonyl]-1-{[2-fluoro-4-(trifluoromethyl)phenyl]methyl}-4-hydroxy-2-oxo-1,2-dihydro-3-pyridinyl)carbonyl]glycine

123a) Methyl 1-{[2-fluoro-4-(trifluoromethyl)phenyl]methyl}-4-hydroxy-6-oxo-1,6-dihydro-3-pyridinecarboxylate. Into a 250 mL round-bottomed flask were added acetondimethylacetate (1.74 g, 10 mmol), acetic anhydride (1.9 ml, 20 mmol), and trimethylorthformate (1.06 g, 10 mmol). The resulted reaction mixture was stirred and heated at 120° C. for two hours, and cooled down to room temperature. After removing all volatile material in-vacuo, the residue was dissolved with THF (100 mL). {[2-fluoro-4-(trifluoromethyl)phenyl]methyl}amine (1.93 g, 10 mmol) was added slowly. The resulted reaction mixture was stirred at room temperature overnight. Sodium hydride (0.48 g, 20 mmol) was added portionwise. Reaction mixture was stirred at room temperature for 30 minutes. LCMS showed that the reaction was complete. Solvent was removed. The reaction mixture was cooled down to 0° C., and added ethyl acetate (100 mL) and 30 ml of 10% HCl (to adjust pH of aqueous layer around 2). Two layers were separated, and the aqueous layer was extracted with ethyl acetate (75 mL×2). Combined organic layers were dried and solvent was removed. Crude product (1.55 g) was obtained and used without further purification.

123b) N-Cyclohexyl-1-{[2-fluoro-4-(trifluoromethyl)phenyl]methyl}-4-hydroxy-6-oxo-1,6-dihydro-3-pyridinecarboxamide. Into a 2 mL microwave reaction vessel were added methyl 1-{[2-fluoro-4-(trifluoromethyl)phenyl]methyl}-4-hydroxy-6-oxo-1,6-dihydro-3-pyridinecarboxylate (345 mg, 1 mmol) and cyclohexylamine (99 mg, 1 mmol). The resulted mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated to give desired product (272 mg, 66%), which was used without further purification.

123c) N-[(5-[(Cyclohexylamino)carbonyl]-1-{[2-fluoro-4-(trifluoromethyl)phenyl]methyl}-4-hydroxy-2-oxo-1,2-dihydro-3-pyridinyl)carbonyl]glycine. Into a 2 mL microwave reaction vessel were added N-cyclohexyl-1-{[2-fluoro-4-(trifluoromethyl)phenyl]methyl}-4-hydroxy-6-oxo-1,6-dihydro-3-pyridinecarboxamide (272 mg, 0.66 mmol), DIEA (0.115 mL, 0.66 mmol), and ethyl N-(oxomethylidene)glycinate (85 mg, 0.66 mmol) in chloroform (2 mL). Reaction mixture was heated at 120° C. for 30 minutes. LCMS showed that the reaction was complete. Chloroform was removed. Ethyl alcohol (4 mL) and 800 uL of 2N NaOH solution were added to the residue. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Product (79 mg, 23%) was obtained. MS (ES+): m/z [M+H]⁺=514.3; ¹H NMR (400 MHz, DMSO-d₆) δ ppm 1.31 (br. s., 4H) 1.54 (br. s., 2H) 1.66 (br. s., 2H) 1.84 (br. s., 3H) 3.79 (s, 1H) 4.07 (br. s., 2H) 5.37 (s, 2H) 7.42 (s, 1H) 7.56 (s, 1H) 7.73 (d, J=12.13 Hz, 1H) 8.04 (br. s., 1H) 8.76 (s, 1H) 10.23 (br. s., 1 H) 12.97 (s, 1H)

### Example 124

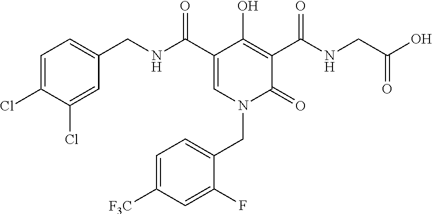

### N-[(5-({[(3,4-Dichlorophenyl)methyl]amino}carbonyl)-1-{[2-fluoro-4-(trifluoromethyl)phenyl]methyl}-4-hydroxy-2-oxo-1,2-dihydro-3-pyridinyl)carbonyl]glycine

124a) N-[(3,4-Dichlorophenyl)methyl]-1-{[2-fluoro-4-(trifluoromethyl)phenyl]methyl}-4-hydroxy-6-oxo-1,6-dihydro-3-pyridinecarboxamide. Into a 2 mL microwave reaction vessel were added methyl 1-{[2-fluoro-4-(trifluoromethyl)phenyl]methyl}-4-hydroxy-6-oxo-1,6-dihydro-3-pyridinecarboxylate (345 mg, 1 mmol) and [(3,4-dichlorophenyl)methyl]amine (176 mg, 1 mmol). The resulted reaction mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated to give crude product (402 mg, 82%), which was used without further purification.

124b) N-[(5-({[3,4-Dichlorophenyl)methyl]amino}carbonyl)-1-{[2-fluoro-4-(trifluoromethyl)phenyl]methyl}-4-hydroxy-2-oxo-1,2-dihydro-3-pyridinyl)carbonyl]glycine. Into a 5 ml microwave reaction vessel were added N-[(3,4-dichlorophenyl)methyl]-1-{[2-fluoro-4-(trifluoromethyl)phenyl]methyl}-4-hydroxy-6-oxo-1,6-dihydro-3-pyridinecarboxamide (323 mg, 0.66 mmol), DIEA (0.115 mL, 0.66 mmol), and ethyl N-(oxomethylidene)glycinate (85 mg, 0.66 mmol) in chloroform (2 mL). Reaction mixture was heated at 120° C. in microwave reactor for 1 hour. LCMS showed that the reaction was complete. Chloroform was removed. The residue was dissolved with ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (43 mg, 11%) was obtained. MS (ES+): m/z [M+H]⁺=590.1; ¹H NMR (400 MHz, DMSO-d₆) δ ppm 4.07 (s, 2H) 4.49 (d, J=6.32 Hz, 2H) 5.37 (s, 2H) 7.33 (dd, J=8.34, 2.02 Hz, 1H) 7.43 (s, 1H) 7.53-7.63 (m, 3H) 7.73 (d, J=10.61 Hz, 1H) 8.79 (s, 2H) 10.20 (br. s., 1H) 12.96 (s, 1H)

### Example 125

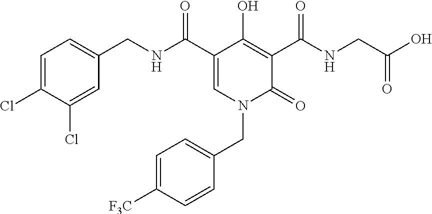

### N-[(5-({[(3,4-Dichlorophenyl)methyl]amino}carbonyl)-4-hydroxy-2-oxo-1-{[4-trifluoromethyl)phenyl]methyl}-1,2-dihydro-3-pyridinyl)carbonyl]glycine

125a) Methyl 4-hydroxy-6-oxo-1-{[4-(trifluoromethyl)phenyl]methyl}-1,6-dihydro-3-pyridinecarboxylate. Into a 250 mL round-bottomed flask were added acetondimethylacetate (1.74 g, 10 mmol), acetic anhydride (1.9 ml, 20 mmol), and trimethylorthformate (1.06 g, 10 mmol). The resulted reaction mixture was stirred and heated at 120° C. for two hours, and cooled down to room temperature. After removing all volatile material under reduced pressure, the residue was dissolved with THF (100 mL). 4-(trifluoromethyl)phenyl]methyl}amine (1.75 g, 10 mmol) was added slowly. The resulted reaction mixture was stirred at room temperature overnight. Sodium hydride (0.48 g, 20 mmol) was added portionwise. Reaction mixture was stirred at room temperature for 30 minutes. LCMS showed that the reaction was complete. Solvent was removed. The reaction mixture was cooled down to 0° C., and added ethyl acetate (100 mL) and 30 ml of 10% HCl solution (to adjust pH of aqueous layer around 2). Two layers were separated, and the aqueous layer was extracted with ethyl acetate (75 mL×2). Combined organic layers were dried and concentrated. Desired product (2.77 g, 85%) was obtained and was used without further purification.

125b) N-[(3,4-Dichlorophenyl)methyl]-4-hydroxy-6-oxo-1-{[4-(trifluoromethyl)phenyl]methyl}-1,6-dihydro-3-pyridinecarboxamide. Into a 2 mL microwave reaction vessel were added methyl 4-hydroxy-6-oxo-1-{[4-(trifluoromethyl)phenyl]methyl}-1,6-dihydro-3-pyridinecarboxylate (262 mg, 0.8 mmol) and [(3,4-dichlorophenyl)methyl]amine (141 mg, 0.8 mmol). The resulted reaction mixture was heated at 100° C. and stirred in microwave reactor for one hour. LCMS showed that the reaction was complete. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated. Crude product was purified with Gilson preparative HPLC to give desired product (173 mg, 46%).

125c) N-[(5-({[(3,4-Dichlorophenyl)methyl]amino}carbonyl)-4-hydroxy-2-oxo-1-{[4-(trifluoromethyl)phenyl]methyl}-1,2-dihydro-3-pyridinyl)carbonyl]glycine. Into a 5 ml microwave reaction vessel were added N-[(3,4-dichlorophenyl)methyl]-4-hydroxy-6-oxo-1-{[4-(trifluoromethyl)phenyl]methyl}-1,6-dihydro-3-pyridinecarboxamide (173 mg, 0.37 mmol), DIEA (0.064 mL, 0.37 mmol), and ethyl N-(oxomethylidene)glycinate (47 mg, 0.37 mmol) in chloroform (2 mL). Reaction mixture was then heated at 120° C. in microwave reactor for 1 hour. LCMS showed that the reaction was complete. Chloroform was removed. The residue was dissolved with ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (68 mg, 32%) was obtained. MS (ES+): m/z [M+H]⁺=572.2; ¹H NMR (400 MHz, DMSO-d₆) δ ppm 4.09 (d, J=5.56 Hz, 2H) 4.48 (d, J=6.06 Hz, 2H) 5.35 (s, 2H) 7.33 (dd, J=8.21, 1.89 Hz, 1H) 7.50 (d, J=7.83 Hz, 2H) 7.55-7.63 (m, 2H) 7.72 (d, J=8.59 Hz, 2H) 8.80 (s, 2H) 10.28 (s, 1H)

### Example 126

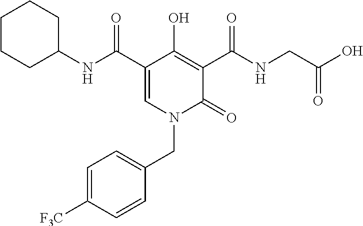

### N-[(5-[(Cyclohexylamino)carbonyl]-4-hydroxy-2-oxo-1-{[4-(trifluoromethyl)phenyl]methyl}-1,2-dihydro-3-pyridinyl)carbonyl]glycine

126a) N-Cyclohexyl-4-hydroxy-6-oxo-1-{[4-(trifluoromethyl)phenyl]methyl}-1,6-dihydro-3-pyridinecarboxamide. Into a 2 mL microwave reaction vessel were added methyl 4-hydroxy-6-oxo-1-{[4-(trifluoromethyl)phenyl]methyl}-1,6-dihydro-3-pyridinecarboxylate (262 mg, 0.8 mmol) and cyclohexylamine (79 mg, 0.8 mmol). The resulted reaction mixture was heated at 100° C. and stirred in microwave reactor for one hour. LCMS showed the reaction was complete. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated. The residue was then purified with Gilson preparative HPLC to give desired product (197 mg, 62%).

126b) N-[(5-[(Cyclohexylamino)carbonyl]-4-hydroxy-2-oxo-1-{[4-(trifluoromethyl)phenyl]methyl}-1,2-dihydro-3-pyridinyl)carbonyl]glycine. Into a 5 ml microwave reaction vessel were added N-cyclohexyl-4-hydroxy-6-oxo-1-{[4-(trifluoromethyl)phenyl]methyl}-1,6-dihydro-3-pyridinecarboxamide (145 mg, 0.37 mmol), DIEA (0.064 mL, 0.37 mmol), and ethyl N-(oxomethylidene)glycinate (47 mg, 0.37 mmol) in chloroform (2 mL). Reaction was then heated at 120° C. in microwave reactor for 1 hour. LCMS showed the reaction was complete. Chloroform was removed. Residue was dissolved with ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Reaction mixture was concentrated and the residue was dissolved in DMSO. DMSO solution was filtered and purified with Gilson preparative HPLC. Desired product (82 mg, 45%) was obtained. MS (ES+): m/z [M+H]⁺=496.2; ¹H NMR (400 MHz, DMSO-d₆) δ 6 ppm 1.16 (s, 1H) 1.31 (br. s., 4 H) 1.53 (br. s., 1H) 1.66 (br. s., 2H) 1.83 (br. s., 2H) 3.79 (br. s., 1H) 4.09 (br. s., 2H) 5.35 (s, 2 H) 7.50 (s, 2H) 7.71 (s, 2H) 8.75 (s, 1H) 10.31 (s, 1H)

### Example 127

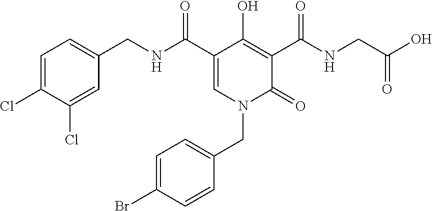

### N-{[1-[(4-Bromophenyl)methyl]-5-({[(3,4-dichlorophenyl)methyl]amino}carbonyl)-4-hydroxy-2-oxo-1,2-dihydro-3-pyridinyl]carbonyl}glycine

127a) Methyl 1-[(4-bromophenyl)methyl]-4-hydroxy-6-oxo-1,6-dihydro-3-pyridinecarboxylate. Into a 250 mL round-bottomed flask were added acetondimethylacetate (1.74 g, 10 mmol), acetic anhydride (0.94 ml, 10 mmol), and trimethylorthformate (1.06 g, 10 mmol). The resulted reaction mixture was stirred and heated at 120° C. for two hours, and cooled down to room temperature. After removing all volatile material under reduced pressure, the residue was dissolved with THF (100 mL). [(4-bromophenyl)methyl]amine (1.86 g, 10 mmol) was added slowly. Reaction mixture was stirred at room temperature overnight. Sodium hydride (0.48 g, 20 mmol) was added portionwise. Reaction mixture was stirred at room temperature for 30 minutes. LCMS showed the reaction was complete. The reaction mixture was concentrated and cooled down to 0° C., and added ethyl acetate (100 mL) and 30 ml of 10% HCl (to adjusted pH of aqueous layer to 2). Two layers were separated. The aqueous layer was extracted with ethyl acetate (75 mL×2). Combined organic layers were dried and concentrated. Crude product (2.07 g, 61%) was obtained and used without purification.

127b) 1-[(4-Bromophenyl)methyl]-N-[(3,4-dichlorophenyl)methyl]-4-hydroxy-6-oxo-1,6-dihydro-3-pyridinecarboxamide. Into a 2 mL microwave reaction vessel were added methyl 1-[(4-bromophenyl)methyl]-4-hydroxy-6-oxo-1,6-dihydro-3-pyridinecarboxylate (338 mg, 1.0 mmol) and [(3,4-dichlorophenyl)methyl]amine (176 mg, 1.0 mmol). The resulted reaction mixture was heated at 100° C. and stirred in microwave reactor for two hours. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated to give product (93 mg, 19%), which was used for next step without further purification.

127c) N-{[1-[(4-Bromophenyl)methyl]-5-({[(3,4-dichlorophenyl)methyl]amino}carbonyl)-4-hydroxy-2-oxo-1,2-dihydro-3-pyridinyl]carbonyl}glycine. Into a 5 ml microwave reaction vessel were added 1-[(4-bromophenyl)methyl]-N-[(3,4-dichlorophenyl)methyl]-4-hydroxy-6-oxo-1,6-dihydro-3-pyridinecarboxamide (93 mg, 0.19 mmol), DIEA (0.087 mL, 0.5 mmol), and ethyl N-(oxomethylidene)glycinate (64.6 mg, 0.5 mmol) in chloroform (1 mL). Reaction mixture was then heated at 120° C. in microwave reactor for 1 hour. LCMS showed the reaction was complete. Chloroform was removed. The residue was dissolved with ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (37 mg, 13%) was obtained. MS (ES+): m/z [M+H]⁺=582.0; ¹H NMR (400 MHz, DMSO-d₆) δ ppm 4.09 (br. s., 2H) 4.48 (s, 2 H) 5.22 (s, 2H) 7.33 (s, 1H) 7.27 (d, J=8.59 Hz, 2H) 7.53-7.63 (m, 4H) 8.73 (m, 2H) 10.31 (s, 1H) 13.0 (br. s., 1H)

### Example 128

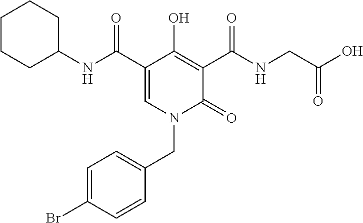

### N-({1-[(4-Bromophenyl)methyl]-5-[(cyclohexylamino)carbonyl]-4-hydroxy-2-oxo-1,2-dihydro-3-pyridinyl}carbonyl)glycine

128a) Methyl 1-[(4-bromophenyl)methyl]-4-hydroxy-6-oxo-1,6-dihydro-3-pyridinecarboxylate. Into a 2 mL microwave reaction vessel were added methyl 1-[(4-bromophenyl)methyl]-4-hydroxy-6-oxo-1,6-dihydro-3-pyridinecarboxylate (338 mg, 1 mmol) and cyclohexanamine (99 mg, 1 mmol). The resulted reaction mixture was heated at 100° C. and stirred in microwave reactor for two hours. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated to give product (220 mg, 54%), which was used for next step without further purification.

128b) N-({1-[(4-Bromophenyl)methyl]-5-[(cyclohexylamino)carbonyl]-4-hydroxy-2-oxo-1,2-dihydro-3-pyridinyl}carbonyl)glycine. Into a 5 ml microwave reaction vessel were added 1-[(4-bromophenyl)methyl]-N-cyclohexyl-4-hydroxy-6-oxo-1,6-dihydro-3-pyridinecarboxamide (203 mg, 0.5 mmol), DIEA (0.087 mL, 0.5 mmol), and ethyl N-(oxomethylidene)glycinate (64.6 mg, 0.5 mmol) in chloroform (1 mL). Reaction mixture was then heated at 120° C. in microwave reactor for 1 hour. LCMS showed that the reaction was complete. Chloroform was removed. The residue was dissolved with ethyl alcohol (2 mL) and added 500 uL of 2N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (15 mg, 6%) was obtained. MS (ES+): m/z [M+H]⁺=506.1 ¹H NMR (400 MHz, DMSO-d₆) δ ppm 1.23 (br. s., 2H) 1.30 (br. s., 4H) 1.53 (br. s., 1H) 1.66 (br. s., 2H) 1.75 (s, 1H) 1.83 (br. s., 1H) 3.79 (br. s., 1H) 4.10 (d, J=5.56 Hz, 2H) 5.23 (s, 2H) 7.26 (d, J=8.34 Hz, 2H) 7.56 (q, J=4.63 Hz, 1H) 7.56 (d, J=8.59 Hz, 1H) 7.99 (s, 1H) 8.70 (s, 1H) 10.34 (br. s., 1H) 12.98 (br. s., 1H)

### Example 129

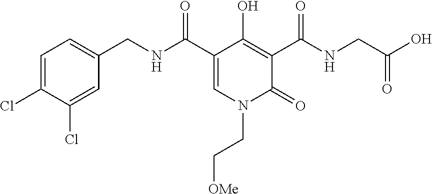

### N-({5-({[(3,4-Dichlorophenyl)methyl]amino}carbonyl)-4-hydroxy-1-[2-(methyloxy)ethyl]-2-oxo-1,2-dihydro-3-pyridinyl}carbonyl)glycine

129a) Methyl 4-hydroxy-1-[2-(methyloxy)ethyl]-6-oxo-1,6-dihydro-3-pyridinecarboxylate. Into a 250 mL round-bottomed flask were added acetondimethylacetate (1.74 g, 10 mmol), acetic anhydride (0.94 ml, 10 mmol), and trimethylorthformate (1.06 g, 10 mmol). The resulted reaction mixture was stirred and heated at 120° C. for two hours, and cooled down to room temperature. After removing all volatile material under reduced pressure, the residue was dissolved with THF (100 mL). [2-(methyloxy)ethyl]amine (0.75 g, 10 mmol) was added slowly. The reaction mixture was stirred at room temperature overnight. Sodium hydride (0.48 g, 20 mmol) was added portionwise. The reaction mixture was stirred at room temperature for 30 minutes. LCMS showed that the reaction was complete. The reaction mixture was concentrated and cooled down to 0° C., and added ethyl acetate (100 mL) and 30 ml of 10% HCl solution (to adjust pH of aqueous layer to 2). Two layers were separated, and aqueous layer was extracted with ethyl acetate (75 mL×2). Combined organic layers were dried and concentrated. The desired product (1.6 g, 70%) was obtained and used without further purification.

129b) N-[(3,4-Dichlorophenyl)methyl]-4-hydroxy-1-[2-(methyloxy)ethyl]-6-oxo-1,6-dihydro-3-pyridinecarboxamide. Into a 2 mL microwave reaction vessel were added methyl 4-hydroxy-1-[2-(methyloxy)ethyl]-6-oxo-1,6-dihydro-3-pyridinecarboxylate (227 mg, 1.0 mmol) and [(3,4-dichlorophenyl)methyl]amine (176 mg, 1.0 mmol). The resulted reaction mixture was heated at 100° C. and stirred in microwave reactor for two hours. Reaction mixture was dissolved with ethyl acetate (35 mL) and 5% HCl solution (10 mL). Two layers were separated. Organic layer was then washed with 5% HCl solution (10 mL) again, and with brine (10 mL). Organic layer was dried and concentrated to give crude product. The residue was purified with Gilson preparative HPLC to give desired product (112 mg, 30%).

129c) N-({5-({[(3,4-Dichlorophenyl)methyl]amino}carbonyl)-4-hydroxy-1-[2-(methyloxy)ethyl]-2-oxo-1,2-dihydro-3-pyridinyl}carbonyl)glycine. Into a 5 ml microwave reaction vessel were added N-[(3,4-dichlorophenyl)methyl]-4-hydroxy-1-[2-(methyloxy)ethyl]-6-oxo-1,6-dihydro-3-pyridinecarboxamide (112 mg, 0.30 mmol), DIEA (0.159 mL, 0.91 mmol), and ethyl N-(oxomethylidene)glycinate (118 mg, 0.91 mmol) in chloroform (2 mL). Reaction mixture was then heated at 120° C. in microwave reactor for 1 hour. LCMS showed that the reaction was complete. Chloroform was removed. The residue was dissolved in ethyl alcohol (2 mL) and added 200 uL of 10N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (50 mg, 35%) was obtained. MS (ES+): m/z [M+H]⁺=472.3 ¹H NMR (400 MHz, DMSO-d₆) δ ppm 3.24 (s, 3H) 3.58 (s, 2H) 4.12 (d, J=5.81 Hz, 2H) 4.21 (s, 2H) 4.49 (s, 2H) 7.32 (dd, J=8.08, 2.02 Hz, 1H) 7.57-7.64 (m, 2H) 8.47 (s, 1H) 8.74 (s, 1H) 10.39 (s, 1H) 13.01 (s, 1H)

### Example 130

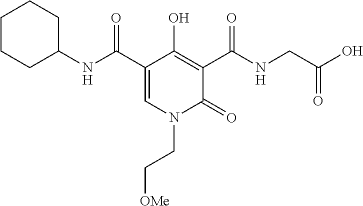

### N-({5-[(Cyclohexylamino)carbonyl]-4-hydroxy-1-[2-(methyloxy)ethyl]-2-oxo-1,2-dihydro-3-pyridinyl}carbonyl)glycine

130a) N-Cyclohexyl-4-hydroxy-1-[2-(methyloxy)ethyl]-6-oxo-1,6-dihydro-3-pyridinecarboxamide. Into 2 mL microwave reaction vessel were added methyl 4-hydroxy-1-[2-(methyloxy)ethyl]-6-oxo-1,6-dihydro-3-pyridinecarboxylate (227 mg, 1.0 mmol) and cyclohexanamine (99 mg, 1.0 mmol). The resulted reaction mixture was heated at 100° C. and stirred in microwave reactor for two hours. Reaction mixture was dissolved with ethyl acetate (35 mL) and 5% HCl solution (10 mL). Two layers were separated. Organic layer was then washed with 5% HCl solution (10 mL) again, and then with brine (10 mL). Organic layer was dried and concentrated to give crude product (268 mg, 91%), which was used for next step with further purification.

130b) N-({5-[(Cyclohexylamino)carbonyl]-4-hydroxy-1-[2-(methyloxy)ethyl]-2-oxo-1,2-dihydro-3-pyridinyl}carbonyl)glycine. Into a 5 ml microwave reaction vessel were added N-cyclohexyl-4-hydroxy-1-[2-(methyloxy)ethyl]-6-oxo-1,6-dihydro-3-pyridinecarboxamide (268 mg, 0.91 mmol), DIEA (0.159 mL, 0.91 mmol), and ethyl N-(oxomethylidene)glycinate (118 mg, 0.91 mmol) in chloroform (2 mL). Reaction mixture was heated at 120° C. in microwave reactor for 1 hour. LCMS showed that the reaction was complete. Chloroform was removed. The residue was dissolved with ethyl alcohol (2 mL) and added 200 uL of 10 N NaOH solution. The resulted reaction mixture was stirred at room temperature for 30 minutes. LCMS showed that the reaction was complete. Reaction mixture was concentrated and then diluted with water (2 mL). The pH of resulted solution was adjusted with 6N HCl to pH around 1-2. Solvent was removed and the residue was dissolved in DMSO. DMSO solution was filtered and purified with Gilson preparative HPLC. Desired product (39 mg, 11%) was obtained. MS (ES+): m/z [M+H]⁺=396.0; ¹H NMR (400 MHz, DMSO-d₆) δ ppm 1.32 (d, J=10.86 Hz, 5H) 1.54 (br. s., 1H) 1.66 (br. s., 2H) 1.83 (br. s., 2H) 3.24 (s, 3H) 3.58 (t, J=5.18 Hz, 2H) 3.80 (br. s., 1H) 4.12 (d, J=5.31 Hz, 2H) 4.21 (t, J=5.31 Hz, 2H) 7.98 (br. s., 1H) 8.44 (s, 1H) 10.41 (br. s., 1H) 13.00 (br. s., 1H)

### Example 131

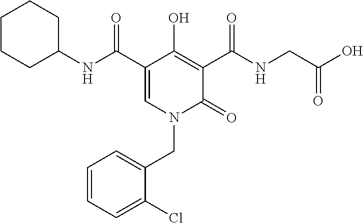

### N-({1-[(2-Chlorophenyl)methyl]-5-[(cyclohexylamino)carbonyl]-4-hydroxy-2-oxo-1,2-dihydro-3-pyridinyl}carbonyl)glycine

131a) Methyl 1-[(2-chlorophenyl)methyl]-4-hydroxy-6-oxo-1,6-dihydro-3-pyridinecarboxylate. Under an inert atmosphere tris(methyloxy)methane (4.87 g, 45.9 mmol) was added to a stirred solution of dimethyl 3-oxopentanedioate (8 g, 45.9 mmol) in acetic anhydride (8.68 ml, 92 mmol) and the contents heated (120° C., 2 hr). Volatiles were removed in-vacuo after cooling to touch. The residue was dissolved in tetrahydrofuran (48 ml) and to the solution was added 1-(2-chlorophenyl)methanamine (6.5 g, 45.9 mmol) with stirring at rt overnight. Sodium hydride (2.75 g, 114.7 mmol) was added to the stirred, cooled solution with in portions with nitrogen gas. The cooling was removed and solution stirred at rt 2 hr before the THF was removed and the product partitioned between ethyl acetate (125 ml) and 2N HCl (20 ml). After adjusting pH to 5 the aqueous was extracted with EtOAc (70 ml, 2×) and the combined organic dried and concentrated to produce an orange-brown oil. Trituration with diethyl ether (200 ml) overnight produced a yellow solid dried in-vacuo to yield the title compound as a yellow solid. Yield 11 g, 81.5%, MS (ES+): [M+H]⁺=294.1, ¹H NMR (400 MHz, DMSO-d₆) δ ppm 3.79 (s, 3H), 5.21 (s, 2 H), 5.76 (s, 1H), 6.92 (dd, J=7.20, 2.15 Hz, 1H), 7.25-7.39 (m, J=7.58, 7.33, 7.20, 7.20 Hz, 2H), 7.50 (dd, J=7.33, 1.77 Hz, 1H), 8.56 (s, 1H), 10.93 (br. s., 1H)

131b) 1-[(2-Chlorophenyl)methyl]-N-cyclohexyl-4-hydroxy-6-oxo-1,6-dihydro-3-pyridinecarboxamide. A neat mixture of methyl 1-[(2-chlorophenyl)methyl]-4-hydroxy-6-oxo-1,6-dihydro-3-pyridinecarboxylate (294 mg, 1 mmol) and cyclohexylamine (694 mg, 7 mmol) was heated in a microwave reactor to 120° C. for 1 hour. The residue was partitioned between ethyl acetate (60 ml) and 2.5N HCl (20 ml). The aqueous was extracted with EtOAc (30 ml, 2×) and the combined organic was dried and concentrated in-vacuo to produce the titled compound in 90% conversion as an orange-brown oil with (80% purity by LC MS) that was used as is for the next step. MS (ES+): [M+H]⁺=360.1.

131c) N-({1-[(2-Chlorophenyl)methyl]-5-[(cyclohexylamino)carbonyl]-4-hydroxy-2-oxo-1,2-dihydro-3-pyridinyl}carbonyl)glycine. Ethyl N-(oxomethylidene)glycinate (72 mg, 554 mmol) was added to a solution of N-({1-[(2-chlorophenyl)methyl]-5-[(cyclohexylamino)carbonyl]-4-hydroxy-2-oxo-1,2-dihydro-3-pyridinyl}carbonyl)glycine (200 mg, 554 mmol) and diisopropylethylamine (143 mg, 1.109 mmol) in chloroform (2.3 ml) and the contents heated in a microwave reactor to 120° C. for 45 min. The chloroform was removed in-vacuo and the residue dissolved in ethanol (6 ml) and 6N sodium hydroxide (2 ml). After stirring 1 hour at rt the mixture was diluted with ethyl acetate (20 ml) and washed with 1N HCl (6 ml). The product precipitated from the organic upon standing at rt overnight and was dried in-vacuo to yield the title compound as a white solid. Yield 40 mg, 15.3%, MS (ES+): [M+H]⁺=462.1, ¹H NMR (400 MHz, DMSO-d₆) δ ppm 0.95-1.45 (m, 6H), 1.55 (d, J=11.37 Hz, 1H), 1.61-1.74 (m, 2H), 1.82 (dd, J=8.84, 2.27 Hz, 2H), 3.79 (br. s., 1H), 4.09 (d, J=5.56 Hz, 2H), 5.33 (s, 2H), 7.04 (dd, J=7.45, 1.39 Hz, 1H), 7.28-7.40 (m, J=7.26, 7.01, 7.01, 7.01, 1.52 Hz, 2H), 7.51 (dd, J=7.71, 1.64 Hz, 1H), 8.02 (d, J=7.07 Hz, 1H), 8.63 (s, 1H), 10.25-10.35 (m, 1H), 12.97 (br. s., 1H)

### Example 132

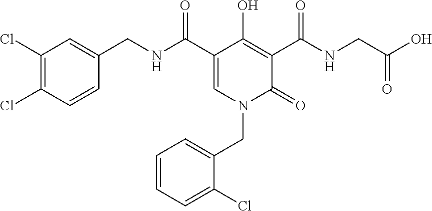

### N-{[1-[(2-Chlorophenyl)methyl]-5-({[(3,4-dichlorophenyl)methyl]amino}carbonyl)-4-hydroxy-2-oxo-1,2-dihydro-3-pyridinyl]carbonyl}glycine

132a) 1-[(2-Chlorophenyl)methyl]-N-[(3,4-dichlorophenyl)methyl]-4-hydroxy-6-oxo-1,6-dihydro-3-pyridinecarboxamide. A neat mixture of methyl 1-[(2-chlorophenyl)methyl]-4-hydroxy-6-oxo-1,6-dihydro-3-pyridinecarboxylate (132a, 294 mg, 1 mmol) and 1-(3,4-dichlorophenyl)methanamine (1.23 g, 7 mmol) was heated in a microwave reactor to 120° C. for 1 hour. The residue was partitioned between ethyl acetate (60 ml) and 2.5N HCl (20 ml). The aqueous was extracted with EtOAc (30 ml, 2×) and the combined organic was dried and concentrated in-vacuo to produce a brown oil. The residue was dissolved in dimethyl sulfoxide and purified by reverse phase HPLC (7 ml, 3×, 0.1% TFA acetonitrile, 0.1% TFA water). The title compound was obtained as a semi-solid and carried on as is. MS (ES+): [M+H]⁺=437.0.

132b) N-{[1-[(2-Chlorophenyl)methyl]-5-({[(3,4-dichlorophenyl)methyl]amino}carbonyl)-4-hydroxy-2-oxo-1,2-dihydro-3-pyridinyl]carbonyl}glycine. Ethyl N-(oxomethylidene)glycinate (1.47 g, 11.4 mmol) was added to a solution of 1-[(2-chlorophenyl)methyl]-N-[(3,4-dichlorophenyl)methyl]-4-hydroxy-6-oxo-1,6-dihydro-3-pyridinecarboxamide (500 mg, 1.14 mmol) and diisopropylethylamine (295 mg, 2.28 mmol) in chloroform (10 ml) and the contents heated in a microwave reactor to 120° C. for 45 min. The chloroform was removed in-vacuo and the residue dissolved in methyl sulfoxide (6 ml) and 6N sodium hydroxide (2 ml). The pH was adjusted to 5 with 6N HCl and the solution stirred at rt until precipitation complete. The solid was collected and dissolved in DMSO and purified by HPLC (0.1% TFA acetonitrile, 0.1% TFA water) to yield the title compound as a white solid. Yield 40 mg, 6.11% (3 steps), MS (ES+): [M+H]⁺=538.0, ¹H NMR (400 MHz, DMSO-d₆) δ ppm 4.09 (d, J=5.56 Hz, 2H), 4.48 (d, J=6.32 Hz, 2H), 5.32 (s, 2H), 7.06 (dd, J=7.33, 1.77 Hz, 1H), 7.23-7.43 (m, 3H), 7.51 (dd, J=7.58, 1.77 Hz, 1 H), 7.58 (s, 1H), 7.59 (d, J=6.32 Hz, 1H), 8.66 (s, 1H), 8.78 (t, J=6.32 Hz, 1H), 10.27 (t, J=5.31 Hz, 1H), 12.97 (br. s., 1H)

### Example 133

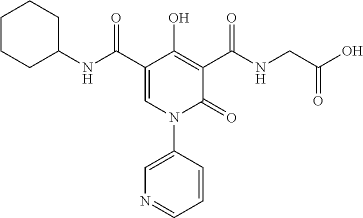

### N-({5-[(Cyclohexylamino)carbonyl]-4-hydroxy-2-oxo-2H-1,3′-bipyridin-3-yl}carbonyl)glycine

133a) Methyl 4-hydroxy-2-oxo-2H-1,3′-bipyridine-5-carboxylate. Under an inert atmosphere tris(methyloxy)methane (914 mg, 8.61 mmol) was added to a stirred solution of dimethyl 3-oxopentanedioate (1.5 g, 8.61 mmol) in acetic anhydride (1.63 ml, 17.23 mmol) and the contents heated (120° C., 2 hr). Volatiles were removed in-vacuo after cooling to touch. The residue was dissolved in tetrahydrofuran (50 ml) and to the solution was added 3-pyridinamine (811 mg, 8.61 mmol) with stirring at rt overnight. Sodium hydride (517 mg, 21.53 mmol) was added to the stirred, cooled solution with in portions with nitrogen gas. The cooling was removed and solution stirred at rt 2 hr before the THF was removed and the product partitioned between ethyl acetate (125 ml) and 2N HCl (20 ml). After adjusting pH to 5 the aqueous was extracted with EtOAc (70 ml, 2×) and the combined organic layers were dried and concentrated to produce the title compound as an orange-brown oil that was carried on as is. MS (ES+): [M+H]⁺=247.0.

133b) N-Cyclohexyl-4-hydroxy-2-oxo-2H-1,3′-bipyridine-5-carboxamide. A neat mixture of methyl 4-hydroxy-2-oxo-2H-1,3′-bipyridine-5-carboxylate (500 mg, 2.03 mmol) and cyclohexylamine (1.4 g, 14.2 mmol) was heated in a microwave reactor to 120° C. for 30 min. The residue was dissolved in ethyl acetate (40 ml) and 2.5N HCl (10 ml). Product remained in both layers. The solvents were removed and the residue dissolved in methyl sulfoxide and purified by HPLC (acetonitrile, 0.1% NH₄OH water). The title compound was obtained as a semi-solid and was used as is for the next step. MS (ES+): [M+H]⁺=314.1.

133c) N-({5-[(Cyclohexylamino)carbonyl]-4-hydroxy-2-oxo-2H-1,3′-bipyridin-3-yl}carbonyl)glycine. Ethyl N-(oxomethylidene)glycinate (515 mg, 3.99 mmol) was added to a solution of N-cyclohexyl-4-hydroxy-2-oxo-2H-1,3′-bipyridine-5-carboxamide (500 mg, 1.6 mmol) and diisopropylethylamine (412 mg, 3.19 mmol) in chloroform (10 ml) and the contents heated in a microwave reactor to 120° C. for 45 min. The chloroform was removed in-vacuo and the residue dissolved in ethanol (6 ml) and 6N sodium hydroxide (2 ml) and stirred at rt 1 hour. The solution was diluted with ethyl acetate (20 ml) and washed with 1N HCl (6 ml). Product remained in both layers. The solvents were removed and the residue dissolved in methyl sulfoxide (21 ml) and purified by HPLC (7 ml, 3×, acetonitrile, 0.1% NH₄OH water) to yield the title compound as a white solid. Yield 75 mg, 11.1% (3 steps), MS (ES+): [M+H]⁺=415.2, ¹H NMR (400 MHz, DMSO-d₆) δ ppm 1.15-1.46 (m, 6H), 1.56 (d, J=12.13 Hz, 1H), 1.63-1.73 (m, 2H), 1.79-1.89 (m, 2H), 3.80 (br. s., 1H), 4.12 (d, J=5.56 Hz, 2H), 7.63 (dd, 1H), 8.02 (ddd, J=8.21, 2.40, 1.52 Hz, 1H), 8.08 (br. s., 1H), 8.34 (s, 1H), 8.70 (dd, J=4.80, 1.26 Hz, 1H), 8.73 (d, J=2.27 Hz, 1H), 10.25 (br. s., 1H), 13.03 (br. s., 1H)

### Example 134

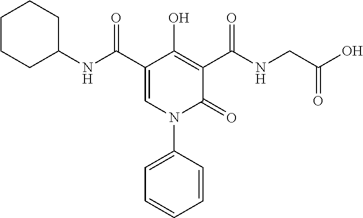

### N-({5-[(Cyclohexylamino)carbonyl]-4-hydroxy-2-oxo-1-phenyl-1,2-dihydro-3-pyridinyl}carbonyl)glycine

134a) Methyl 4,6-dihydroxy-1-phenyl-1,6-dihydro-3-pyridinecarboxylate. Under an inert atmosphere tris(methyloxy)methane (914 mg, 8.61 mmol) was added to a stirred solution of dimethyl 3-oxopentanedioate (1.5 g, 8.61 mmol) in acetic anhydride (1.63 ml, 17.23 mmol) and the contents heated (120° C., 2 hr). Volatiles were removed in-vacuo after cooling to touch. The residue was dissolved in tetrahydrofuran (50 ml) and to the solution was added aniline (802 mg, 8.61 mmol) with stirring at rt overnight. Sodium hydride (517 mg, 21.53 mmol) was added to the stirred, cooled solution with in portions with nitrogen gas. The cooling was removed and solution stirred at rt 2 hr before the THF was removed and the product partitioned between ethyl acetate (125 ml) and 2N HCl (20 ml). After adjusting pH to 5 the aqueous was extracted with EtOAc (70 ml, 2×) and the combined organic dried and concentrated to produce the title compound as an orange-brown oil that was carried on as is. MS (ES+): [M+H]⁺=246.1.

134b) N-Cyclohexyl-4-hydroxy-6-oxo-1-phenyl-1,6-dihydro-3-pyridinecarboxamide. A neat mixture of methyl 4,6-dihydroxy-1-phenyl-1,6-dihydro-3-pyridinecarboxylate (500 mg, 2.03 mmol) and cyclohexylamine (1.4 g, 14.2 mmol) was heated in a microwave reactor to 120° C. for 45 min. The residue was partition between ethyl acetate (40 ml) and 10% HCl (10 ml). The aqueous was extracted with EtOAc (30 ml, 2×). The combined organic was concentrated to yield an orange-brown residue that was carried on without further purification. MS (ES+): [M+H]⁺=312.2.

134c) N-({5-[(Cyclohexylamino)carbonyl]-4-hydroxy-2-oxo-1-phenyl-1,2-dihydro-3-pyridinyl}carbonyl)glycine. Ethyl N-(oxomethylidene)glycinate (83 mg, 0.640 mmol) was added to a solution of N-cyclohexyl-4-hydroxy-6-oxo-1-phenyl-1,6-dihydro-3-pyridinecarboxamide (100 mg, 0.320 mmol) and diisopropylethylamine (83 mg, 0.640 mmol) in chloroform (10 ml) and the contents heated in a microwave reactor to 130° C. for 45 min, the reaction was not complete. More ethyl N-(oxomethylidene)glycinate (83 mg, 0.640 mmol) was added and the system heated in a microwave reactor to 130° C. for 45 min. The chloroform was removed in-vacuo and the residue dissolved in DMSO (6 ml) and 6N sodium hydroxide (2 ml) and stirred at rt overnight. 6N hydrochloric acid was added to ˜pH 5 and a white precipitate was formed. The collected solid was dissolved in methyl sulfoxide and purified by HPLC (0.1% TFA acetonitrile, 0.1% TFA water) to yield the title compound as a white solid. Yield 23 mg, 17.2%, MS (ES+): [M+H]⁺=414.2, ¹H NMR (400 MHz, DMSO-d₆) δ ppm 1.14-1.43 (m, 5H), 1.56 (d, J=11.87 Hz, 1H), 1.61-1.74 (m, 2H),1.83 (d, J=3.03 Hz, 2H), 3.70-3.90 (m, 1H), 4.12 (d, J=5.31 Hz, 2H), 7.40-7.63 (m, 5 H), 8.07 (d, J=6.57 Hz, 1H), 8.25 (s, 1H), 10.33 (br. s., 1H), 13.02 (br. s., 1H)

### Example 135

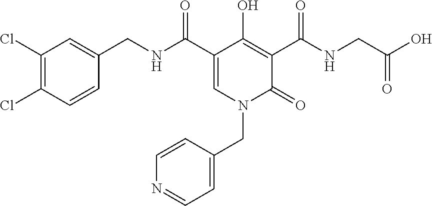

### N-{[5-({[(3,4-Dichlorophenyl)methyl]amino}carbonyl)-4-hydroxy-2-oxo-2H-1,3′-bipyridin-3-yl]carbonyl}glycine

135a) N-[(3,4-Dichlorophenyl)methyl]-4-hydroxy-2-oxo-2H-1,3′-bipyridine-5-carboxamide. A neat mixture of methyl 4-hydroxy-2-oxo-2H-1,3′-bipyridine-5-carboxylate (133a, 500 mg, 2.03 mmol) and (3,4-dichlorophenyl)methylamine (2.5 g, 14.2 mmol) was heated in a microwave reactor to 120° C. for 30 min. The residue was dissolved in DMSO and purified by HPLC (acetonitrile, 0.1% NH₄OH water) the product was carried on without calculating a yield, MS (ES+): [M+H]⁺=390.0, ¹H NMR (400 MHz, DMSO-d₆) δ ppm 4.24 (d, J=6.06 Hz, 2H), 4.47 (d, J=6.06 Hz, 1H), 5.78 (s, 4H), 7.13-7.37 (m, 1H), 7.49 (d, J=1.77 Hz, 1H), 7.55-7.66 (m, 2 H), 8.01 (dt, J=9.03, 1.55 Hz, 1H), 8.37-8.50 (m, 1H), 8.70 (dd, J=16.80, 2.91 Hz, 1H), 9.06 (t, J=5.94 Hz, 1H)

135b) N-{[5-({[(3,4-Dichlorophenyl)methyl]amino}carbonyl)-4-hydroxy-2-oxo-2H-1,3′-bipyridin-3-yl]carbonyl}glycine. Ethyl N-(oxomethylidene)glycinate (347 mg, 2.69 mmol) was added to a solution of N-[(3,4-dichlorophenyl)methyl]-4-hydroxy-2-oxo-2H-1,3′-bipyridine-5-carboxamide (500 mg, 1.28 mmol) and diisopropylethylamine (346 mg, 2.82 mmol) in chloroform (10 ml) and the contents heated in a microwave reactor to 130° C. for 45 min, The chloroform was removed in-vacuo and the residue dissolved in DMSO (6 ml) and 6N sodium hydroxide (2 ml) and stirred at rt overnight. 6N hydrochloric acid was added to adjust the pH to 5 and a white precipitate was formed. The collected solid was dissolved in methyl sulfoxide and purified by HPLC (0.1% TFA acetonitrile, 0.1% TFA water) to yield the trifluoroacetate salt of the title compound as a white solid. Yield 30 mg, 3.83% (2 steps), MS (ES+): [M+H]⁺=491.0, ¹H NMR (400 MHz, DMSO-d₆) δ ppm 4.12 (d, J=5.56 Hz, 2H), 4.49 (d, J=6.06 Hz, 2H), 7.33 (dd, J=8.34, 2.02 Hz, 1 H), 7.51-7.71 (m, 3H), 8.00 (dt, J=9.09, 1.52 Hz, 1H), 8.39 (s, 1H), 8.69 (dd, J=4.80, 1.26 Hz, 1 H), 8.71 (d, J=2.02 Hz, 1H), 8.86 (br. s., 1H), 10.23 (br. s., 1H), 13.02 (br. s., 1H)

### Example 136

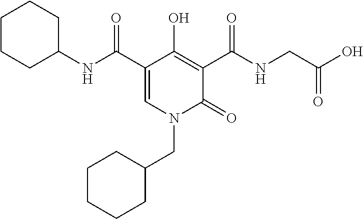

### N-{[5-[(Cyclohexylamino)carbonyl]-1-(cyclohexylmethyl)-4-hydroxy-2-oxo-1,2-dihydro-3-pyridinyl]carbonyl}glycine

136a) Methyl 1-(cyclohexylmethyl)-4-hydroxy-6-oxo-1,6-dihydro-3-pyridinecarboxylate. Under an inert atmosphere tris(methyloxy)methane (914 mg, 8.61 mmol) was added to a stirred solution of dimethyl 3-oxopentanedioate (1.5 g, 8.61 mmol) in acetic anhydride (1.63 ml, 17.23 mmol) and the contents heated (120° C., 2 hr). Volatiles were removed in-vacuo after cooling to touch. The residue was dissolved in tetrahydrofuran (50 ml) and to the solution was added 1-cyclohexylmethanamine (975 mg, 8.61 mmol) with stirring at rt overnight. Sodium hydride (517 mg, 21.53 mmol) was added to the stirred, cooled solution with in portions with nitrogen gas. The cooling was removed and solution stirred at rt 2 hr before the THF was removed and the product partitioned between ethyl acetate (125 ml) and 2N HCl (20 ml). After adjusting pH to 5 the aqueous was extracted with EtOAc (70 ml, 2×) and the combined organic dried and concentrated to produce the title compound as an orange-brown oil that was carried on as is. MS (ES+): [M+H]⁺=266.1.

136b) N-Cyclohexyl-1-(cyclohexylmethyl)-4-hydroxy-6-oxo-1,6-dihydro-3-pyridinecarboxamide. A neat mixture of methyl 1-(cyclohexylmethyl)-4-hydroxy-6-oxo-1,6-dihydro-3-pyridinecarboxylate (500 mg, 1.88 mmol) and cyclohexylamine (1787 mg, 1.88 mmol) was heated in a microwave reactor to 120° C. for 45 min. The residue was partitioned between ethyl acetate (125 ml) and 10% HCl (40 ml). The aqueous was extracted with EtOAc (30 ml, 2×). The combined organic was dried and concentrated to yield an orange-brown residue that was carried on as is. MS (ES+): [M+H]⁺=333.2.

136c) N-{[5-[Cyclohexylamino)carbonyl]-1-(cyclohexylmethyl)-4-hydroxy-2-oxo-1,2-dihydro-3-pyridinyl]carbonyl}glycine. Ethyl N-(oxomethylidene)glycinate (874 mg, 6.77 mmol) was added to a solution of N-cyclohexyl-1-(cyclohexylmethyl)-4-hydroxy-6-oxo-1,6-dihydro-3-pyridinecarboxamide (500 mg, 1.5 mmol) and diisopropylethylamine (428 mg, 3.31 mmol) in chloroform (10 ml) and the contents heated in a microwave reactor to 120° C. for 45 min, The chloroform was removed in-vacuo and the residue dissolved in DMSO (6 ml) and 6N sodium hydroxide (2 ml) and stirred at rt overnight. 6N hydrochloric acid was added to adjust the pH to 5 and a white precipitate was formed. The collected solid was dissolved in methyl sulfoxide and purified by HPLC (0.1% TFA acetonitrile, 0.1% TFA water) to yield the title compound as a pale yellow solid. Yield 119 mg, 17.3%, MS (ES+): [M+H]⁺=434.2, ¹H NMR (400 MHz, DMSO-d₆) δ ppm 0.97 (d, J=11.87 Hz, 2H), 1.07-1.41 (m, 10H), 1.54-1.77 (m, 8H), 1.82 (d, J=9.60 Hz, 2 H), 3.85-3.92 (m, 2H), 4.16 (ddd, J=14.08, 6.88, 6.57 Hz, 2H), 7.95 (d, J=7.58 Hz, 1H), 8.45 (s, 1H), 10.48 (t, J=5.43 Hz, 1H)

### Example 137

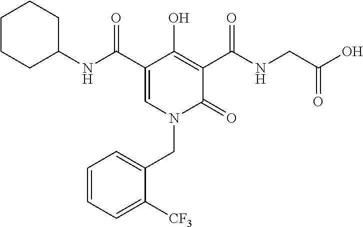

### N-[(5-[(Cyclohexylamino)carbonyl]-4-hydroxy-2-oxo-1-{[2-(trifluoromethyl)phenyl]methyl}-1,2-dihydro-3-pyridinyl)carbonyl]glycine

137a) Methyl 4-hydroxy-6-oxo-1-{[2-(trifluoromethyl)phenyl]methyl}-1,6-dihydro-3-pyridinecarboxylate. Under an inert atmosphere tris(methyloxy)methane (914 mg, 8.61 mmol) was added to a stirred solution of dimethyl 3-oxopentanedioate (1.5 g, 8.61 mmol) in acetic anhydride (1.63 ml, 17.23 mmol) and the contents heated (120° C., 2 hr). Volatiles were removed in-vacuo after cooling to touch. The residue was dissolved in tetrahydrofuran (50 ml) and to the solution was added 1-[2-(trifluoromethyl)phenyl]methanamine (1.5 g, 8.61 mmol) with stirring at rt overnight. Sodium hydride (517 mg, 21.53 mmol) was added to the stirred, cooled solution with in portions with nitrogen gas. The cooling was removed and solution stirred at rt 2 hr before the THF was removed and the product partitioned between ethyl acetate (125 ml) and 2N HCl (20 ml). After adjusting pH to 5 the aqueous was extracted with EtOAc (70 ml, 2×) and the combined organic layers were dried and concentrated to produce the title compound as an orange-brown oil that was carried on as is. MS (ES+): [M+H]⁺=328.1.

137b) N-Cyclohexyl-4-hydroxy-6-oxo-1-{[2-(trifluoromethyl)phenyl]methyl}-1,6-dihydro-3-pyridinecarboxamide. A neat mixture of methyl 4-hydroxy-6-oxo-1-{[2-(trifluoromethyl)phenyl]methyl}-1,6-dihydro-3-pyridinecarboxylate (500 mg, 1.53 mmol) and cyclohexylamine (1.06 g, 10.7 mmol) was heated in a microwave reactor to 120° C. for 45 min. The residue was partitioned between ethyl acetate (125 ml) and 10% HCl (40 ml). The aqueous was extracted with EtOAc (30 ml, 2×). The product precipitated from both layers. The collected solid was dried in-vacuo to yield the title compound as a pale yellow solid. The product was carried on as is. Yield: 402 mg, 64.7%, MS (ES+): [M+H]⁺=395.2.

137c) N-[(5-[(Cyclohexylamino)carbonyl]-4-hydroxy-2-oxo-1-{[2-(trifluoromethyl)phenyl]methyl}-1,2-dihydro-3-pyridinyl)carbonyl]glycine. Ethyl N-(oxomethylidene)glycinate (687 mg, 5.32 mmol) was added to a solution of N-cyclohexyl-4-hydroxy-6-oxo-1-{[2-(trifluoromethyl)phenyl]methyl}-1,6-dihydro-3-pyridinecarboxamide (420 mg, 1.06 mmol) and diisopropylethylamine (413 mg, 3.19 mmol) in chloroform (10 ml) and the contents heated in a microwave reactor to 125° C. for 60 min. To the reaction mixture was added 6M NaOH (2.5 ml) and the biphasic solution was rapidly stirred for 1 hour. The chloroform was removed in-vacuo and the residue dissolved into DMSO (5 ml) and the pH adjusted to 5 with 6N HCl. The solution was stirred at rt 1 hr and the solid collected and dried in-vacuo to yield the title compound as a pale yellow solid. Yield 367 mg, 68.9%, MS (ES+): [M+H]⁺=496.3, ¹H NMR (400 MHz, DMSO-d₆) δ ppm 1.12-1.40 (m, 6H), 1.55 (d, J=12.88 Hz, 1H), 1.67 (dd, J=8.97, 3.66 Hz, 2H), 1.83 (d, J=9.09 Hz, 2H), 3.73-3.85 (m, 1H), 4.01 (d, J=5.56 Hz, 2H), 5.44 (s, 2 H), 6.97 (d, J=7.83 Hz, 1H), 7.50 (t, J=7.58 Hz, 1H), 7.62 (t, J=7.58 Hz, 1H), 7.79 (d, J=7.58 Hz, 1H), 8.59 (s, 1H), 10.31 (br. s., 1H)

### Example 138

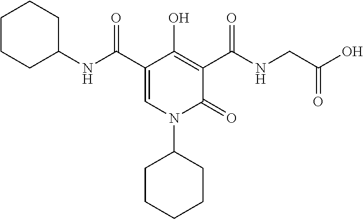

### N-({1-Cyclohexyl-5-[(cyclohexylamino)carbonyl]-4-hydroxy-2-oxo-1,2-dihydro-3-pyridinyl}carbonyl)glycine

138a) Methyl 1-cyclohexyl-4-hydroxy-6-oxo-1,6-dihydro-3-pyridinecarboxylate. Into a 500 mL round-bottomed flask were added methyl 1,3-acetondicarboxylate (5 g, 28.7 mmol) and trimethyl orthformate (3.05 g, 28.7 mmol). Acetic anhydride (5.42 ml, 57.4 mmol) was then added. The mixture was stirred and heated at 120° C. for two hours, then cooled down to room temperature. After removing all volatile materials under reduced pressure, the residue was dissolved with THF (30 mL). Cyclohexylamine (2.85 g, 28.7 mmol) was added slowly. The reaction mixture was stirred at room temperature for overnight. LCMS showed unclyclized intermediate. Sodium hydride (0.827 g, 34.5 mmol) was added portionwise and the mixture was stirred at rt for 30 min. After removal of THF, the residue was dissolved in 15 mL DMSO. The DMSO solution was slowly dropped into 500 mL acidic ice-water (0.1 M HCl) with vigorously stirring. The solid was precipitated out, washed with water for a few times and air-dried under vacuum for 2 days. Crude product was obtained (3.51 g, 96% LCMS purity, 47% estimated yield) and used for next step without further purification. MS (ES+): m/z [M+H]⁺=251.8; ¹H NMR (400 MHz, DMSO-d₆) δ ppm 1.00-1.94 (m, 10H) 3.81 (s, 3H) 4.53-4.65 (m, 1H) 5.70 (s, 1H) 8.26 (s, 1H) 10.77 (s, 1H).

138b) N,1-Dicyclohexyl-4-hydroxy-6-oxo-1,6-dihydro-3-pyridinecarboxamide. Methyl 1-cyclohexyl-4-hydroxy-6-oxo-1,6-dihydro-3-pyridinecarboxylate (0.261 g×96%=0.251 g, 1 mmol) and cyclohexylamine (0.248 g, 2.5 mmol) in a microwave tube was mixed and heated at 100° C. for 30 min in a microwave machine. LCMS of the mixture showed 68% of desired product peak plus 23% of acid by-product peak. The mixture was added EtOAc and 0.1 M HCl solution for work-up. The organic layers from extraction were combined, dried over Na₂SO₄ and concentrated to a solid. The solid weighted 0.256 g. LCMS of the crude product showed 76% of purity (17% of acid by product). The estimated yield is 61%. MS (ES+): m/z [M+H]⁺=319.0. The compound was used for next step without purification.

138c) N-({1-Cyclohexl-5-[cyclohexylamino)carbonyl]-4-hydroxy-2-oxo-1,2-dihydro-3-pyridinyl}carbonyl)glycine. Into a microwave tube were added N,1-dicyclohexyl-4-hydroxy-6-oxo-1,6-dihydro-3-pyridinecarboxamide (237 mg×76%=180 mg, 0.565 mmol), ethyl N-(oxomethylidene)glycinate (88 mg, 0.678 mmol), and DIEA (0.128 mL, 0.735 mmol) in chloroform (1 mL) to give a brown suspension. The mixture was heated at 120° C. for 50 min in a microwave machine. LCMS showed that the reaction was complete. The reaction mixture was transferred into a flask and concentrated into a residue. Ethanol (6 mL) and 1.1 mL of 2M NaOH solution were added. The resulted mixture was stirred at room temperature for 30 min. LCMS showed that the reaction was complete. Reaction mixture was concentrated and then added 2 mL of water and adjusted pH=5 using 6N HCl solution. Reaction mixture was concentrated and the residue was dissolved in DMSO (5 mL). DMSO solution was filtered and purified with Gilson preparative HPLC. Solid product (10 mg, 100% LCMS purity) was obtained. MS (ES+): m/z [M+H]⁺=420.2; ¹H NMR (400 MHz, DMSO-d₆) δ ppm 1.19-1.43 (m, 8H) 1.49-1.74 (m, 6H) 1.74-1.93 (m, 6H) 3.79 (br. s., 1H) 4.12 (d, J=5.56 Hz, 2H) 4.65 (t, J=12.51 Hz, 1H) 7.98 (d, J=7.33 Hz, 1H) 8.39 (s, 1H) 10.52 (br. s., 1H) 13.02 (br. s., 1H)

### Example 139

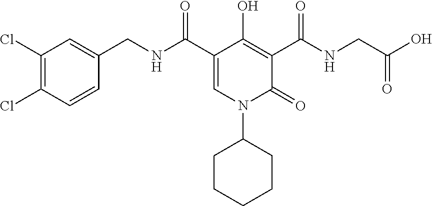

### N-{[1-Cyclohexyl-5-({[(3,4-dichlorophenyl)methyl]amino}carbonyl)-4-hydroxy-2-oxo-1,2-dihydro-3-pyridinyl]carbonyl}glycine

139a) 1-Cyclohexyl-N-[(3,4-dichlorophenyl)methyl]-4-hydroxy-6-oxo-1,6-dihydro-3-pyridinecarboxamide. Methyl 1-cyclohexyl-4-hydroxy-6-oxo-1,6-dihydro-3-pyridinecarboxylate (0.157 g×96%=0.151 g, 0.6 mmol) and [(3,4-dichlorophenyl)methyl]amine (0.264 g, 2.5 mmol) in a microwave tube was mixed and heated at 120° C. for 30 min in a microwave machine. The mixture was added EtOAc and 1 M HCl solution for work-up. The organic layers from extraction were combined, dried over Na₂SO₄ and concentrated to a solid. The residue was dissolved in 3 mL DMSO and subjected to Gilson preparative HPLC purification. The purified product weighted 54 mg (97% LCMS purity, 22.1% yield). MS (ES+): m/z [M+H]⁺=394.9. ¹H NMR (400 MHz, DMSO-d₆) δ ppm 0.94-2.04 (m, 10H) 4.48 (d, J=5.81 Hz, 2H) 4.63 (t, J=12.13 Hz, 1H) 5.66 (s, 1H) 7.32 (d, J=10.11 Hz, 1H) 7.54-7.68 (m, 2H) 8.32 (s, 1H) 9.05 (t, J=5.94 Hz, 1H) 12.63 (br. s., 1H)

139b) N-{[1-Cyclohexyl-5-({[(3,4-dichlorophenyl)methyl]amino}carbonyl)-4-hydroxy-2-oxo-1,2-dihydro-3-pyridinyl]carbonyl}glycine. Into a microwave tube were added 1-cyclohexyl-N-[(3,4-dichlorophenyl)methyl]-4-hydroxy-6-oxo-1,6-dihydro-3-pyridinecarboxamide (54 mg×97%=52.4 mg, 0.137 mmol), ethyl N-(oxomethylidene)glycinate (21 mg, 0.164 mmol), and DIEA (0.031 mL, 0.178 mmol) in chloroform (1 mL) to give a brown suspension. The mixture was heated at 120° C. for 30 min in a microwave machine. LCMS showed that the reaction was complete. The reaction mixture was transferred into a flask and concentrated into a residue. Ethanol (3 mL) and 0.1 mL of 2M NaOH solution were added. The resulting mixture was stirred at room temperature for 30 min. LCMS showed that the reaction was complete. The mixture was added 6N HCl solution dropwise to acidify the solution. The product was precipitated out at pH=5. The solution containing solid product was added iced water and stirred. The solid was filtered, washed with water for a few times and dried under vacuum. The crude solid product was dissolved in 6 mL DMSO. The DMSO solution was filtered and subjected to Gilson preparative HPLC purification. The pure product weighted 15 mg (100% LCMS purity, 22% yield). MS (ES+): m/z [M+H]⁺=496.2; ¹H NMR (400 MHz, DMSO-d₆) d ppm 0.28-2.20 (m, 10H) 4.12 (d, J=5.56 Hz, 2H) 4.48 (d, J=6.06 Hz, 2H) 4.65 (br. s., 1H) 7.32 (dd, J=8.34, 2.02 Hz, 1H) 7.50-7.68 (m, 2H) 8.42 (s, 1H) 8.75 (br. s., 1H) 10.50 (br. s., 1H) 13.02 (s, 1H).

### Example 140

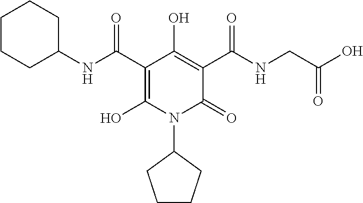

### N-({5-[(Cyclohexylamino)carbonyl]-1-cyclopentyl-4,6-dihydroxy-2-oxo-1,2-dihydro-3-pyridinyl}carbonyl)glycine

140a) 7-(Cyclopentylamino)-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (2.6 g, 11.27 mmol) was stirred in chloroform (25 mL) and cooled over an ice bath. N,N-diisopropylethylamine (2.222 ml, 12.84 mmol) in chloroform (10 mL) was added dropwise, followed by a solution of cyclopentylamine (1.093 g, 12.84 mmol) in chloroform (15 mL). Cooling was removed and the mixture was stirred at room temperature for two hours. The solvent was evaporated and the residue was dissolved in ethyl acetate, washed with water, dried and evaporated to give the title compound (3.0 g, 84%) ¹H NMR (400 MHz, DMSO-d₆) δ ppm 9.11 (s, 1H), 5.03-5.45 (m, 1H), 3.70-4.15 (m, J=91.70 Hz, 1H), 1.91 (s, 2H), 1.41-1.77 (m, 12H).

140b) Methyl 1-cyclopentyl-2,4-dihydroxy-6-oxo-1,6-dihydro-3-pyridinecarboxylate. 7-(Cyclopentylamino)-2,2-dimethyl-4H,5H-pyrano[4,3-d][1,3]dioxin-4,5-dione (3.0 g, 10.74 mmol) in methanol (5 ml) was treated with 25% Sodium methoxide in methanol (5 ml, 21.88 mmol) and the mixture was heated at 70° C. for 3 hours. The mixture was acidified with 1 molar hydrochloric acid and extracted with ethyl acetate (×2). The combined extracts were washed with 1 molar hydrochloric acid and evaporated to a dark oil, that was purified by flash chromatography (0-5% methanol in dichloromethane) to give the title compound (970 mg, 36%) ¹H NMR (400 MHz, DMSO-d₆) δ ppm 3.96 (tt, J=13.58, 6.73 Hz, 1H), 3.70 (s, 1H), 3.65 (s, 3H), 1.72-1.87 (m, 2 H), 1.57-1.68 (m, 2H), 1.43-1.56 (m, 2H), 1.29-1.42 (m, 2H).

140c) Methyl 1-cyclopentyl-5-({[2-(ethyloxy)-2-oxoethyl]amino}carbonyl)-2,4-dihydroxy-6-oxo-1,6-dihydro-3-pyridinecarboxylate. A mixture of methyl 1-cyclopentyl-2,4-dihydroxy-6-oxo-1,6-dihydro-3-pyridinecarboxylate (967 mg, 3.82 mmol), diisopropylethylamine (0.727 ml, 4.20 mmol) and ethyl isocyanatoacetate (0.471 ml, 4.20 mmol) in chloroform (60 mL) was sealed in a pressure flask under nitrogen and heated at 100° C. for 75 minutes. The mixture was washed with 1 molar hydrochloric acid (×2), dried and evaporated to an oil, which was purified by flash chromatography (0-5% methanol in dichloromethane) to give a white solid from diethyl ether (560 mg, 38%) ¹H NMR (400 MHz, DMSO-d6) δ ppm 10.17 (br. s., 1H), 5.31-5.48 (m, 1H), 4.13 (q, J=7.07 Hz, 2H), 4.08 (d, J=5.56 Hz, 2H), 3.76 (s, 3H).

140d) N-({5-[(Cyclohexylamino)carbonyl]-1-cyclopentyl-4,6-dihydroxy-2-oxo-1,2-dihydro-3-pyridinyl}carbonyl)glycine. A mixture of methyl 1-cyclopentyl-5-({[2-(ethyloxy)-2-oxoethyl]amino}carbonyl)-2,4-dihydroxy-6-oxo-1,6-dihydro-3-pyridinecarboxylate (210 mg, 0.549 mmol) and cyclohexylamine (0.075 ml, 0.659 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that recrystallized from ethanol to give the title compound (114 mg, 49%) ¹H NMR (400 MHz, DMSO-d6) δ ppm 12.99 (s, 1H), 9.80 (br. s., 2H), 5.22-5.50 (m, 1H), 4.09 (d, J=5.56 Hz, 2H), 3.75-3.89 (m, 1H), 1.99-2.12 (m, 2H), 1.81-1.95 (m, 4H), 1.71-1.82 (m, 2H), 1.62-1.72 (m, 2H), 1.50-1.61 (m, J=9.98, 4.93 Hz, 3H), 1.30-1.45 (m, 4H), 1.15-1.29 (m, J=7.07 Hz, 1H).

### Example 141

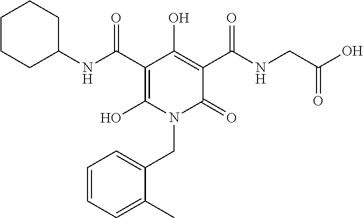

### N-({5-[(Cyclohexylamino)carbonyl]-4,6-dihydroxy-1-[(2-methylphenyl)methyl]-2-oxo-1,2-dihydro-3-pyridinyl}carbonyl)glycine

141a) 2,2-Dimethyl-7-{[(2-methylphenyl)methyl]amino}-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was taken up in chloroform (25 mL) and cooled over an ice bath. N,N-Diisopropylethylamine (2.56 ml, 14.82 mmol) in chloroform (10 mL) was added dropwise, followed by a solution of 2-methylbenzylamine (1.796 g, 14.82 mmol) in chloroform (15 mL). Cooling was removed and the mixture was stirred at room temperature for two hours. The chloroform was evaporated and water added. A solid formed that was collected, washed with water, methanol and hexane then dried to give the title compound (2.44 g, 52%) ¹H NMR (400 MHz, DMSO-d6) δ ppm 9.44 (s, 1 H), 5.29 (d, J=43.70 Hz, 1H), 4.44 (d, J=33.35 Hz, 2H), 2.30 (s, 3H), 1.64 (s, 6H).

141b) Methyl 2,4-dihydroxy-1-[(2-methylphenyl)methyl]-6-oxo-1,6-dihydro-3-pyridinecarboxylate. 2,2-Dimethyl-7-{[(2-methylphenyl)methyl]amino}-4H,5H-pyrano[4,3-d][1,3]dioxin-4,5-dione (2.44 g, 7.74 mmol) in Methanol (5 ml) was treated with 25% Sodium methoxide in methanol (5.08 ml, 22.21 mmol) and the mixture was heated at 70° C. for 3 hours. The mixture solidified after 30 minutes heating. The mixture was cooled, taken up in ethyl acetate and washed with 1 molar hydrochloric acid. The aqueous solution was extracted with ethyl acetate and the combined extracts washed with 1 molar hydrochloric acid, dried and evaporated to an off white solid that was slurried in diethyl ether, collected, washed with hexane and dried to give the title compound (2.1 g, 94%) ¹H NMR (400 MHz, DMSO-d6) δ ppm 12.90 (s, 1H), 7.17 (d, 1H), 7.05-7.15 (m, 2H), 6.65 (d, J=6.82 Hz, 1H), 5.36 (s, 1H), 5.02 (s, 2H), 3.74 (s, 3H), 2.34 (s, 3 H).

141c) Methyl 5-({[2-(ethyloxy)-2-oxoethyl]amino}carbonyl)-2,4-dihydroxy-1-[(2-methylphenyl)methyl]-6-oxo-1,6-dihydro-3-pyridinecarboxylate. A mixture of methyl 2,4-dihydroxy-1-[(2-methylphenyl)methyl]-6-oxo-1,6-dihydro-3-pyridinecarboxylate (2.02 g, 6.98 mmol), diisopropylethylamine (1.329 ml, 7.68 mmol) and Ethyl isocyanatoacetate (0.862 ml, 7.68 mmol) in chloroform (120 mL) was sealed in a pressure flask under nitrogen and heated at 110° C. for 3 hours. The mixture was washed with 1 molar hydrochloric acid (×2), dried and evaporated to an oil, diethyl ether was added and the mixture stirred o/n. The off white solid was collected, washed with diethyl ether & hexane and dried in vacuo to give the title compound (2.13 g, 73%) ¹H NMR (400 MHz, DMSO-d6) δ ppm 10.10 (s, 1H), 7.14-7.19 (m, 1H), 7.03-7.14 (m, J=14.27, 7.33, 7.20, 1.52 Hz, 2H), 6.75 (d, J=7.07 Hz, 1H), 5.04 (s, 2H), 4.12 (q, J=7.07 Hz, 2 H), 4.06 (d, J=5.31 Hz, 2H), 3.73 (s, 3H), 2.36 (s, 3H), 1.20 (t, J=7.20 Hz, 3H).

141d) N-({5-[(Cyclohexylamino)carbonyl]-4,6-dihydroxy-1-[(2-methylphenyl)methyl]-2-oxo-1,2-dihydro-3-pyridinyl}carbonyl)glycine. A mixture of methyl 5-({[2-(ethyloxy)-2-oxoethyl]amino}carbonyl)-2,4-dihydroxy-1-[(2-methylphenyl)methyl]-6-oxo-1,6-dihydro-3-pyridinecarboxylate (310 mg, 0.741 mmol) and cyclohexylamine (0.102 ml, 0.889 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a foam that recrystallized from ethanol to give the title compound (131 mg, 39%) ¹H NMR (400 MHz, DMSO-d6) δ ppm 13.04 (br. s., 1H), 9.82 (s, 1H), 9.59 (s, 1H), 7.19 (d, 1H), 7.04-7.16 (m, 2H), 6.73 (d, J=7.07 Hz, 1H), 5.07 (s, 2H), 4.10 (d, J=5.31 Hz, 2H), 3.72-3.94 (m, 1H), 2.36 (s, 3H), 1.79-1.91 (m, J=8.08 Hz, 2H), 1.62-1.72 (m, 2H), 1.49-1.60 (m, 1H), 1.29-1.47 (m, 4H), 1.15-1.28 (m, 1H).

### Example 142

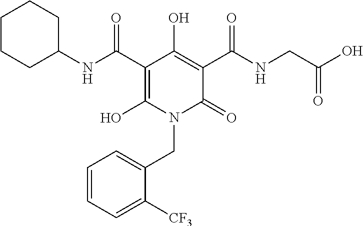

### N-[(5-[(Cyclohexylamino)carbonyl]-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmol) and cyclohexylamine (0.087 ml, 0.762 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a solid that recrystallized from ethanol to give the title compound (251 mg, 77%) ¹H NMR (400 MHz, DMSO-d6) δ ppm 9.84 (s, 1H), 9.57 (s, 1H), 7.77 (d, J=7.58 Hz, 1H), 7.58 (dd, J=7.58 Hz, 1H), 7.47 (dd, J=7.71 Hz, 1H), 7.01 (d, J=7.83 Hz, 1H), 5.28 (s, 2H), 4.10 (d, J=5.56 Hz, 2H), 3.75-3.89 (m, J=7.07 Hz, 1H), 1.78-1.92 (m, 2H), 1.61-1.73 (m, 2H), 1.50-1.60 (m, J=12.63 Hz, 1H), 1.28-1.47 (m, 4H), 1.14-1.27 (m, 1H).

### Example 143

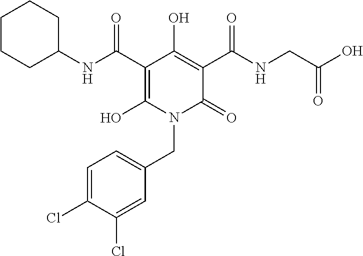

### N-({5-[(Cyclohexylamino)carbonyl]-1-[(3,4-dichlorophenyl)methyl]-4,6-dihydroxy-2-oxo-1,2-dihydro-3-pyridinyl}carbonyl)glycine

143a) 7-{[(3,4-Dichlorophenyl)methyl]-amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was taken up in chloroform (25 mL) and cooled over an ice bath. N,N-diisopropylethylamine (2.56 ml, 14.82 mmol) in chloroform (10 mL) was added dropwise, followed by a solution of 3,4-dichlorobenzylamine (1.976 ml, 14.82 mmol) in chloroform (15 mL). Cooling was removed and the mixture was stirred at room temperature for two hours. The chloroform was evaporated and water added. A solid formed that was collected, washed with water and methanol and dried to give the title compound (3.87 g, 71%) ¹H NMR (400 MHz, DMSO-d6) δ ppm 9.52 (s, 1H), 7.55-7.78 (m, 2H), 7.36 (dd, J=8.34, 2.02 Hz, 1H), 5.08-5.49 (m, 1H), 4.25-4.68 (m, 2H), 1.64 (s, 6H).

143b) Methyl 1-[(3,4-dichlorophenyl)methyl]-2,4-dihydroxy-6-oxo-1,6-dihydro-3-pyridinecarboxylate. 7-{[(3,4-Dichlorophenyl)methyl]amino}-2,2-dimethyl-4H,5H-pyrano[4,3-d][1,3]dioxin-4,5-dione (3.89 g, 10.51 mmol) in methanol (5 ml) was treated with 25% Sodium methoxide in methanol (5.0 ml, 21.88 mmol) and the mixture was heated at 70° C. for 3 hours. The mixture solidified after 30 minutes heating. The mixture was cooled, taken up in ethyl acetate and washed with 1 molar hydrochloric acid. The aqueous solution was extracted with ethyl acetate and the combined extracts washed with 1 molar hydrochloric acid, dried and evaporated to an off white solid that was slurried in diethyl ether, collected, washed with hexane and dried to give the title compound (3.3 g, 91%) ¹H NMR (400 MHz, DMSO-d6) δ ppm 12.89 (s, 1H), 7.58 (d, J=8.34 Hz, 1H), 7.49 (d, J=2.02 Hz, 1H), 7.20 (dd, J=8.34, 1.77 Hz, 1H), 5.31 (s, 1H), 5.03 (s, 2H), 3.75 (s, 3H).

143c) Methyl 1-[(3,4-dichlorophenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(3,4-dichlorophenyl)methyl]-2,4-dihydroxy-6-oxo-1,6-dihydro-3-pyridinecarboxylate (3.4 g, 9.88 mmol), diisopropylethylamine (1.880 ml, 10.87 mmol) and ethyl isocyanatoacetate (1.219 ml, 10.87 mmol) in chloroform (100 mL) was sealed in a pressure flask under nitrogen and heated at 100° C. for 75 minutes. LCMS showed reaction complete. The mixture was washed with 1 molar hydrochloric acid (×2), dried and evaporated to an oil, diethyl ether was added and the mixture stirred o/n. The off white solid was collected, washed with diethyl ether & hexane and dried in vacuo. The mother liquors were purified by flash chromatography (0-6% methanol in dichloromethane) to give the title compound (3.5 g, 75%) ¹H NMR (400 MHz, DMSO-d6) δ ppm 10.13 (t, J=4.55 Hz, 1H), 7.55 (d, J=8.34 Hz, 1H), 7.49 (d, J=2.02 Hz, 1H), 7.24 (dd, J=8.34, 2.02 Hz, 1H), 5.01 (s, 2H), 4.11 (q, J=7.07 Hz, 2H), 4.04 (d, J=5.31 Hz, 2H), 3.66 (s, 3H), 1.20 (t, J=7.07 Hz, 3H)

143d) N-({5-[(Cyclohexylamino)carbonyl]-1-[(3,4-dichlorophenyl)methyl]-4,6-dihydroxy-2-oxo-1,2-dihydro-3-pyridinyl}carbonyl)glycine. A mixture of methyl 1-[(3,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.634 mmol) and cyclohexylamine (0.087 ml, 0.761 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a solid that recrystallized from acetonitrile to give the title compound as a tan solid (170 mg, 52%) ¹H NMR (400 MHz, DMSO-d6) δ ppm 13.02 (br. s., 1H), 9.84 (s, 1H), 9.55 (s, 1H), 7.57 (d, J=8.34 Hz, 1H), 7.55 (d, J=2.02 Hz, 1H), 7.24 (dd, J=8.34, 2.02 Hz, 1H), 5.08 (s, 2H), 4.10 (d, J=5.56 Hz, 2H), 3.69-3.90 (m, 1H), 1.77-2.01 (m, 2H), 1.61-1.75 (m, 2H), 1.50-1.62 (m, J=12.13 Hz, 1H), 1.28-1.46 (m, 4H), 1.12-1.28 (m, 1H).

### Example 144

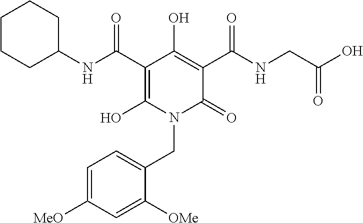

### N-({1-{[2,4-Bis(methyloxy)phenyl]methyl}-5-[(cyclohexylamino)carbonyl]-4,6-dihydroxy-2-oxo-1,2-dihydro-3-pyridinyl}carbonyl)glycine

144a) 7-({[2,4-Bis(methyloxy)phenyl]methyl}amino)-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was taken up in chloroform and cooled over an ice bath. N,N-diisopropylethylamine (2.56 ml, 14.82 mmol) in chloroform was added dropwise, followed by a solution of 2,4-dimethoxybenzylamine (2.226 ml, 14.82 mmol) in chloroform. Cooling was removed and the mixture was stirred at room temperature for two hours. The solvent was evaporated and the residue was slurried in water, collected washed with methanol, hexane and dried to give the title compound (3.97 g, 74%) ¹H NMR (400 MHz, DMSO-d6) δ ppm 9.32 (s, 1H), 7.19 (d, J=8.34 Hz, 1H), 6.59 (s, 1H), 6.52 (d, J=8.34 Hz, 1H), 5.25 (d, J=36.88 Hz, 1H), 4.30 (d, J=44.97 Hz, 2H), 3.17 (d, J=5.31 Hz, 6H), 1.63 (s, 6H).

144b) Methyl 1-{[2,4-bis(methyloxy)phenyl]methyl}-2,4-dihydroxy-6-oxo-1,6-dihydro-3-pyridinecarboxylate. 7-({[2,4-Bis(methyloxy)phenyl]methyl}amino)-2,2-dimethyl-4H,5H-pyrano[4,3-d][1,3]dioxin-4,5-dione (3.97 g, 10.99 mmol) in methanol (5 ml) was treated with 25% Sodium methoxide in methanol (5.0 ml, 21.88 mmol) and the mixture was heated at 70° C. for 3 hours. The mixture was acidified with 1 molar hydrochloric acid and extracted with ethyl acetate (×2). The combine extracts were washed with 1 molar hydrochloric acid and evaporated to a solid (2.6 g, 70.6%) ¹H NMR (400 MHz, DMSO-d6) δ ppm 12.88 (s, 1H), 6.56 (d, J=2.27 Hz, 1H), 6.45-6.53 (m, 1H), 6.41 (dd, 1H), 5.39 (s, 1H), 4.93 (s, 2H), 3.82 (s, 3H), 3.74 (s, 3H), 3.72 (s, 3H).

144c) Methyl 1-{[2,4-bis(methyloxy)phenyl]methyl}-5-({[2-(ethyloxy)-2-oxoethyl]amino}carbonyl)-2,4-dihydroxy-6-oxo-1,6-dihydro-3-pyridinecarboxylate. A mixture of methyl 1-{[2,4-bis(methyloxy)phenyl]methyl}-2,4-dihydroxy-6-oxo-1,6-dihydro-3-pyridinecarboxylate (2.6 g, 7.75 mmol), diisopropylethylamine (1.409 ml, 8.14 mmol) and ethyl isocyanatoacetate (0.913 ml, 8.14 mmol) in chloroform (100 mL) was sealed in a pressure flask under nitrogen and heated at 110° C. for 75 minutes. LCMS showed reaction complete. The mixture was washed with 1 molar hydrochloric acid (×2), dried and evaporated to an oil, diethyl ether was added and the mixture stirred o/n. The off white solid was collected, washed with diethyl ether & hexane and dried in vacuo (2.35 g, 65%) ¹H NMR (400 MHz, DMSO-d6) δ ppm 10.10 (s, 1H), 6.58 (d, J=8.34 Hz, 1H), 6.55 (d, J=2.53 Hz, 1H), 6.41 (dd, J=8.34, 2.27 Hz, 1H), 4.97 (s, 2H), 4.12 (q, J=7.07 Hz, 2H), 4.06 (d, J=5.31 Hz, 2H), 3.82 (s, 3H), 3.74 (s, 3H), 3.72 (s, 3H), 1.20 (t, J=7.07 Hz, 3H).

144d) N-({1-{[2,4-bis(methyloxy)phenyl]methyl}-5-[(cyclohexylamino)carbonyl]-4,6-dihydroxy-2-oxo-1,2-dihydro-3-pyridinyl}carbonyl)glycine. A mixture of methyl 1-{[2,4-bis(methyloxy)phenyl]methyl}-5-({[2-(ethyloxy)-2-oxoethyl]amino}carbonyl)-2,4-dihydroxy-6-oxo-1,6-dihydro-3-pyridinecarboxylate (300 mg, 0.646 mmol) and cyclohexylamine (0.089 ml, 0.775 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a foam then purified by flash chromatography (0-5% methanol in dichloromethane+0.5% acetic acid). The product containing fractions were evaporated to give the title compound (111 mg, 34%) ¹H NMR (400 MHz, DMSO-d6) δ ppm 13.03 (br. s., 1H), 9.81 (s, 1H), 9.61 (s, 1H), 6.48-6.72 (m, 2H), 6.41 (d, J=8.34 Hz, 1H), 4.98 (s, 2H), 4.09 (d, J=4.80 Hz, 2H), 3.76-3.94 (m, 4H), 3.72 (s, 3 H), 1.79-2.07 (m, 2H), 1.60-1.74 (m, 2H), 1.46-1.60 (m, 1H), 1.29-1.44 (m, 4H), 1.14-1.29 (m, J=6.57 Hz, 1H).

### Example 145

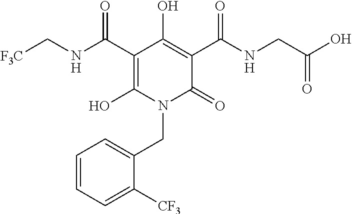

### N-[(4,6-Dihydroxy-2-oxo-5-{[(2,2,2-trifluoroethyl)amino]carbonyl}-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmol), N,N-diisopropylethylamine (0.132 ml, 0.762 mmol) and (2,2,2-trifluoroethyl)amine hydrochloride (103 mg, 0.762 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a foam that recrystallized from acetonitrile to give the title compound (165 mg, 51%) ¹H NMR (400 MHz, DMSO-d6) δ ppm 13.04 (br. s., 1H), 9.96 (s, 1H), 9.67 (s, 1H), 7.77 (d, J=7.83 Hz, 1H), 7.58 (dd, J=7.45 Hz, 1H), 7.47 (dd, J=7.58 Hz, 1H), 7.05 (d, J=7.83 Hz, 1H), 5.30 (s, 2H), 4.16-4.35 (m, 2H), 4.11 (d, J=3.03 Hz, 2H).

### Example 146

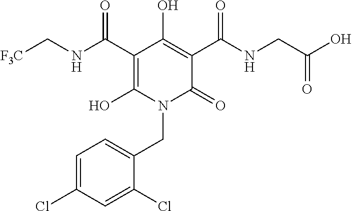

### N-[(1-[(2,4-dichlorophenyl)methyl]-4,6-dihydroxy-2-oxo-5-{[(2,2,2-trifluoroethyl)amino]carbonyl}-1,2-dihydro-3-pyridinyl)carbonyl]glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.634 mmol), N,N-diisopropylethylamine (0.132 ml, 0.761 mmol) and (2,2,2-trifluoroethyl)amine hydrochloride (103 mg, 0.761 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a foam that recrystallized from ethanol to give the title compound (244 mg, 75%) ¹H NMR (400 MHz, DMSO-d6) δ ppm 13.09 (br. s., 1H), 9.95 (s, 1H), 9.66 (s, 1H), 7.66 (d, J=2.27 Hz, 1H), 7.33 (dd, J=8.34, 2.27 Hz, 1H), 7.01 (d, J=8.34 Hz, 1H), 5.13 (s, 2H), 4.15-4.36 (m, 2H), 4.12 (d, J=3.28 Hz, 2H).

### Example 147

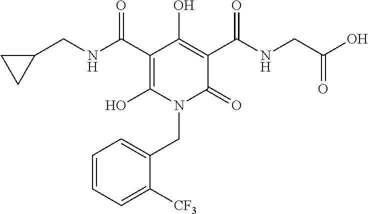

### N-[(5-{[(Cyclopropylmethyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmol) and (cyclopropylmethyl)amine (0.066 ml, 0.762 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a solid that recrystallized from ethanol to give the title compound (113 mg, 37%) ¹H NMR (400 MHz, DMSO-d6) δ ppm 13.00 (br. s., 1H), 9.87 (s, 1H), 9.72 (s, 1H), 7.77 (d, J=7.58 Hz, 1H), 7.58 (dd, J=7.45 Hz, 1H), 7.47 (dd, J=7.58 Hz, 1H), 7.02 (d, J=7.83 Hz, 1H), 5.29 (s, 2H), 4.10 (d, J=5.56 Hz, 2H), 3.24 (dd, J=6.32 Hz, 2H), 0.95-1.16 (m, 1H), 0.39-0.58 (m, 2H), 0.13-0.34 (m, 2H).

### Example 148

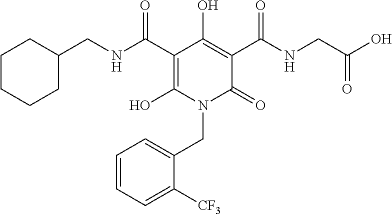

### N-[(5-{[Cyclohexylmethyl)amino]carbonyl}-4,6-dihydroxy-2-oxo-1-{[2-(trifluoromethyl)phenyl]methyl}-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-{[2-(trifluoromethyl)phenyl]methyl}-1,6-dihydro-3-pyridinecarboxylate (300 mg, 0.635 mmol) and (cyclohexylmethyl)amine (0.099 ml, 0.762 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a foam that recrystallized from acetonitrile to give the title compound (150 mg, 75%) ¹H NMR (400 MHz, DMSO-d₆) δ ppm 12.99 (br. s., 1H), 9.87 (s, 1H), 9.65 (s, 1H), 7.77 (d, J=7.58 Hz, 1H), 7.58 (dd, J=7.45 Hz, 1H), 7.47 (dd, J=7.58 Hz, 1H), 7.02 (d, J=7.83 Hz, 1 H), 5.29 (s, 2H), 4.09 (d, J=5.56 Hz, 2H), 3.24 (dd, J=6.32 Hz, 2H), 1.42-1.84 (m, 6H), 1.03-1.36 (m, 3H), 0.85-1.03 (m, 2H).

### Example 149

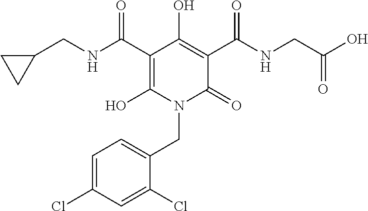

### N-({5-{[(Cyclopropylmethyl)amino]carbonyl}-1-[(2,4-dichlorophenyl)methyl]-4,6-dihydroxy-2-oxo-1,2-dihydro-3-pyridinyl}carbonyl)glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.634 mmol) and (cyclopropylmethyl)amine (0.066 ml, 0.761 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a solid that was recrystallized from acetic acid to give N-({5-{[(cyclopropylmethyl)amino]carbonyl}-1-[(2,4-dichlorophenyl)methyl]-4,6-dihydroxy-2-oxo-1,2-dihydro-3-pyridinyl}carbonyl)glycine (70 mg, 0.145 mmol, 22.80% yield) ¹H NMR (400 MHz, DMSO-d6) δ ppm 13.00 (br. s., 1H), 9.87 (s, 1H), 9.71 (s, 1H), 7.67 (d, J=2.27 Hz, 1H), 7.34 (dd, J=8.46, 2.15 Hz, 1H), 6.96 (d, J=8.59 Hz, 1H), 5.12 (s, 2H), 4.10 (d, J=5.31 Hz, 2H), 3.24 (t, J=6.32 Hz, 2H), 0.92-1.45 (m, J=15.28, 12.19, 7.39, 4.80 Hz, 1H), 0.48 (td, 2H), 0.28 (td, J=5.68, 4.55, 4.42 Hz, 1H).

### Example 150

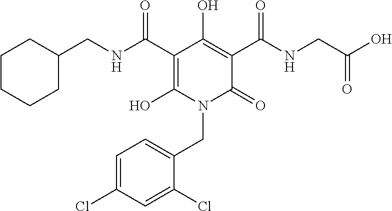

### N-({5-{[(Cyclohexylmethyl)amino]carbonyl}-1-[(2,4-dichlorophenyl)methyl]-4,6-dihydroxy-2-oxo-1,2-dihydro-3-pyridinyl}carbonyl)glycine

A mixture of methyl 1-[(2,4-dichlorophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.634 mmol) and (cyclohexylmethyl)amine (0.099 ml, 0.761 mmol) in chloroform (6.0 mL) was sealed in a pressure tube and heated in a microwave reactor at 150° C. for 30 minutes. The reaction mixture was washed with 1 molar hydrochloric acid and evaporated. The residue was dissolved in ethanol (5 mL) treated with 6 molar sodium hydroxide (3 ml) and stirred overnight. The mixture was partitioned between ethyl acetate and 1 molar hydrochloric acid, the aqueous extracted with ethyl acetate and the combined extracts washed with 1 molar hydrochloric acid. The solution was dried and evaporated to a solid that recrystallized from ethanol to give N-({5-{[(cyclohexylmethyl)amino]carbonyl}-1-[(2,4-dichlorophenyl)methyl]-4,6-dihydroxy-2-oxo-1,2-dihydro-3-pyridinyl}carbonyl)glycine (120 mg, 0.228 mmol, 36.0% yield) ¹H NMR (400 MHz, DMSO-d6) δ ppm 13.01 (br. s., 1H), 9.86 (s, 1H), 9.65 (s, 1H), 7.67 (d, J=2.02 Hz, 1H), 7.34 (dd, J=8.34, 2.27 Hz, 1H), 6.96 (d, J=8.34 Hz, 1H), 5.11 (s, 2H), 4.09 (d, J=5.31 Hz, 2H), 3.24 (dd, J=6.44 Hz, 2H), 1.39-1.89 (m, 6H), 1.03-1.35 (m, 3H), 0.82-1.04 (m, 2H).

### Example 151

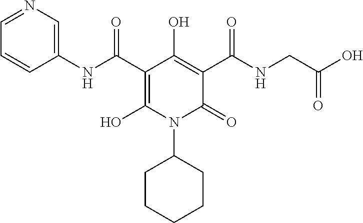

### N-({1-Cyclohexyl-4,6-dihydroxy-2-oxo-5-[(3-pyridinylamino)carbonyl]-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and 3-aminopyridine (78 mg, 0.833 mmol) in chloroform (5 ml) was sealed in a pressure flask and heated at 150° C. for 30 minutes. The reaction mixture was evaporated, dissolved in ethanol (3 mL) and treated with 6 molar sodium hydroxide (3 ml) and stirred until hydrolysis was complete. The mixture was acidified with 1 molar hydrochloric acid which produced an insoluble solid that was collected, washed with water and recrystallised from acetic acid to give N-({1-cyclohexyl-4,6-dihydroxy-2-oxo-5-[(3-pyridinylamino)carbonyl]-1,2-dihydro-3-pyridinyl}carbonyl)glycine (260 mg, 0.604 mmol, 80% yield) ¹H NMR (400 MHz, DMSO-d6) δ ppm 12.41 (s, 1H), 9.76 (s, 1H), 9.17 (s, 1 H), 8.57 (d, J=5.05 Hz, 1H), 8.49 (d, J=8.59 Hz, 1H), 7.86 (dd, J=8.59, 5.31 Hz, 1H), 4.74-5.25 (m, 1H), 4.09 (d, J=4.04 Hz, 2H), 2.41 (q, J=11.03 Hz, 2H), 1.82 (d, J=12.13 Hz, 2H), 1.48-1.74 (m, 3H), 1.31 (q, J=12.55 Hz, 2H), 1.03-1.24 (m, 1H).

### Example 152

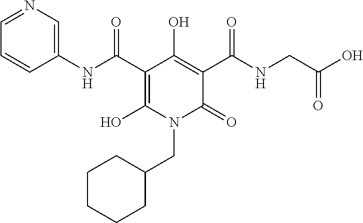

### N-({1-(Cyclohexylmethyl)-4,6-dihydroxy-2-oxo-5-[(3-pyridinylamino)carbonyl]-1,2-dihydro-3-pyridinyl}carbonyl)glycine

152a) 7-[(Cyclohexylmethyl)amino]-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (12 g, 52.0 mmol) was stirred in dichloromethane (300 mL) and cooled over an ice bath. N,N-diisopropylethylamine (9.90 ml, 57.2 mmol) in dichloromethane (100 mL) was added dropwise, followed by a solution of (cyclohexylmethyl)amine (7.44 ml, 57.2 mmol) in dichloromethane (100 mL). Cooling was removed and the mixture was stirred at room temperature for one hour. LCMS indicated that the reaction was complete. The solvent was evaporated and the residue was dissolved in ethyl acetate, washed with water, dried and evaporated to give 7-[(cyclohexylmethyl)amino]-2,2-dimethyl-4H,5H-pyrano[4,3-d][1,3]dioxin-4,5-dione (14.0 g, 45.6 mmol, 88% yield) ¹H NMR (400 MHz, DMSO-d6) δ ppm 9.15 (d, J=48.25 Hz, 1H), 5.27 (d, J=68.46 Hz, 1H), 2.87-3.22 (m, 2H), 1.59-1.82 (m, 11H), 1.36-1.56 (m, 1H), 1.07-1.31 (m, 3H), 0.75-1.06 (m, 2H).

152b) Methyl 1-(cyclohexylmethyl)-2,4-dihydroxy-6-oxo-1,6-dihydro-3-pyridinecarboxylate. 7-[(Cyclohexylmethyl)amino]-2,2-dimethyl-4H,5H-pyrano[4,3-d][1,3]dioxin-4,5-dione (14.0 g, 45.6 mmol) in methanol (30 ml) was treated with 25% Sodium methoxide in methanol (31.2 ml, 137 mmol) and the mixture was heated under reflux for 2 hours and the mixture was heated under reflux for 2 hours to give a thick solid. The solid was stirred with 1 molar hydrochloric acid for 1 hour, collected, washed with 1 molar hydrochloric acid, water and hexane then dried to give methyl 1-(cyclohexylmethyl)-2,4-dihydroxy-6-oxo-1,6-dihydro-3-pyridinecarboxylate (9.18 g, 32.6 mmol, 71.6% yield) ¹H NMR (400 MHz, DMSO-d6) δ ppm 12.77 (s, 1H), 5.30 (s, 1H), 3.75 (s, 3H), 3.70 (d, J=7.07 Hz, 2H), 1.55-1.79 (m, 4H), 1.51 (d, J=12.38 Hz, 2H), 1.03-1.25 (m, 3H), 0.73-1.04 (m, 2H).

152c) Methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate. A mixture of methyl 1-(cyclohexylmethyl)-2,4-dihydroxy-6-oxo-1,6-dihydro-3-pyridinecarboxylate (9.18 g, 32.6 mmol), diisopropylethylamine (6.21 ml, 35.9 mmol) and ethyl isocyanatoacetate (4.03 ml, 35.9 mmol) in chloroform (150 ml) was sealed in a pressure flask under nitrogen and heated at 110° C. for 75 minutes. The mixture was washed with 1 molar hydrochloric acid (×2), dried and evaporated to an oil, which gave methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (10.45 g, 25.5 mmol, 78% yield) as a white solid from MTBE, ¹H NMR (400 MHz, DMSO-d6) δ ppm 10.09 (s, 1H), 4.13 (q, 2H), 4.10 (d, J=6.06 Hz, 2H), 3.82 (s, 3H), 3.79 (d, J=7.07 Hz, 2H), 1.62-1.80 (m, 3H), 1.48-1.62 (m, 3H), 1.21 (t, J=7.07 Hz, 3H), 1.06-1.18 (m, 3H), 0.97 (q, J=11.62 Hz, 2H).

152d) N-({1-(Cyclohexylmethyl)-4,6-dihydroxy-2-oxo-5-[(3-pyridinylamino)carbonyl]-1,2-dihydro-3-pyridinyl}carbonyl)glycine. A mixture of methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (300 mg, 0.731 mmol) and 3-aminopyridine (76 mg, 0.804 mmol) in chloroform (5 ml) was sealed in a pressure flask and heated at 150° C. for 30 minutes. The reaction mixture was evaporated, dissolved in ethanol (3 mL) and treated with 6 molar sodium hydroxide (3 ml) and stirred until hydrolysis was complete. The mixture was acidified with 1 molar hydrochloric acid which produced an insoluble solid that was collected, washed with water and recrystallised from acetic acid to give N-({1-(cyclohexylmethyl)-4,6-dihydroxy-2-oxo-5-[(3-pyridinylamino)carbonyl]-1,2-dihydro-3-pyridinyl}carbonyl)glycine (300 mg, 0.675 mmol, 92% yield) ¹H NMR (400 MHz, DMSO-d6) δ ppm 12.51 (s, 1H), 9.96 (s, 1H), 9.17 (s, 1H), 8.48 (dd, J=5.18, 1.14 Hz, 1H), 8.38 (d, J=8.84 Hz, 1H), 7.76 (dd, J=8.34, 5.05 Hz, 1H), 4.06 (d, J=5.05 Hz, 2H), 3.81 (d, J=7.33 Hz, 2H), 1.86 (none, 1H), 1.71-1.85 (m, 1H), 1.63-1.71 (m, J=5.56 Hz, 2H), 1.57 (d, J=13.89 Hz, 3H), 1.08-1.21 (m, 3H), 0.90-1.07 (m, 2H).

### Example 153

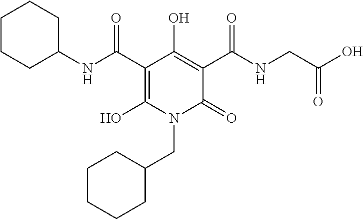

### N-{[5-[(Cyclohexylamino)carbonyl]-1-(cyclohexylmethyl)-4,6-dihydroxy-2-oxo-1,2-dihydro-3-pyridinyl]carbonyl}glycine

A mixture of methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (300 mg, 0.731 mmol) and cyclohexylamine (0.092 ml, 0.804 mmol) in chloroform (5 ml) was sealed in a pressure flask and heated at 150° C. for 30 minutes. The reaction mixture was evaporated, dissolved in ethanol (3 mL) treated with 6 molar sodium hydroxide (3 ml) and stirred until hydrolysis was complete. The mixture was diluted with 1 molar hydrochloric acid to afford a solid that was collected, washed with water then recrystallized from acetic acid to give N-{[5-[(cyclohexylamino)carbonyl]-1-(cyclohexylmethyl)-4,6-dihydroxy-2-oxo-1,2-dihydro-3-pyridinyl]carbonyl}glycine (150 mg, 0.334 mmol, 45.7% yield) ¹H NMR (400 MHz, DMSO-d6) δ ppm 12.96 (br. s., 1H), 9.71 (br. s., 2H), 4.09 (d, J=5.56 Hz, 2H), 3.60-3.89 (m, 3H), 1.80-1.90 (m, J=7.83 Hz, 2H), 1.62-1.79 (m, 5H), 1.48-1.62 (m, J=11.12 Hz, 4H), 1.29-1.45 (m, 4H), 1.05-1.29 (m, 4H), 0.96 (q, J=11.37 Hz, 2H).

### Example 154

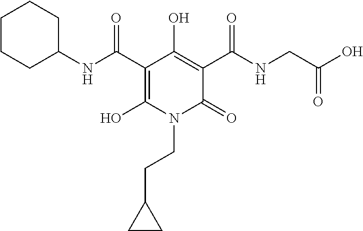

### N-{[5-[(cyclohexylamino)carbonyl]-1-(2-cyclopropylethyl)-4,6-dihydroxy-2-oxo-1,2-dihydro-3-pyridinyl]carbonyl}glycine

154a) 7-[(2-Cyclopropylethyl)amino]-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was stirred in chloroform (35 mL) and cooled over an ice bath. 2-cyclopropylethylamine hydrochloride (1.262 g, 14.82 mmol) was added, followed by N,N-diisopropylethylamine (5.13 ml, 29.6 mmol) in chloroform (15 mL). Cooling was removed and the mixture was stirred at room temperature for two hours. The solution was washed with water, evaporated and the residue crystallised from ethyl acetate-hexane to give the title compound (3.16 g, 76%) ¹H NMR (400 MHz, DMSO-d6) δ ppm 9.15 (d, J=67.20 Hz, 1H), 5.26 (d, J=78.31 Hz, 1H), 3.29-3.45 (m, 1H), 3.12-3.30 (m, J=6.06 Hz, 1H), 1.64 (d, J=6.57 Hz, 6H), 1.42 (d, J=6.57 Hz, 2H), 0.62-0.90 (m, 1H), 0.29-0.57 (m, 2H), −0.06-0.19 (m, 2H).

154b) Methyl 1-(2-cyclopropylethyl)-2,4-dihydroxy-6-oxo-1,6-dihydro-3-pyridinecarboxylate. 7-[(2-Cyclopropylethyl)amino]-2,2-dimethyl-4H,5H-pyrano[4,3-d][1,3]dioxin-4,5-dione (3.16 g, 11.31 mmol) in methanol (5 ml) was treated with 25% Sodium methoxide in methanol (5 ml, 21.88 mmol) and the mixture was heated under reflux for 3 hours. The mixture was acidified with 1 molar hydrochloric acid and extracted with ethyl acetate (×2). The combined extracts were washed with 1 molar hydrochloric acid and evaporated to a solid that was slurried in diethyl ether and collected tom give the title compound (2.5 g, 87%) ¹H NMR (400 MHz, DMSO-d6) δ ppm 12.77 (s, 1H), 5.31 (s, 1H), 3.92 (t, J=7.20 Hz, 2H), 3.75 (s, 3H), 1.40 (td, J=7.16 Hz, 2H), 0.56-0.80 (m, 1H), 0.38 (dt, J=7.96, 5.68, 4.04 Hz, 2H), −0.14-0.09 (m, 2 H).

154c) Methyl 1-(2-cyclopropylethyl)-5-({[2-(ethyloxy)-2-oxoethyl]amino}carbonyl)-2,4-dihydroxy-6-oxo-1,6-dihydro-3-pyridinecarboxylate. A mixture of methyl 1-(2-cyclopropylethyl)-2,4-dihydroxy-6-oxo-1,6-dihydro-3-pyridinecarboxylate (2.5 g, 9.87 mmol), diisopropylethylamine (1.879 ml, 10.86 mmol) and ethyl isocyanatoacetate (1.218 ml, 10.86 mmol) in chloroform (100 mL) was sealed in a pressure flask under nitrogen and heated at 110oC for 75 minutes. LCMS showed reaction complete. The mixture was washed with 1 molar hydrochloric acid (×2), dried and evaporated to an oil, diethyl ether was added and the mixture stirred o/n. The off white solid was collected, washed with diethyl ether & hexane and dried in vacuo (1.75 g, 46%) ¹H NMR (400 MHz, DMSO-d6) δ ppm 10.12 (s, 1H), 4.13 (q, J=7.16 Hz, 2H), 4.09 (d, J=5.56 Hz, 2H), 4.00 (t, 2H), 3.79 (s, 3H), 1.45 (dt, 2H), 1.21 (t, J=6.95 Hz, 3H), 0.62-0.74 (m, 1H), 0.35-0.42 (m, 2 H), −0.04-0.03 (m, 2H).

154d) N-{[5-[(Cyclohexylamino)carbonyl]-1-(2-cyclopropylethyl)-4,6-dihydroxy-2-oxo-1,2-dihydro-3-pyridinyl]carbonyl}glycine. A mixture of methyl 1-(2-cyclopropylethyl)-5-({[2-(ethyloxy)-2-oxoethyl]amino}carbonyl)-2,4-dihydroxy-6-oxo-1,6-dihydro-3-pyridinecarboxylate (300 mg, 0.785 mmol) and cyclohexylamine (0.099 ml, 0.863 mmol) in chloroform (5 ml) was sealed in a pressure flask and heated at 150° C. for 30 minutes. The reaction mixture was evaporated, dissolved in ethanol (3 mL) treated with 6 molar sodium hydroxide (3 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-{[5-[(cyclohexylamino)carbonyl]-1-(2-cyclopropylethyl)-4,6-dihydroxy-2-oxo-1,2-dihydro-3-pyridinyl]carbonyl}glycine (70 mg, 0.166 mmol, 21.17% yield) ¹H NMR (400 MHz, DMSO-d6) δ ppm 12.98 (br. s., 1H), 9.76 (br. s., 2H), 4.09 (d, J=5.81 Hz, 2H), 3.90-4.05 (m, 2H), 3.74-3.89 (m, 1H), 1.78-1.89 (m, 2H), 1.62-1.74 (m, 2H), 1.56 (d, J=11.87 Hz, 1H), 1.29-1.51 (m, 6H), 1.14-1.30 (m, 1H), 0.57-0.76 (m, 1H), 0.30-0.46 (m, 2H), 0.00 (td, J=5.05, 4.29 Hz, 2H).

### Example 155

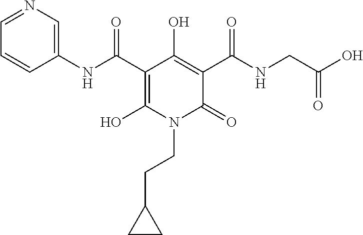

### N-({1-(2-cyclopropylethyl)-4,6-dihydroxy-2-oxo-5-[(3-pyridinylamino)carbonyl]-1,2-dihydro-3-pyridinyl}carbonyl)glycine

A mixture of methyl 1-(2-cyclopropylethyl)-5-({[2-(ethyloxy)-2-oxoethyl]amino}carbonyl)-2,4-dihydroxy-6-oxo-1,6-dihydro-3-pyridinecarboxylate (300 mg, 0.785 mmol) and 3-aminopyridine (81 mg, 0.863 mmol) in chloroform (5 ml) was sealed in a pressure flask and heated at 150° C. for 30 minutes. The reaction mixture was evaporated, dissolved in ethanol (3 mL) treated with 6 molar sodium hydroxide (3 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({1-(2-cyclopropylethyl)-4,6-dihydroxy-2-oxo-5-[(3-pyridinylamino)carbonyl]-1,2-dihydro-3-pyridinyl}carbonyl)glycine (190 mg, 0.456 mmol, 58.2% yield) ¹H NMR (400 MHz, DMSO-d6) δ ppm 12.44 (br. s., 2H), 9.77 (s, 1H), 9.23 (d, J=2.27 Hz, 1H), 8.59 (dd, J=5.31, 1.01 Hz, 1H), 8.54 (d, J=8.59 Hz, 1H), 7.91 (dd, J=8.59, 5.56 Hz, 1H), 4.08 (s, 2H), 4.04 (t, 2H), 1.48 (dt, J=7.16 Hz, 2H), 0.61-0.78 (m, 1H), 0.41 (dt, J=7.96, 5.68, 4.04 Hz, 2H), 0.03 (dt, J=5.12, 4.42 Hz, 1H).

### Example 156

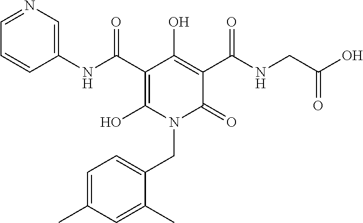

### N-({1-[(2,4-Dimethylphenyl)methyl]-4,6-dihydroxy-2-oxo-5-[(3-pyridinylamino)carbonyl]-1,2-dihydro-3-pyridinyl}carbonyl)glycine

156a) 7-{[(2,4-Dimethylphenyl)methyl]-amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was stirred in chloroform (25 mL) and cooled over an ice bath. N,N-diisopropylethylamine (2.56 ml, 14.82 mmol) in chloroform (10 mL) was added dropwise, followed by a solution of 2,4-dimethylbenzylamine (2.003 g, 14.82 mmol) in chloroform (15 mL). Cooling was removed and the mixture was stirred at room temperature for two hours. The solvent was evaporated and the residue was slurried in water-methanol, collected, washed with methanol then hexane and dried to give the title compound (3.97 g, 81%) ¹H NMR (400 MHz, DMSO-d6) δ ppm 9.38 (s, 1H), 7.10-7.22 (m, J=14.40, 7.83 Hz, 1H), 6.87-7.08 (m, 2H), 5.27 (d, J=36.88 Hz, 1H), 4.38 (d, J=34.86 Hz, 2H), 2.26 (s, 6H), 1.63 (s, 6H).

156b) Methyl 1-[(2,4-dimethylphenyl)methyl]-2,4-dihydroxy-6-oxo-1,6-dihydro-3-pyridinecarboxylate. 7-{[(2,4-Dimethylphenyl)methyl]amino}-2,2-dimethyl-4H,5H-pyrano[4,3-d][1,3]dioxin-4,5-dione (3.97 g, 12.05 mmol) in methanol (5 ml) was treated with 25% Sodium methoxide in methanol (5.0 ml, 21.88 mmol) and the mixture was heated at 70° C. for 3 hours. The mixture was acidified with 1 molar hydrochloric acid and extracted with ethyl acetate (×2). The combine extracts were washed with 1 molar hydrochloric acid and evaporated to a solid (2.72 g, 74.4%) ¹H NMR (400 MHz, DMSO-d6) δ ppm 12.88 (s, 1H), 6.98 (s, 1H), 6.89 (d, J=7.83 Hz, 1 H), 6.56 (d, J=7.58 Hz, 1H), 5.38 (s, 1H), 4.97 (s, 2H), 3.74 (s, 3H), 2.30 (s, 3H), 2.22 (s, 3H).

156c) Methyl 1-[(2,4-dimethylphenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(2,4-dimethylphenyl)methyl]-2,4-dihydroxy-6-oxo-1,6-dihydro-3-pyridinecarboxylate (2.7 g, 8.90 mmol), diisopropylethylamine (1.617 ml, 9.35 mmol) and ethyl isocyanatoacetate (1.049 ml, 9.35 mmol) in chloroform (100 mL) was sealed in a pressure flask under nitrogen and heated at 110° C. for 75 minutes. LCMS showed reaction complete. The mixture was washed with 1 molar hydrochloric acid (×2), dried and evaporated to a solid, that was slurried in diethyl ether, collected and dried to give the title compound (3.04 g, 79%) ¹H NMR (400 MHz, DMSO-d6) δ ppm 10.09 (s, 1H), 6.97 (s, 1H), 6.88 (d, J=7.07 Hz, 1H), 6.65 (d, J=7.83 Hz, 1H), 5.01 (s, 2H), 4.12 (q, J=7.07 Hz, 2H), 4.07 (d, J=5.05 Hz, 2H), 3.75 (s, 3H), 2.32 (s, 3H), 2.22 (s, 3H), 1.20 (t, J=7.20 Hz, 3H).

156d) N-({1-[(2,4-Dimethylphenyl)methyl]-4,6-dihydroxy-2-oxo-5-[(3-pyridinylamino)carbonyl]-1,2-dihydro-3-pyridinyl}carbonyl)glycine. A mixture of methyl 1-[(2,4-dimethylphenyl)methyl]-54 {[2-(ethyloxy)-2-oxoethyl]amino}carbonyl)-2,4-dihydroxy-6-oxo-1,6-dihydro-3-pyridinecarboxylate (300 mg, 0.694 mmol) and 3-aminopyridine (71.8 mg, 0.763 mmol) in chloroform (5 ml) was sealed in a pressure flask and heated at 150° C. for 30 minutes. The reaction mixture was evaporated, the residue dissolved in ethanol (3 mL), treated with 6 molar sodium hydroxide (2 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({1-[(2,4-dimethylphenyl)methyl]-4,6-dihydroxy-2-oxo-5-[(3-pyridinylamino)carbonyl]-1,2-dihydro-3-pyridinyl}carbonyl)glycine (177 mg, 0.379 mmol, 54.7% yield) ¹H NMR (400 MHz, DMSO-d6) δ ppm 12.60 (s, 1H), 10.00 (s, 1H), 9.29 (d, J=2.02 Hz, 1H), 8.52 (d, J=5.56 Hz, 1H), 8.48 (ddd, J=8.65, 2.21, 1.01 Hz, 1H), 7.88 (dd, J=8.59, 5.56 Hz, 1H), 6.99 (s, 1H), 6.88 (d, J=7.83 Hz, 1 H), 6.64 (d, J=7.83 Hz, 1H), 5.07 (s, 2H), 4.05 (d, J=2.27 Hz, 2H), 2.34 (s, 3H), 2.22 (s, 3H).

### Example 157

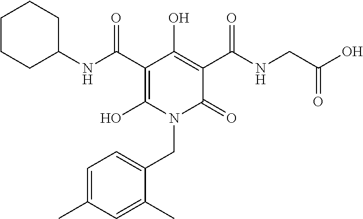

### N-({5-[(Cyclohexylamino)carbonyl]-1-[(2,4-dimethylphenyl)methyl]-4,6-dihydroxy-2-oxo-1,2-dihydro-3-pyridinyl}carbonyl)glycine

A mixture of methyl 1-[(2,4-dimethylphenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.694 mmol) and cyclohexylamine (0.087 ml, 0.763 mmol) in chloroform (5 ml) was sealed in a pressure flask and heated at 150° C. for 30 minutes. The reaction mixture was evaporated, the residue dissolved in ethanol (3 mL) treated with 6 molar sodium hydroxide (2 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({5-[(cyclohexylamino)carbonyl]-1-[(2,4-dimethylphenyl)methyl]-4,6-dihydroxy-2-oxo-1,2-dihydro-3-pyridinyl}carbonyl)glycine (210 mg, 0.445 mmol, 64.2% yield) ¹H NMR (400 MHz, DMSO-d6) δ ppm 13.04 (br. s., 1H), 9.81 (br. s., 1H), 9.58 (br. s., 1H), 7.00 (s, 1H), 6.89 (d, J=8.08 Hz, 1H), 6.62 (d, J=7.83 Hz, 1H), 5.02 (s, 2 H), 4.10 (d, J=5.56 Hz, 2H), 3.63-3.90 (m, J=5.56 Hz, 1H), 2.32 (s, 3H), 2.22 (s, 3H), 1.76-1.90 (m, 2H), 1.60-1.74 (m, 2H), 1.55 (d, J=11.87 Hz, 1H), 1.29-1.45 (m, 4H), 1.10-1.29 (m, 1H).

### Example 158

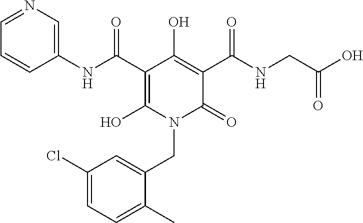

### N-({1-[(5-Chloro-2-methylphenyl)methyl]-4,6-dihydroxy-2-oxo-5-[(3-pyridinylamino)carbonyl]-1,2-dihydro-3-pyridinyl}carbonyl)glycine

158a) 7-{[(5-Chloro-2-methylphenyl)methyl]amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was stirred in chloroform (25 mL) and cooled over an ice bath. N,N-diisopropylethylamine (2.56 ml, 14.82 mmol) in chloroform (10 mL) was added dropwise, followed by a solution of 5-chloro-2-methylbenzylamine (2.306 g, 14.82 mmol) in chloroform (15 mL). Cooling was removed and the mixture was stirred at room temperature for two hours. The solvent was evaporated and the residue was slurried in water, collected, washed with methanol then hexane and dried to give the title compound (3.79 g, 73%) ¹H NMR (400 MHz, DMSO-d6) δ ppm 9.42 (s, 1H), 7.05-7.48 (m, 3H), 5.32 (d, J=64.93 Hz, 1H), 4.44 (d, J=28.55 Hz, 2H), 2.29 (s, 3 H), 1.64 (s, 6H).

158b) Methyl 1-[(5-chloro-2-methylphenyl)methyl]-2,4-dihydroxy-6-oxo-1,6-dihydro-3-pyridinecarboxylate. 7-{[(5-Chloro-2-methylphenyl)methyl]amino}-2,2-dimethyl-4H,5H-pyrano[4,3-d][1,3]dioxin-4,5-dione (3.79 g, 10.84 mmol) in methanol (5 ml) was treated with 25% Sodium methoxide in methanol (5 ml, 21.88 mmol) and the mixture was heated at 70° C. for 3 hours. The mixture was acidified with 1 molar hydrochloric acid and extracted with ethyl acetate (×2). The combine extracts were washed with 1 molar hydrochloric acid and evaporated to an oil. Trituration with diethyl ether gave the title compound (3.27 g, 93%) ¹H NMR (400 MHz, DMSO-d6) δ ppm 12.91 (s, 1H), 7.02-7.42 (m, 2H), 6.63 (s, 1H), 5.35 (s, 1H), 4.99 (s, 2H), 3.75 (s, 3 H), 2.33 (s, 3H).

158c) Methyl 1-[(5-chloro-2-methylphenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(5-chloro-2-methylphenyl)methyl]-2,4-dihydroxy-6-oxo-1,6-dihydro-3-pyridinecarboxylate (3.27 g, 10.10 mmol), diisopropylethylamine (1.835 ml, 10.61 mmol) and ethyl isocyanatoacetate (1.190 ml, 10.61 mmol) in chloroform (100 mL) was sealed in a pressure flask under nitrogen and heated at 110° C. for 75 minutes. LCMS showed reaction complete. The mixture was washed with 1 molar hydrochloric acid (×2), dried and evaporated to an oil, diethyl ether was added and the mixture triturated to give the title compound as an off white solid (3.21 g, 70%) ¹H NMR (400 MHz, DMSO-d6) δ ppm 10.19 (t, J=5.56 Hz, 1H), 7.18 (d, 1H), 7.15 (dd, 1 H), 6.70 (d, J=2.02 Hz, 1H), 4.95 (s, 2H), 4.11 (q, J=7.16 Hz, 2H), 4.03 (d, J=5.56 Hz, 2H), 3.63 (s, 3H), 2.34 (s, 3H), 1.19 (t, J=7.07 Hz, 3H).

158d) N-({1-[(5-chloro-2-methylphenyl)methyl]-4,6-dihydroxy-2-oxo-5-[(3-pyridinylamino)carbonyl]-1,2-dihydro-3-pyridinyl}carbonyl)glycine. A mixture of methyl 1-[(5-chloro-2-methylphenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.662 mmol) and 3-aminopyridine (68.6 mg, 0.729 mmol) in chloroform (5 ml) was sealed in a pressure flask and heated at 150° C. for 30 minutes. The reaction mixture was evaporated, the residue dissolved in ethanol (3 mL), treated with 6 molar sodium hydroxide (2 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({1-[(5-chloro-2-methylphenyl)methyl]-4,6-dihydroxy-2-oxo-5-[(3-pyridinylamino)carbonyl]-1,2-dihydro-3-pyridinyl}carbonyl)glycine (239 mg, 0.491 mmol, 74.1% yield) ¹H NMR (400 MHz, DMSO-d6) δ ppm 12.60 (s, 1H), 10.00 (s, 1H), 9.29 (d, J=2.02 Hz, 1H), 8.52 (d, J=5.56 Hz, 1H), 8.48 (ddd, J=8.65, 2.21, 1.01 Hz, 1H), 7.88 (dd, J=8.59, 5.56 Hz, 1H), 6.99 (s, 1H), 6.88 (d, J=7.83 Hz, 1 H), 6.64 (d, J=7.83 Hz, 1H), 5.07 (s, 2H), 4.05 (d, J=2.27 Hz, 2H), 2.34 (s, 3H), 2.22 (s, 3H).

### Example 159

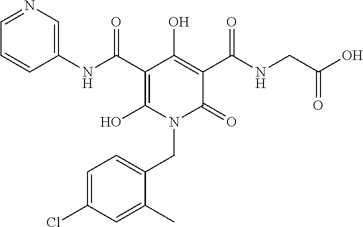

### N-({1-[(4-Chloro-2-methylphenyl)methyl]-4,6-dihydroxy-2-oxo-5-[(3-pyridinylamino)carbonyl]-1,2-dihydro-3-pyridinyl}carbonyl)glycine

159a) 7-{[(4-Chloro-2-methylphenyl)methyl]amino}-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3.0 g, 13.01 mmol) was stirred in chloroform (25 mL) and cooled over an ice bath. N,N-diisopropylethylamine (2.56 ml, 14.82 mmol) in chloroform (10 mL) was added dropwise, followed by a solution of 4-chloro-2-methylbenzylamine (2.306 g, 14.82 mmol) in chloroform (15 mL). Cooling was removed and the mixture was stirred at room temperature for two hours, giving a solid. The solid was collected, washed with dichloromethane then hexane and dried to give the title compound (3.49 g, 67%) ¹H NMR (400 MHz, DMSO-d6) δ ppm 9.45 (d, J=24.50 Hz, 1H), 7.03-7.49 (m, J=21.47 Hz, 3H), 5.29 (d, J=44.21 Hz, 1H), 4.42 (d, J=27.03 Hz, 2H), 2.30 (s, 3 H), 1.64 (s, 6H).

159b) Methyl 1-[(4-chloro-2-methylphenyl)methyl]-2,4-dihydroxy-6-oxo-1,6-dihydro-3-pyridinecarboxylate. 7-{[(4-Chloro-2-methylphenyl)methyl]amino}-2,2-dimethyl-4H,5H-pyrano[4,3-d][1,3]dioxin-4,5-dione (3.49 g, 9.98 mmol) in methanol (5 ml) was treated with 25% Sodium methoxide in methanol (5 ml, 21.88 mmol) and the mixture was heated at 70° C. for 3 hours. The mixture was acidified with 1 molar hydrochloric acid and extracted with ethyl acetate (×2). The combine extracts were washed with 1 molar hydrochloric acid and evaporated to a solid that was slurried in diethyl ether, collected and dried to give the title compound (2.7 g, 84%) ¹H NMR (400 MHz, DMSO-d6) δ ppm 12.89 (s, 1H), 7.27 (d, J=2.02 Hz, 1H), 7.16 (dd, J=8.34, 2.02 Hz, 1H), 6.69 (d, J=8.08 Hz, 1H), 5.32 (s, 1H), 4.97 (s, 2H), 3.74 (s, 3H), 2.35 (s, 3H).

159c) Methyl 1-[(4-chloro-2-methylphenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(4-chloro-2-methylphenyl)methyl]-2,4-dihydroxy-6-oxo-1,6-dihydro-3-pyridinecarboxylate (2.7 g, 8.34 mmol), diisopropylethylamine (1.587 ml, 9.17 mmol) and ethyl isocyanatoacetate (1.029 ml, 9.17 mmol) in chloroform (100 mL) was sealed in a pressure flask under nitrogen and heated at 110° C. for 75 minutes. LCMS showed reaction complete. The mixture was washed with 1 molar hydrochloric acid (×2), dried and evaporated to a foam, triturated with diethyl ether to give the title compound as a solid (3.18 g, 84%) ¹H NMR (400 MHz, DMSO-d6) δ ppm 10.08 (s, 1H), 7.26 (d, J=1.77 Hz, 1H), 7.14 (dd, J=8.34, 2.02 Hz, 1H), 6.79 (d, J=8.34 Hz, 1H), 5.00 (s, 2H), 4.12 (q, J=7.07 Hz, 2H), 4.06 (d, J=5.05 Hz, 2H), 3.72 (s, 3H), 2.37 (s, 3H), 1.20 (t, J=7.07 Hz, 3H).

159d) N-({1-[(4-Chloro-2-methylphenyl)methyl]-4,6-dihydroxy-2-oxo-5-[(3-pyridinylamino)carbonyl]-1,2-dihydro-3-pyridinyl}carbonyl)glycine. A mixture of methyl 1-[(4-chloro-2-methylphenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.662 mmol) and 3-aminopyridine (68.6 mg, 0.729 mmol) in chloroform (5 ml) was sealed in a pressure flask and heated at 150° C. for 30 minutes. The reaction mixture was evaporated, the residue dissolved in ethanol (3 mL), treated with 6 molar sodium hydroxide (2 ml) and stirred until hydrolysis was complete. The mixture was diluted with 1 molar hydrochloric acid to afford a solid that was collected, washed with water then recrystallized from acetic acid to give N-({1-[(4-chloro-2-methylphenyl)methyl]-4,6-dihydroxy-2-oxo-5-[(3-pyridinylamino)carbonyl]-1,2-dihydro-3-pyridinyl}carbonyl)glycine (191 mg, 0.392 mmol, 59.2% yield) ¹H NMR (400 MHz, DMSO-d6) δ ppm 12.60 (s, 1H), 10.00 (s, 1H), 9.29 (d, J=2.02 Hz, 1H), 8.52 (d, J=5.56 Hz, 1H), 8.48 (ddd, J=8.65, 2.21, 1.01 Hz, 1H), 7.88 (dd, J=8.59, 5.56 Hz, 1H), 6.99 (s, 1H), 6.88 (d, J=7.83 Hz, 1H), 6.64 (d, J=7.83 Hz, 1H), 5.07 (s, 2 H), 4.05 (d, J=2.27 Hz, 2H), 2.34 (s, 3H), 2.22 (s, 3H).

### Example 160

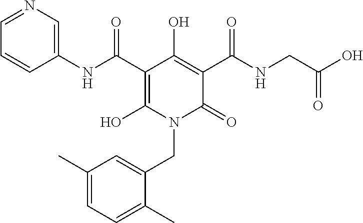

### N-({1-[(2,5-Dimethylphenyl)methyl]-4,6-dihydroxy-2-oxo-5-[(3-pyridinylamino)carbonyl]-1,2-dihydro-3-pyridinyl}carbonyl)glycine

160a) 7-{[(2,5-Dimethylphenyl)methyl]amino}-2,2-dimethyl-4H,5H-pyrano[4,3-d][1,3]dioxin-4,5-dione. 7-chloro-2,2-dimethyl-4H,5H-pyrano[4,3-d][1,3]dioxin-4,5-dione (3.0 g, 13.01 mmol) was stirred in chloroform (25 mL) and cooled over an ice bath. N,N-diisopropylethylamine (2.56 ml, 14.82 mmol) in chloroform (10 mL) was added dropwise, followed by a solution of 2,5-dimethylbenzylamine (2.003 g, 14.82 mmol) in chloroform (15 mL). Cooling was removed and the mixture was stirred at room temperature for two hours. The solvent was evaporated and the residue was slurried in water, collected, washed with methanol then hexane and dried to give the title compound (3.36 g, 69%) ¹H NMR (400 MHz, DMSO-d6) δ ppm 9.37 (s, 1H), 6.85-7.28 (m, J=17.18 Hz, 3H), 5.29 (d, J=49.77 Hz, 1H), 4.39 (d, J=34.10 Hz, 2H), 2.26 (s, 6H), 1.64 (s, 6H).

160b) Methyl 1-[(2,5-dimethylphenyl)methyl]-2,4-dihydroxy-6-oxo-1,6-dihydro-3-pyridinecarboxylate. 7-{[(2,5-dimethylphenyl)methyl]amino}-2,2-dimethyl-4H,5H-pyrano[4,3-d][1,3]dioxin-4,5-dione (3.3 g, 10.02 mmol) in methanol (5 ml) was treated with 25% Sodium methoxide in methanol (5 ml, 21.88 mmol) and the mixture was heated at 70° C. for 3 hours. The mixture was acidified with 1 molar hydrochloric acid and extracted with ethyl acetate (×2). The combine extracts were washed with 1 molar hydrochloric acid and evaporated to a solid that was slurried in diethyl ether and collected to give the title compound (2.8 g, 92%). ¹H NMR (400 MHz, DMSO-d6) δ ppm 12.90 (s, 1H), 7.05 (d, J=7.58 Hz, 1H), 6.93 (d, J=7.58 Hz, 1H), 6.46 (s, 1H), 5.38 (s, 1H), 4.98 (s, 2H), 3.75 (s, 3H), 2.29 (s, 3H), 2.16 (s, 3H).

160c) Methyl 1-[(2,5-dimethylphenyl)methyl]-5-({[2-(ethyloxy)-2-oxoethyl]amino}carbonyl)-2,4-dihydroxy-6-oxo-1,6-dihydro-3-pyridinecarboxylate. A mixture of methyl 1-[(2,5-dimethylphenyl)methyl]-2,4-dihydroxy-6-oxo-1,6-dihydro-3-pyridinecarboxylate (2.8 g, 9.23 mmoles), diisopropylethylamine (1.757 ml, 10.15 mmol) and ethyl isocyanatoacetate (1.139 ml, 10.15 mmol) in chloroform (100 mL) was sealed in a pressure flask under nitrogen and heated at 110° C. for 75 minutes. LCMS showed reaction complete. The mixture was washed with 1 molar hydrochloric acid (×2), dried and evaporated to a solid, the product triturated with diethyl ether to give the title compound (3.03 g, 76%). ¹H NMR (400 MHz, DMSO-d6) δ ppm 10.08 (s, 1 H), 7.05 (d, J=7.83 Hz, 1H), 6.92 (d, 1H), 6.56 (s, 1H), 5.02 (s, 2H), 4.12 (q, J=7.07 Hz, 2H), 4.08 (d, J=5.05 Hz, 2H), 3.77 (s, 3H), 2.31 (s, 3H), 2.16 (s, 3H), 1.20 (t, J=7.07 Hz, 3H).

160d) N-({1-[(2,5-Dimethylphenyl)methyl]-4,6-dihydroxy-2-oxo-5-[(3-pyridinylamino)carbonyl]-1,2-dihydro-3-pyridinyl}carbonyl)glycine. A mixture of methyl 1-[(2,5-dimethylphenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.694 mmol) and 3-aminopyridine (71.8 mg, 0.763 mmol) in chloroform (5 ml) was sealed in a pressure flask and heated at 150° C. for 30 minutes. The reaction mixture was evaporated, the residue dissolved in ethanol (3 mL), treated with 6 molar sodium hydroxide (2 ml) and stirred until hydrolysis was complete. The mixture was diluted with 1 molar hydrochloric acid to afford a solid that was collected, washed with water then recrystallized from acetic acid to give N-({1-[(2,5-dimethylphenyl)methyl]-4,6-dihydroxy-2-oxo-5-[(3-pyridinylamino)carbonyl]-1,2-dihydro-3-pyridinyl}carbonyl)glycine (143 mg, 0.307 mmol, 44.2% yield). ¹H NMR (400 MHz, DMSO-d6) δ ppm 12.67 (s, 1H), 12.30 (br. s., 1H), 10.14 (s, 1H), 9.28 (s, 1H), 8.44 (d, J=4.80 Hz, 1H), 8.40 (d, J=8.59 Hz, 1H), 7.79 (dd, J=8.34, 5.31 Hz, 1H), 7.05 (d, J=7.58 Hz, 1H), 6.90 (d, J=7.58 Hz, 1H), 6.52 (s, 1H), 5.05 (s, 2H), 4.02 (d, J=5.05 Hz, 2H), 2.31 (s, 3H), 2.15 (s, 3H).

### Example 161

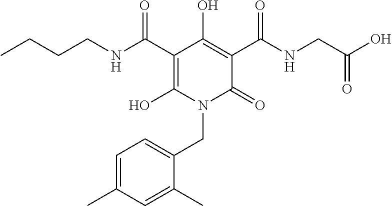

### N-({5-[(Butylamino)carbonyl]-1-[(2,4-dimethylphenyl)methyl]-4,6-dihydroxy-2-oxo-1,2-dihydro-3-pyridinyl}carbonyl)glycine

A mixture of methyl 1-[(2,4-dimethylphenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.694 mmol) and n-butylamine (0.075 ml, 0.763 mmol) in chloroform (5 ml) was sealed in a pressure flask and heated at 150° C. for 30 minutes. The reaction mixture was evaporated, the residue dissolved in ethanol (3 mL), treated with 6 molar sodium hydroxide (2 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({5-[(butylamino)carbonyl]-1-[(2,4-dimethylphenyl)methyl]-4,6-dihydroxy-2-oxo-1,2-dihydro-3-pyridinyl}carbonyl)glycine (191 mg, 0.429 mmol, 61.8% yield) ¹H NMR (400 MHz, DMSO-d6) δ ppm 9.87 (s, 1H), 9.60 (s, 1H), 6.99 (s, 1H), 6.89 (d, J=8.08 Hz, 1H), 6.64 (d, J=7.83 Hz, 1H), 5.02 (s, 2H), 4.09 (d, J=5.56 Hz, 2H), 3.36 (dt, J=6.65 Hz, 2H), 2.33 (s, 3H), 2.22 (s, 3H), 1.42-1.63 (m, 2H), 1.32 (tq, J=7.43 Hz, 2 H), 0.90 (t, J=7.33 Hz, 3H).

### Example 162

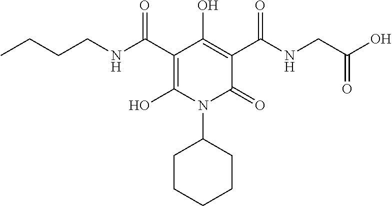

### N-({5-[(Butylamino)carbonyl]-1-cyclohexyl-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and n-butylamine (0.082 ml, 0.833 mmol) in chloroform (5 ml) was sealed in a pressure flask and heated at 150° C. for 30 minutes. The reaction mixture was evaporated, the residue was dissolved in ethanol (3 mL) treated with 6 molar sodium hydroxide (3 ml) and stirred until hydrolysis was complete. The mixture was acidified with 1 molar hydrochloric acid to give a solid that was collected, washed with 1 molar hydrochloric acid and water. The solid was recrystallized from acetic acid, however about 10% starting material persisted. The solid was purified by prep. HPLC (25-95% acetonitrile-water-0.1% TFA) to give N-({5-[(butylamino)carbonyl]-1-cyclohexyl-4,6-dihydroxy-2-oxo-1,2-dihydro-3-pyridinyl}carbonyl)glycine (110 mg, 0.269 mmol, 35.5% yield) as a white solid ¹H NMR (400 MHz, DMSO-d6) δ ppm 12.99 (s, 1H), 9.75 (br. s., 2H), 4.65-5.04 (m, 1H), 4.08 (d, J=5.81 Hz, 2H), 3.36 (dt, J=6.82 Hz, 2H), 2.26-2.43 (m, 2H), 1.80 (d, J=12.63 Hz, 2H), 1.47-1.70 (m, 5H), 1.22-1.39 (m, 4H), 1.14 (q, J=12.88 Hz, 1H), 0.90 (t, J=7.45 Hz, 3H).

### Example 163

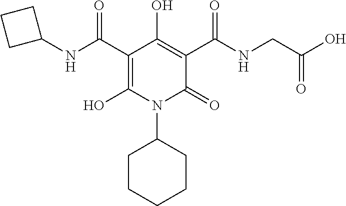

### N-({5-[(Cyclobutylamino)carbonyl]-1-cyclohexyl-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and cyclobutylamine (0.071 ml, 0.833 mmol) in chloroform (40 ml) was sealed in a pressure flask and heated at 150° C. for 30 minutes. The reaction mixture was evaporated, the residue was dissolved in ethanol (3 mL) treated with 6 molar sodium hydroxide (2 ml) and stirred until hydrolysis was complete. The mixture was acidified with 1 molar hydrochloric acid to give a solid that was collected, washed with 1 molar hydrochloric acid and water. The solid was recrystallized from acetic acid to give N-({5-[(cyclobutylamino)carbonyl]-1-cyclohexyl-4,6-dihydroxy-2-oxo-1,2-dihydro-3-pyridinyl}carbonyl)glycine (230 mg, 0.565 mmol, 74.6% yield) ¹H NMR (400 MHz, DMSO-d6) δ ppm 12.75 (br. s., 1H), 4.66-5.17 (m, 1H), 4.37 (dt, J=16.04, 8.08 Hz, 1H), 4.08 (d, J=5.56 Hz, 2H), 2.18-2.44 (m, 4H), 2.09 (dt, J=18.88, 9.38 Hz, 2H), 1.80 (d, J=12.63 Hz, 2H), 1.68-1.76 (m, 2H), 1.50-1.68 (m, 3H), 1.30 (q, J=12.88 Hz, 2H), 1.14 (q, J=12.63 Hz, 1H).

### Example 164

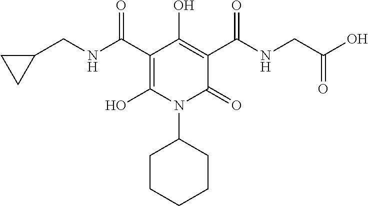

### N-[(1-Cyclohexyl-5-{[(cyclopropylmethyl)amino]carbonyl}-4,6-dihydroxy-2-oxo-1,2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and (cyclopropyl)methylamine (0.072 ml, 0.833 mmol) in chloroform (5 ml) was sealed in a pressure flask and heated at 150° C. for 30 minutes. The reaction mixture was evaporated, the residue was dissolved in ethanol (3 mL) treated with 6 molar sodium hydroxide (2 ml) and stirred until hydrolysis was complete. The mixture was acidified with 1 molar hydrochloric acid to give a solid that was collected, washed with 1 molar hydrochloric acid and water. The solid was recrystallized from acetic acid to give N-[(1-cyclohexyl-5-{[(cyclopropylmethyl)amino]carbonyl}-4,6-dihydroxy-2-oxo-1,2-dihydro-3-pyridinyl)carbonyl]glycine (135 mg, 0.331 mmol, 43.8% yield) ¹H NMR (400 MHz, DMSO-d6) δ ppm 12.98 (br. s., 1H), 9.73 (br. s., 2H), 4.84 (s, 1H), 4.08 (d, J=5.56 Hz, 2H), 3.23 (t, J=6.32 Hz, 2H), 2.19-2.46 (m, 2H), 1.80 (d, J=12.63 Hz, 2H), 1.50-1.70 (m, 3H), 1.30 (q, J=13.05 Hz, 2H), 0.98-1.22 (m, 2H), 0.39-0.58 (m, 2H), 0.28 (td, J=5.87, 4.48, 4.29 Hz, 2H).

### Example 165

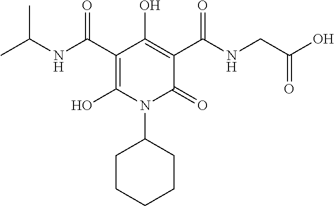

### N-[(1-Cyclohexyl-4,6-dihydroxy-5-{[(1-methylethyl)amino]carbonyl}-2-oxo-[2-dihydro-3-pyridinyl)carbonyl]glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and isopropylamine (0.051 ml, 0.833 mmol) in chloroform (5 ml) was sealed in a pressure flask and heated at 150° C. for 30 minutes. The reaction mixture was evaporated, the residue was dissolved in ethanol (3 mL) treated with 6 molar sodium hydroxide (3 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-[(1-cyclohexyl-4,6-dihydroxy-5-{[(1-methylethyl)amino]carbonyl}-2-oxo-1,2-dihydro-3-pyridinyl)carbonyl]glycine (198 mg, 0.501 mmol, 66.2% yield) ¹H NMR (400 MHz, DMSO-d6) δ ppm 12.99 (br. s., 1H), 9.62 (br. s., 2H), 4.84 (s, 1H), 3.86-4.27 (m, 3H), 2.34 (q, J=10.86 Hz, 2 H), 1.80 (d, J=12.63 Hz, 2H), 1.48-1.70 (m, 3H), 0.99-1.41 (m, 9H).

### Example 166

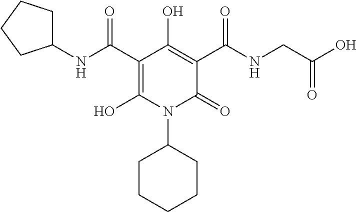

### N-({1-Cyclohexyl-5-[(cyclopentylamino)carbonyl]-4,6-dihydroxy-2-oxo-1,2-dihydro-3-pyridinyl}carbonyl)glycine

A mixture of methyl 1-cyclohexyl-5-({[2-(ethyloxy)-2-oxoethyl]amino}carbonyl)-2,4-dihydroxy-6-oxo-1,6-dihydro-3-pyridinecarboxylate (300 mg, 0.757 mmol) and cyclopentylamine (0.082 ml, 0.833 mmol) in chloroform (5 ml) was sealed in a pressure flask and heated at 150° C. for 30 minutes. The reaction mixture was evaporated, the residue was dissolved in ethanol (3 mL) treated with 6 molar sodium hydroxide (3 ml) and stirred until hydrolysis was complete. The mixture was partitioned between ethyl acetate and 1 molar hydrochloric acid, the aqueous extracted with ethyl acetate and the combined extracts washed with 1 molar hydrochloric acid. The solution was dried and evaporated to give a solid that was recrystallized from acetic acid to give N-({1-cyclohexyl-5-[(cyclopentylamino)carbonyl]-4,6-dihydroxy-2-oxo-1,2-dihydro-3-pyridinyl}carbonyl)glycine (195 mg, 0.463 mmol, 61.1% yield) ¹H NMR (400 MHz, DMSO-d6) δ ppm 12.98 (br. s., 1H), 9.82 (br. s., 2H), 4.84 (s, 1H), 4.21 (dt, J=13.39, 6.82 Hz, 1H), 4.08 (d, J=5.56 Hz, 2H), 2.17-2.46 (m, 2H), 1.87-2.07 (m, 2H), 1.79 (d, J=12.63 Hz, 2H), 1.43-1.74 (m, 9H), 1.30 (q, J=12.88 Hz, 2H), 1.14 (q, J=12.63 Hz, 1H).

### Example 167

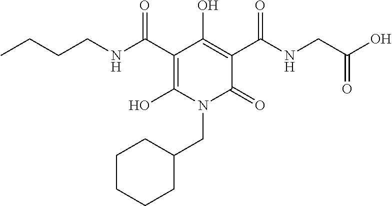

### N-{[5-[(Butylamino)carbonyl]-1-(cyclohexylmethyl)-4,6-dihydroxy-2-oxo-1,2-dihydro-3-pyridinyl]carbonyl}glycine

A mixture of methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (300 mg, 0.731 mmol) and n-butylamine (0.079 ml, 0.804 mmol) in chloroform (5 ml) was sealed in a pressure flask and heated at 150° C. for 30 minutes. The reaction mixture was evaporated, the residue was dissolved in ethanol (3 mL) treated with 6 molar sodium hydroxide (3 ml) and stirred until hydrolysis was complete. The mixture was acidified with 1 molar hydrochloric acid to give a solid that was collected, washed with 1 molar hydrochloric acid and water. The solid was recrystallized from acetic acid to give N-{[5-[(butylamino)carbonyl]-1-(cyclohexylmethyl)-4,6-dihydroxy-2-oxo-1,2-dihydro-3-pyridinyl]carbonyl}glycine (206 mg, 0.486 mmol, 66.6% yield) ¹H NMR (400 MHz, DMSO-d6) δ ppm 12.66 (br. s., 1H), 9.59 (br. s., 2H), 4.08 (d, J=5.56 Hz, 2H), 3.76 (d, J=7.33 Hz, 2H), 3.36 (dt, J=6.82 Hz, 2H), 1.61-1.81 (m, 3H), 1.41-1.62 (m, 5H), 1.32 (dq, J=14.97, 7.39 Hz, 2H), 1.06-1.21 (m, 3H), 0.94-1.05 (m, 2H), 0.90 (t, J=7.33 Hz, 3H).

### Example 168

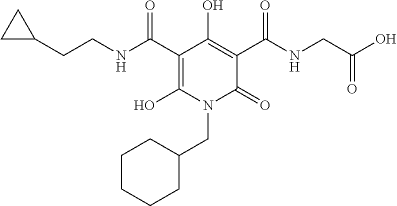

### N-[(1-(cyclohexylmethyl)-5-{[(2-cyclopropylethyl)amino]carbonyl}-4,6-dihydroxy-2-oxo-1,2-dihydro-3-pyridinyl)carbonyl]glycine

A mixture of methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (300 mg, 0.731 mmol), N,N-diisopropylethylamine (0.139 ml, 0.804 mmol) and 2-cyclopropylethylamine hydrochloride (98 mg, 0.804 mmol) in chloroform (5 ml) was sealed in a pressure flask and heated at 150oC. for 30 minutes. The reaction mixture was washed with 1 molar hydrochloric acid (×2), dried and evaporated. The residue was dissolved in ethanol (3 mL) treated with 6 molar sodium hydroxide (3 ml) and stirred until hydrolysis was complete. The mixture was acidified with 1 molar hydrochloric acid to give a solid that was collected, washed with 1 molar hydrochloric acid and water. The solid was recrystallized from acetic acid to give N-[(1-(cyclohexylmethyl)-5-{[(2-cyclopropylethyl)amino]carbonyl}-4,6-dihydroxy-2-oxo-1,2-dihydro-3-pyridinyl)carbonyl]glycine (117 mg, 0.269 mmol, 36.8% yield) ¹H NMR (400 MHz, DMSO-d6) δ ppm 12.94 (br. s., 1H), 4.08 (d, J=5.56 Hz, 2H), 3.76 (d, J=7.33 Hz, 2H), 3.43 (dt, J=6.74 Hz, 2H), 1.62-1.81 (m, 3H), 1.51-1.62 (m, 3H), 1.47 (dt, J=6.99 Hz, 2H), 1.06-1.23 (m, 3H), 0.85-1.04 (m, 2H), 0.58-0.78 (m, 1H), 0.42 (dt, 2H), 0.08 (dt, J=5.18, 4.29 Hz, 2H).

### Example 169

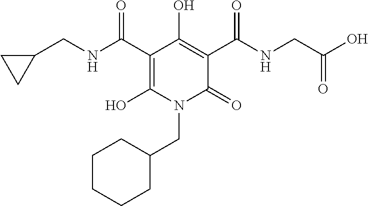

### N-[(1-(Cyclohexylmethyl)-5-{[(cyclopropylmethyl)amino]carbonyl}-4,6-dihydroxy-2-oxo-1,2-dihydro-3-pyridinyl)carbonyl]glycine

A mixture of methyl 1-(cyclohexylmethyl)-5-({[2-(ethyloxy)-2-oxoethyl]amino}carbonyl)-2,4-dihydroxy-6-oxo-1,6-dihydro-3-pyridinecarboxylate (300 mg, 0.731 mmol) and cyclopropylmethylamine (0.070 ml, 0.804 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid to give a solid that was collected, washed with 1 molar hydrochloric acid and water. The solid was recrystallized from acetic acid to give N-[(1-(cyclohexylmethyl)-5-{[(cyclopropylmethyl)amino]carbonyl}-4,6-dihydroxy-2-oxo-1,2-dihydro-3-pyridinyl)carbonyl]glycine (249 mg, 0.591 mmol, 81% yield) ¹H NMR (400 MHz, DMSO-d6) δ ppm 12.84 (br. s., 1H), 9.75 (br. s., 2H), 4.09 (d, J=5.56 Hz, 2H), 3.77 (d, J=7.33 Hz, 2H), 3.23 (dd, J=6.32 Hz, 2H), 1.62-1.82 (m, 3H), 1.56 (d, J=12.88 Hz, 3H), 1.04-1.26 (m, 4H), 0.97 (q, J=11.71 Hz, 2H), 0.41-0.55 (m, 2H), 0.28 (td, J=6.00, 4.48, 4.17 Hz, 2H).

### Example 170

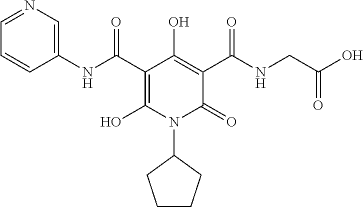

### N-({1-Cyclopentyl-4,6-dihydroxy-2-oxo-5-[(3-pyridinylamino)carbonyl]-1,2-dihydro-3-pyridinyl}carbonyl)glycine

A mixture of methyl 1-cyclopentyl-5-({[2-(ethyloxy)-2-oxoethyl]amino}carbonyl)-2,4-dihydroxy-6-oxo-1,6-dihydro-3-pyridinecarboxylate (300 mg, 0.785 mmol) and 3-aminopyridine (81 mg, 0.863 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid to give a solid that was collected, washed with 1 molar hydrochloric acid and water. The solid was recrystallized from acetic acid to give N-({1-cyclopentyl-4,6-dihydroxy-2-oxo-5-[(3-pyridinylamino)carbonyl]-1,2-dihydro-3-pyridinyl}carbonyl)glycine (240 mg, 0.576 mmol, 73.5% yield) ¹H NMR (400 MHz, DMSO-d6) δ ppm 12.38 (s, 1H), 9.71 (s, 1H), 9.20 (s, 1 H), 8.61 (d, J=5.56 Hz, 1H), 8.55 (d, J=9.60 Hz, 1H), 7.94 (dd, J=8.46, 5.43 Hz, 1H), 5.45 (tt, 1 H), 4.10 (d, J=2.78 Hz, 2H), 2.00-2.19 (m, 2H), 1.85-2.02 (m, 2H), 1.70-1.86 (m, 2H), 1.44-1.68 (m, 2H).

### Example 171

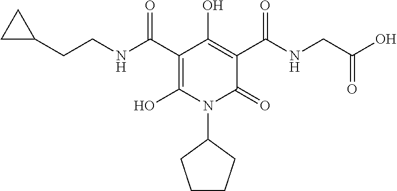

### N-[(1-Cyclopentyl-5-{[(2-cyclopropylethyl)amino]carbonyl}-4,6-dihydroxy-2-oxo-1,2-dihydro-3-pyridinyl)carbonyl]glycine

A mixture of methyl 1-cyclopentyl-5-({[2-(ethyloxy)-2-oxoethyl]amino}carbonyl)-2,4-dihydroxy-6-oxo-1,6-dihydro-3-pyridinecarboxylate (300 mg, 0.785 mmol), diisopropylethylamine (0.149 ml, 0.863 mmol) and 2-cyclopropylethylamine hydrochloride (105 mg, 0.863 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(1-cyclopentyl-5-{[(2-cyclopropylethyl)amino]carbonyl}-4,6-dihydroxy-2-oxo-1,2-dihydro-3-pyridinyl)carbonyl]glycine (277 mg, 0.680 mmol, 87% yield) ¹H NMR (400 MHz, DMSO-d6) δ ppm 12.84 (br. s., 1H), 9.75 (br. s., 2H), 4.09 (d, J=5.56 Hz, 2H), 3.77 (d, J=7.33 Hz, 2H), 3.23 (dd, J=6.32 Hz, 2H), 1.62-1.82 (m, 3H), 1.56 (d, J=12.88 Hz, 3H), 1.04-1.26 (m, 4H), 0.97 (q, J=11.71 Hz, 2H), 0.41-0.55 (m, 2H), 0.28 (td, J=6.00, 4.48, 4.17 Hz, 2H).

### Example 172

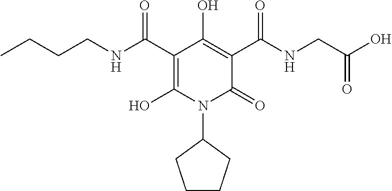

### N-({5-[(Butylamino)carbonyl]-1-cyclopentyl-4,6-dihydroxy-2-oxo-1,2-dihydro-3-pyridinyl}carbonyl)glycine

A mixture of methyl 1-cyclopentyl-5-({[2-(ethyloxy)-2-oxoethyl]amino}carbonyl)-2,4-dihydroxy-6-oxo-1,6-dihydro-3-pyridinecarboxylate (300 mg, 0.785 mmol) and n-butylamine (0.085 ml, 0.863 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-({5-[(butylamino)carbonyl]-1-cyclopentyl-4,6-dihydroxy-2-oxo-1,2-dihydro-3-pyridinyl}carbonyl)glycine (190 mg, 0.481 mmol, 61.2% yield) ¹H NMR (400 MHz, DMSO-d6) δ ppm 12.98 (s, 1H), 9.90 (s, 1H), 9.62 (s, 1H), 5.38 (tt, 1H), 4.09 (d, J=5.56 Hz, 2H), 3.36 (dt, 2H), 1.96-2.18 (m, 2H), 1.83-1.97 (m, 2H), 1.67-1.82 (m, 2H), 1.43-1.65 (m, 4H), 1.32 (ft, J=14.97, 7.39 Hz, 2H), 0.90 (t, J=7.33 Hz, 3H).

### Example 173

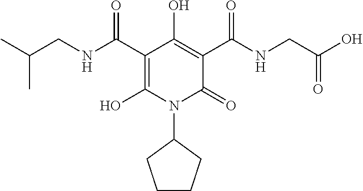

### N-[(1-Cyclopentyl-4,6-dihydroxy-5-{[(2-methylpropyl)amino]carbonyl}-2-oxo-1,2-dihydro-3-pyridinyl)carbonyl]glycine

A mixture of methyl 1-cyclopentyl-5-({[2-(ethyloxy)-2-oxoethyl]amino}carbonyl)-2,4-dihydroxy-6-oxo-1,6-dihydro-3-pyridinecarboxylate (300 mg, 0.785 mmol) and isobutylamine (0.086 ml, 0.863 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(1-cyclopentyl-4,6-dihydroxy-5-{[(2-methylpropyl)amino]carbonyl}-2-oxo-1,2-dihydro-3-pyridinyl)carbonyl]glycine (254 mg, 0.642 mmol, 82% yield) ¹H NMR (400 MHz, DMSO-d6) δ ppm 12.88 (br. s., 1H), 9.72 (br. s., 2H), 5.38 (tt, 1H), 4.09 (d, J=5.56 Hz, 2H), 3.21 (dd, J=6.32 Hz, 2H), 1.97-2.17 (m, 2H), 1.82-1.95 (m, 3H), 1.70-1.81 (m, 2H), 1.45-1.66 (m, 2H), 0.90 (d, J=6.82 Hz, 6H).

### Example 174

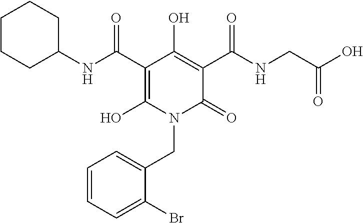

### N-({1-[(2-Bromophenyl)methyl]-5-[(cyclohexylamino)carbonyl]-4,6-dihydroxy-2-oxo-1,2-dihydro-3-pyridinyl}carbonyl)glycine

A mixture of methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.621 mmol) and cyclohexylamine (0.078 ml, 0.683 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-({1-[(2-bromophenyl)methyl]-5-[(cyclohexylamino)carbonyl]-4,6-dihydroxy-2-oxo-1,2-dihydro-3-pyridinyl}carbonyl)glycine (244 mg, 0.467 mmol, 75% yield) ¹H NMR (400 MHz, DMSO-d6) δ ppm 12.48 (br. s., 1H), 9.84 (s, 1 H), 9.58 (s, 1H), 7.66 (dd, J=7.83, 1.26 Hz, 1H), 7.30 (ddd, J=7.45, 1.26 Hz, 1H), 7.21 (ddd, J=7.64, 1.64 Hz, 1H), 6.85 (d, J=6.32 Hz, 1H), 5.09 (s, 2H), 4.10 (d, J=5.56 Hz, 2H), 3.83 (s, 1 H), 1.76-1.89 (m, 2H), 1.66 (d, J=9.35 Hz, 2H), 1.55 (d, J=12.38 Hz, 1H), 1.28-1.46 (m, 4H), 1.12-1.29 (m, 1H).

### Example 175

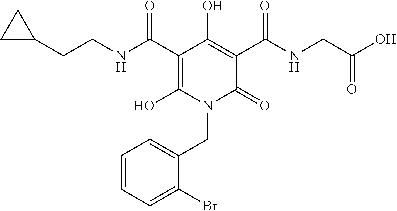

### N-[(1-[(2-bromophenyl)methyl]-5-{[(2-cyclopropylethyl)amino]carbonyl}-4,6-dihydroxy-2-oxo-1,2-dihydro-3-pyridinyl)carbonyl]glycine

A mixture of methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.621 mmol), diisopropylethylamine (0.118 ml, 0.683 mmol) and 2-cyclopropylethylamine hydrochloride (83 mg, 0.683 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(1-[(2-bromophenyl)methyl]-5-{[(2-cyclopropylethyl)amino]carbonyl}-4,6-dihydroxy-2-oxo-1,2-dihydro-3-pyridinyl)carbonyl]glycine (206 mg, 0.405 mmol, 65.3% yield) ¹H NMR (400 MHz, DMSO-d6) δ ppm 12.97 (br. s., 1H), 9.88 (s, 1H), 9.64 (s, 1H), 7.65 (dd, J=7.96, 1.14 Hz, 1H), 7.30 (ddd, J=7.52, 1.14 Hz, 1H), 7.21 (ddd, J=7.64, 1.64 Hz, 1H), 6.84 (dd, J=7.58, 1.26 Hz, 1H), 5.09 (s, 2H), 4.09 (d, J=5.56 Hz, 2H), 3.45 (dt, J=6.74 Hz, 2H), 1.47 (dt, J=7.07 Hz, 2H), 0.56-0.83 (m, 1H), 0.42 (dt, 2H), 0.08 (dt, J=5.18, 4.29 Hz, 2H).

### Example 176

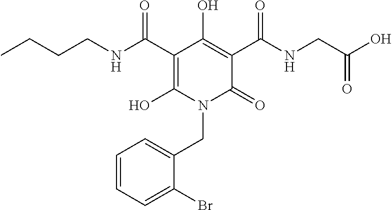

### N-({1-[(2-Bromophenyl)methyl]-5-[(butylamino)carbonyl]-4,6-dihydroxy-2-oxo-1,2-dihydro-3-pyridinyl}carbonyl)glycine

A mixture of methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.621 mmol) and n-butylamine (0.067 ml, 0.683 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-({1-[(2-bromophenyl)methyl]-5-[(butylamino)carbonyl]-4,6-dihydroxy-2-oxo-1,2-dihydro-3-pyridinyl}carbonyl)glycine (215 mg, 0.433 mmol, 69.8% yield) ¹H NMR (400 MHz, DMSO-d6) δ ppm 12.87 (br. s., 1H), 9.88 (s, 1H), 9.61 (s, 1H), 7.65 (dd, J=7.83, 1.26 Hz, 1H), 7.30 (ddd, J=7.52, 1.14 Hz, 1H), 7.21 (ddd, J=7.64, 1.64 Hz, 1H), 6.85 (dd, J=7.83, 1.26 Hz, 1H), 5.10 (s, 2H), 4.09 (d, J=5.56 Hz, 2H), 3.37 (dt, 2H), 1.54 (tt, 2 H), 1.32 (tq, J=7.43 Hz, 2H), 0.90 (t, J=7.45 Hz, 3H).

### Example 177

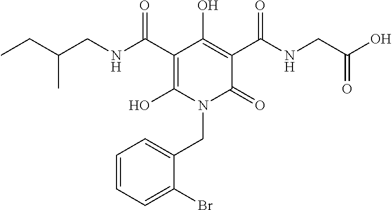

### N-[(1-[(2-Bromophenyl)methyl]-4,6-dihydroxy-5-{[(2-methylpropyl)amino]carbonyl}-2-oxo-1,2-dihydro-3-pyridinyl)carbonyl]glycine

A mixture of methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (300 mg, 0.621 mmol) and isobutylamine (0.068 ml, 0.683 mmol) in chloroform (5 ml) was sealed in a pressure flask and heated at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(1-[(2-bromophenyl)methyl]-4,6-dihydroxy-5-{[(2-methylpropyl)amino]carbonyl}-2-oxo-1,2-dihydro-3-pyridinyl)carbonyl]glycine (215 mg, 0.433 mmol, 69.8% yield) ¹H NMR (400 MHz, DMSO-d6) δ ppm 12.97 (br. s., 1H), 9.87 (s, 1H), 9.66 (s, 1H), 7.65 (dd, J=7.96, 1.14 Hz, 1H), 7.30 (ddd, J=7.58, 1.26 Hz, 1H), 7.21 (ddd, J=7.64, 1.64 Hz, 1H), 6.85 (dd, J=7.58, 1.26 Hz, 1H), 5.09 (s, 2H), 4.10 (d, J=5.56 Hz, 2H), 3.22 (dd, J=6.44 Hz, 2H), 1.72-1.96 (m, 1H), 0.90 (d, J=6.57 Hz, 6H).

### Example 178

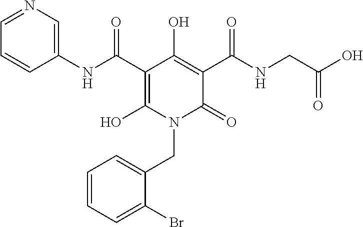

### N-({1-[(2-Bromophenyl)methyl]-4,6-dihydroxy-2-oxo-5-[(3-pyridinylamino)carbonyl]-1,2-dihydro-3-pyridinyl}carbonyl)glycine

A mixture of methyl 1-[(2-bromophenyl)methyl]-5-({[2-(ethyloxy)-2-oxoethyl]amino}carbonyl)-2,4-dihydroxy-6-oxo-1,6-dihydro-3-pyridinecarboxylate (220 mg, 0.455 mmol) and 3-aminopyridine (57 mg, 0.606 mmol) in chloroform (5 ml) was sealed in a pressure flask and heated at 150° C. for 30 minutes in a microwave reactor. The mixture was evaporated, then stirred in ethanol (3 ml) and 6 molar sodium hydroxide solution (3 ml) until hydrolysis was complete. The mixture was acidified with 1 molar hydrochloric acid to give a solid that was collected, washed with 1 molar hydrochloric acid and water. The solid was recrystallized from acetic acid to give N-({1-[(2-bromophenyl)methyl]-4,6-dihydroxy-2-oxo-5-[(3-pyridinylamino)carbonyl]-1,2-dihydro-3-pyridinyl}carbonyl)glycine (150 mg, 0.290 mmol, 63.7% yield) ¹H NMR (400 MHz, DMSO-d6) δ ppm 12.53 (s, 1H), 9.95 (s, 1H), 9.34 (d, J=1.77 Hz, 1 H), 8.44-8.69 (m, 2H), 7.88-8.03 (m, 1H), 7.64 (dd, J=7.96, 1.14 Hz, 1H), 7.29 (ddd, J=7.52, 1.14 Hz, 1H), 7.19 (ddd, J=7.71, 1.52 Hz, 1H), 6.82 (d, J=6.57 Hz, 1H), 5.13 (s, 2H), 4.05 (s, 2 H).

### Example 179

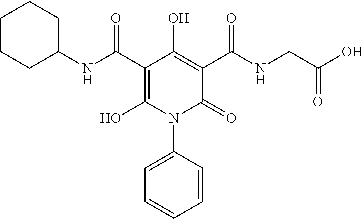

### N-({5-[(Cyclohexylamino)carbonyl]-4,6-dihydroxy-2-oxo-1-phenyl-1,2-dihydro-3-pyridinyl}carbonyl)glycine

179a) 2,2-Dimethyl-7-(phenylamino)-4H,5H-pyrano[4,3-d][1,3]dioxin-4,5-dione. 7-Chloro-2,2-dimethyl-4H,5H-pyrano[4,3-d][1,3]dioxin-4,5-dione (5 g, 21.68 mmol) was stirred in dichloromethane (50 mL) and cooled over an ice bath. N,N-diisopropylethylamine (4.13 ml, 23.85 mmol) in dichloromethane (25 mL) was added dropwise, followed by a solution of aniline (2.173 ml, 23.85 mmol) in dichloromethane (25 mL). Cooling was removed and the mixture was stirred at room temperature for one hour. LCMS indicated that the reaction was complete. The solution was washed with water, dried and evaporated to give 2,2-dimethyl-7-(phenylamino)-4H,5H-pyrano[4,3-d][1,3]dioxin-4,5-dione (5.6 g, 19.49 mmol, 90% yield) ¹H NMR (400 MHz, DMSO-d6) δ ppm 11.01 (s, 1H), 7.49-7.62 (m, 1H), 7.41-7.51 (m, 1H), 7.31-7.41 (m, 2H), 7.17-7.32 (m, 1H), 5.34 (s, 1H), 1.67 (s, 6H).

179b) Methyl 2,4-dihydroxy-6-oxo-1-phenyl-1,6-dihydro-3-pyridinecarboxylate. 2,2-Dimethyl-7-(phenylamino)-4H,5H-pyrano[4,3-d][1,3]dioxin-4,5-dione (5.6 g, 19.49 mmol) was treated with 25% Sodium methoxide in methanol (31.7 ml, 139 mmol) and the mixture was heated under reflux for 5 hours. The mixture was poured onto ice and acidified with 1 molar hydrochloric acid to give a solid that was collected, washed with water and dried to give methyl 2,4-dihydroxy-6-oxo-1-phenyl-1,6-dihydro-3-pyridinecarboxylate (2.3 g, 8.80 mmol, 45.2% yield) ¹H NMR (400 MHz, DMSO-d6) δ ppm 12.98 (s, 1H), 7.42-7.49 (m, 2H), 7.34-7.42 (m, 1H), 7.16 (d, J=7.07 Hz, 2H), 5.43 (s, 1H), 3.73 (s, 3H).

179c) Methyl 5-({[2-(ethyloxy)-2-oxoethyl]amino}carbonyl)-2,4-dihydroxy-6-oxo-1-phenyl-1,6-dihydro-3-pyridinecarboxylate. A mixture of methyl 2,4-dihydroxy-6-oxo-1-phenyl-1,6-dihydro-3-pyridinecarboxylate (2.3 g, 8.80 mmol), diisopropylethylamine (1.676 ml, 9.68 mmol) and ethyl isocyanatoacetate (1.086 ml, 9.68 mmol) in tetrachloroethylene (50 ml) and N,N-Dimethylacetamide (DMA) (20 ml) was heated at 110° C. for 30 minutes. The solvent was evaporated, the residue dissolved in ethyl acetate, washed with 1N hydrochloric acid. The aqueous was extracted with ethyl acetate and the combined organic solutions washed with 1N hydrochloric acid and evaporated to near dryness. MTBE and hexane was added, the resultant solid collected, washed with MTBE and hexane and dried to give methyl 5-({[2-(ethyloxy)-2-oxoethyl]amino}carbonyl)-2,4-dihydroxy-6-oxo-1-phenyl-1,6-dihydro-3-pyridinecarboxylate (1.64 g, 4.20 mmol, 47.7% yield) ¹H NMR (400 MHz, DMSO-d6) δ ppm 11.28 (br. s., 1H), 10.02 (s, 1 H), 7.42-7.51 (m, 2H), 7.34-7.41 (m, J=7.33, 7.33 Hz, 1H), 7.18-7.23 (m, 2H), 4.12 (q, J=7.07 Hz, 2H), 4.05 (d, J=5.56 Hz, 2H), 3.73 (s, 3H), 1.19 (t, J=7.20 Hz, 3H).

179d) N-({5-[(Cyclohexylamino)carbonyl]-4,6-dihydroxy-2-oxo-1-phenyl-1,2-dihydro-3-pyridinyl}carbonyl)glycine. A mixture of methyl 5-({[2-(ethyloxy)-2-oxoethyl]amino}carbonyl)-2,4-dihydroxy-6-oxo-1-phenyl-1,6-dihydro-3-pyridinecarboxylate (300 mg, 0.769 mmol) and cyclohexylamine (0.097 ml, 0.845 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-({5-[(cyclohexylamino)carbonyl]-4,6-dihydroxy-2-oxo-1-phenyl-1,2-dihydro-3-pyridinyl}carbonyl)glycine (160 mg, 0.373 mmol, 48.5% yield) ¹H NMR (400 MHz, DMSO-d6) δ ppm 13.01 (s, 1H), 9.74 (s, 1H), 9.61 (s, 1H), 7.37-7.62 (m, 3H), 7.29 (d, J=7.07 Hz, 2H), 4.10 (d, J=5.56 Hz, 2H), 3.69-4.00 (m, 1H), 1.75-1.99 (m, 2H), 1.59-1.73 (m, J=3.54 Hz, 2H), 1.54 (d, J=12.13 Hz, 1H), 1.28-1.44 (m, 4H), 1.08-1.27 (m, 1H).

### Example 180

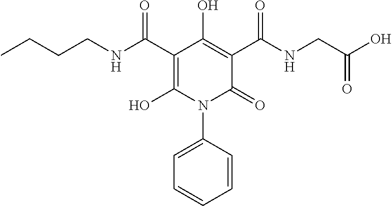

### N-({5-[(Butylamino)carbonyl]-4,6-dihydroxy-2-oxo-1-phenyl-1,2-dihydro-3-pyridinyl}carbonyl)glycine

A mixture of methyl 5-({[2-(ethyloxy)-2-oxoethyl]amino}carbonyl)-2,4-dihydroxy-6-oxo-1-phenyl-1,6-dihydro-3-pyridinecarboxylate (300 mg, 0.769 mmol) and n-butylamine (0.084 ml, 0.845 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, the solid collected and recrystallized from acetic acid to give N-({5-[(butylamino)carbonyl]-4,6-dihydroxy-2-oxo-1-phenyl-1,2-dihydro-3-pyridinyl}carbonyl)glycine (135 mg, 0.335 mmol, 43.5% yield) ¹H NMR (400 MHz, DMSO-d6) δ ppm 12.98 (s, 1H), 9.81 (s, 1H), 9.59 (s, 1H), 7.36-7.67 (m, 3H), 7.18-7.36 (m, 2H), 4.09 (d, J=5.56 Hz, 2H), 3.36 (dt, J=6.74 Hz, 2H), 1.53 (tt, J=7.20 Hz, 2H), 1.31 (tq, J=7.43 Hz, 2H), 0.90 (t, J=7.45 Hz, 3H).

### Example 181

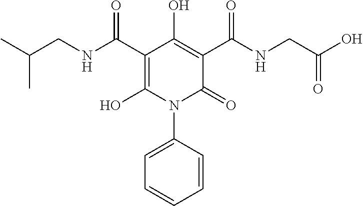

### N-[(4,6-Dihydroxy-5-{[(2-methylpropyl)amino]carbonyl}-2-oxo-1-phenyl-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-phenyl-1,6-dihydro-3-pyridinecarboxylate (300 mg, 0.769 mmol) and isobutylamine (84 uL, 0.845 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(4,6-dihydroxy-5-{[(2-methylpropyl)amino]carbonyl}-2-oxo-1-phenyl-1,2-dihydro-3-pyridinyl)carbonyl]glycine (120 mg, 0.297 mmol, 38.7% yield) ¹H NMR (400 MHz, DMSO-d6) δ ppm 12.96 (s, 1H), 9.79 (s, 1H), 9.64 (s, 1H), 7.37-7.60 (m, 3H), 7.10-7.36 (m, 2H), 4.09 (d, J=5.56 Hz, 2H), 3.21 (dd, J=6.44 Hz, 2H), 1.73-1.96 (m, 1H), 0.90 (d, J=6.82 Hz, 6H).

### Example 182

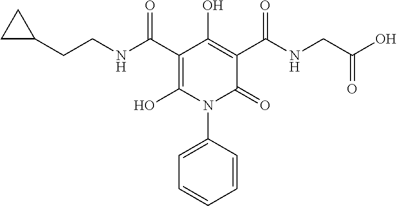

### N-[(5-{[(2-cyclopropylethyl)amino]carbonyl}-4,6-dihydroxy-2-oxo-1-phenyl-1,2-dihydro-3-pyridinyl)carbonyl]glycine

A mixture of methyl 5-({[2-(ethyloxy)-2-oxoethyl]amino}carbonyl)-2,4-dihydroxy-6-oxo-1-phenyl-1,6-dihydro-3-pyridinecarboxylate (300 mg, 0.769 mmol), diisopropylethylamine (0.160 ml, 0.922 mmol) and cyclopropylethylamine hydrochloride (110 mg, 0.845 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, extracted with ethyl acetate, the organic solution washed with 1 molar hydrochloric acid, dried and evaporated to a solid that recrystallized from acetic acid to give N-[(5-{[(2-cyclopropylethyl)amino]carbonyl}-4,6-dihydroxy-2-oxo-1-phenyl-1,2-dihydro-3-pyridinyl)carbonyl]glycine (140 mg, 0.337 mmol, 43.9% yield) ¹H NMR (400 MHz, DMSO-d6) δ ppm 12.96 (s, 1H), 9.81 (s, 1H), 9.62 (s, 1H), 7.35-7.60 (m, 3H), 7.13-7.39 (m, 2H), 4.08 (d, J=5.56 Hz, 2H), 3.43 (dt, J=6.57 Hz, 2H), 1.46 (dt, J=6.99 Hz, 2H), 0.57-0.82 (m, 1H), 0.31-0.53 (m, 2H), −0.05-0.22 (m, 2H).

### Example 183

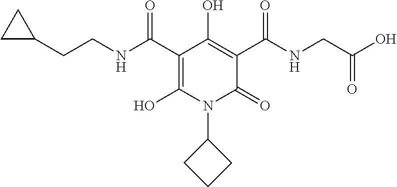

### N-[(1-Cyclobutyl-5-{[(2-cyclopropylethyl)amino]carbonyl}-4,6-dihydroxy-2-oxo-1,2-dihydro-3-pyridinyl)carbonyl]glycine

183a) 7-(Cyclobutylamino)-2,2-dimethyl-4H,5H-pyrano[4,3-d][1,3]dioxin-4,5-dione. 7-Chloro-2,2-dimethyl-4H,5H-pyrano[4,3-d][1,3]dioxin-4,5-dione (3 g, 13.01 mmol) was stirred in dichloromethane (50 mL) and cooled over an ice bath. N,N-diisopropylethylamine (2.476 ml, 14.31 mmol) in dichloromethane (25 mL) was added dropwise, followed by a solution of cyclobutylamine (1.222 ml, 14.31 mmol) in dichloromethane (25 mL). Cooling was removed and the mixture was stirred at room temperature for one hour. LCMS indicated that the reaction was complete. The mixture was washed with water, dried and evaporated to give 7-(cyclobutylamino)-2,2-dimethyl-4H,5H-pyrano[4,3-d][1,3]dioxin-4,5-dione (3.4 g, 12.82 mmol, 99% yield) ¹H NMR (400 MHz, DMSO-d6) δ ppm 9.35 (s, 1H), 5.04-5.29 (m, 1H), 3.83-4.31 (m, 1H), 2.22-2.35 (m, J=5.81 Hz, 2H), 1.90-2.08 (m, 2H), 1.58-1.81 (m, 8H).

183b) Methyl 1-cyclobutyl-2,4-dihydroxy-6-oxo-1,6-dihydro-3-pyridinecarboxylate. 7-(Cyclobutylamino)-2,2-dimethyl-4H,5H-pyrano[4,3-d][1,3]dioxin-4,5-dione (3.4 g, 12.82 mmol) was treated with 25% Sodium methoxide in methanol (21.16 ml, 93 mmol) and the mixture was heated under reflux. The mixture became very thick and methanol (20 ml) was added, reflux continued for 2 hours. The mixture was acidified with 1 molar hydrochloric acid and ice to give a light orange solid that was collected, washed with 1 molar hydrochloric acid and hexane. Dried to give methyl 1-cyclobutyl-2,4-dihydroxy-6-oxo-1,6-dihydro-3-pyridinecarboxylate (2.02 g, 8.44 mmol, 65.9% yield) ¹H NMR (400 MHz, DMSO-d6) δ ppm 12.69 (s, 1H), 5.14-5.47 (m, 2H), 3.75 (s, 3H), 2.81 (ddd, J=19.83, 9.85, 9.73 Hz, 2H), 2.15 (ddd, J=8.34 Hz, 2H), 1.73-1.87 (m, 1 H), 1.59-1.73 (m, 1H).

183c) Methyl 1-cyclobutyl-5-({[2-(ethyloxy)-2-oxoethyl]amino}carbonyl)-2,4-dihydroxy-6-oxo-1,6-dihydro-3-pyridinecarboxylate. A mixture of methyl 1-cyclobutyl-2,4-dihydroxy-6-oxo-1,6-dihydro-3-pyridinecarboxylate (2.02 g, 8.44 mmol), diisopropylethylamine (1.607 ml, 9.29 mmol) and ethyl isocyanatoacetate (1.042 ml, 9.29 mmol) in tetrachloroethylene (30 ml) and N,N-Dimethylacetamide (DMA) (10 ml) was heated at 110° C. for 30 minutes. The solvent was evaporated, the residue dissolved in ethyl acetate, washed with 1N hydrochloric acid. The aqueous was extracted with ethyl acetate and the combined organic solutions washed with 1N hydrochloric acid and evaporated to near dryness. Hexane was added, solid collected, washed with hexane and dried to give methyl 1-cyclobutyl-5-({[2-(ethyloxy)-2-oxoethyl]amino}carbonyl)-2,4-dihydroxy-6-oxo-1,6-dihydro-3-pyridinecarboxylate (830 mg, 2.253 mmol, 26.7% yield) ¹H NMR (400 MHz, DMSO-d6) δ ppm 10.15 (s, 1H), 5.33 (dt, J=17.87, 8.87 Hz, 1H), 4.13 (q, J=7.07 Hz, 2H), 4.08 (d, J=5.56 Hz, 2H), 3.74 (s, 3H), 2.90 (dt, J=21.03, 9.69 Hz, 2H), 2.07-2.21 (m, 2H), 1.74-1.87 (m, 1H), 1.63-1.74 (m, 1H), 1.21 (t, J=7.07 Hz, 3H).

183d) N-[(1-Cyclobutyl-5-{[(2-cyclopropylethyl)amino]carbonyl}-4,6-dihydroxy-2-oxo-1,2-dihydro-3-pyridinyl)carbonyl]glycine. A mixture of methyl 1-cyclobutyl-5-({[2-(ethyloxy)-2-oxoethyl]amino}carbonyl)-2,4-dihydroxy-6-oxo-1,6-dihydro-3-pyridinecarboxylate (200 mg, 0.543 mmol), diisopropylethylamine (0.113 ml, 0.652 mmol) and cyclopropylethylamine hydrochloride (77 mg, 0.597 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, to give a solid that recrystallized from acetic acid to give N-[(1-cyclobutyl-5-{[(2-cyclopropylethyl)amino]carbonyl}-4,6-dihydroxy-2-oxo-1,2-dihydro-3-pyridinyl)carbonyl]glycine (37 mg, 0.094 mmol, 17.32% yield) ¹H NMR (400 MHz, DMSO-d6) δ ppm 12.97 (s, 1H), 9.89 (s, 1H), 9.62 (s, 1H), 5.31 (dt, J=17.94, 8.97 Hz, 1H), 4.09 (d, J=5.56 Hz, 2H), 3.44 (dt, J=6.74 Hz, 2H), 2.75-3.06 (m, J=19.33, 9.66, 9.66, 2.02 Hz, 2H), 2.09-2.34 (m, J=10.99, 8.53, 8.53, 2.27 Hz, 2H), 1.77-1.91 (m, 1H), 1.63-1.78 (m, 1H), 1.33-1.57 (m, J=6.91, 6.91, 6.91 Hz, 2H), 0.59-0.76 (m, 1H), 0.33-0.50 (m, 2H), 0.01-0.14 (m, 2H).

### Example 184

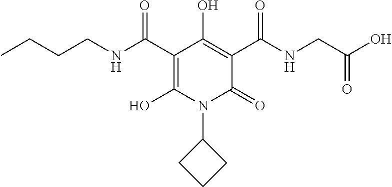

### N-({5-[(Butylamino)carbonyl]-1-cyclobutyl-4,6-dihydroxy-2-oxo-1,2-dihydro-3-pyridinyl}carbonyl)glycine

A mixture of methyl 1-cyclobutyl-5-({[2-(ethyloxy)-2-oxoethyl]amino}carbonyl)-2,4-dihydroxy-6-oxo-1,6-dihydro-3-pyridinecarboxylate (200 mg, 0.543 mmol) and n-butylamine (0.059 ml, 0.597 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid to afford a solid that recrystallized from acetic acid to give N-({5-[(butylamino)carbonyl]-1-cyclobutyl-4,6-dihydroxy-2-oxo-1,2-dihydro-3-pyridinyl}carbonyl)glycine (70 mg, 0.184 mmol, 33.8% yield) ¹H NMR (400 MHz, DMSO-d6) δ ppm 12.97 (s, 1H), 9.87 (s, 1H), 9.59 (s, 1H), 5.31 (dt, J=18.00, 9.06 Hz, 1H), 4.09 (d, J=5.56 Hz, 2H), 3.22-3.60 (m, 2H), 2.73-3.04 (m, J=19.14, 9.63, 9.63, 2.53 Hz, 2H), 2.05-2.29 (m, J=11.24, 11.24, 5.56, 2.53 Hz, 2H), 1.77-1.90 (m, 1H), 1.64-1.77 (m, 1H), 1.54 (dt, J=14.46, 7.29 Hz, 2H), 1.32 (td, J=14.91, 7.33 Hz, 2H), 0.91 (t, J=7.33 Hz, 3H).

### Example 185

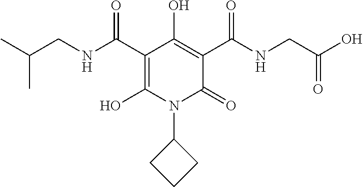

### N-[(1-Cyclobutyl-4,6-dihydroxy-5-{[(2-methylpropyl)amino]carbonyl}-2-oxo-1,2-dihydro-3-pyridinyl)carbonyl]glycine

A mixture of methyl 1-cyclobutyl-5-({[2-(ethyloxy)-2-oxoethyl]amino}carbonyl)-2,4-dihydroxy-6-oxo-1,6-dihydro-3-pyridinecarboxylate (200 mg, 0.543 mmol) and isobutylamine (0.059 ml, 0.597 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, the solid collected and recrystallized from acetic acid to give N-[(1-cyclobutyl-4,6-dihydroxy-5-{[(2-methylpropyl)amino]carbonyl}-2-oxo-1,2-dihydro-3-pyridinyl)carbonyl]glycine (130 mg, 0.341 mmol, 62.8% yield) ¹H NMR (400 MHz, DMSO-d6) δ ppm 12.98 (s, 1H), 9.85 (s, 1H), 9.67 (s, 1H), 5.31 (dt, J=17.94, 8.97 Hz, 1 H), 4.09 (d, J=5.56 Hz, 2H), 3.21 (dd, J=6.44 Hz, 2H), 2.72-3.00 (m, J=19.20, 9.60, 9.60, 2.53 Hz, 2H), 2.08-2.30 (m, 2H), 1.78-1.97 (m, 2H), 1.59-1.79 (m, 1H), 0.91 (d, J=6.57 Hz, 6H).

### Example 186

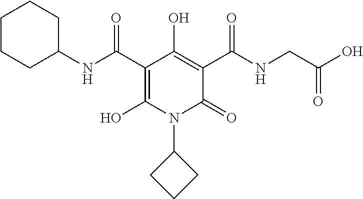

### N-({1-Cyclobutyl-5-[cyclohexylamino)carbonyl]-4,6-dihydroxy-2-oxo-1,2-dihydro-3-pyridinyl}carbonyl)glycine

A mixture of methyl 1-cyclobutyl-5-({[2-(ethyloxy)-2-oxoethyl]amino}carbonyl)-2,4-dihydroxy-6-oxo-1,6-dihydro-3-pyridinecarboxylate (200 mg, 0.543 mmol) and cyclohexylamine (0.068 ml, 0.597 mmol) in chloroform (5 ml) was sealed in a pressure flask and heated in a microwave reactor at 150° C. for 30 minutes. The mixture was cooled and evaporated, then stirred in ethanol (3 ml) and 6 molar sodium hydroxide solution (3 ml) until hydrolysis was complete. The mixture was acidified with 1 molar hydrochloric acid, the solid collected and recrystallized from acetic acid to give N-({1-cyclobutyl-5-[(cyclohexylamino)carbonyl]-4,6-dihydroxy-2-oxo-1,2-dihydro-3-pyridinyl}carbonyl)glycine (170 mg, 0.417 mmol, 77% yield) ¹H NMR (400 MHz, DMSO-d6) δ ppm 12.99 (s, 1H), 9.81 (s, 1H), 9.65 (s, 1H), 5.32 (dt, J=17.87, 9.22, 9.03 Hz, 1 H), 4.09 (d, J=5.81 Hz, 2H), 3.67-3.93 (m, 1H), 2.88 (ddd, J=21.09, 9.85, 9.73 Hz, 2H), 2.06-2.30 (m, 2H), 1.77-1.91 (m, 3H), 1.60-1.77 (m, 3H), 1.56 (d, J=11.87 Hz, 1H), 1.30-1.47 (m, 4H), 1.16-1.30 (m, 1H).

### Examples 187-233

The title compounds were prepared according to the procedures outlined for the synthesis of compounds 1-92 and 140-186 and by substituting the appropriate reagents when required. All products were subjected to purification and provided satisfactory analytical data.

| Example | | 1H NMR (400 MHz, DMSO- | |
|---|---|---|---|
| # | Name | d6)* δ ppm | [M + H]⁺ |
| 187 | 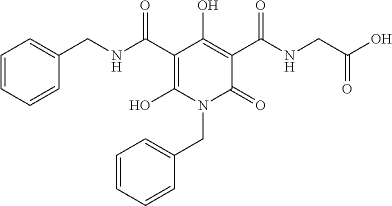 | 4.09 (d, J = 5.56 Hz, 2 H) 4.58 (d, J = 6.06 Hz, 2 H) 5.11 (s, 2 H) 7.19-7.40 (m, 10 H) 9.84 (br. s., 1 H) 10.00 (br. s., 1 H) 13.00 (br. s., 1 H) | 452.1 |
| | N-[(4,6-dihydroxy-2-oxo-1-(phenylmethyl)-5- | | |
| | {[(phenylmethyl)amino]carbonyl}-1,2-dihydro-3- | | |
| | pyridinyl)carbonyl]glycine | | |
| 188 | 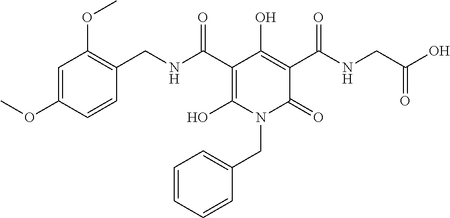 | 3.75 (s, 3 H) 3.83 (s, 3 H) 4.09 (d, J = 5.56 Hz, 2 H) 4.45 (d, J = 5.81 Hz, 2 H) 5.10 (s, 2 H) 6.50 (dd, J = 8.34, 2.27 Hz, 1 H) 6.60 (d, J = 2.53 Hz, 1 H) 7.16 (d, J = 8.34 Hz, 1 H) 7.20- 7.37 (m, 5 H) 9.86 (br. s., 2 H) 12.99 (br. s., 1 H) | 511.8 |
| | N-{[5-[({[2,4- | | |
| | bis(methyloxy)phenyl]methyl}amino)carbonyl]- | | |
| | 4,6-dihydroxy-2-oxo-1-(phenylmethyl)-1,2- | | |
| | dihydro-3-pyridinyl]carbonyl}glycine | | |
| 189 | 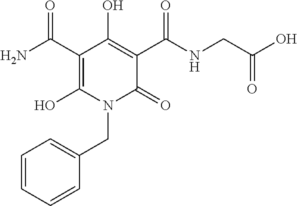 | 4.07 (d, 2 H) 5.10 (s, 2 H) 7.17-7.37 (m, 5 H) 8.94 (br. s., 1 H) 9.13 (br. s., 1 H) 9.99 (br. s., 1 H) 12.93 (br. s., 1 H) | 361.9 |
| | N-{[5-(aminocarbonyl)-4,6-dihydroxy-2-oxo-1- | | |
| | (phenylmethyl)-1,2-dihydro-3- | | |
| | pyridinyl]carbonyl}glycine | | |
| 190 | 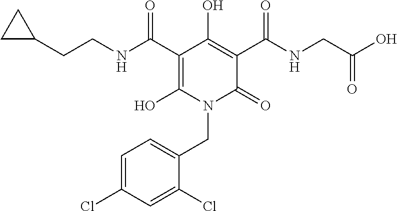 | 13.00 (br. s., 1 H), 9.88 (s, 1 H), 9.65 (s, 1 H), 7.66 (d, J = 2.27 Hz, 1 H), 7.33 (dd, J = 8.46, 2.15 Hz, 1 H), 6.95 (d, J = 8.59 Hz, 1 H), 5.11 (s, 2 H), 4.09 (d, J = 5.56 Hz, 2 H), 3.44 (dt, J = 6.65 Hz, 2 H), 1.47 (dt, J = 6.91 Hz, 2 H), 0.55-0.83 (m, 1 H), 0.27- 0.52 (m, 2 H), −0.05-0.22 (m, 2 H) | 498.1 |
| | N-({5-{[(2-cyclopropylethyl)amino]carbonyl}-1- | | |
| | [(2,4-dichlorophenyl)methyl]-4,6-dihydroxy-2- | | |
| | oxo-1,2-dihydro-3-pyridinyl}carbonyl)glycine | | |
| 191 | 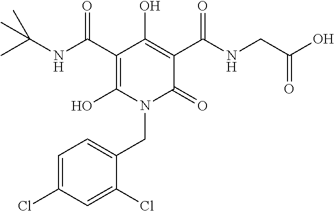 | 13.06 (br. s., 1 H), 9.83 (s, 1 H), 9.72 (s, 1 H), 7.67 (d, J = 2.27 Hz, 1 H), 7.34 (dd, J = 8.34, 2.27 Hz, 1 H), 6.95 (d, J = 8.59 Hz, 1 H), 5.10 (s, 2 H), 4.11 (d, J = 5.56 Hz, 2 H), 1.41 (s, 9 H) | 485.9 |
| | N-[(1-[(2,4-dichlorophenyl)methyl]-5-{[(1,1- | | |
| | dimethylethyl)amino]carbonyl}-4,6-dihydroxy-2- | | |
| | oxo-1,2-dihydro-3-pyridinyl)carbonyl]glycine | | |
| 192 | 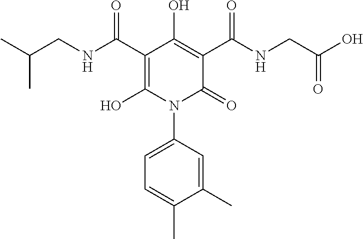 | 12.99 (s, 1 H), 9.77 (s, 1 H), 9.64 (s, 1 H), 7.23 (d, J = 8.08 Hz, 1 H), 7.04 (s, 1 H), 6.98 (dd, J = 7.96, 1.64 Hz, 1 H), 4.09 (d, J = 5.31 Hz, 2 H), 3.20 (dd, J = 6.32 Hz, 2 H), 2.27 (s, 3 H), 2.24 (s, 3 H), 1.85 (dt, J = 20.08, 13.39, 6.69 Hz, 1 H), 0.89 (d, J = 6.57 Hz, 6 H) | 431.9 |
| | N-[(1-(3,4-dimethylphenyl)-4,6-dihydroxy-5- | | |
| | {[(2-methylpropyl)amino]carbonyl}-2-oxo-1,2- | | |
| | dihydro-3-pyridinyl)carbonyl]glycine | | |
| 193 | 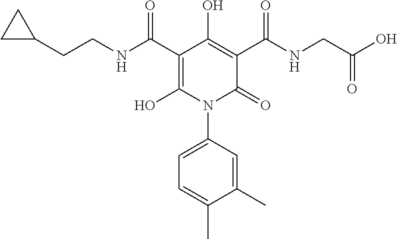 | 12.97 (s, 1 H), 9.80 (s, 1 H), 9.60 (s, 1 H), 7.23 (d, J = 8.08 Hz, 1 H), 7.03 (s, 1 H), 6.98 (d, J = 7.83 Hz, 1 H), 4.08 (d, J = 5.31 Hz, 2 H), 3.43 (dt, J = 6.40 Hz, 2 H), 2.27 (s, 3 H), 2.24 (s, 3 H), 1.45 (dt, J = 7.07 Hz, 2 H), 0.61-0.76 (m, 1 H), 0.33-0.46 (m, 2 H), −0.00-0.15 (m, J = 4.80, 4.80, 4.80 Hz, 2 H) | 444.1 |
| | N-{[5-{[(2-cyclopropylethyl)amino]carbonyl}-1- | | |
| | (3,4-dimethylphenyl)-4,6-dihydroxy-2-oxo-1,2- | | |
| | dihydro-3-pyridinyl]carbonyl}glycine | | |
| 194 | 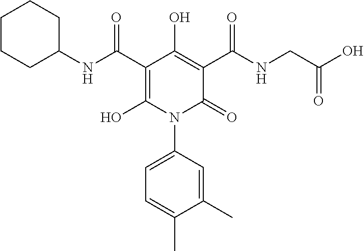 | 12.94 (s, 1 H), 8.85-10.25 (m, 2 H), 7.23 (d, J = 7.83 Hz, 1 H), 7.04 (s, 1 H), 6.98 (dd, J = 7.96, 1.64 Hz, 1 H), 4.09 (d, J = 5.56 Hz, 2 H), 3.66- 3.90 (m, 1 H), 2.27 (s, 3 H), 2.24 (s, 3 H), 1.77-1.89 (m, 2 H), 1.59-1.74 (m, 2 H), 1.54 (d, J = 12.13 Hz, 1 H), 1.28-1.44 (m, 4 H), 1.07- 1.27 (m, J = 10.86 Hz, 1 H) | 458.0 |
| | N-{[5-[(cyclohexylamino)carbonyl]-1-(3,4- | | |
| | dimethylphenyl)-4,6-dihydroxy-2-oxo-1,2- | | |
| | dihydro-3-pyridinyl]carbonyl}glycine | | |
| 195 | 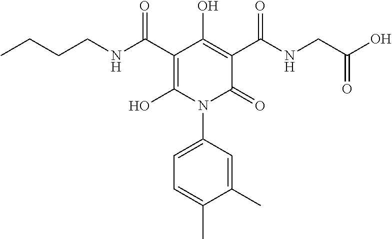 | 12.96 (s, 1 H), 9.78 (s, 1 H), 9.57 (s, 1 H), 7.23 (d, J = 8.08 Hz, 1 H), 7.03 (s, 1 H), 6.98 (dd, J = 7.83, 1.77 Hz, 1 H), 4.08 (d, J = 5.56 Hz, 2 H), 3.35 (dt, J = 6.82 Hz, 2 H), 2.27 (s, 3 H), 2.24 (s, 3 H), 1.52 (tt, J = 14.59, 7.23 Hz, 2 H), 1.31 (tq, J = 7.42, 7.20 Hz, 2 H), 0.89 (t, J = 7.33 Hz, 3 H) | 431.9 |
| | N-{[5-[(butylamino)carbonyl]-1-(3,4- | | |
| | dimethylphenyl)-4,6-dihydroxy-2-oxo-1,2- | | |
| | dihydro-3-pyridinyl]carbonyl}glycine | | |
| 196 | 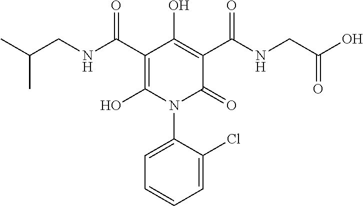 | 13.04 (s, 1 H), 9.82 (s, 1 H), 9.65 (s, 1 H), 7.57-7.85 (m, 1 H), 7.35-7.57 (m, 3 H), 4.10 (d, J = 5.56 Hz, 2 H), 3.22 (dd, J = 6.32 Hz, 2 H), 1.57- 2.09 (m, J = 20.15, 13.45, 6.82 Hz, 1 H), 0.90 (d, J = 6.82 Hz, 6 H) | 438.1 |
| | N-[(1-(2-chlorophenyl)-4,6-dihydroxy-5-{[(2- | | |
| | methylpropyl)amino]carbonyl}-2-oxo-1,2- | | |
| | dihydro-3-pyridinyl)carbonyl]glycine | | |
| 197 | 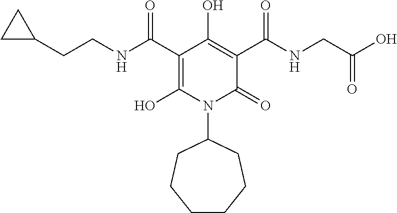 | 12.97 (s, 1 H), 8.32-10.63 (m, 2 H), 5.02 (s, 1 H), 4.08 (d, J = 5.81 Hz, 2 H), 3.43 (dt, J = 6.74 Hz, 2 H), 2.14-2.39 (m, J = 10.19, 10.19, 10.19 Hz, 2 H), 1.62-1.80 (m, 4 H), 1.35-1.62 (m, 8, H) 0.62- 0.79 (m, 1 H), 0.36-0.48 (m, 2 H), 0.01-0.14 (m, 2 H) | 436.1 |
| | N-[(1-cycloheptyl-5-{[(2- | | |
| | cyclopropylethyl)amino]carbonyl}-4,6- | | |
| | dihydroxy-2-oxo-1,2-dihydro-3- | | |
| | pyridinyl)carbonyl]glycine | | |
| 198 | 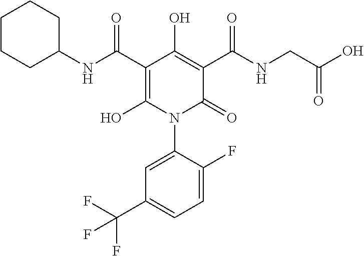 | 13.04 (br. s., 1 H), 9.85 (s, 1 H), 9.57 (s, 1 H), 8.05 (d, J = 6.57 Hz, 1 H), 7.84-7.99 (m, 1 H), 7.68 (dd, J = 8.97 Hz, 1 H), 4.11 (d, J = 5.05 Hz, 2 H), 3.75-3.95 (m, 1 H), 1.74- 1.92 (m, 2 H), 1.60-1.74 (m, 2 H), 1.55 (d, J = 12.38 Hz, 1 H), 1.27-1.46 (m, 4 H), 1.05-1.28 (m, J = 9.35 Hz, 1 H) | 516.2 |
| | N-({5-[(cyclohexylamino)carbonyl]-1-[2-fluoro- | | |
| | 5-(trifluoromethyl)phenyl]-4,6-dihydroxy-2-oxo- | | |
| | 1,2-dihydro-3-pyridinyl}carbonyl)glycine | | |
| 199 | 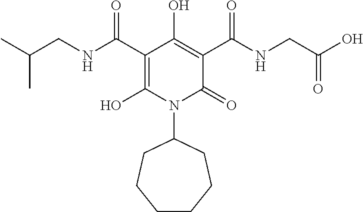 | 12.97 (s, 1 H), 9.79 (br. s., 2 H), 5.02 (s, 1 H), 4.08 (d, J = 5.81 Hz, 2 H), 3.21 (dd, J = 6.44 Hz, 2 H), 2.15-2.44 (m, J = 9.68, 9.68, 9.68 Hz, 2 H), 1.79-1.94 (m, J = 13.52, 13.52, 6.82 Hz, 1 H), 1.62- 1.79 (m, 4 H), 1.36-1.62 (m, 6 H), 0.91 (d, J = 6.57 Hz, 6 H) | 424.0 |
| | N-[(1-cycloheptyl-4,6-dihydroxy-5-{[(2- | | |
| | methylpropyl)amino]carbonyl}-2-oxo-1,2- | | |
| | dihydro-3-pyridinyl)carbonyl]glycine | | |
| 200 | 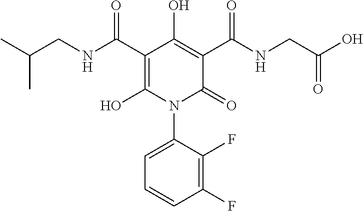 | 13.04 (s, 1 H), 9.85 (s, 1 H), 9.68 (s, 1 H), 7.50-7.78 (m, 1 H), 7.13-7.42 (m, 2 H), 4.10 (d, J = 5.31 Hz, 2 H), 3.22 (dd, J = 6.44 Hz, 2 H), 1.87 (dq, J = 13.39, 6.69 Hz, 1 H), 0.90 (d, J = 6.57 Hz, 6 H) | 440.0 |
| | N-[(1-(2,3-difluorophenyl)-4,6-dihydroxy-5-{[(2- | | |
| | methylpropyl)amino]carbonyl}-2-oxo-1,2- | | |
| | dihydro-3-pyridinyl)carbonyl]glycine | | |
| 201 | 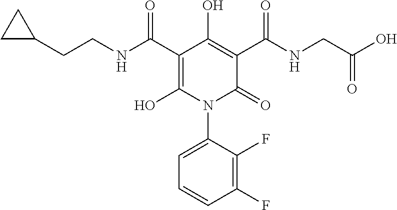 | 13.05 (s, 1 H), 9.86 (s, 1 H), 9.67 (s, 1 H), 7.59 (ddd, J = 17.56, 7.83, 2.15 Hz, 1 H), 7.14-7.44 (m, 2 H), 4.10 (d, J = 5.05 Hz, 2 H), 3.45 (dt, J = 6.57 Hz, 2 H), 1.42 (dt, 2 H), 0.56-0.93 (m, 1 H), 0.28- 0.53 (m, 2 H), −0.14-0.21 (m, 2 H) | 451.9 |
| | N-{[5-{[(2-cyclopropylethyl)amino]carbonyl}-1- | | |
| | (2,3-difluorophenyl)-4,6-dihydroxy-2-oxo-1,2- | | |
| | dihydro-3-pyridinyl]carbonyl}glycine | | |
| 202 | 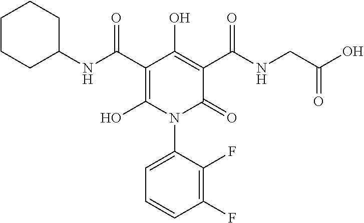 | 13.03 (s, 1 H), 9.85 (s, 1 H), 9.59 (s, 1 H), 7.45-7.78 (m, 1 H), 7.10-7.46 (m, 2 H), 4.11 (d, J = 5.31 Hz, 2 H), 3.64- 3.96 (m, 1 H), 1.78-1.89 (m, 2 H), 1.60-1.73 (m, 2 H), 1.55 (d, J = 12.38 Hz, 1 H), 1.28-1.47 (m, 4 H), 1.12- 1.28 (m, 1 H) | 466.0 |
| | N-{[5-[(cyclohexylamino)carbonyl]-1-(2,3- | | |
| | difluorophenyl)-4,6-dihydroxy-2-oxo-1,2- | | |
| | dihydro-3-pyridinyl]carbonyl}glycine | | |
| 203 | 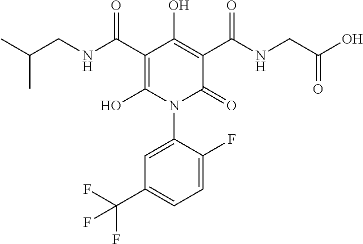 | 13.04 (s, 1 H), 9.85 (s, 1 H), 9.67 (s, 1 H), 8.05 (dd, J = 6.57, 2.02 Hz, 1 H), 7.82- 8.01 (m, 1 H), 7.68 (dd, J = 9.09 Hz, 1 H), 4.10 (d, J = 5.31 Hz, 2 H), 3.23 (dd, J = 6.44 Hz, 2 H), 1.66-2.10 (m, J = 13.42, 13.42, 6.79, 6.79 Hz, 1 H), 0.90 (d, J = 6.57 Hz, 6 H) | 490.1 |
| | N-[(1-[2-fluoro-5-(trifluoromethyl)phenyl]-4,6- | | |
| | dihydroxy-5-{[(2-methylpropyl)amino]carbonyl}- | | |
| | 2-oxo-1,2-dihydro-3-pyridinyl)carbonyl]glycine | | |
| 204 | 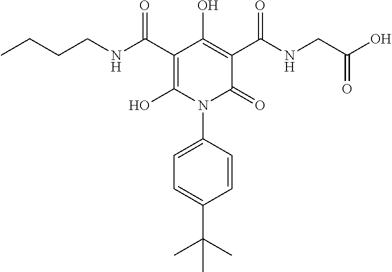 | 13.00 (s, 1 H) 9.80 (s, 1 H) 9.59 (s, 1 H) 7.49 (ddd, J = 8.84, 2.53, 2.27 Hz, 2 H) 7.19 (d, J = 8.59 Hz, 2 H) 4.09 (d, J = 5.56 Hz, 2 H) 1.46- 1.58 (m, 2 H) 1.33 (s, 9 H) 1.24-1.39 (m, 4 H) 0.89 (t, J = 7.33 Hz, 3 H) | 460.2 |
| | N-({5-[(butylamino)carbonyl]-1-[4-(1,1- | | |
| | dimethylethyl)phenyl]-4,6-dihydroxy-2-oxo-1,2- | | |
| | dihydro-3-pyridinyl}carbonyl)glycine | | |
| 205 | 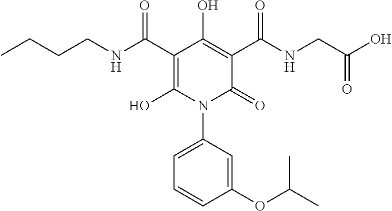 | 12.99 (s, 1 H) 9.80 (s, 1 H) 9.58 (s, 1 H) 7.35 (t, J = 8.08 Hz, 1 H) 6.93-7.01 (m, 1 H) 6.86-6.91 (m, 1 H) 6.81 (d, J = 7.83 Hz, 1 H) 4.60 (qq, J = 5.98 Hz, 1 H) 4.08 (d, J = 5.56 Hz, 2 H) 1.44-1.58 (m, 2 H) 1.27 (d, J = 6.06 Hz, 6 H) 1.23-1.38 (m, 4 H) 0.90 (t, J = 7.33 Hz, 3 H) | 462.1 |
| | N-[(5-[(butylamino)carbonyl]-4,6-dihydroxy-1- | | |
| | {3-[(1-methylethyl)oxy]phenyl}-2-oxo-1,2- | | |
| | dihydro-3-pyridinyl)carbonyl]glycine | | |
| 206 | 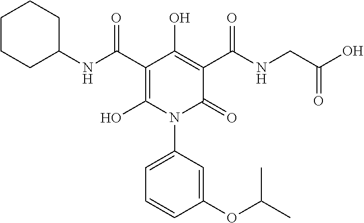 | 13.01 (s, 1 H) 9.73 (s, 1 H) 9.60 (s, 1 H) 7.35 (t, J = 8.08 Hz, 1 H) 6.97 (dd, J = 8.08, 2.02 Hz, 1 H) 6.89 (s, 1 H) 6.81 (d, J = 7.58 Hz, 1 H) 4.60 (qq, J = 6.02 Hz, 1 H) 4.09 (d, J = 5.56 Hz, 2 H) 3.82 (br. s., 1 H) 1.29-1.94 (m, 10 H) 1.27 (d, J = 6.06 Hz, 6 H) | 488.2 |
| | N-[(5-[(cyclohexylamino)carbonyl]-4,6- | | |
| | dihydroxy-1-{3-[(1-methylethyl)oxy]phenyl}-2- | | |
| | oxo-1,2-dihydro-3-pyridinyl)carbonyl]glycine | | |
| 207 | 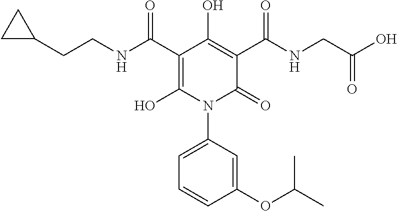 | 12.98 (br. s., 1 H) 9.81 (br. s., 1 H) 9.61 (br. s., 1 H) 7.35 (t, J = 8.08 Hz, 1 H) 6.96 (dt, J = 8.34, 1.26 Hz, 1 H) 6.88 (s, 1 H) 6.80 (d, J = 8.08 Hz, 1 H) 4.51-4.66 (m, 1 H) 4.08 (d, J = 5.56 Hz, 2 H) 3.43 (q, J = 6.65 Hz, 2 H) 1.46 (q, J = 6.91 Hz, 2 H) 1.27 (d, J = 6.06 Hz, 6 H) 0.61-0.76 (m, 1 H) 0.38-0.46 (m, 2 H) 0.03-0.11 (m, 2 H) | 474.2 |
| | N-[(5-{[(2-cyclopropylethyl)amino]carbonyl}- | | |
| | 4,6-dihydroxy-1-{3-[(1-methylethyl)oxy]phenyl}- | | |
| | 2-oxo-1,2-dihydro-3-pyridinyl)carbonyl]glycine | | |
| 208 | 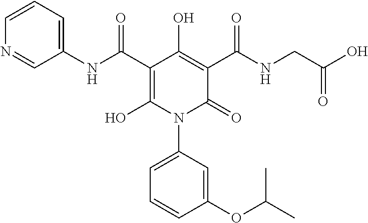 | 12.58 (br. s., 1 H) 10.03 (br. s., 1 H) 9.23 (br. s., 1 H) 8.40- 8.46 (m, 1 H) 8.37 (d, J = 8.08 Hz, 1 H) 7.68-7.82 (m, 1 H) 7.32 (t, J = 8.08 Hz, 1 H) 6.85-6.96 (m, 1 H) 6.65- 6.80 (m, 2 H) 4.60 (spt, J = 5.98 Hz, 1 H) 4.01 (d, J = 5.05 Hz, 2 H) 1.27 (d, J = 6.06 Hz, 6 H) | 482.8 |
| | N-({4,6-dihydroxy-1-{3-[(1- | | |
| | methylethyl)oxy]phenyl}-2-oxo-5-[(3- | | |
| | pyridinylamino)carbonyl]-1,2-dihydro-3- | | |
| | pyridinyl}carbonyl)glycine | | |
| 209 | 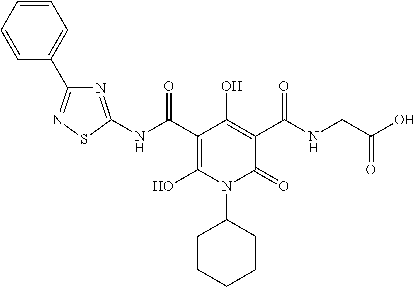 | CHLOROFORM-d + D6- DMSO-1 drop) 16.16 (s, 1 H), 13.40 (s, 1 H), 8.69-9.06 (m, 1 H), 8.01-8.36 (m, 2 H), 7.35-7.51 (m, 3 H), 4.96 (br. s., 1 H), 4.03-4.27 (m, J = 5.05 Hz, 2 H), 2.40 (br. s., 2 H), 1.84 (d, J = 13.14 Hz, 2 H), 1.54-1.75 (m, 3 H), 1.35 (q, J = 12.72 Hz, 2 H), 1.09- 1.28 (m, 1 H) | 514.2 |
| | N-[(1-cyclohexyl-4,6-dihydroxy-2-oxo-5-{[(3- | | |
| | phenyl-1,2,4-thiadiazol-5-yl)amino]carbonyl}- | | |
| | 1,2-dihydro-3-pyridinyl)carbonyl]glycine | | |
| 210 | 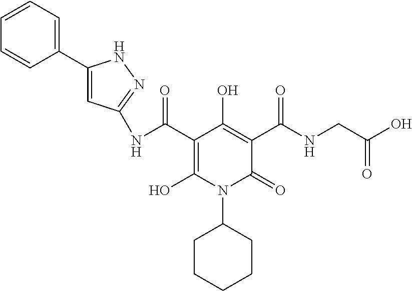 | 13.08 (br. s., 2 H), 12.15 (br. s., 1 H), 9.64 (s, 1 H), 7.78 (d, J = 7.07 Hz, 2 H), 7.47 (dd, J = 7.58 Hz, 2 H), 7.38 (dd, J = 7.33 Hz, 1 H), 6.94 (s, 1 H), 4.72-5.11 (m, 1 H), 4.12 (d, J = 4.80 Hz, 2 H), 2.35- 2.44 (m, J = 11.12 Hz, 2 H), 1.83 (d, J = 11.62 Hz, 2 H), 1.55-1.71 (m, 3 H), 1.33 (q, J = 12.72 Hz, 2 H), 1.18 (q, J = 13.39 Hz, 1 H) | 496.2 |
| | N-[(1-cyclohexyl-4,6-dihydroxy-2-oxo-5-{[(5- | | |
| | phenyl-1H-pyrazol-3-yl)amino]carbonyl}-1,2- | | |
| | dihydro-3-pyridinyl)carbonyl]glycine | | |
| 211 | 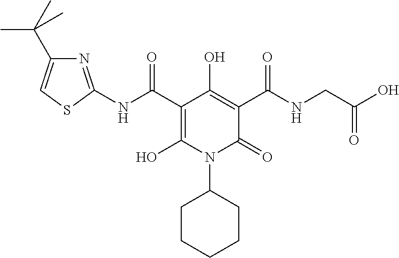 | 14.54 (br. s., 1 H), 10.09 (s, 1 H), 6.89 (s, 1 H), 4.92 (t, J = 12.00 Hz, 1 H), 4.03 (d, J = 4.80 Hz, 2 H), 2.36-2.49 (m, 2 H), 1.81 (d, J = 12.38 Hz, 2 H), 1.65 (d, J = 11.87 Hz, 1 H), 1.55 (d, J = 10.61 Hz, 2 H), 1.23-1.40 (m, 11 H), 1.01- 1.23 (m, 1 H) | 494.0 |
| | N-{[1-cyclohexyl-5-({[4-(1,1-dimethylethyl)-1,3- | | |
| | thiazol-2-yl]amino}carbonyl)-4,6-dihydroxy-2- | | |
| | oxo-1,2-dihydro-3-pyridinyl]carbonyl}glycine | | |
| 212 | 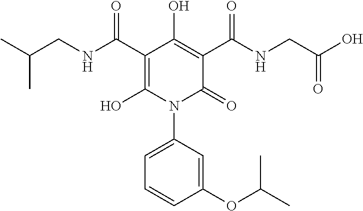 | 13.00 (br. s., 1 H) 9.78 (br. s., 1 H) 9.64 (br. s., 1 H) 7.35 (t, J = 8.08 Hz, 1 H) 6.94-7.00 (m, 1 H) 6.89 (s, 1 H) 6.81 (d, J = 7.83 Hz, 1 H) 4.60 (spt, J = 6.02 Hz, 1 H) 4.09 (d, J = 5.56 Hz, 2 H) 3.21 (t, J = 6.32 Hz, 2 H) 1.77-1.93 (m, 1 H) 1.27 (d, J = 5.81 Hz, 6 H) 0.90 (d, J = 6.57 Hz, 6 H). | 462.1 |
| | N-[(4,6-dihydroxy-1-{3-[(1- | | |
| | methylethyl)oxy]phenyl}-5-{[(2- | | |
| | methylpropyl)amino]carbonyl}-2-oxo-1,2- | | |
| | dihydro-3-pyridinyl)carbonyl]glycine | | |
| 213 | 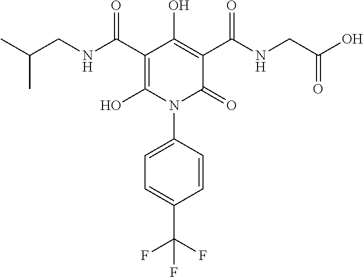 | 13.01 (br. s., 1 H) 9.80 (br. s., 1 H) 9.65 (br. s., 1 H) 7.87 (d, J = 8.34 Hz, 2 H) 7.58 (d, J = 8.08 Hz, 2 H) 4.09 (d, J = 5.31 Hz, 2 H) 3.21 (t, J = 6.44 Hz, 2 H) 1.76-1.94 (m, 1 H) 0.89 (d, J = 6.57 Hz, 6 H) | 472.0 |
| | N-({4,6-dihydroxy-5-{[(2- | | |
| | methylpropyl)amino]carbonyl}-2-oxo-1-[4- | | |
| | (trifluoromethyl)phenyl]-1,2-dihydro-3- | | |
| | pyridinyl}carbonyl)glycine | | |
| 214 | 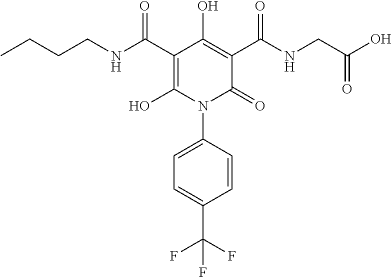 | (MeOD) 7.82 (d, J = 8.34 Hz, 2 H) 7.48 (d, J = 8.08 Hz, 2 H) 4.18 (s, 2 H) 3.46 (t, J = 7.07 Hz, 2 H) 1.63 (tt, J = 7.33 Hz, 2H) 1.43 (dq, J = 14.97, 7.39 Hz, 1 H) 0.98 (t, J = 7.33 Hz, 3 H) | 471.9 |
| | N-({5-[(butylamino)carbonyl]-4,6-dihydroxy-2- | | |
| | oxo-1-[4-(trifluoromethyl)phenyl]-1,2-dihydro-3- | | |
| | pyridinyl}carbonyl)glycine | | |
| 215 | 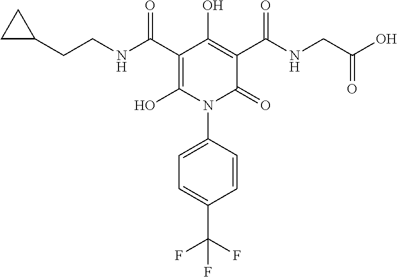 | 13.00 (br. s., 1 H) 9.82 (br. s., 1 H) 9.63 (br. s., 1 H) 7.88 (d, J = 8.34 Hz, 2 H) 7.58 (d, J = 7.83 Hz, 2 H) 4.09 (d, J = 5.05 Hz, 2 H) 3.39-3.50 (m, 2 H) 1.46 (q, J = 6.74 Hz, 2 H) 0.60-0.76 (m, 1 H) 0.33- 0.48 (m, 2 H) −0.01-0.13 (m, 2 H) | 483.8 |
| | N-({5-{[(2-cyclopropylethyl)amino]carbonyl}- | | |
| | 4,6-dihydroxy-2-oxo-1-[4- | | |
| | (trifluoromethyl)phenyl]-1,2-dihydro-3- | | |
| | pyridinyl}carbonyl)glycine | | |
| 216 | 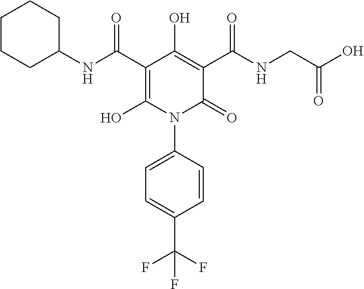 | 13.02 (br. s., 1 H) 9.77 (br. s., 1 H) 9.60 (br. s., 1 H) 7.87 (d, J = 8.59 Hz, 2 H) 7.58 (d, J = 8.34 Hz, 2 H) 4.10 (d, J = 5.31 Hz, 2 H) 3.73-3.91 (m, 1 H) 1.77-1.89 (m, 2 H) 1.47-1.71 (m 3 H) 1.27- 1.43 (m, 4 H) 1.11-1.28 (m, 1 H) | 498.0 |
| | N-({5-[(cyclohexylamino)carbonyl]-4,6- | | |
| | dihydroxy-2-oxo-1-[4-(trifluoromethyl)phenyl]- | | |
| | 1,2-dihydro-3-pyridinyl}carbonyl)glycine | | |
| 217 | 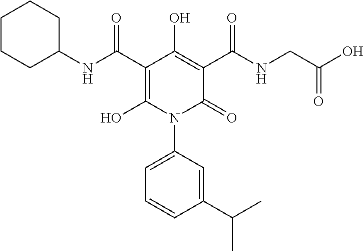 | 12.99 (br. s., 1 H) 9.75 (br. s., 1 H) 9.60 (br. s., 1 H) 7.36- 7.42 (m, 1 H) 7.27-7.35 (m, 1 H) 7.16 (s, 1 H) 7.09 (d, J = 7.83 Hz, 1 H) 4.09 (d, J = 5.56 Hz, 2 H) 3.74-3.90 (m, 1 H) 2.93 (qq, J = 6.95 Hz, 1 H) 1.76-1.94 (m, 2 H) 1.60- 1.72 (m, 2 H) 1.48-1.60 (m, 1 H) 1.24-1.44 (m, 5 H) 1.22 (d, J = 6.82 Hz, 6 H) | 472.0 |
| | N-({5-[(cyclohexylamino)carbonyl]-4,6- | | |
| | dihydroxy-1-[3-(1-methylethyl)phenyl]-2-oxo- | | |
| | 1,2-dihydro-3-pyridinyl}carbonyl)glycine | | |
| 218 | 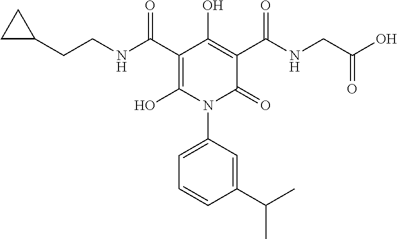 | 12.98 (br. s., 1 H) 9.82 (br. s., 1 H) 9.61 (br. s., 1 H) 7.39 (t, J = 7.71 Hz, 1 H) 7.26-7.35 (m, 1 H) 7.15 (s, 1 H) 7.08 (d, J = 7.83 Hz, 1 H) 4.08 (d, J = 5.56 Hz, 2 H) 3.43 (q, J = 6.57 Hz, 2 H) 2.92 (qq, J = 6.86 Hz, 1 H) 1.46 (q, J = 6.91 Hz, 2 H) 1.21 (d, J = 6.82 Hz, 6 H) 0.60-0.76 (m, 1 H) 0.38-0.46 (m, 2 H) 0.04-0.12 (m, 2 H) | 457.9 |
| | N-({5-{[(2-cyclopropylethyl)amino]carbonyl}- | | |
| | 4,6-dihydroxy-1-[3-(1-methylethyl)phenyl]-2- | | |
| | oxo-1,2-dihydro-3-pyridinyl}carbonyl)glycine | | |
| 219 | 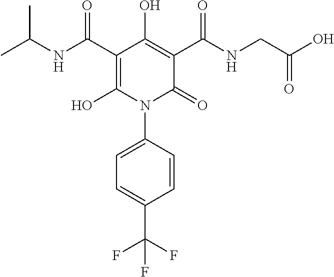 | 13.02 (br. s., 1 H) 9.78 (br. s., 1 H) 9.48 (br. s., 1 H) 7.87 (d, J = 8.59 Hz, 2 H) 7.58 (d, J = 8.34 Hz, 2 H) 4.10 (d, J = 5.31 Hz, 2 H) 4.03-4.15 (m, 1 H) 1.21 (d, J = 6.57 Hz, 6 H) | 457.8 |
| | N-({4,6-dihydroxy-5-{[(1- | | |
| | methylethyl)amino]carbonyl}-2-oxo-1-[4- | | |
| | (trifluoromethyl)phenyl]-1,2-dihydro-3- | | |
| | pyridinyl}carbonyl)glycine | | |
| 220 | 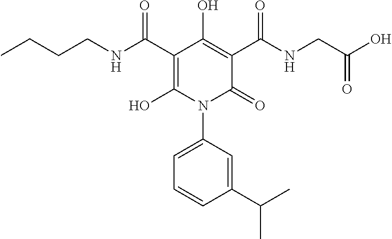 | 12.94 (br. s., 1 H) 9.82 (br. s., 1 H) 9.60 (br. s., 1 H) 7.39 (t, J = 7.71 Hz, 1 H) 7.25-7.34 (m, 1 H) 7.13 (s, 1 H) 7.06 (d, J = 7.58 Hz, 1 H) 4.06 (d, J = 5.31 Hz, 2 H) 3.27-3.40 (m, 2 H) 2.92 (qq, J = 6.85, 6.69 Hz, 1 H) 1.45-1.58 (m, 2 H) 1.26-1.37 (m, 2 H) 1.21 (d, J = 6.82 Hz, 6 H) 0.89 (t, J = 7.33 Hz, 3 H) | 445.9 |
| | N-({5-[(butylamino)carbonyl]-4,6-dihydroxy-1- | | |
| | [3-(1-methylethyl)phenyl]-2-oxo-1,2-dihydro-3- | | |
| | pyridinyl}carbonyl)glycine | | |
| 221 | 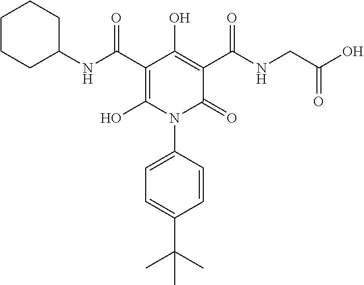 | 13.02 (br. s., 1 H) 9.73 (br. s., 1 H) 9.64 (br. s., 1 H) 7.41- 7.57 (m, 2 H) 7.19 (d, J = 7.33 Hz, 2 H) 4.09 (d, J = 4.80 Hz, 2 H) 3.82 (br. s., 1 H) 1.74- 1.91 (m, 2 H) 1.59-1.72 (m, 2 H) 1.33 (s, 9 H) 1.10-1.59 (m, 6 H) | 486.1 |
| | N-({5-[(cyclohexylamino)carbonyl]-1-[4-(1,1- | | |
| | dimethylethyl)phenyl]-4,6-dihydroxy-2-oxo-1,2- | | |
| | dihydro-3-pyridinyl}carbonyl)glycine | | |
| 222 | 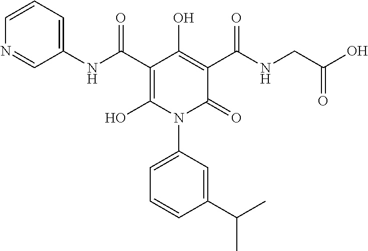 | 12.57 (br. s., 1 H) 10.04 (br. s., 1 H) 9.22 (br. s., 1 H) 8.40- 8.45 (m, 1 H) 8.38 (d, J = 8.08 Hz, 1 H) 7.75 (dd, J = 8.08, 5.56 Hz, 1 H) 7.36 (t, J = 7.71 Hz, 1 H) 7.26 (d, J = 7.83 Hz, 1 H) 7.05 (s, 1 H) 6.99 (d, J = 7.83 Hz, 1 H) 4.00 (d, J = 5.05 Hz, 2 H) 2.92 (spt, J = 6.99 Hz, 1 H) 1.22 (d, J = 6.82 Hz, 6 H) | 467.2 |
| | N-({4,6-dihydroxy-1-[3-(1-methylethyl)phenyl]- | | |
| | 2-oxo-5-[(3-pyridinylamino)carbonyl]-1,2- | | |
| | dihydro-3-pyridinyl}carbonyl)glycine | | |
| 223 | 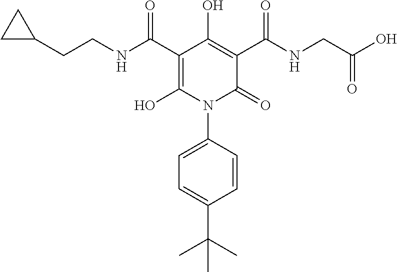 | 12.99 (br. s., 1 H) 9.82 (br. s., 1 H) 9.63 (br. s., 1 H) 7.45- 7.53 (m, 2 H) 7.19 (d, J = 8.59 Hz, 2 H) 4.08 (d, J = 5.31 Hz, 2 H) 3.43 (q, J = 6.57 Hz, 2 H) 1.46 (q, J = 6.82 Hz, 2 H) 1.33 (s, 9 H) 0.60-0.76 (m, 1 H) 0.36-0.46 (m, 2 H) 0.03- 0.10 (m, 2 H) | 472.0 |
| | N-({5-{[(2-cyclopropylethyl)amino]carbonyl}-1- | | |
| | [4-(1,1-dimethylethyl)phenyl]-4,6-dihydroxy-2- | | |
| | oxo-1,2-dihydro-3-pyridinyl}carbonyl)glycine | | |
| 224 | 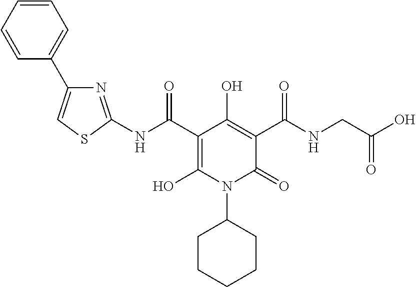 | 13.75 (s, 1 H), 10.02 (s, 1 H), 7.82-8.01 (m, 2 H), 7.63 (s, 1 H), 7.44 (dd, J = 7.58 Hz, 2 H), 7.34 (dd, J = 7.33 Hz, 1 H), 4.72-5.17 (m, 1 H), 4.05 (d, J = 4.04 Hz, 2 H), 2.37-2.48 (m, 2 H), 1.82 (d, J = 12.13 Hz, 2 H), 1.66 (d, J = 12.13 Hz, 1 H), 1.57 (d, J = 11.12 Hz, 2 H), 1.32 (q, J = 12.80 Hz, 2 H), 1.10-1.25 (m, 1 H) | |
| | N-[(1-cyclohexyl-4,6-dihydroxy-2-oxo-5-{[(4- | | |
| | phenyl-1,3-thiazol-2-yl)amino]carbonyl}-1,2- | | |
| | dihydro-3-pyridinyl)carbonyl]glycine | | |
| 225 | 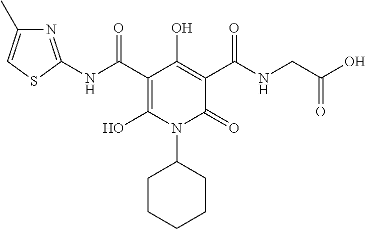 | 14.88 (s, 1 H), 10.20 (s, 1 H), 6.90 (s, 1 H), 4.93 (t, J = 12.00 Hz, 1 H), 4.02 (d, J = 5.31 Hz, 2 H), 2.37-2.49 (m, 2 H), 2.30 (s, 3 H), 1.81 (d, J = 12.38 Hz, 2 H), 1.65 (d, J = 11.87 Hz, 1 H), 1.53 (d, J = 10.11 Hz, 2 H), 1.30 (q, J = 12.72 Hz, 2 H), 1.15 (q, J = 12.21 Hz, 1 H) | 450.7 |
| | N-[(1-cyclohexyl-4,6-dihydroxy-5-{[(4-methyl- | | |
| | 1,3-thiazol-2-yl)amino]carbonyl}-2-oxo-1,2- | | |
| | dihydro-3-pyridinyl)carbonyl]glycine | | |
| 226 | 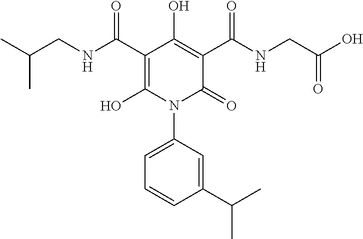 | 12.99 (br. s., 1 H) 9.80 (br. s., 1 H) 9.64 (br. s., 1 H) 7.40 (t, J = 7.71 Hz, 1 H) 7.26-7.35 (m, 1 H) 7.16 (s, 1 H) 7.09 (d, J = 7.83 Hz, 1 H) 4.09 (d, J = 5.56 Hz, 2 H) 3.21 (td, J = 6.13, 2.65 Hz, 2 H) 2.93 (spt, J = 6.86 Hz, 1 H) 1.75- 1.94 (m, 1 H) 1.22 (d, J = 7.07 Hz, 6 H) 0.90 (d, J = 6.57 Hz, 6 H) | 446.0 |
| | N-[(4,6-dihydroxy-1-[3-(1-methylethyl)phenyl]- | | |
| | 5-{[(2-methylpropyl)amino]carbonyl}-2-oxo-1,2- | | |
| | dihydro-3-pyridinyl)carbonyl]glycine | | |
| 227 | 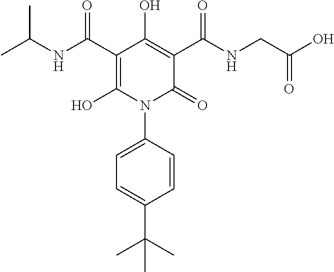 | 13.00 (br. s., 1 H) 9.75 (br. s., 1 H) 9.52 (br. s., 1 H) 7.49 (d, J = 8.59 Hz, 2 H) 7.19 (d, J = 8.59 Hz, 2 H) 4.09 (d, J = 5.31 Hz, 2 H) 4.01-4.16 (m, 1 H) 1.33 (s, 9 H) 1.20 (d, J = 6.57 Hz, 6 H) | 445.9 |
| | N-[(1-[4-(1,1-dimethylethyl)phenyl]-4,6- | | |
| | dihydroxy-5-{[(1-methylethyl)amino]carbonyl}- | | |
| | 2-oxo-1,2-dihydro-3-pyridinyl)carbonyl]glycine | | |
| 228 | 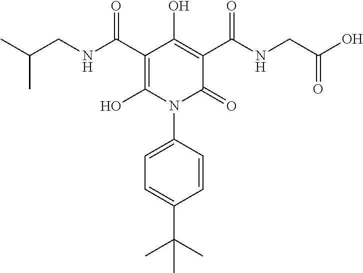 | 13.00 (br. s., 1 H) 9.79 (br. s., 1 H) 9.66 (br. s., 1 H) 7.49 (d, J = 8.34 Hz, 2 H) 7.20 (d, J = 8.59 Hz, 2 H) 4.09 (d, J = 5.31 Hz, 2 H) 3.21 (t, J = 6.19 Hz, 2 H) 1.75-1.91 (m, 1 H) 1.33 (s, 9 H) 0.90 (d, J = 6.82 Hz, 6 H) | 460.1 |
| | N-[(1-[4-(1,1-dimethylethyl)phenyl]-4,6- | | |
| | dihydroxy-5-{[(2-methylpropyl)amino]carbonyl}- | | |
| | 2-oxo-1,2-dihydro-3-pyridinyl)carbonyl]glycine | | |
| 229 | 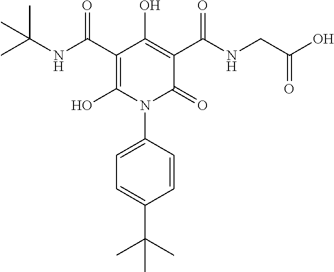 | 13.02 (br. s., 1 H) 9.75 (br. s., 2 H) 7.40-7.54 (m, 2 H) 7.19 (d, J = 8.34 Hz, 2 H) 4.10 (d, J = 5.56 Hz, 2 H) 1.40 (s, 9 H) 1.33 (s, 9 H) | 460.1 |
| | N-({5-{[(1,1-dimethylethyl)amino]carbonyl}-1- | | |
| | [4-(1,1-dimethylethyl)phenyl]-4,6-dihydroxy-2- | | |
| | oxo-1,2-dihydro-3-pyridinyl}carbonyl)glycine | | |
| 230 | 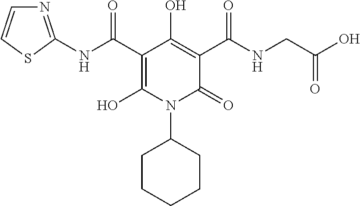 | 14.48 (s, 1 H), 10.12 (s, 1 H), 7.63 (d, J = 4.04 Hz, 1 H), 7.31 (d, J = 3.79 Hz, 1 H), 4.93 (t, J = 12.00 Hz, 1 H), 4.03 (d, J = 4.80 Hz, 2 H), 2.38-2.48 (m, 2 H), 1.81 (d, J = 12.38 Hz, 2 H), 1.65 (d, J = 12.38 Hz, 1 H), 1.54 (d, J = 10.61 Hz, 2 H), 1.30 (q, J = 12.97 Hz, 2 H) 1.16 (q, J = 12.72 Hz, 1 H) | 437.0 |
| | N-({1-cyclohexyl-4,6-dihydroxy-2-oxo-5-[(1,3- | | |
| | thiazol-2-ylamino)carbonyl]-1,2-dihydro-3- | | |
| | pyridinyl}carbonyl)glycine | | |
| 231 | 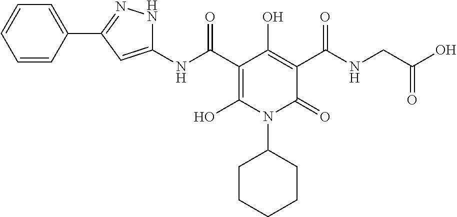 | 9.62 (br. s., 1 H), 7.77 (d, J = 7.07 Hz, 2 H), 7.47 (dd, J = 7.58 Hz, 2 H), 7.37 (dd, J = 7.45 Hz, 1 H), 6.94 (s, 1 H), 4.89 (s, 1 H), 4.12 (d, J = 5.05 Hz, 2 H), 2.19-2.46 (m, 2 H) 1.83 (d, J = 12.38 Hz, 2 H), 1.65 (d, J = 9.09 Hz, 3 H), 1.33 (q, J = 13.05 Hz, 2 H), 1.06-1.26 (m, 1 H) | 496.1 |
| | N-[(1-cyclohexyl-4,6-dihydroxy-2-oxo-5-{[(3- | | |
| | phenyl-1H-pyrazol-5-yl)amino]carbonyl}-1,2- | | |
| | dihydro-3-pyridinyl)carbonyl]glycine | | |
| 232 | 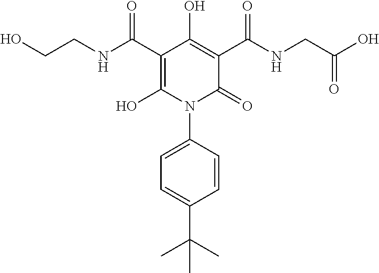 | 13.00 (br. s., 1 H) 9.79 (br. s., 1 H) 9.66 (br. s., 1 H) 7.45- 7.53 (m, 2 H) 7.19 (d, J = 8.34 Hz, 2 H) 4.09 (d, J = 5.31 Hz, 2 H) 3.54 (t, J = 5.56 Hz, 2 H) 3.42 (q, J = 4.88 Hz, 2 H) 1.33 (s, 9 H) | 447.9 |
| | N-[(1-[4-(1,1-dimethylethyl)phenyl]-4,6- | | |
| | dihydroxy-5-{[(2-hydroxyethyl)amino]carbonyl}- | | |
| | 2-oxo-1,2-dihydro-3-pyridinyl)carbonyl]glycine | | |
| 233 | 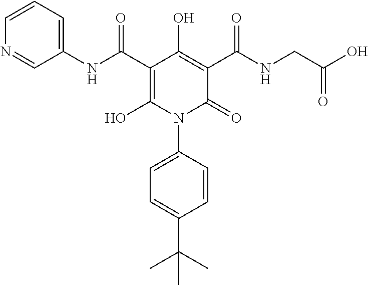 | 12.61 (br. s., 1 H) 10.00 (br. s., 1 H) 9.25 (br. s., 1 H) 8.46 (d, J = 5.31 Hz, 1 H) 8.41 (d, J = 8.59 Hz, 1 H) 7.80 (dd, J = 8.34, 5.31 Hz, 1 H) 7.38- 7.52 (m, 2 H) 7.12 (d, J = 8.34 Hz, 2 H) 4.02 (d, J = 3.79 Hz, 2 H) 1.34 (s, 9 H) | 480.9 |
| | N-({1-[4-(1,1-dimethylethyl)phenyl]-4,6- | | |
| | dihydroxy-2-oxo-5-[(3-pyridinylamino)carbonyl]- | | |
| | 1,2-dihydro-3-pyridinyl}carbonyl)glycine | | |

*Unless otherwise stated.

### Examples 234-257

The title compounds were prepared according to the procedures outlined for the synthesis of compounds 93-139 and by substituting the appropriate reagents when required. All products were subjected to purification and provided satisfactory analytical data.

| Example | | 1H NMR (400 MHz, DMSO- | |
|---|---|---|---|
| # | Name | d6)* δ ppm | [M + H]⁺ |
| 234 | 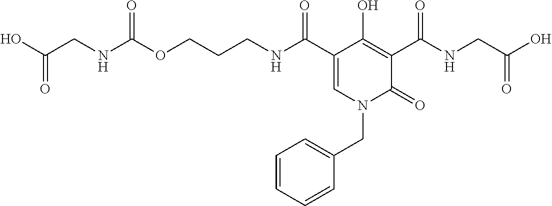 | 1.77 (t, J = 2.53 Hz, 1 H) 1.84 (s, 1 H) 2.11 (dt, J = 4.86, 2.49 Hz, 1 H) 2.51 (s, 1 H) 3.43 (s, 2 H) 3.77 (d, J = 6.06 Hz, 2 H) 4.03-4.12 (m, 2 H) 4.14 (d, J = 5.56 Hz, 2 H) 5.18 (s, 2 H) 7.28-7.39 (m, 5 H) 8.53 (s, 1 H) | 505.1 |
| | [({[3-({[5-{[(carboxymethyl)amino]carbonyl}-4- | | |
| | hydroxy-6-oxo-1-(phenylmethyl)-1,6-dihydro-3- | | |
| | pyridinyl]carbonyl}amino)propyl]oxy}carbonyl) | | |
| | amino]acetic acid | | |
| 235 | 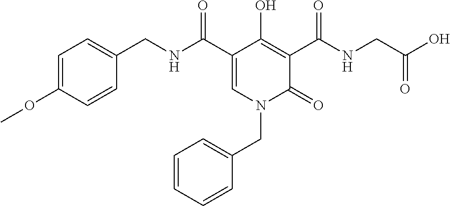 | 3.72 (s, 3 H) 4.09 (d, J = 5.31 Hz, 2 H) 4.42 (d, J = 6.06 Hz, 2 H) 5.26 (s, 2 H) 6.88 (d, J = 8.84 Hz, 1 H) 6.85-6.97 (m, 1 H) 7.23-7.32 (m, 5 H) 7.34-7.38 (m, 2 H) 8.56 (br. s., 1 H) 8.71 (s, 1 H) 10.37 (br. s., 1 H) 12.96 (br. s., 1 H) | 465.8 |
| | N-{[4-hydroxy-5-[({[4- | | |
| | (methyloxy)phenyl]methyl}amino)carbonyl]-2- | | |
| | oxo-1-(phenylmethyl)-1,2-dihydro-3- | | |
| | pyridinyl]carbonyl}glycine | | |
| 236 | 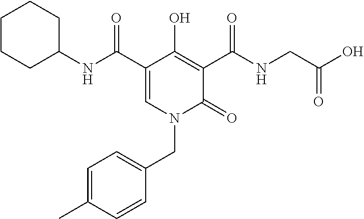 | 1.30 (m, 5 H) 1.55 (m, 1 H) 1.65 (br. s., 2 H) 1.83 (br. s., 2 H) 2.27 (s, 3 H) 3.78 (br. s., 1 H) 4.10 (d, J = 5.81 Hz, 2 H) 5.20 (s, 2 H) 6.55 (s, 1 H) 7.18 (q, J = 8.08 Hz, 4 H) 7.99 (s, 1 H) 8.64 (s, 1 H) 10.39 (s, 1 H) 12.98 (br. s., 1 H) | 442.3 |
| | N-({5-[(cyclohexylamino)carbonyl]-4-hydroxy-1- | | |
| | [(4-methylpheny)methyl]-2-oxo-1,2-dihydro-3- | | |
| | pyridinyl}carbonyl)glycine | | |
| 237 | 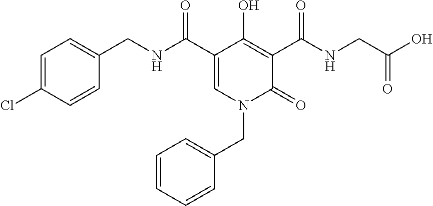 | 4.10 (d, J = 5.56 Hz, 2 H) 4.48 (d, J = 6.06 Hz, 2 H) 5.26 (s, 2 H) 7.27-7.39 (m, 9 H) 8.72 (s, 2 H) 10.36 (br. s., 1 H) 12.97 (br. s., 1 H) | 470.1 |
| | N-{[5-({[(4- | | |
| | chlorophenyl)methyl]amino}carbonyl)-4- | | |
| | hydroxy-2-oxo-1-(phenylmethyl)-1,2-dihydro-3- | | |
| | pyridinyl]carbonyl}glycine | | |
| 238 | 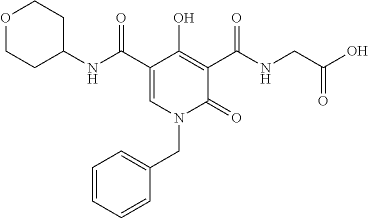 | (DMF) 1.87 (s, 2 H) 1.96 (br. s., 2 H) 2.21 (br. s., 2 H) 3.79- 3.91 (m, 2 H) 4.25 (d, J = 11.12 Hz, 1 H) 4.26 (br. s., 1 H) 4.53 (d, J = 5.56 Hz, 2 H) 5.69 (s, 2 H) 7.70-7.82 (m, 5 H) 8.49 (br. s., 1 H) 9.11 (s, 1 H) 10.79 (br. s., 1 H) 13.42 (br. s., 1 H) | 430.2 |
| | N-({4-hydroxy-2-oxo-1-(phenylmethyl)-5- | | |
| | [(tetrahydro-2H-pyran-4-ylamino)carbonyl]-1,2- | | |
| | dihydro-3-pyridinyl}carbonyl)glycine | | |
| 239 | 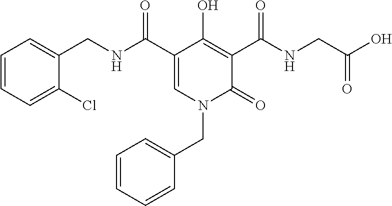 | 3.17 (d, J = 4.29 Hz, 1 H) 4.11 (d, J = 5.56 Hz, 2 H) 4.57 (d, J = 6.06 Hz, 2 H) 5.26 (s, 2 H) 7.27-7.39 (m, 5 H) 7.33 (dq, J = 14.40, 7.07 Hz, 3 H) 7.41- 7.53 (m, 1 H) 8.73 (s, 2 H) 10.36 (s, 1 H) 12.98 (br. s., 1 H) | 470.1 |
| | N-{[5-({[(2- | | |
| | chlorophenyl)methyl]amino}carbonyl)-4- | | |
| | hydroxy-2-oxo-1-(phenylmethyl)-1,2-dihydro-3- | | |
| | pyridinyl]carbonyl}glycine | | |
| 240 | 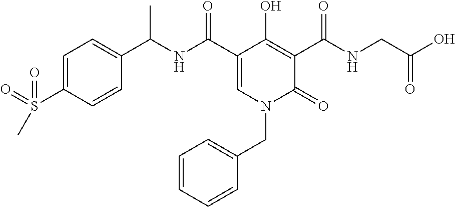 | 1.49 (d, J = 7.07 Hz, 3 H) 3.20 (s, 3 H) 4.11 (d, J = 5.56 Hz, 2 H) 5.19 (d, J = 7.07 Hz, 1 H) 5.16 (s, 1 H) 5.24 (s, 2 H) 7.25-7.38 (m, 5 H) 7.65 (d, J = 8.34 Hz, 2 H) 7.88 (d, J = 8.34 Hz, 1 H) 7.88 (t, J = 4.29 Hz, 1 H) 8.57 (br. s., 1 H) 8.64 (s, 1 H) 10.36 (br. s., 1 H) 12.98 (br. s., 1 H) | 528.2 |
| | N-{[4-hydroxy-5-[({1-[4- | | |
| | (methylsulfonyl)phenyl]ethyl}amino)carbonyl]-2- | | |
| | oxo-1-(phenylmethyl)-1,2-dihydro-3- | | |
| | pyridinyl]carbonyl}glycine | | |
| 241 | 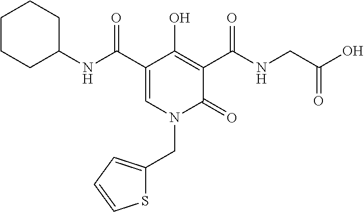 | 1.17-1.39 (m, 6 H), 1.55 (d, J = 10.36 Hz, 1 H), 1.66 (dd, J = 8.84, 4.29 Hz, 2 H), 1.78- 1.85 (m, 2 H), 4.13 (d, J = 5.81 Hz, 2 H), 5.43 (s, 2 H), 7.00 (dd, J = 5.05, 3.28 Hz, 1 H), 7.23 (dd, J = 3.41, 1.14 Hz, 1 H) 7.49 (dd, J = 5.18, 1.14 Hz, 1 H), 7.97 (d, J = 7.83 Hz, 1 H), 8.71 (s, 1 H), 10.39 (t, J = 5.81 Hz, 1 H), 12.97 (br. s., 1 H) | 434.1 |
| | N-{[5-[(cyclohexylamino)carbonyl]-4-hydroxy-2- | | |
| | oxo-1-(2-thienylmethyl)-1,2-dihydro-3- | | |
| | pyridinyl]carbonyl}glycine | | |
| 242 | 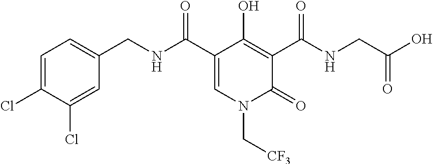 | 4.13 (d, J = 5.56 Hz, 2 H) 4.49 (d, J = 6.06 Hz, 2 H) 5.03 (s, 2 H) 7.32 (dd, J = 8.34, 2.02 Hz, 1 H) 7.55-7.60 (m, 2 H) 8.31 (s, 1 H) 8.65 (s, 1 H) 8.80 (br. s., 1 H) 10.23 (br. s., 1 H) | 496.1 |
| | N-{[5-({[(3,4- | | |
| | dichlorophenyl)methyl]amino}carbonyl)-4- | | |
| | hydroxy-2-oxo-1-(2,2,2-trifluoroethyl)-1,2- | | |
| | dihydro-3-pyridinyl]carbonyl}glycine | | |
| 243 | 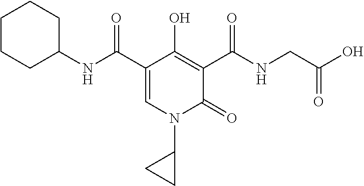 | 0.85-0.98 (m, 2 H) 1.04 (d, J = 5.81 Hz, 3 H) 1.30 (br. s., 5 H) 1.53 (br. s., 1 H) 1.64 (br. s., 2 H) 1.83 (br. s., 2 H) 3.77 (br. s., 1 H) 4.12 (d, J = 5.56 Hz, 2 H) 7.95 (s, 1 H) 8.25 (s, 1 H) 10.47 (s, 1 H) 12.99 (br. s., 1 H) | 378.0 |
| | N-({5-[(cyclohexylamino)carbonyl]-1- | | |
| | cyclopropyl-4-hydroxy-2-oxo-1,2-dihydro-3- | | |
| | pyridinyl}carbonyl)glycine | | |
| 244 | 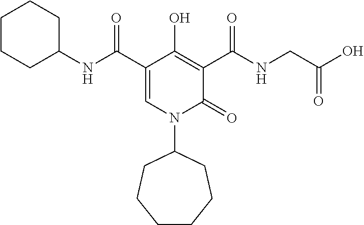 | 1.10-1.40 (br. s., 5 H) 1.42- 1.72 (m, 8 H) 1.72-1.95 (br. s., 7 H) 3.78 (br. s., 1 H) 4.12 (d, J = 5.56 Hz, 2 H) 4.77 (br. s., 1 H) 7.99 (s, 1 H) 8.39 (s, 1 H) 10.52 (s, 1 H) 13.01 (br. s., 1 H) | 434.3 |
| | N-({1-cycloheptyl-5- | | |
| | [(cyclohexylamino)carbonyl]-4-hydroxy-2-oxo- | | |
| | 1,2-dihydro-3-pyridinyl}carbonyl)glycine | | |
| 245 | 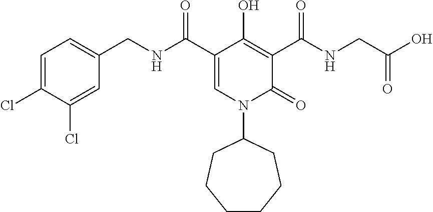 | 1.61 (br. s., 5 H) 1.86 (d, J = 8.34 Hz, 8 H) 4.12 (d, J = 5.56 Hz, 2 H) 4.48 (s, 2 H) 4.78 (br. s., 1 H) 7.32 (dd, J = 8.59, 2.02 Hz, 1 H) 7.59 (d, J = 6.06 Hz, 1 H) 7.58 (s, 1 H) 8.42 (s, 1 H) 8.74 (s, 1 H) 10.50 (s, 1 H) 13.00 (br. s., 1 H) | 510 |
| | N-{[1-cycloheptyl-5-({[(3,4- | | |
| | dichlorophenyl)methyl]amino}carbonyl)-4- | | |
| | hydroxy-2-oxo-1,2-dihydro-3- | | |
| | pyridinyl]carbonyl}glycine | | |
| 246 | 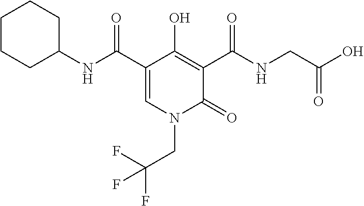 | 1.32 (d, J = 8.59 Hz, 5 H) 1.54 (br. s., 1 H) 1.66 (br. s., 2 H) 1.83 (br. s., 2 H) 3.80 (br. s., 1 H) 4.13 (d, J5.56 Hz, 2 H) 5.05 (d, J = 9.35 Hz, 2 H) 8.04 (br. s., 1 H) 8.60 (s, 1 H) 10.26 (br. s., 1 H) 13.03 (br. s., 1 H) | 420.1 |
| | N-{[5-[(cyclohexylamino)carbonyl]-4-hydroxy-2- | | |
| | oxo-1-(2,2,2-trifluoroethyl)-1,2-dihydro-3- | | |
| | pyridinyl]carbonyl}glycine | | |
| 247 | 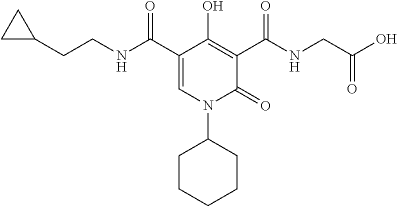 | 0.06 (d, J = 14.40 Hz, 1 H) 0.06 (dd, J = 4.93, 1.39 Hz, 1 H) 0.41 (dt, J = 9.79, 3.95 Hz, 1 H) 0.41 (d, J = 6.32 Hz, 1 H) 0.64-0.75 (m, 1 H) 1.25 (d, J = 13.14 Hz, 1 H) 1.41 (q, J = 7.24 Hz, 4 H) 1.64 (br. s., 3 H) 1.83 (br. s., 4 H) 4.12 (d, J = 5.56 Hz, 2 H) 4.66 (br. s., 1 H) 8.18 (s, 1 H) 8.41 (s, 1 H) 10.49-10.60 (m, 1 H) 13.00 (br. s., 1 H) | 406.2 |
| | N-[(1-cyclohexyl-5-{[(2- | | |
| | cyclopropylethyl)amino]carbonyl}-4-hydroxy-2- | | |
| | oxo-1,2-dihydro-3-pyridinyl)carbonyl]glycine | | |
| 248 | 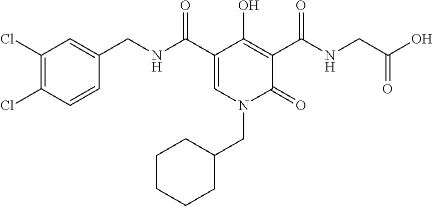 | 0.98 (t, J = 11.49 Hz, 2 H), 1.06-1.24 (m, 3 H), 1.47- 1.79 (m, 6 H), 3.88 (d, J = 7.07 Hz, 2 H), 4.12 (d, J = 5.56 Hz, 2 H), 4.48 (d, J = 6.06 Hz, 2 H), 7.33 (dd, J = 8.21, 2.15 Hz, 1 H), 7.54-7.62 (m, 2 H), 8.48 (s, 1 H), 8.73 (t, J = 6.06 Hz, 1 H), 10.44 (t, J = 5.43 Hz, 1 H), 12.97 (br. s., 1 H) | 510.1 |
| | N-{[1-(cyclohexylmethyl)-5-({[(3,4- | | |
| | dichlorophenyl)methyl]amino}carbonyl)-4- | | |
| | hydroxy-2-oxo-1,2-dihydro-3- | | |
| | pyridinyl]carbonyl}glycine | | |
| 249 | 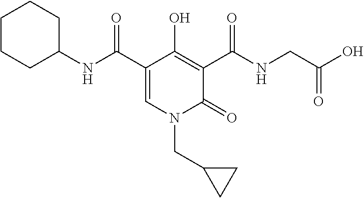 | 0.39 (q, J = 4.88 Hz, 2 H), 0.46- 0.54 (m, 2 H), 1.16-1.43 (m, 6 H), 1.55 (d, J = 12.13 Hz, 1 H), 1.67 (dd, J = 8.97, 3.66 Hz, 2 H), 1.80-1.85 (m, 2 H), 3.80 (dd, J = 7.83, 3.54 Hz, 1 H), 3.90 (d, J = 7.07 Hz, 2 H), 4.12 (d, J = 5.56 Hz, 2 H), 7.98 (d, J = 7.83 Hz, 1 H), 8.58 (s, 1 H), 10.46 (t, J = 5.31 Hz, 1 H), 12.98 (br. s., 1 H) | 392.2 |
| | N-{[5-[(cyclohexylamino)carbonyl]-1- | | |
| | (cyclopropylmethyl)-4-hydroxy-2-oxo-1,2- | | |
| | dihydro-3-pyridinyl]carbonyl}glycine | | |
| 250 | 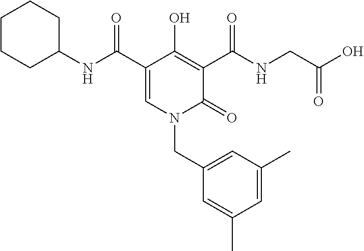 | 1.15-1.43 (m, 6 H), 1.55 (d, J = 11.62 Hz, 1 H), 1.62-1.72 (m, 2 H), 1.77-1.86 (m, 2 H), 2.24 (s, 6 H), 4.11 (d, J = 5.56 Hz, 2 H), 5.16 (s, 2 H), 6.89 (s, 2 H), 6.93 (s, 1 H), 8.00 (d, J = 7.83 Hz, 1 H), 8.62 (s, 1 H), 10.38 (t, J = 4.93 Hz, 1 H), 12.98 (br. s., 1 H) | 456.1 |
| | N-({5-[(cyclohexylamino)carbonyl]-1-[(3,5- | | |
| | dimethylphenyl)methyl]-4-hydroxy-2-oxo-1,2- | | |
| | dihydro-3-pyridinyl}carbonyl)glycine | | |
| 251 | 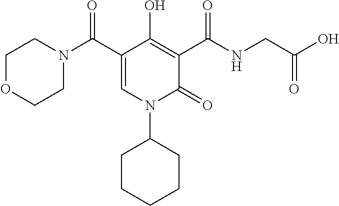 | 1.22 (br. s., 1 H) 1.38 (d, J = 12.13 Hz, 2 H) 1.65 (br. s., 1 H) 1.66-1.78 (m, 4 H) 1.84 (m, 2H) 3.29 (br. s., 2 H) 3.59 (m, 6 H) 4.08 (d, J = 5.56 Hz, 2 H) 4.69 (d, J = 3.79 Hz, 1 H) 8.07 (s, 1 H) 10.46 (t, J = 5.43 Hz, 1 H) 12.94 (br. s., 1 H) | 408.1 |
| | N-{[1-cyclohexyl-4-hydroxy-5-(4- | | |
| | morpholinylcarbonyl)-2-oxo-1,2-dihydro-3- | | |
| | pyridinyl]carbonyl}glycine | | |
| 252 | 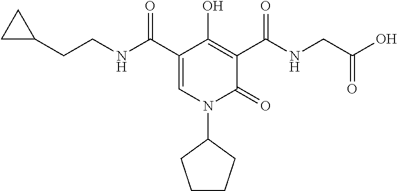 | 0.06 (dd, J = 4.93, 1.39 Hz, 2 H) 0.41 (ddd, J = 9.73, 4.04, 3.92 Hz, 2 H) 0.69 (dd, J = 12.63, 2.78 Hz, 1 H) 1.41 (q, J = 6.91 Hz, 2 H) 1.65- 1.81 (m, 8 H) 2.01-2.13 (m, 2 H) 4.12 (d, J = 5.56 Hz, 2 H) 5.05 (t, J = 7.20 Hz, 1 H) 8.13- 8.24 (m, 1 H) 8.40 (s, 1 H) 10.41-10.52 (m, 1 H) 12.98 (br. s., 1 H) | 392.2 |
| | N-[(1-cyclopentyl-5-{[(2- | | |
| | cyclopropylethyl)amino]carbonyl}-4-hydroxy-2- | | |
| | oxo-1,2-dihydro-3-pyridinyl)carbonyl]glycine | | |
| 253 | 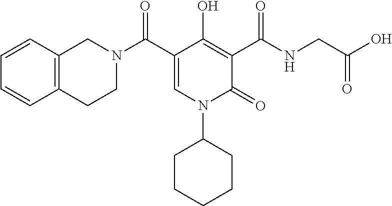 | 1.20 (br. s., 1 H) 1.36 (br. s., 2 H) 1.74 (br. s., 6 H) 2.83 (br. s., 2 H) 3.54 (s, 1 H) 4.10 (d, J = 5.56 Hz, 2 H) 4.52 (s, 1 H) 4.75 (br. s., 2 H) 7.18 (br. s., 1 H) 7.17 (d, J = 2.27 Hz, 2 H) 7.22 (d, J = 12.63 Hz, 1 H) 8.07 (s, 1 H) 10.49 (s, 1 H) 12.94 (br. s., 1 H) 16.36 (s, 1 H) | 453.9 |
| | N-{[1-cyclohexyl-5-(3,4-dihydro-2(1H)- | | |
| | isoquinolinylcarbonyl)-4-hydroxy-2-oxo-1,2- | | |
| | dihydro-3-pyridinyl]carbonyl}glycine | | |
| 254 | 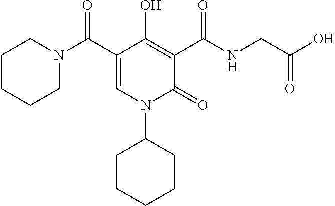 | 1.22 (br. s., 1 H) 1.30-1.58 m., 6 H) 1.60-1.67 (m, 4 H) 1.70-1.90 (m, 5 H) 3.22 (br. s., 2 H) 3.55 (br. s., 2 H) 4.08 (d, J = 5.56 Hz, 2 H) 4.69 (d, J = 4.55 Hz, 1 H) 8.04 (s, 1 H) 10.48 (t, J = 5.43 Hz, 1 H) 12.93 (br. s., 1 H) 16.27 (s, 1 H) | 406.5 |
| | N-{[1-cyclohexyl-4-hydroxy-2-oxo-5-(1- | | |
| | piperidinylcarbonyl)-1,2-dihydro-3- | | |
| | pyridinyl]carbonyl}glycine | | |
| 255 | 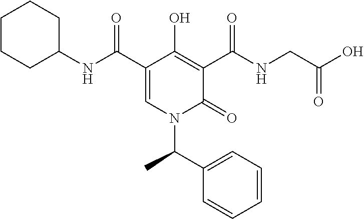 | 1.01-1.38 (m, 6 H), 1.70 (d, J = 7.33 Hz, 8 H), 3.63-3.81 (m, 1 H), 3.88 (d, J = 5.05 Hz, 2 H), 6.17 (q, J = 6.82 Hz, 1 H), 7.27-7.43 (m, 5 H), 8.17 (s, 1 H), 8.63 (br. s., 1 H), 10.45 (br. s., 1 H) | 442.2 |
| | N-({5-[(cyclohexylamino)carbonyl]-4-hydroxy-2- | | |
| | oxo-1-[(1R)-1-phenylethyl]-1,2-dihydro-3- | | |
| | pyridinyl}carbonyl)glycine | | |
| 256 | 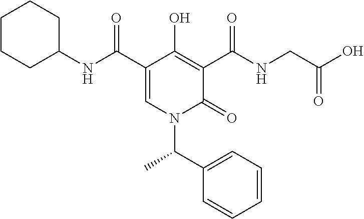 | 0.76-1.43 (m, 6 H), 1.43- 1.86 (m, 8 H), 3.72 (br. s., 1 H), 4.00 (d, J = 5.31 Hz, 2 H), 6.10-6.19 (m, 1 H), 7.27- 7.41 (m, 5 H), 8.21 (s, 1 H), 8.32 (br. s., 1 H), 10.45 (br. s., 1 H) | 442.2 |
| | N-({5-[(cyclohexylamino)carbonyl]-4-hydroxy-2- | | |
| | oxo-1-[(1S)-1-phenylethyl]-1,2-dihydro-3- | | |
| | pyridinyl}carbonyl)glycine | | |
| 257 | 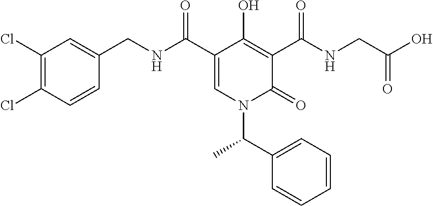 | 1.66 (d, J = 7.33 Hz, 3 H), 3.77 (d, J = 4.80 Hz, 2 H), 4.41 (d, J = 6.57 Hz, 2 H), 6.16 (q, J = 6.99 Hz, 1 H), 7.24-7.32 (m, 4 H), 7.33-7.40 (m, 2 H), 7.52 (d, J = 2.02 Hz, 1 H), 7.55 (d, J = 8.34 Hz, 1 H), 8.13 (s, 1 H), 9.58 (br. s., 1 H), 10.36 (br. s., 1 H) | 518.2 |
| | N-({5-({[(3,4- | | |
| | dichlorophenyl)methyl]amino}carbonyl)-4- | | |
| | hydroxy-2-oxo-1-[(1S)-1-phenylethyl]-1,2- | | |
| | dihydro-3-pyridinyl}carbonyl)glycine | | |

*Unless otherwise stated.

### Biological Background:

The following references set out information about the target enzymes, HIF prolyl hydroxylases, and methods and materials for measuring inhibition of same by small molecules.
M. Hirsilä, P. Koivunen, V. Günzler, K. I. Kivirikko, and J. Myllyharju “Characterization of the Human Prolyl 4-Hydroxylases That Modify the Hypoxia-inducible Factor” J. Biol. Chem., 2003, 278, 30772-30780.
C. Willam, L. G. Nicholls, P. J. Ratcliffe, C. W. Pugh, P. H. Maxwell “The prolyl hydroxylase enzymes that act as oxygen sensors regulating destruction of hypoxia-inducible factor α” Advan. Enzyme Regul., 2004, 44, 75-92
M. S. Wiesener, J. S. Jürgensen, C. Rosenberger, C. K. Scholze, J. H. Hörstrup, C. Warnecke, S. Mandriota, I. Bechmann, U. A. Frei, C. W. Pugh, P. J. Ratcliffe, S. Bachmann, P. H. Maxwell, and K.-U. Eckardt “Widespread hypoxia-inducible expression of HIF-2-A in distinct cell populations of different organs” FASEB J., 2003, 17, 271-273.
S. J. Klaus, C. J. Molineaux, T. B. Neff, V. Guenzler-Pukall, I. Lansetmo Parobok, T. W. Seeley, R. C. Stephenson “Use of hypoxia-inducible factor α (HIFα) stabilizers for enhancing erythropoiesis” PCT Int. Appl. (2004), WO 2004108121 A1
C. Warnecke, Z. Zaborowska, J. Kurreck, V. A. Erdmann, U. Frei, M. Wiesener, and K.-U. Eckardt “Differentiating the functional role of hypoxia-inducible factor (HIF)-1α and HIF-2α (EPAS-1) by the use of RNA interference: erythropoietin is a HIF-2α target gene in Hep3B and Kelly cells” FASEB J., 2004, 18, 1462-1464.
   For the expression of EGLN3 see:
R. K. Bruick and S. L. McKnight “A Conserved Family of Prolyl-4-Hydroxylases That Modify HIF” Science, 2001, 294, 1337-1340.
   For the expression of HIF2α-CODD see:
a) P. Jaakkola, D. R. Mole, Y.-M. Tian, M. I. Wilson, J. Gielbert, S. J. Gaskell, A. von Kriegsheim, H. F. Hebestreit, M. Mukherji, C. J. Schofield, P. H. Maxwell, C. W. Pugh, P, J. Ratcliffe “Targeting of HIF-α to the von Hippel-Lindau Ubiquitylation Complex by O₂-Regulated Prolyl Hydroxylation” Science, 2001, 292, 468-472.
b) M. Ivan, K. Kondo, H. Yang, W. Kim, J. Valiando, M. Ohh, A. Salic, J. M. Asara, W. S. Lane, W. G. Kaelin Jr. “HIFα Targeted for VHL-Mediated Destruction by Proline Hydroxylation: Implications for O₂Sensing” Science, 2001, 292, 464-468.
   For the expression of VHL, elongin b and elongin c see:
A. Pause, S. Lee, R. A. Worrell, D. Y. T. Chen, W. H. Burgess, W. M. Linehan, R. D. Klausner “The von Hippel-Lindau tumor-suppressor gene product forms a stable complex with human CUL-2, a member of the Cdc53 family of proteins” Proc. Natl. Acad. Sci. USA, 1997, 94, 2156-2161.

### Biological Assay(s)

### EGLN3 Assay

### Materials:

His-MBP-EGLN3 (6H isMBPAttB1EGLN3(1-239)) was expressed in E. Coli and purified from an amylase affinity column. Biotin-VBC [6HisSumoCysVHL(2-213), 6H is SumoElonginB(1-118), and 6HisSumoElonginC(1-112)] and His-GB1-HIF2α-CODD (6H isGB1tevHIF2A(467-572)) were expressed from E. Coli.

### Method:

Cy5-labelled HIF2α CODD, and a biotin-labeled VBC complex were used to determine EGLN3 inhibition. EGLN3 hydroxylation of the Cy5CODD substrate results in its recognition by the biotin-VBC. Addition of a Europium/streptavidin (Eu/SA) chelate results in proximity of Eu to Cy5 in the product, allowing for detection by energy transfer. A ratio of Cy5 to Eu emission (LANCE Ratio) is the ultimate readout, as this normalized parameter has significantly less variance than the Cy5 emission alone.

Then 50 nL of inhibitors in DMSO (or DMSO controls) were stamped into a 384-well low volume Corning NBS plate, followed by addition of 2.5 μL of enzyme [50 mL buffer (50 mM HEPES/50 mM KCl)+1 mL of a 10 mg/mL BSA in buffer+6.25 μL of a 10 mg/mL FeCl₂ solution in water+100 μL of a 200 mM solution of ascorbic acid in water+15.63 μL EGLN3] or control [50 mL buffer+1 mL of a 10 mg/mL BSA in buffer+6.25 μL of a 10 mg/mL FeCl₂ solution in water+100 μL of a 200 mM solution of ascorbic acid in water]. Following a 3 minutes incubation, 2.5 μL of substrate [50 mL Buffer+68.6 μL biotin-VBC+70.4 μL Eu (at 710 μg/mL stock)+91.6 μL Cy5CODD+50 μL of a 20 mM solution of 2-oxoglutaric acid in water+0.3 mM CHAPS] was added and incubated for 30 minutes. The plate was loaded into a PerkinElmer Viewlux for imaging. For dose response experiments, normalized data were fit by ABASE/XC50 using the equation y=a+(b−a)/(1+(10̂x/10̂c)̂d), where a is the minimum % activity, b is the maximum % activity, c is the pIC₅₀, and d is the Hill slope.

The IC₅₀ for exemplified compounds in the EGLN3 assay ranged from approximately 1-100 nanomolar. This range represents the data accumulated as of the time of the filing of this initial application. Later testing may show variations in IC₅₀ data due to variations in reagents, conditions and variations in the method(s) used from those given herein above. So this range is to be viewed as illustrative, and not a absolute set of numbers.

### Measure Epo Protein Produced by Hep3B Cell Line Using ELISA Method.

Hep3B cells obtained from the American Type Culture Collection (ATCC) are seeded at 2×10̂4 cells/well in Dulbecco's Modified Eagle Medium (DMEM)+10% FBS in 96-well plates. Cells are incubated at 37degC/5% CO2/90% humidity (standard cell culture incubation conditions). After overnight adherence, medium is removed and replaced with DMEM without serum containing test compound or DMSO negative control. Following 48 hours incubation, cell culture medium is collected and assayed by ELISA to quantitate Epo protein.

The EC₅₀ for exemplar compounds in the Hep3B ELISA assay ranged from approximately 1-20 micromolar using the reagents and under the conditions outlined herein above. This range represents the data accumulated as of the time of the filing of this initial application. Later testing may show variations in EC₅₀ data due to variations in reagents, conditions and variations in the method(s) used from those given herein above. So this range is to be viewed as illustrative, and not a absolute set of numbers.

These compound are believed to be useful in therapy as defined above and to not have unacceptable or untoward effects when used in compliance with a permited therapeutic regime.

The foregoing examples and assay have been set forth to illustrate the invention, not limit it. What is reserved to the inventors is to be determined by reference to the claims.

1. A compound of formula (I): 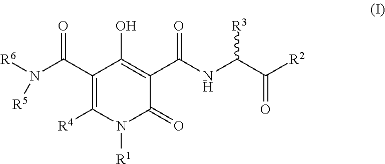
wherein:
R¹ is H, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈cycloalkenyl, C₃-C₈ heterocycloalkyl, C₁₋C₁₀alkyl C₃-C₈ heterocycloalkyl, aryl, C₁₋C₁₀alkyl-aryl, heteroaryl or C₁₋C₁₀alkyl-heteroaryl;
R² is —NR⁷R⁸ or —OR⁹;
R³ is H or C₁₋C₄alkyl;
R⁴ is hydrogen or OH;
R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl; or R⁵ and R⁶ taken together with the nitrogen to which they are attached form a 5- or 6- or 7-membered saturated ring optionally containing one other heteroatom selected from oxygen, nitrogen and sulphur;
R² and R⁸ are each independently selected from the group consisting of hydrogen, C₁₋C₁₀ alkyl, C₂₋C₁₀ alkenyl, C₂₋C₁₀ alkynyl, C₃-C₈ cycloalkyl, C₃-C₈ heterocycloalkyl, aryl and heteroaryl;
R⁹ is H or a cation, or C₁₋C₁₀allyl which is unsubstituted or substituted with one or more substituents independently selected from the group consisting of C₃-C₆ cycloalkyl, heterocycloalkyl, aryl, and heteroaryl;
where any carbon or heteroatom of R¹, R³, R⁵, R⁶, R², R⁸, R⁹ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —NR⁷R⁸, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁷, and R⁸ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt or solvate thereof.

2. A compound according to claim 1 wherein:
R¹ is selected from the group consisting of hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —OR⁹;
R³ is H or C₁₋C₄alkyl;
R⁴ is H or OH;
R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl;
R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R⁵, R⁶ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —CONR⁷R⁸, —N(R²)C(O)R¹⁰, —N(R²)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁷, and R⁸ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₈ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt or solvate thereof.

3. A compound according to claim 2 wherein:
R¹ and is selected from the group consisting of hydrogen, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —OR⁹;
R³ is H;
R⁴ is hydrogen or OH;
R⁵ and R⁶ are each independently selected from the group consisting of hydrogen, —C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R⁹ is H or a cation;
where any carbon or heteroatom of R¹, R⁵, R⁶ is unsubstituted or, where possible, is substituted with one or more substituents independently selected from C₁-C₆ alkyl, aryl, heteroaryl, halogen, —OR¹⁰, —NR⁷R⁸, cyano, nitro, —C(O)R¹⁰, —C(O)OR¹⁰, —SR¹⁰, —S(O)R¹⁰, —S(O)₂R¹⁰, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁷, and R⁸ are the same as defined above and R¹⁰ is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt or solvate thereof.

4. A compound according to claim 1 which is:
N-[(1-cyclohexyl-5-{[(2-cyclopropylethyl)amino]carbonyl}-4,6-dihydroxy-2-oxo-1,2-dihydro-3-pyridinyl)carbonyl]glycine;
N-({1-cyclohexyl-5-[(cyclohexylamino)carbonyl]-4,6-dihydroxy-2-oxo-1,2-dihydro-3-pyridinyl}carbonyl)glycine;
N-[(1-cyclohexyl-4,6-dihydroxy-5-{[(2-methylpropyl)amino]carbonyl}-2-oxo-1,2-dihydro-3-pyridinyl)carbonyl]glycine;
N-({1-[(2-chlorophenyl)methyl]-5-[(cyclohexylamino)carbonyl]-4,6-dihydroxy-2-oxo-1,2-dihydro-3-pyridinyl}carbonyl)glycine;
N-{[1-cyclopentyl-5-({[(3,4-dichlorophenyl)methyl]amino}carbonyl)-4-hydroxy-2-oxo-1,2-dihydro-3-pyridinyl]carbonyl}glycine;
N-{[1-[(2-chlorophenyl)methyl]-5-({[1-(4-chlorophenyl)-1-methylethyl]amino}carbonyl)-4-hydroxy-2-oxo-1,2-dihydro-3-pyridinyl]carbonyl}glycine;
N-[(1-[(2-chlorophenyl)methyl]-4-hydroxy-5-{[(1-methylethyl)amino]carbonyl}-2-oxo-1,2-dihydro-3-pyridinyl)carbonyl]glycine
or a pharmaceutically acceptable salt or solvate thereof.

5. A method for treating anemia in a mammal, which method comprises administering an effective amount of a compound of formula (I) or a salt or solvate thereof according to claim 1 to a mammalian suffering from anemia which can be treated by inhibiting HIF prolyl hydroxylases.

6. A pharmaceutical composition comprising a compound of formula (I) or a salt, solvate, according to claim 1 and one or more of pharmaceutically acceptable carriers, diluents and excipients.

7. A process for preparing a compound of formula (I) 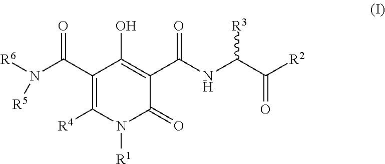
wherein:
R¹ is H, C₁₋C₁₀alkyl, C₂₋C₁₀alkenyl, C₂₋C₁₀alkynyl, C₃-C₈cycloalkyl, C₁₋C₁₀alkyl-C₃-C₈cycloalkyl, C₅-C₈cycloalkenyl, C₁₋C₁₀alkyl-C₅-C₈ cycloalkenyl, C₃-C₈ heterocycloalkyl, C₁₋C₁₀alkyl-C₃-C₈ heterocycloalkyl, aryl, C₁₋C₁₀alkyl-aryl, heteroaryl or C₁₋C₁₀alkyl-heteroaryl;
R² is —NR⁷R⁸ or —OR⁹;
R³ is H or C₁₋C₄alkyl;
R⁴ is hydrogen or OH;
R⁵ and R⁶ are each independently selected from the group consisting of hydrogen, C₁-C₁₀ alkyl, C₃-C₈cycloalkyl, C₁-C₁₀ alkyl-C₃-C₈cycloalkyl, C₃-C₈heterocycloalkyl, C₁-C₁₀ alkyl-C₃-C₈heterocycloalkyl, aryl, C₁₋C₁₀alkyl-aryl, heteroaryl, C₁₋C₁₀alkyl-heteroaryl; or R⁵ and R⁶ taken together with the nitrogen to which they are attached form a 5- or 6- or 7-membered saturated ring optionally containing one other heteroatom selected from oxygen, nitrogen and sulphur;
R⁷ and R⁸ are each independently selected from the group consisting of hydrogen, C₁₋C₁₀ alkyl, C₂₋C₁₀ alkenyl, C₂₋C₁₀ alkynyl, C₃-C₈ cycloalkyl, C₃-C₈ heterocycloalkyl, aryl and heteroaryl;
R⁹ is H or a cation, or C₁₋C₁₀alkyl which is unsubstituted or substituted with one or more substituents independently selected from the group consisting of C₃-C₆ cycloalkyl, heterocycloalkyl, aryl, and heteroaryl;
where any carbon or heteroatom of R¹, R³, R⁵, R⁶, R⁷, R⁸, R⁹ is unsubstituted or, where possible, is substituted with one or more substituents independently selected from C₁-C₆ alkyl, aryl, heteroaryl, halogen, —OR¹⁶, —NR⁷R⁸, cyano, nitro, —C(O)R¹⁰, —C(O)OR¹⁰, —S(O)R¹⁰, —S(O)₂R¹⁰, —NR⁷R⁸, —CONR⁷R⁸, —N(R⁷)C(O)R¹⁰, —N(R⁷)C(O)OR¹⁰, —OC(O)NR⁷R⁸, —N(R⁷)C(O)NR⁷R⁸, —SO₂NR⁷R⁸, —N(R⁷)SO₂R¹⁰, C₁-C₁₀ alkenyl, C₁-C₁₀ alkynyl, C₃-C₆ cycloalkyl, C₃-C₆ heterocycloalkyl, aryl or heteroaryl group, wherein R⁶, and R⁷ are the same as defined above and R¹² is hydrogen, C₁₋C₁₀alkyl, C₂₋C₁₀alkenyl, C₂₋C₁₀alkynyl, —CO(C₁-C₄ alkyl), —CO(aryl), —CO(heteroaryl), —CO(C₃-C₆ cycloalkyl), —CO(C₃-C₆ heterocycloalkyl), —SO₂(C₁-C₄ alkyl), C₃-C₈ cycloalkyl, C₃-C₈heterocycloalkyl, C₆-C₁₄ aryl, C₁₋C₁₀alkyl-aryl, heteroaryl, and C₁₋C₁₀alkyl-heteroaryl;
or a pharmaceutically acceptable salt or solvate thereof;
comprising treating a compound of formula A: 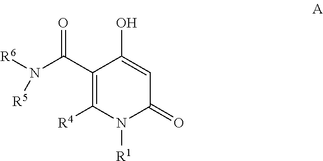
wherein R¹, R⁴, R⁵ and R⁶ are the same as for those groups in formula (I) with an ethyl 2-isocyanatocarboxylate and an appropriate base, such as di-isopropylethylamine, in an appropriate solvent, such as dichloromethane, under either conventional thermal conditions or by microwave irradiation, to form a compound of formula (B) wherein R¹, R³, R⁴, R⁵ and R⁶ are the same as for those groups in formula (I); 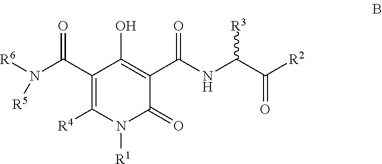
and treating the compound of formula (B) with an alkali such as sodium hydroxide, in an appropriate solvent, such as aqueous ethanol, at a suitable temperature such as room temperature, to form a compound of formula (I) where R² is —OH.
